(19) United States
(12) Patent Application Publication
(10) Pub. No.: US 2020/0087629 A1
(43) Pub. Date: Mar. 19, 2020

(54) COMPOSITIONS AND METHODS FOR REPROGRAMMING CELLS AND FOR SOMATIC CELL NUCLEAR TRANSFER USING DUXC EXPRESSION

(71) Applicants: FRED HUTCHINSON CANCER RESEARCH CENTER, Seattle, WA (US); UNIVERSITY OF UTAH RESEARCH FOUNDATION, Salt Lake City, UT (US)

(72) Inventors: Stephen J TAPSCOTT, Seattle, WA (US); Jennifer L WHIDDON, Seattle, WA (US); Peter G HENDRICKSON, Salt Lake City, UT (US); Edward J GROW, Salt Lake City, UT (US); Bradley R CAIRNS, Salt Lake City, UT (US)

(21) Appl. No.: 16/342,856

(22) PCT Filed: Oct. 19, 2017
(86) PCT No.: PCT/IB2017/056514

§ 371 Date: Apr. 17, 2019

Related U.S. Application Data
(60) Provisional application No. 62/410,078, filed on Oct. 19, 2016.

(51) Int. Cl. C12N 5/074; A61K 35/28; C12N 15/90; C12N 5/0735; C12N 5/073

(52) U.S. Cl. CPC C12N 5/0696; A61K 35/28; C12N 15/90; C12N 5/0606; C12N 5/0605; C12N 2506/45; C12N 2501/998; C12N 2501/603; C12N 2501/602; C12N 2501/604; C12N 2501/606; C12N 5/0604

(57) ABSTRACT

It was found that DUXC family proteins were efficient activators of EGA and that DUXC proteins could be used in methods in the reprogramming of cells to a totipotent state and to increase the efficiency of somatic cell nuclear transfer (SCNT). Accordingly, aspects of the disclosure relate to a method for reprogramming a cell into a totipotent state, the method comprising expressing a DUXC family protein in the cell. Further aspects of the disclosure relate to a method for making a host cell nuclear transfer (SCNT) embryo comprising expressing a DUXC protein in a somatic cell and transferring the nucleus of the somatic cell to an enucleated oocyte, thereby making a SCNT embryo.

This application is a national phase application under 35 U.S.C. § 371 of International Application No. PCT/IB2017/056514, filed Oct. 19, 2017 which claims the benefit of priority to U.S. Provisional Patent Application Ser. No. 62/410,078, filed Oct. 19, 2016, each of which is hereby incorporated by reference in its entirety.

This invention was made with government support under AR045203 awarded by the National Institutes of Health. The government has certain rights in the invention.

### 1. Field of the Invention

This invention relates to the field of molecular biology and medicine.

### 2. Description of Related Art

During the first several days of life, mammalian embryos survive by using components deposited in the egg, but soon must accomplish a profound shift from maternal to a zygotic control of development. Embryonic genome activation (EGA) is the process by which the preimplantation embryo initiates zygotic transcription. Mature sperm and oocytes are transcriptionally quiescent, and EGA allows for the production of gene products not present in the egg. As such, EGA is a naturally occurring reprogramming event that initiates an embryonic developmental program after the fusion of terminally differentiated gametes.

EGA gene products help a totipotent embryo develop into a morula, and this transient state exists before the onset of pluripotency several cell divisions later in the blastocyst. Notably, EGA in mammals occurs in the absence of pluripotency transcription factors (TFs) such as Oct4, Sox2, and Nanog, which are not significantly maternally deposited. Blocking transcription arrests embryos at the EGA stage—which in humans and cows is the 4- to 8-cell stage and in mouse at the 2-cell stage—highlighting the importance of EGA for developmental competence.

Despite it's critical role in development, little is understood mechanistically about the process of EGA in mammals. In particular, both the DNA sequence-specific TFs and the regulatory regions—such as enhancers and promoters—that control EGA are not identified. EGA initiates a precise gene-expression program, which indicates that TFs must be controlling RNA polymerase specificity. Because of the technical limitation of small cell numbers necessitated by early embryo stages, it has been challenging to identify TF-bound EGA regulatory regions in vivo.

Therefore, there is a need in the art for more information about the EGA process and mechanisms to activate this process to increase the efficiency of reprogramming and cloning for the purposes of human therapy and animal breeding and reproduction.

### SUMMARY

It was found that DUXC family proteins were efficient activators of EGA and that DUXC proteins could be used in methods in the reprogramming of cells to a totipotent state and to increase the efficiency of somatic cell nuclear transfer (SCNT). Accordingly, aspects of the disclosure relate to a method for reprogramming a cell into a totipotent state, the method comprising expressing a DUXC family protein in the cell.

In some embodiments, the cell is a differentiated cell. In some embodiments, the cell is a somatic cell. In some embodiments, the cell is a cell type described herein. In some embodiments, the cell is an iPSC cell.

In some aspects, the disclosure relates to activating an EGA state in a cell, the method comprising expressing a DUXC family protein in the cell.

The totipotent state may comprise a state in which the cell is capable of differentiating into both embryonic and extraembryonic tissue (eg. inner cell mass and trophectoderm, respectively). In some embodiments, the totipotent state is further defined as an early cleavage-like state. In some embodiments, the early cleavage like state comprises a cell having a two-cell or four-cell phenotype. In some embodiments, the early cleavage like state comprises activation of 3 or more cleavage-stage genes and/or gene families. In some embodiments, the early cleavage like state comprises activation of at least or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, or 70 (or any derivable range therein) cleavage-stage genes. In some embodiments, the early cleavage like state comprises an increased expression of a ZSCAN gene, such as ZSCAN4 and ZSCAN5. In some embodiments, the early cleavage-like state comprises downregulation of one or more pluripotent factors. In some embodiments, the poluripotency factors comprise OCT4. In some embodiments, the early cleavage like state comprises dissolution of chromocenters. In some embodiments, the early cleavage like state comprises activation of retrotransposons. In some embodiments, the retrotransposons comprise ERVL or MaLR retrotransposons or homologs or orthologs thereof.

In some embodiments, the method further comprises expressing one or more of OCT3/4, Sox2, Klf4, and c-Myc. In some embodiments, the method further comprises expressing or administering a DNA methyltransferase (DNMT) protein or activator thereof, a histone dimethylase activator, and/or a H3K9 methyltransferase inhibitor to the cell. In some embodiments, the DNA methyltransferase protein comprises DNA methyltransferase 3a or 3b (DNMT3a/b). In some embodiments, the histone demethylase activator is a Kdm4 histone demethylase activator. In some embodiments, the cell is a human, non-human primate, mouse, dog, cow, sheep, or horse cell. Non-human primates include, for example, macaques sp., monkeys, apes, chimpanzees, gorillas, orangutans, marmosets, tamarins, spider monkeys, owl monkeys, vervet monkeys, squirrel monkeys, and baboons.

In some embodiments, the DUXC protein is of the same animal type as the cell. In some embodiments, the DUXC protein is of a different animal type as the animal type of the cell. In some embodiments, the cell is a human cell and the DUXC protein comprises DUX4; the cells is a mouse cell and the DUXC protein comprises mouse DUX; the cell is a cow cell and the DUXC protein comprises cow DUXC; the cell is a canine cell and the DUXC protein comprises canine DUXC; the cell is a horse cell and the DUXC protein comprises horse DUXC; the cell is a sloth cell and the DUXC protein comprises sloth DUXC; the cell is an elephant cell and the DUXC protein comprises elephant DUXC; or the cell is a pig cell and the DUXC protein comprises pig DUXC.

In some embodiments, expressing a protein comprises transferring a DUXC polypeptide or nucleic acid encoding a DUXC polypeptide into the cell. In some embodiments, the method comprises transferring a DUXC RNA into the cell. In some embodiments, the method comprises transferring a DUXC DNA into the cell. In some embodiments, the DUXC RNA is transferred into the cell by injection of the RNA. In some embodiments, the DUXC DNA is transferred into the cell by injection of the DNA. In some embodiments, the DUXC nucleic acid is transferred into the cell by a method known in the art and/or described herein.

In some embodiments, a DUXC polypeptide comprising the sequence of a DUXC polypeptide disclosed herein is expressed in the cell. In some embodiments, a nucleic acide encoding a DUXC polypeptide disclosed herein is expressed in the cell.

In some embodiments, the method further comprises differentiating the cell. In some embodiments, the cell is differentiated into an extraembryonic cell, an embryonic cell, or a derivative thereof. In some embodiments, the differentiated cell is one known in the art or described herein. In some embodiments, the extraembryonic cell comprises a placental cell, yolk sac cell, extraembryonic endoderm cell, or a derivative thereof. In some embodiments, the embryonic cell comprises a mesoderm cell, ectoderm, endoderm cell cell, or a derivative thereof. In some embodiments, the differentiated cells comprise a blood cell, a neural cell, a bone cell, or a skin cell.

Further aspects of the disclosure relate to a method for making a host cell nuclear transfer (SCNT) embryo comprising expressing a DUXC protein in a somatic cell and transferring the nucleus of the somatic cell to an enucleated oocyte, thereby making a SCNT embryo. As shown in FIG. 31 of the application, DUX-expressing mESC can regain totipotency using a chimera assay, in which the cells incorporate into both the trophectoderm and the inner-cell mass. Therefore, the methods of the disclosure allow for incorporation of DUXC expressing cells in into both embryonic and extraembryonic tissue.

In some embodiments, the method further comprises stimulating the oocyte. In some embodiments, the method further comprises expressing one or more of OCT3/4, Sox2, Klf4, or c-Myc in the somatic cell. In some embodiments, the method further comprises administering or expressing a DNMT protein or activator thereof, a histone dimethylase activator, and/or a H3K9 methyltransferase inhibitor to or in the the somatic cell. In some embodiments, the DNMT protein comprises 3a or 3b (DNMT3a/b). In some embodiments, the histone demethylase activator is a Kdm4 histone demethylase activator.

In some embodiment, expressing a protein comprises transferring a DUXC polypeptide or nucleic acid encoding a DUXC polypeptide into the cell. In some embodiments, the method comprises transferring a DUXC RNA into the cell. In some embodiments, the method comprises transferring a DUXC DNA into the cell. In some embodiments, the DUXC RNA is transferred into the cell by injection of the RNA. In some embodiments, the DUXC DNA is transferred into the cell by injection of the DNA. In some embodiments, the DUXC RNA or DNA is transferred into the cell by a method known in the art and/or described herein.

In some embodiments, the method further comprises culturing the SCNT embryo. In some embodiments, the method further comprises isolating stem cells from the cultured SCNT embryo. In some embodiments, the method further comprises implanting the SCNT embryo into a host.

In some embodiments, the host is a mammal. In some embodiments, the host is a laboratory mammal. In some embodiments, the host is an agricultural mammal. In some embodiments, the host is a human, non-human primate, cow, a pig, a rabbit, a mouse, a rat, a horse, or a dog. In some embodiments, the host is a non-human animal. In some embodiments, the host is one described herein.

Further aspects relate to an animal clone prepared by a method of the disclosure.

Yet further aspects relate to a method for inducing a naïve cell from a primed cell, the method comprising expressing a protein containing a DUXC double homeodomain in the primed cell. In some embodiments, the primed cell is an induced pluripotent cell. In some embodiments, the primed or naïve cell is further defined as having a cell characteristic described in this disclosure. In some embodiments, the primed or naïve cell is further defined as not having a cell characteristic described in this disclosure.

Further aspects relate to an isolated totipotent cell comprising an exogenous gene encoding for a DUXC protein. In some embodiments, the totipotent cell is further defined as having or not having a cell characteristic described in this disclosure. In some embodiments, the DUXC protein comprises DUX4, mouse DUX, cow DUXC, canine DUXC, horse DUXC, sloth DUXC, elephant DUXC, or pig DUXC.

Further aspects relate to a method for treating a disease in a subject, the method comprising administering a stem cell of the disclosure, a stem cell produced by the methods of the disclosure, a totipotent cell of the disclosure, a totipotent cell produced by the methods of the disclosure, or the progeny thereof to the subject. In some embodiments, the stem cell is isogenic. In some embodiments, the stem cell is autogenic. In some embodiments, a progeny of the stem cell is administered to the subject, wherein the progeny comprises a differentiated cell. In some embodiments, the differentiated cell is an extraembryonic endoderm cell, an embryonic cell, or a derivative thereof. In some embodiments, the extraembryonic cell comprises a placental cell, yolk sac cell, extraembryonic endoderm cell, or a derivative thereof. In some embodiments, the embryonic cell comprises a mesoderm cell, ectoderm, endoderm cell, or a derivative thereof. In some embodiments, the differentiated cells comprise a blood cell, a neural cell, a bone cell, or a skin cell. In some embodiments, the differentiated cell is one that is described herein. In some embodiments, the disease is selected from an autoimmune disease, a neurodegenerative disease, or cancer. In some embodiments, the disease is one described herein. In some embodiments, the disease is diabetes, rheumatoid arthritis, Parkinson's disease, Alzheimer's disease, osteoarthritis, stroke and traumatic brain injury, learning disability, spinal cord injury, heart infection, baldness, impairment of the hearing, vision impairment, cornea impairment, amyotrophic lateral sclerosis, Crohn's disease, wound healing, or male infertility.

Further aspects relate to a SCNT embryo comprising exogenous expression of a DUXC protein. In some embodiments, the DUXC protein comprises DUX4, mDUX, cow DUX, canine DUX, horse DUX, sloth DUX, elephant DUX, or pig DUX.

Further aspects relate to a method for generating human extraembryonic tissue in vitro, the method comprising differentiating the cells or the disclosure or cells derived from the methods of the disclosure into extraembryonic cells. In some embodiments, the cells are placental cells.

As used herein the specification, “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present disclosure will become apparent from the following detailed description. It should be understood, however, that the detailed description and the specific examples, while indicating preferred embodiments of the disclosure, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A-F. Mouse DUX (mDUX) and human DUX4 (hDUX4) activate an early embryo gene signature in muscle cells of their respective species. (a) mDUX transcriptome in C2C12 mouse muscle cells: red dots are genes affected more than absolute(log2FoldChange)>=2 and adjusted p-value<=0.05. (b) GSEA: gene set is 2C-like gene signature, x-axis is log2FoldChange-ranked mDUX transcriptome. Green line is running enrichment score(ES); ES increases when a gene in the mDUX transcriptome is also in the 2C-like gene set; ES decreases when a gene isn't in the 2C-like gene set. Increases are also indicated by vertical black bars. Enrichment score at the peak normalized by gene set size is NES. Negative control: FIG. 6. (c) Direct targets are defined by RNA-seq (absolute(log2FoldChange)>=2 and adjusted p-value<=0.05) and ChIP-seq (peak within one kilobase +/− of transcriptional start site, TSS). Shown are the 30 genes in the 2C-like state gene signature out of 67 total mDUX direct targets. (d) Homeodomain alignments (%=amino acid identity, *=four predicted DNA-contacting residues, cDUXC=canine DUXC). (SEQ ID NO:1-6) (e) GSEA: gene set is the top 500 most upregulated genes in hDUX4-expres sing human cells, x-axis is log2FoldChange-ranked mDUX transcriptome in mouse cells. This cross-species comparison required limiting both gene set and transcriptome to 1:1 mouse-to-human orthologues. The opposite comparison is in FIG. 7B. (f) GSEA: gene set is the human orthologues of the mouse 2C-like gene signature, x-axis is log2FoldChange-ranked hDUX4 transcriptome in human muscle cells. Both gene set and transcriptome are limited to 1:1 mouse-to-human orthologues. Note: mouse 2C-like gene signature has 469 genes total, 297 gene have simple 1:1 mouse-to-human orthology.

FIG. 2A-B. Despite binding motif divergence and general transcriptome divergence, hDUX4 transcriptome in mouse muscle cells is enriched for the 2C-like gene signature. (a) Comparison of mDUX and hDUX4 binding motifs as determined by MEME. Note the divergence in the first half of the motif and the conservation of the second half of the motif. (SEQ ID NO:7-8) (b) GSEA: gene set is the mouse 2C-like gene signature, x-axis is the log2FoldChange-ranked hDUX4 transcriptome in mouse cells. Since the mouse 2C-like gene signature and this hDUX4 transcriptome were both identified in mouse cells, neither gene set nor transcriptome was limited to genes with 1:1 mouse-to-human orthology.

FIG. 3A-E. mDUX, but not hDUX4, activates transcription of repetitive elements characteristic of the early embryo in mouse muscle cells. (a) Expression levels of repeats during mDUX expression in mouse cells. Each dot is a repeatName as defined by RepeatMasker. Red color indicates differential expression at absolute log2-Foldchange>=1 and adjusted p-value<=0.05. Number in parentheses is log2-FoldChange. (b) Same as (a) for hDUX4-expressing mouse muscle cells. (c) Same as (a) for hDUX4-expressing human muscle cells, data previously published. (d) Luciferase assay showing mDUX induction of luciferase using a 2C-active MERV-L element active, which contains a match to the mDUX motif. (e) Black bars are counts of genes in the 2C-like gene signature that are MERV-L promoted and activated by the indicated factor. White bars are genes detected by RNAseq, but are not upregulated. Gray bars are genes with no reads by RNAseq.

FIG. 4A-B. hDUX4 bound repetitive elements that also have RNAseq reads that connect the ChIP-seq peak to an annotated exon in mouse muscle cells. (a) LTR-family distribution of bound elements with RNAseq reads that connect the element to an annotated exon. (b) Two examples of hDUX4 binding an LTR to induce novel transcription. Repeat=black box.

FIG. 5A-F. Transcriptional divergence between hDUX4 and mDUX maps to the two DNA-binding homeodomains (HD). (a) Cartoons of chimeric proteins; MMH is the two mDUX homeodomains and the hDUX4 C-terminus; MHM is mDUX with homeodomain 2 (HD2) from hDUX4; HMM is mDUX with homeodomain 1 (HD1) from hDUX4. (b-d) RT-qPCR data for 2C-like genes in mouse muscle cells of various classes. (b) 2C-like genes with MERV-L promoters. (c) 2C-like genes with conventional promoters that are induced by hDUX4 and mDUX. (d) 2C-like genes with conventional promoters that are induced only by mDUX. (e) Cartoons of reciprocal set of chimeric proteins; HHM is the two hDUX4 homeodomains and the mDUX C-terminus; HMH is hDUX4 with HD2 from mDUX; MHH is hDUX4 with HD1 from mDUX. (f) RT-qPCR data for hDUX4-target genes in human rhabdomyosarcoma cells.

FIG. 6. Negative control for GSEA. (a) As a critical negative control, GSEA was used to assess enrichment of the 2C-like state gene signature in a transcriptome where one does not expect to find enrichment. The transcriptome used was a published dataset representing the MyoD transcriptome when expressed lentivirally in mouse embryonic fibroblasts. MyoD has no known role in the 2C mouse embryo, rather it is the master regulator of muscle lineage specification. That this graph peaks near the center of the x-axis indicates that the majority of the 2C-like state genes are unaffected by MyoD (vertical hash mark). This contrasts distinctly with the taller, left-shifted peak seen in FIG. 1B, for example. GSEA determined p-values by permuting the transcriptome 1,000 times, hence the report of “p-value<0.001”. It seems likely that with more permutations there would be more distinction between the p-value reported for this transcriptome and the p-values reported elsewhere in this study.

FIG. 7A-B. Zscan4c, a ZSCAN family member, is a direct target of mDUX. (a) ChIP-seq and RNA-seq coverage near the Zscan4c locus. Black rectangle shows location of 450 bp sequence (chr7:11,005,309-11,005,758) that was synthesized and cloned upstream of luciferase to create the Zscan4c reporter. Find Individual Motif Occurrences (FIMO) identified two mDUX binding motifs that overlap the Zscan4c reporter region. Figure prepared with Integrative Genomics Viewer. (b) Luciferase assay data using reporter that includes 450 bp DNA under the mDUX ChIP-seq peak near the TSS of Zscan4c and either mDUX or an empty vector.

FIG. 8. Reciprocal GSEA showing mDUX and hDUX4 activate orthologous genes in their respective species. Making the opposite comparison as the graph in main text FIG. 1E, this GSEA shows that the 500 genes most upregulated by mDUX were significantly enriched in the genes most upregulated by hDUX4. The x-axis is the log2FoldChange-ranked hDUX4 transcriptome. This analysis compared mDUX-expressing mouse cells to hDUX4-expressing human cells. Since this comparison is between species, both gene set and transcriptome to genes were limited with simple 1:1 mouse-to-human orthologues.

FIG. 9A-D. RNA-seq and ChIP-seq data for hDUX4 expressed in mouse muscle cells. (a) Comparison of hDUX4 binding motifs in mouse and human muscle cells as determined by MEME. (SEQ ID NO:9-10) (b) hDUX4 transcriptome in mouse muscle cells. Red dots are genes affected more than absolute(log2FoldChange)>=2 and adjusted p-value<=0.05 are shown in red. (c) Comparison of transcriptome induced by hDUX4 and mDUX in mouse muscle cells. Only genes for which there are reads in both data sets are included: 13,515 genes total. Spearman's rank correlation coefficient is 0.1812. (d) bovine orthologue DUXC, activated many of the same key EGA genes in bovine fibroblast.

FIG. 10A-C. Distribution of transcribed repeats broken down by repFamily. (a) mDUX-expressing mouse muscle cells. (b) hDUX4-expressing mouse muscle cells. (c) re-analyzed data from hDUX4-expressing human muscle cells.

FIG. 11A-C. ChIP-seq supports mDUX, but not hDUX4, binding to MERV-L in mouse muscle cells. (a) mDUX and hDUX4 ChIP-seq coverage in mouse muscle cells at a MERV-L LTR. (b) 26% of the 8187 total mDUX binding sites identified fall within LTR elements, which is 2-fold more than expected if these binding sites were evenly distributed across the genome. Both ERVK and ERVL elements contributed to the enrichment. Although hDUX4 binding sites are not overrepresented in LTR elements in mouse cells (compare third bar to second bar), hDUX4 has 1.7-fold more binding sites in ERVL-MaLRs than expected by genomic distribution. Previously published hDUX4 binding site distribution in human muscle cells shown for comparison. (c) The MERV-L LTR consensus sequence carries a match to the mDUX binding motif (q-value=0.0132) (SEQ ID NO:11).

FIG. 12. Luciferase assay with (HUMAN)ZSCAN4 promoter. To confirm that the chimeric proteins were expressed and stable, chimeras were tested by luciferase assay on a reporter that responds to both hDUX4 and mDUX (J. Whiddon, unpublished data). Such a reporter is the published (HUMAN)ZSCAN4 promoter driving luciferase⁶, which has four good matches to the hDUX4 binding motif and two good matches to the mDUX binding motif.

FIG. 13A-C. mDUX binding sites were identified using two complementary ChIP-seq approaches. (a) Cartoons of antibodies and chimera combinations used in ChIP-seq. (SEQ ID NO:12-13) (b) Amount of overlapping peaks by genomic coordinates. (c) De novo motif prediction for peaks called from mDUX_A-19 and MMH_MO488/489.

FIG. 14A-B. Naïve marker (A) and DUX4 and DUX4 target ZSCAN4 (B) expression in FSHD2 primed, quiescent, and naïve iPS cell.

FIG. 15A-B. Naïve marker expression in Doxycycline inducible DUX4CA control iPS cell line. (A) DOX was treated for either 14 hrs or 24 hrs. (B) DOX was treated for 8 hrs then removed for 16 hrs for one DOX pulse.

FIG. 16A-B. CHAF1A suppresses D4Z4 and DUX4 expression in human FSHD2 myoblasts 16A shows knockdown of CHAF1A and CHAF1B by siRNA transfection in cultured human FSHD2 myoblasts is associated with dramatic de-repression of DUX4 and the activation of the DUX4 target gene ZSCAN4. This is accompanied by loss of H3K9me3 and H3K9me2 at the D4Z4 region (16B). These data demonstrate that inhibiting CHAF1 leads to DUX4 expression.

FIG. 17A-F. Transcriptional changes in developing human oocytes and pre-implantation embryos. (a) Graphical summary of the human oocyte and embryonic stages (and cell numbers) collected (left panel), and depiction of the laser and mechanical separation of day 5-6 blastocysts into ICM) and mural trophectoderm (right panel). (b) Comparisons to published single cell datasets of relative read coverage (from TSS to TTS) at annotated genes and exons. (c) Principal component analysis (PCA) of all oocyte and embryonic stages based on the highest 50% of all expressed genes (>1 mean FPKM). (d) Statistically determined k-means clusters based on the highest 50% all expressed genes (left panel). Clusters 1, 4, and 7 include the notable developmental genes FIGLA, ZSCAN4, and NANOG, respectively (right panel). (e) The top five transcription factor motifs from the HT SELEX collection enriched in a 5 kb upstream window of the 738 genes in cluster 4. (SEQ ID NO:14) (f) Single cell expression data (RPKM) for hDUX4 acquired from Yan et al. 2013.

FIG. 18A-D. A cleavage-specific transcriptional program is activated in iPSCs following hDUX4 expression. (a) Heatmap depicting the top 25 induced genes in human pluripotent stem cells following 14 hrs post hDUX4 induction [2 biological replicates per condition], alongside their embryonic expression. Bold font indicates genes within Cluster 4 (see FIG. 17D). The bottom row represents the median embryonic expression of all 297 genes upregulated following hDUX4 expression. (b) Browser snapshot of ZSCAN4 expression during embryonic development (blue) as well as in muscle and pluripotent stem cells (PSC) before and after induction of hDUX4. In each system, the TSS overlaps with a hDUX4 ChIP-seq peak identified in multiple replicates (black). The dashed line is used to indicate a ˜2 kb window around the ZSCAN4 promoter region. (c) The ZSCAN4 promoter (from FIG. 17B), and multiple modified versions (top), were cloned into luciferase vectors and cotransfected into human pluripotent stem cells with GFP, DURA, or hDUX4 mRNA expression constructs and evaluated for luciferase intensity after 24 hrs (bottom) [4 biological replicates per condition]. (d) hDUX4 induction of repetitive elements in human iPS cells (predominantly MLT2A1/HERVL, top left panel), and their stage-specificity in embryos (top right panel). Browser snapshot of hDUX4 ChlPseq and RNAseq of a typical MLT2A-driven HERVL (bottom left panel). The predicted hDUX4 binding motif is strongly enriched in transcribed MLT2 elements but not in related, but unaffected, variants (right bottom panel). Statistics determined using an unpaired t-test. Error bars, s.d.

FIG. 19A-G. A DUX4 ortholog in mouse, mDux, activates a cleavage-specific transcriptional program in mouse ES cells. (a) Sequence level comparison of mDUX with hDUX4 (top) and its relative expression/enrichment in preimplantation mouse embryonic cells (Deng et al. 2014) and ‘2C-like’ cells (Ishiuchi et al. 2015) (bottom). (b) Top 15 differentially-expressed genes and repetitive elements following transient ectopic mDux expression in mouse embryonic stem cells (mESCs). (c) Relative expression of mDux-induced genes in preimplantation mouse embryonic cells (Deng et al. 2014) and ‘2C-like’ cells (Ishiuchi et al. 2015). (d) Diagram of TET-inducible lentiviral constructs stably integrated into mESCs (left) and their effect (via administration of doxycycline) on the reactivation of a stably integrated MERVL::GFP reporter transgene measured by flow cytometry (right) [4 biological replicates per condition; 200,000 cells per replicate]. (e) Diagram of mDux-induced cell populations used for RNA sequencing experiments (f) Comparison of the transcriptional profiles generated from panel f. (g) Dot plot comparing differential expression of all genes in the mDux-induced MERVL::GFP subpopulation with an uninduced MERVL::GFP subpopulation described previously (Ishiuchi et al. 2015).

FIG. 20A-B. mDux expression converts mESCs to a ‘2C-like’ state. (a) Immunofluorescence quantifying the loss of pluripotency, exemplified by the loss of OCT4 protein and chromocenters in mESCs following ectopic mDux expression (n >100). (b) Summary of mESC metastability and the molecular features that define the 2-cell embryo and ‘2C-like’ cell state.

FIG. 21A-C. Induction of ‘2C-like’ cells following CAF-1 depletion requires mDUX. (a) mDux is highly upregulated following CAF-1 depletion (top). Area-proportional Venn diagram displays the large overlap of mDux-induced genes with those upregulated following Chaf1a knockdown (Ishiuchi et al. 2015)(bottom). Notably, mDUX upregulated genes display higher median upregulation than other upregulated genes (right). (b) Dot plot depicting the strong correlation of gene expression changes in the mDux-induced GFP^{pos} versus GFP^{neg} cells, and Chaf1a-depletion induced GFP^{pos} versus GFP^{neg} cells. (c) Flow cytometry plots used to quantify GFP^{pos} cells following Chaf1a knockdown alone and in combination with mDux knockdown, using two separate siRNA pools (si-mDux P1/P2) [3 biological replicates per condition; 100,000 cells per replicate]. Experiment performed in biological triplicate and replicated three times. Statistics determined using an unpaired t-test. Error bars, s.d.

FIG. 22A-C. mDux expression converts the chromatin landscape of mESCs to a state resembling an early 2-cell embryo. (a) ATAC-seq signal in mDux-induced GFP^{neg} and GFP^{pos} cells and comparison to early embryonic stages (Wu et al. 2016), centered on the differential regions identified in two biological replicates. (b) Line graph displaying the unique broad ATAC signal across regions gained in mDux-induced GFP^{pos} cells matching that observed in early 2-cell embryos. (c) Pie charts displaying the distribution of ATAC-seq peaks across genomic features (top) and their overlap with MERVL/MT2_Mm elements (bottom). Pvalue refers to a statistical enrichment over random.

FIG. 23A-B. mDux binds directly to MERVL elements and other cleavage-specific gene promoters, and locally opens chromatin. (a) A predicted mDUX binding motif centrally enriched at the summit of the top 1500 identified ChIP-seq peaks. Analysif of Motif Enrichment (AME) identifies predicted motif enrichment in MT2_Mm (MERVL) LTR elements and in regions gaining ATAC sensitivity in GFP^{pos} cells. (SEQ ID NO:15) (b) Screen shots of three regions that gain ATAC signal in GFP^{pos} cells. Note the broad ATAC signal through the entire gene body of Zscan4c and the overlap of ATAC signal with mDUX ChIP-seq peaks.

FIG. 24. The DUX4-family of genes defines and drives the unique cleavage stage transcriptional program. (a) A cleavage-specific transcriptional program is activated at EGA in mouse and human by mDUX or hDUX4, respectively. The genes and repetitive elements that are targets of these DUX4-family genes mediate important molecular transitions associated with embryonic reprogramming (SEQ ID NO:16-17).

FIG. 25A-F. (a) Screenshot of the TET3 genomic locus displaying read coverage bias in previous single cell datasets (Yan et al., green; Xue et al., orange). (b) Gene expression correlations using stage average FPKM data; r values are calculated using a spearman rank statistic. S: single cell; P: pooled cells. (c) Bar graphs comparing total exonic transcription (left) and novel transcription (right) measured in base pairs; employing thresholds of >1, >3 or >5 reads per region. Exon transcription includes all exonic base pairs annotated by Ensemble, UCSC, and NONCODE. (d) Bar chart depicting the number of transcript isoforms expressed by developmental stage. (e) A non-canonical NANOG isoform is expressed specifically in the cleavage stage. (f) A non-canonical TET2 isoform is maternally loaded. The red arrow is used to depict the severity of the TET2 truncation with respect to important protein domains [CD-Cys-rich domain; DSBH-Double-stranded β-helix dioxygenase domain]

FIG. 26A-C. (a) An arbitrarily rooted phylogenetic tree of human PRD-class homeodomains; both homeodomains for the ‘double homeobox’ genes are included separately and can be distinguished by the number following the ‘HD’ designation. Orange font indicates genes enriched in the cleavage embryo. Green font is used to delinate mDux; the functional equivalent of hDUX4 in mouse. (b) Single cell expression data (RPKM) for related double homeobox and PRD-like factors acquired from Yan et al. (c) Screenshots from RNA-seq and ChIP-seq experiments demonstrating that DUX4 directly activates DUXA, DUXB, and LEUTX expression via proximal LTR elements.

FIG. 27A-C. (a) RNAseq replicates of induced pluripotent cells (PSCs) following hDUX4 induction (for 14 or 24 hrs) cluster together based on global transcriptional profiles (top). hDUX4 induction consistently changes the expression of 227 genes. Notably, it has no effect on pluripotency (bottom). (b) Box plot displaying the embryonic expression of the 297 genes upregulated (FC>2, FDR<0.01) after 14 hours of ectopic hDUX4 expression in PSCs. (c) Scaled line graphs demonstrating the enriched expression of satellite repeats in the cleavage stage.

FIG. 28A-G. (a) Amino acid sequence level comparison of hDUX4 and Mdux (SEQ ID NO:18-19). (b) Pie chart displaying the conservation level of mDUX target genes determined via expression in mESCs. (c) RNA-seq reads were mapped to the codon altered mDux transgene to show relative expression following induction with doxycycline. (d) Results of a live imaging experiment on MERVL::GFP cells showing that activation of the reporter is dose-dependent. (e) Effects of ectopic mDux expression on repetitive elements in both unsorted and sorted RNA-seq experiments. Notably, mDUX robustly induces transcription from both MERVL elements and pericentromeric satellite repeats (GSAT). (f) MERVL and HERVL repetitive elements are homologous. (g) RT-qPCR data for ‘2c genes activated by mDux in mouse C2C12 [myoblasts] cells. Experiment performed in biological triplicate. Error bars, s.d.

FIG. 29A-E. (a) Venn diagrams showing the degree of overlap in regards to the regions that gain, lose, and maintain ATAC-seq signal between replicate comparison of sorted GFP^{pos} versus GFP^{neg} cells. (b) Effects on adjacent gene expression accompanying changes in chromatin accessibility. (c) Screenshot of a 800 kb region on chromosome 7 encompassing all annotated Zscan4 variants. Broad stretches of open chromatin in mDux-induced cells (resembling the early 2-cell embryo), overlap with ChIP-seq and RNA-seq peaks. (d) Genomic breakdown of HA-mDux ChIP-seq peaks and (e) percent overlap with ATAC-seq gained/lost peaks in mDux induced mESCs. Statistics determined using an unpaired t-test. Error bars, s.d.

FIG. 30A-C. shows alignment of homeodomain 1 (a), homeodomain 2 (SEQ ID NO:20-30) (b), and the C-terminal activation domain (SEQ ID NO:31-41)(c) from various animals (SEQ ID NO:42-52).

FIG. 31. shows the chimera contribution of control mESC or DUX-expressing mESC. mESC were injected into morulas at E3.0 and then contribution to blastocyst lineages (inner cell mass or trophectoderm) was quantified at E4.5. mCherry-transgene was used to mark mESC and DUX-mESC.

FIG. 32A-B. Dux, but not DUX4, activates transcription of repetitive elements characteristic of the early embryo in mouse muscle cells. (a) Example of a Dux ChIP-seq peak in MERV-L (MT2-element in RepBase nomenclature). Track height is 200 reads for all tracks. mm10 genome location is chr15:52,742,953-52,744,319. (b) Luciferase assay comparing the activation of a 2C-active MERV-L element reporter by either Dux, DUX4 or an empty vector. The MERV-L element contains a match to the Dux motif and was mutated as shown in cartoon to the right and the full sequence is in Supplementary FIG. 6d. Activation of the mutated MERV-L reporter is also shown. Data shown are mean fold change over empty vector of 3 cell cultures prepared in parallel for each condition. Error bars are s.e.m. The non-mutated MERV-L reporter activation experiment was repeated on three separate occasions with consistent results. The mutated MERV-L reporter experiment was performed on one occasion (SEQ ID NO:53-54).

FIG. 33. Dux and DUX4 use different types of LTR elements as alternative promoters for protein-coding genes. Histogram showing the number of genes in the 2C-like signature where the indicated factor bound a MERV-L (MT2-type) element based on ChIP-seq data and there was at least one RNA-seq read that connected the ChIP-seq peak range to an annotated exon in mouse muscle cells, termed “Peak-Associated Genes” (PAGs). Cartoon depiction of PAGs that overlap MERV-Ls is to the right. For two examples of PAGs that start in MERV-L (MT2-type) elements.

FIG. 34A-B. Pramef25 is a direct target of Dux. (a) ChIP-seq and RNA-seq coverage near the Pramef25 locus. Black rectangle shows location of 750 bp sequence (mm10; chr4: 143,954,684-143,955,431) that was synthesized and cloned upstream of firefly luciferase to create the Pramef25 reporter. Note that Dux regulates an upstream, unannotated start site of Pramef25. Find Individual Motif Occurrences (FIMO) identified three Dux binding motifs that overlap the Pramef25 reporter region. Figure prepared with UCSC Genome Browser5; track heights given in square brackets are read counts. (b) Luciferase assay comparing the activation of the Pramef25 reporter by either Dux or an empty vector. The original sequences of three predicted Dux binding motifs and the sequences to which they were mutated are shown in cartoon to the right. Data shown are mean fold change over empty vector of 3 cell cultures prepared in parallel for each condition. Error bars are s.e.m. The non-mutated Pramef25 reporter activation experiment was repeated on three separate occasions with consistent results. The mutated Pramef25 reporter experiment was performed on one occasion (SEQ ID NO:55-66).

FIG. 35A-J. Zscan4c is a direct target of Dux and each Zscan4-cluster gene contains a Dux ChIP-seq peak at its TSS (a) ChIP-seq and RNA-seq coverage near the Zscan4c locus. Black rectangle shows location of 450 bp sequence (chr7:11,005,309-11,005,758) that was synthesized and cloned upstream of luciferase to create the Zscan4c reporter. Find Individual Motif Occurrences (FIMO) identified four Dux binding motifs that overlap the Zscan4c reporter region. Figure prepared with Integrative Genomics Viewer6,7; track heights given in square brackets are read counts. (b) Luciferase assay comparing the activation of the Zscan4c reporter by either Dux or an empty vector. The original sequences of four predicted Dux binding motifs and the sequences to which they were mutated are shown in cartoon to the right. Data shown are mean fold change over empty vector of 3 cell cultures prepared in parallel for each condition. Error bars are s.e.m. The non-mutated Zscan4c reporter activation experiment was repeated on three separate occasions with consistent results. The mutated Zscan4c reporter experiment was performed on two occasions. (SEQ ID NO:67-74) (c) UCSC genome browser shot of Zscan4 cluster, showing RNA-seq and ChIP-seq coverage tracks. FIMO track shows locations of predicted Dux binding motifs and MERV-L track shows RepeatMasker MT2_Mm and MERVL-int locations. mm10 genomic coordinates: chr7:10,788,877-11,408,611. Note: The two loci with RNA-seq and ChIP-seq coverage in the absence of a UCSC-annotated Zscan4 gene are annotated as Zscan4 genes in other annotation models. (d) UCSC genome browser shot of Zscan4a, mm10 genomic coordinates: chr7: 10792200-10801100. (e) UCSC genome browser shot of Zscan4b, mm10 genomic coordinates: chr7:10898700-10907000. (f) UCSC genome browser shot of Zscan4c and a MERV-L, mm10 genomic coordinates: chr7:11003700-11030500. The inventors did not find any RNA-seq reads that support splicing between this MERV-L and Zscan4c. (g) UCSC genome browser shot of Zscan4d and a MERV-L, mm10 genomic coordinates: chr7:11159600-11186100. The inventors did not find any RNA-seq reads that support splicing between this MERV-L and Zscan4d. (h) UCSC genome browser shot of Zscan4f, mm10 genomic coordinates: chr7:11395900-11404300. (i) UCSC genome browser shot of MERV-L downstream of Zscan4c (mm10 genomic coordinates: chr7: 11,019,863-11,029,599), zoomed in and rescaled to show ChIP-seq peaks at the LTR portion of the element and RNA-seq read coverage of the internal sequence. Note the scale differences between panels 3i-j and the remainder of the figure. (j) UCSC genome browser shot of MERV-L upstream of Zscan4d (mm10 genomic coordinates: chr7: 11,168,315-11,178,031), zoomed in and rescaled to show ChIP-seq peaks and RNA-seq read coverage. Note the scale differences in panels 3i-j and the remainder of Supplementary FIG. 3.

FIG. 36A-D. Distribution of transcribed LTR repeats broken down by repFamily. (a) Expression levels of repeats during Dux expression in mouse cells compared to un-induced cells of the same cell line, broken down by repeat family. Each dot is a repeatName as defined by RepeatMasker. Red color indicates differential expression at absolute(log2-Foldchange)>=1 and adjusted p-value<=0.05. (b) DUX4-expressing mouse muscle cells compared to un-induced cells of the same cell line. (c) Re-analyzed data from DUX4-expressing human muscle cells compared to un-induced cells of the same cell line. (d) The MERVL_LTR consensus sequence from RepBase carries a match to the Dux binding motif (q-value=0.0132, determined by FIMO) (SEQ ID NO:75).

FIG. 37A-C. Browser shots of Peak-Associated Genes in 2C-like signature that start in MERV-L elements. (a) AF067061. Note that the inventors defined “Peak-associated genes” algorithmically as genes that have a ChIP-seq peak and at least one RNA-seq read that connects the peak location to an annotated exon of the gene. All RNA-seq tracks in this panel have 10,500 read track height. All ChIP-seq tracks in this panel have 153 read track height. (b) B020004J07Rik. All RNA-seq tracks in this panel have 550 read track height. All ChIP-seq tracks in this panel have 90 read track height. (c) Gm8994. All RNA-seq tracks in this panel have 175 read track height. All ChIP-seq tracks in this panel have 80 read track height.

FIG. 38A-B. Distribution of ChIP-seq peak locations according to repeat family in mouse muscle cells expressing either Dux or DUX4. (a) Stacked bar chart shows the distribution of ChIP-seq peak locations for the top 10,000 peaks for each condition. Dux ChIP-seq peaks occurred 2.4-fold more often in LTR elements than expected if these binding sites were evenly distributed across the genome; ERVL elements contributed the most to this overrepresentation with 4.2-fold more peaks in ERVL than expected by chance (see Panel C). DUX4 binding sites were 1.5-fold overrepresented in LTR elements in mouse cells and ERVL-MaLR elements contributed the most to this enrichment with 2.6-fold more peaks in ERVL-MaLR than expected by chance. Note that the vast majority of DUX4-bound ERVL-MaLRs are not shared with Dux. Only 4% of bound ERVL-MaLRs are shared (334/8027 total peak locations). Shown for comparison is DUX4 ChIP-seq peak distribution in human muscle cells, based on re-analysis of previously published data to match computational methods of this study. (b) Grouped bar chart shows the fold enrichment for the ChIP-seq peak distribution shown in (a) compared to genomic distribution of each LTR family as reported by RepeatMasker.

FIG. 39A-D. Dux binding sites were identified using two complementary ChIP-seq approaches. (a) Cartoons of antibodies and chimera combinations used in ChIP-seq. (b) Quantity of overlapping peaks by genomic coordinates for each antibody listed. (c) Top motif is a de novo motif prediction for peaks called from MMH-expres sing cells immunoprecipitated with 50:50 mix of MO488 and MO489 antibodies compared to a mock pull-down. Bottom motif is a de novo motif prediction for peaks called from Dux-expressing cells immunoprecipitated with A-19 antibody compared to a mock pull-down. (SEQ ID NO:76-77) (d) Comparison of MMH transcriptome and Dux transcriptome in mouse muscle cells based on RNA-seq following transgene induction by doxycycline-treatment for 18 hours for MMH_clone6 and 36 hours for Dux_clone15B. These time points were chosen such that they immediately precede the predominant wave of cell death that occurs after prolonged exposure of muscle cells to Dux, so that they are matched functionally if not temporally. Comparator transcriptome for determining differential expression of genes by Dux and MMH was that of firefly luciferase-expressing mouse muscle cells. Data shown are from three cell cultures of each condition. Dux-expressing and luciferase-expressing cells were prepared and sequenced in parallel. MMH-expressing cells were prepared and sequenced at a separate time. Pearson correlation coefficient was 0.7847.

FIG. 40A-C. (a) MA plot showing DUX4-mediated induction of specific repeat elements, by subfamily (left). Mean-scaled expression of the top activated repeats HERVL and MLT2A1 in human oocytes and embryos (right). (b) The overlap of DUX4 ChIP occupied genes with genes enriched in the cleavage-stage embryo and activated by DUX4 overexpression in iPSCs. Overlap statistic calculated by hypergeometric test; only 477 of 739 ‘cleavage genes’ were annotated in GREAT. In the box, genes encoding notable transcription factors (TF), chromatin modifiers (CM), and post-translational modifying enzymes (PTE) in the overlapping population are listed. (c) Diagram summarizing the timing of DUX4 expression and its effects on embryonic gene expression.

FIG. 41A-C. UX binds directly to 2C gene promoters and retrotransposons. (a) Top enriched MGI expression and Gene Ontology (GO) terms identified in the 3,881 genes bound by DUX. (b) Overlap of DUX ChIP occupied genes with genes upregulated in unsorted mESCs after Dux overexpression (left), enriched in 2C-like cells (middle), or driven by MERVL elements (right). Enrichment statistics determined by hypergeometric test. (c) Screenshots demonstrating the overlap of DUX ChIP occupancy with the acquisition of 2-cell-embryo-like open chromatin and gene or MERVL expression (green box).

FIG. 42A-J. DUX4 directly activates the genes and repeat elements that are transiently expressed during the human cleavage stage. (a) Single cell expression data (RPKM) for DUX4 (RNA-seq data from ref. 16). (b) An arbitrarily rooted phylogenetic tree of human paired (PRD) homeodomains; both homeodomains for the ‘double homeobox’ (DUX) factors are included separately and can be distinguished by the number following the ‘HD’ designation. Orange font indicates genes enriched in the cleavage embryo. Green font is used to delineate mouse DUX homeodomains; the functional ortholog of human DUX4. (c) Single cell expression data (RPKM) for notable double homeobox and ‘PRD-like’ genes (RNA-seq data from ref. 16). (d) The overlap of differentially expressed genes in human iPSCs expressing DUX4 (vs. luciferase) for 14 or 24 hrs. (e) Box plot displaying the embryonic expression of the 150 common genes that are upregulated following DUX4 overexpression (for 14 hr or 24 hrs) in iPSCs (f) MA-plot showing repeat element (by subfamily) activation in human iPSCs 24 hrs post DUX4 overexpression (vs luciferase control). (g) The embryonic expression of satellite repeats-HSATII and ACRO1. (h) The overlap of DUX4 ChIP-seq peaks in iPSCs (red) with DUX4 ChIP-seq peaks in myoblasts (MB) from Geng et al., 2012 (light blue). [Overlap statistic calculated by hypergeometric test]. (i) Genome snapshots of cleavage-specific genes directly bound and activated by DUX4 in human iPSCs. (j) The number of repeat element instances uniquely bound by DUX4 for select activated (MLT2A1, MLT2A2, HSATII) and unaffected (LTR7, L1) subfamilies. [Enrichment statistic determined empirically; error bars, s.d.].

FIG. 43A-I. Mouse Dux, a functional ortholog of DUX4, activates a 2C transcriptional program and converts mESCs to a 2C-like state. (a) DUX4 and DUX amino acid sequence alignment. Highlighted in blue, green, and yellow are the two DUX4 homeodomains (HD) and the transactivation domain (TAD), respectively. (SEQ ID NO:78-79) (b) RT-qPCR data for select ‘2C’ genes activated following Dux expression in mouse C2C12 cells [three replicates per condition. Error bars, s.d.]. (c) Results of a live imaging experiment showing the relative gain of GFPpos cells (normalized by total cell surface area) as a function of time post dox-induction. (d) Schematic of the RNA-seq experiments conducted on Dux-expressing mESCs. (e) Overlap of differentially expressed genes (DEGs) from unsorted and sorted populations of Dux-expressing mESCs [Overlap statistic calculated by hypergeometric test]. (f) The normalized average expression of codon altered Dux transgene in our RNA-seq datasets from unsorted and sorted populations (left panel), relative to the normalized expression of endogenous Dux in spontaneously converting 2C-like cells (right panel) (RNA-seq data from ref. 9). (g) MA-plot showing the activation of repetitive elements (by subfamily) in both unsorted and sorted RNA-seq experiments. Notably, Dux expression robustly induces the expression of MERVL elements and pericentromeric major satellite repeats (GSAT). (h) Flow results demonstrating, in an independent HA-tagged clone, the ability of Dux expression to efficiently induce reactivation of the MERVL reporter in mESCs [three biological replicates per condition; error bars, s.d.]. (i) The expression of HA and loss of chromocenters is evaluated by immunofluorescence confirming entry into a 2C-like state. Scale bar, 10 um.

FIG. 44A-G. Dux is necessary for spontaneous and CAF-1-mediated conversion of mESCs to a 2C-like state. (a) A diagram of the Chromatin Assemble Factor (CAF-1) complex. The arrow points to the complex subunit (p150 encoded by the Chaf1a gene) targeted with siRNAs in our experiments. (b) Dot plot depicting the correlation of gene expression changes in the Dux-induced 2C-like cells, and those induced by Chaf1a knockdown (RNA-seq data from ref. 9). (c) Effects of Dux knockdown alone (left panel) and Chaf1a knockdown alone (right panel) on conversion of mESCs to a 2C-like state [three biological replicates per condition. Statistics determined using a two-tailed unpaired t-test, error bars, s.d.]. (d) The normalized average expression of Chaf1a and Dux in negative control (NC) and knockdown mESCs determined by RNA-seq [Error bars, s.d.]. (e) Bar chart showing the fraction of genes upregulated (FC>2, FDR<0.01) in Chaf1a depleted mESCs that are not affected in mESCs depleted for both Chaf1a and Dux. (note: one gene that was upregulated in Chaf1a depleted mESCs became downregulated in mESCs depleted for both Chaf1aand Dux). (f) The normalized average expression of MERVL-int and GSAT repeats in control and knockdown mESCs determined by RNA-seq [Error bars, s.d.]. (g) Screenshots showing the expression of notable genes following knockdown of Chaf1a alone and in combination with knockdown of Dux. (h) Boxplot showing the embryonic expression of the genes upregulated in both Chaf1a-depleted as well as Chaf1a- and Dux-depleted mESCs (termed ‘Dux-independent’) and the genes upregulated only in Chaf1a-depleted cells (termed ‘Dux-dependent’). Center line, median; box limits, upper and lower quartiles; whiskers, 1.5× interquartile range. (RNA-seq data from ref. 27) (i) Summary figure depicting the proposed relationship between CAF-1 and DUX with respect to mESC entry into a 2C-like state.

FIG. 45A-C. Dux-induced 2C-like cells acquire an open chromatin landscape resembling that of an early 2-cell-stage embryo. (a) Heatmap depicting the Pearson correlation of genome-wide ATAC-seq coverage profiles in Dux-induced mESCs and early embryonic developmental stages (Embryo ATAC-seq data from ref. 35). (b) Pie charts depicting the distribution of ATAC-seq gained, lost and common peaks (called after filtering alignment files for unique reads only) at basic genomic features. Inset pie charts indicate the percentage of unique peaks which overlap with MERVL elements (MT2_Mm and MERVL-int). [Enrichment statistic determined empirically]. (c) Boxplot shows the median log2 expression fold change (FC) of the genes neighboring regions of ATAC-seq gained, lost and common signal.

FIG. 46A-D. DUX binds directly to 2C gene promoters and retrotransposons. (a) Heatmap depicting gene clusters exhibiting stage-specific expression in the early mouse embryo (left panel). Overlap of DUX-ChIP occupied genes with each ‘stage-specific’ gene cluster (right panel) [overlap statistics determined by hypergeometric test]. (b) The number of repeat element instances uniquely bound by DUX for select affected (MT2_Mm, ORR1A3-int) and unaffected (L1, IAPEZ-int) subfamilies [enrichment statistics determined empirically; error bars, s.d.]. (c) The percentage of unique ATAC gained, lost, and common regions bound by DUX. (d) A binding motif for DUX predicted by MEME-ChIP based on the top 10,000 peak summits (left panel). This motif differs from that for DUX4, and only shows enrichment in mouse-specific regions of interest (right panel) (SEQ ID NO:80).

### DESCRIPTION OF ILLUSTRATIVE EMBODIMENTS

The inventors found that a eutherian-specific gene, or retrogene in some species, of the DUXC family (DUX4 in humans, Dux in mice) activates hundreds of endogenous genes (e.g. ZSCAN4, ZFP352, KDM4E) and retroviral elements (MERVL/HERVL/MaLR-families) that define the cleavage-specific transcriptional programs in mouse and human. Remarkably, mouse Dux expression potently converted mouse ESCs into two-cell embryo-like (‘2C-like’) cells, measured here by the reactivation of many cleavage-stage genes and repetitive elements, the loss of OCT4 protein and chromocenters, and by the conversion of the chromatin landscape (assessed by ATAC-seq) to a state strongly resembling mouse two-cell embryos. Taken together, the evidence indicates that mouse DUX and human DUX4 function as major drivers of the mammalian early cleavage state.

### I. Definitions

The term “allogeneic” refers to tissues or cells that are genetically dissimilar and hence immunogically incompatible, although from the individuals of the same species.

The term “DUXC” or “DUXC-family” refers to the DUXC gene orthologs in eutheria and the retrogenes derived by the retrotransposition of the DUXC gene in some species. The DUXC-family members can be identified by the presence of two homeodomains that show sequence similarity and the presence of an LLXXL motif encoded in at least one mRNA isoform from the locus.

The phrase “Somatic Cell Nuclear Transfer” or “SCNT” is also commonly referred to as therapeutic or reproductive cloning, is the process by which a somatic cell is fused with an enucleated oocyte. The nucleus of the somatic cell provides the genetic information, while the oocyte provides the nutrients and other energy-producing materials that are necessary for development of an embryo. Once fusion has occurred, the cell is totipotent, and eventually develops into a blastocyst, at which point the inner cell mass is isolated.

The term “nuclear transfer” as used herein refers to a gene manipulation technique allowing identical characteristics and qualities acquired by artificially combining an enucleated oocytes with a cell nuclear genetic material or a nucleus of a somatic cell. In some embodiments, the nuclear transfer procedure is where a nucleus or nuclear genetic material from a donor somatic cell is transferred into an enucleated egg or oocyte (an egg or oocyte from which the nucleus/pronuclei have been removed). The donor nucleus can come from a somatic cell.

The term “nuclear genetic material” refers to structures and/or molecules found in the nucleus which comprise polynucleotides (e.g., DNA) which encode information about the individual. Nuclear genetic material includes the chromosomes and chromatin. The term also refers to nuclear genetic material (e.g., chromosomes) produced by cell division such as the division of a parental cell into daughter cells. Nuclear genetic material does not include mitochondrial DNA.

The term “SCNT embryo” refers to a cell, or the totipotent progeny thereof, of an enucleated oocyte which has been fused with the nucleus or nuclear genetic material of a somatic cell. The SCNT embryo can develop into a blastocyst and develop post-implantation into living offspring. The SCNT embryo can be a 1-cell embryo, 2-cell embryo, 4-cell embryo, or any stage embryo prior to becoming a blastocyst.

The term “parental embryo” is used to refer to a SCNT embryo from which a single blastomere is removed or biopsied. Following biopsy, the remaining parental embryo (the parental embryo minus the biopsied blastomere) can be cultured with the blastomere to help promote proliferation of the blastomere. The remaining, viable parental SCNT embryo may subsequently be frozen for long term or perpetual storage or for future use. Alternatively, the viable parental embryo may be used to create a pregnancy.

The term “donor mammalian cell” or “donor mammalian somatic cell” refers to a somatic cell or a nucleus of cell which is transferred into a recipient oocyte as a nuclear acceptor or recipient.

The term “somatic cell” refers to a plant or animal cell which is not a reproductive cell or reproductive cell precursor. In some embodiments, a differentiated cell is not a germ cell. A somatic cell does not relate to pluiripotent or totipotent cells. In some embodiments the somatic cell is a “non-embryonic somatic cell”, by which is meant a somatic cell that is not present in or obtained from an embryo and does not result from proliferation of such a cell in vitro. In some embodiments the somatic cell is an “adult somatic cell”, by which is meant a cell that is present in or obtained from an organism other than an embryo or a fetus or results from proliferation of such a cell in vitro.

The term “differentiated cell” as used herein refers to any cell in the process of differentiating into a somatic cell lineage or having terminally differentiated. For example, embryonic cells can differentiate into an epithelial cell lining the intestine. Differentiated cells can be isolated from a fetus or a live born animal, for example.

In the context of cell ontogeny, the adjective “differentiated”, or “differentiating” is a relative term meaning a “differentiated cell” is a cell that has progressed further down the developmental pathway than the cell it is being compared with. Thus, stem cells can differentiate to lineage-restricted precursor cells (such as a mesodermal stem cell), which in turn can differentiate into other types of precursor cells further down the pathway (such as an cardiomyocyte precursor), and then to an end-stage differentiated cell, which plays a characteristic role in a certain tissue type, and may or may not retain the capacity to proliferate further.

The term “oocyte” as used herein refers to a mature oocyte which has reached metaphase II of meiosis. An oocyte is also used to describe a female gamete or germ cell involved in reproduction, and is commonly also called an egg. A mature egg has a single set of maternal chromosomes (23, X in a human primate) and is halted at metaphase II. A “hybrid” oocyte has the cytoplasm from a first primate oocyte (termed a“recipient”) but does not have the nuclear genetic material of the recipient; it has the nuclear genetic material from another oocyte, termed a “donor.”

The term “enucleated oocyte” as used herein refers to an oocyte which its nucleus has been removed.

The “recipient mammalian oocyte” as used herein refers to a mammalian oocyte that receives a nucleus from a mammalian nuclear donor cell after removing its original nucleus.

The term “prenatal” refers to existing or occurring before birth. Similarly, the term “postnatal” is existing or occurring after birth.

The term “blastocyst” as used herein refers to a preimplantation embryo in placental mammals (about 3 days after fertilization in the mouse, about 5 days after fertilization in humans) of about 30-150 cells. The blastocyst stage follows the morula stage, and can be distinguished by its unique morphology. The blastocyst consists of a sphere made up of a layer of cells (the trophectoderm), a fluid-filled cavity (the blastocoel or blastocyst cavity), and a cluster of cells on the interior (the inner cell mass, or ICM). The ICM, consisting of undifferentiated cells, gives rise to what will become the fetus if the blastocyst is implanted in a uterus. These same ICM cells, if grown in culture, can give rise to embryonic stem cell lines. At the time of implantation the mouse blastocyst is made up of about 70 trophoblast cells and 30 ICM cells.

The term “blastula” as used herein refers to an early stage in the development of an embryo consisting of a hollow sphere of cells enclosing a fluid-filled cavity called the blastocoel. The term blastula sometimes is used interchangeably with blastocyst.

The term “blastomere” is used throughout to refer to at least one blastomere (e.g., 1, 2, 3, 4, etc) obtained from a preimplantation embryo. The term “cluster of two or more blastomeres” is used interchangeably with “blastomere-derived outgrowths” to refer to the cells generated during the in vitro culture of a blastomere. For example, after a blastomere is obtained from a SCNT embryo and initially cultured, it generally divides at least once to produce a cluster of two or more blastomeres (also known as a blastomere-derived outgrowth). The cluster can be further cultured with embryonic or fetal cells. Ultimately, the blastomere-derived outgrowths will continue to divide. From these structures, ES cells, totipotent stem (TS) cells, and partially differentiated cell types will develop over the course of the culture method.

The term “karyoplast” as used herein refers to a cell nucleus, obtained from the cell by enucleation, surrounded by a narrow rim of cytoplasm and a plasma membrane.

The term “cell couplet” as used herein refers to an enucleated oocyte and a somatic or fetal karyoplast prior to fusion and/or activation.

The term “cleavage pattern” as used herein refers to the pattern in which cells in a very early embryo divide; each species of organism displays a characteristic cleavage pattern that can be observed under a microscope. Departure from the characteristic pattern usually indicates that an embryo is abnormal, so cleavage pattern is used as a criterion for preimplantation screening of embryos.

The term “clone” as used herein refers to an exact genetic replica of a DNA molecule, cell, tissue, organ, or entire plant or animal, or an organism that has the same nuclear genome as another organism.

The term “cloned (or cloning)” as used herein refers to a gene manipulation technique for preparing a new individual unit to have a gene set identical to another individual unit. In the present ivnention, the term “cloned” as used herein refers to a cell, embryonic cell, fetal cell, and/or animal cell has a nuclear DNA sequence that is substantially similar or identical to the nuclear DNA sequence of another cell, embryonic cell, fetal cell, differentiated cell, and/or animal cell. The terms “substantially similar” and “identical” are described herein. The cloned SCNT embryo can arise from one nuclear transfer, or alternatively, the cloned SCNT embryo can arise from a cloning process that includes at least one re-cloning step.

The term “transgenic organism” as used herein refers to an organism into which genetic material from another organism has been experimentally transferred, so that the host acquires the genetic traits of the transferred genes in its chromosomal composition.

The term “embryo splitting” as used herein refers to the separation of an early-stage embryo into two or more embryos with identical genetic makeup, essentially creating identical twins or higher multiples (triplets, quadruplets, etc.).

The term “morula” as used herein refers to the preimplantation embryo 3-4 days after fertilization, when it is a solid mass composed of 12-32 cells (blastomeres). After the eight-cell stage, the cells of the preimplantation embryo begin to adhere to each other more tightly, becoming “compacted”. The resulting embryo resembles a mulberry and is called a morula (Latin: morus=mulberry).

The term “enucleation” as used herein refers to a process whereby the nuclear material of a cell is removed, leaving only the cytoplasm. When applied to an egg, enucleation refers to the removal of the maternal chromosomes, which are not surrounded by a nuclear membrane. The term “enucleated oocyte” refers to an oocyte where the nuclear material or nuclei is removed.

The term “reprogramming” as used herein refers to the process that alters or reverses the differentiation state of a somatic cell, such that the developmental clock of a nucleus is reset; for example, resetting the developmental state of an adult differentiated cell nucleus so that it can carry out the genetic program of an early embryonic cell nucleus, making all the proteins required for embryonic development. In some embodiments, the donor mammalian cell is terminally differentiated prior to the reprogramming by SCNT. Reprogramming as disclosed herein encompasses effective reversion of the differentiation state of a somatic cell to a pluripotent or totipotent cell. Reprogramming generally involves alteration, in RNA expression patterns as well as reversal reversal, of at least some of the heritable patterns of nucleic acid modification (e.g., methylation), chromatin condensation, epigenetic changes, genomic imprinting, etc., that occur during cellular differentiation as a zygote develops into an adult. In somatic cell nuclear transfer (SCNT), components of the recipient oocyte cytoplasm are thought to play an important role in reprogramming the somatic cell nucleus to carry out the functions of an embryonic nucleus.

The term “culturing” as used herein with respect to SCNT embryos refers to laboratory procedures that involve placing an embryo in a culture medium. The SCNT embryo can be placed in the culture medium for an appropriate amount of time to allow the SCNT embryo to remain static but functional in the medium, or to allow the SCNT embryo to grow in the medium. Culture media suitable for culturing embryos are well-known to those skilled in the art. See, e.g., U.S. Pat. No. 5,213,979, entitled “In vitro Culture of Bovine Embryos,” First et al., issued May 25, 1993, and U.S. Pat. No. 5,096,822, entitled “Bovine Embryo Medium,” Rosenkrans, Jr. et al., issued Mar. 17, 1992, incorporated herein by reference in their entireties including all figures, tables, and drawings.

The term “culture medium” is used interchangeably with “suitable medium” and refers to any medium that allows cell proliferation and/or cell viability. The suitable medium need not promote maximum proliferation, only measurable cell proliferation. In some embodiments, the culture medium maintains the cells in a pluripotent or totipotent state.

The term “implanting” as used herein in reference to SCNT embryos as disclosed herein refers to impregnating a surrogate female animal with a SCNT embryo described herein. This technique is well known to a person of ordinary skill in the art. See, e.g., Seidel and Elsden, 1997, Embryo Transfer in Dairy Cattle, W. D. Hoard & Sons, Co., Hoards Dairyman. The embryo may be allowed to develop in utero, or alternatively, the fetus may be removed from the uterine environment before parturition.

The term “exogenous” refers to a substance present in a cell or organism other than its native source. For example, the terms “exogenous nucleic acid” or “exogenous protein” refer to a nucleic acid or protein that has been introduced by a process involving the hand of man into a biological system such as a cell or organism in which it is not normally found or in which it is found in lower amounts. A substance will be considered exogenous if it is introduced into a cell or an ancestor of the cell that inherits the substance. In contrast, the term “endogenous” refers to a substance that is native to the biological system or cell at that time. For instance, “exogenous DUX4/Dux/DUXC” refers to the introduction of DUX4/Dux/DUXC mRNA or cDNA which is not normally found or expressed in the cell or organism at that time.

The term “expression” refers to the cellular processes involved in producing RNA and proteins as applicable, for example, transcription, translation, folding, modification and processing. “Expression products” include RNA transcribed from a gene and polypeptides obtained by translation of mRNA transcribed from a gene.

A “genetically modified” or “engineered” cell refers to a cell into which an exogenous nucleic acid has been introduced by a process involving the hand of man (or a descendant of such a cell that has inherited at least a portion of the nucleic acid). The nucleic acid may for example contain a sequence that is exogenous to the cell, it may contain native sequences (i.e., sequences naturally found in the cells) but in a non-naturally occurring arrangement (e.g., a coding region linked to a promoter from a different gene), or altered versions of native sequences, etc. The process of transferring the nucleic into the cell can be achieved by any suitable technique. Suitable techniques include calcium phosphate or lipid-mediated transfection, electroporation, and transduction or infection using a viral vector. In some embodiments the polynucleotide or a portion thereof is integrated into the genome of the cell. The nucleic acid may have subsequently been removed or excised from the genome, provided that such removal or excision results in a detectable alteration in the cell relative to an unmodified but otherwise equivalent cell.

The term “identity” refers to the extent to which the sequence of two or more nucleic acids or polypeptides is the same. The percent identity between a sequence of interest and a second sequence over a window of evaluation, e.g., over the length of the sequence of interest, may be computed by aligning the sequences, determining the number of residues (nucleotides or amino acids) within the window of evaluation that are opposite an identical residue allowing the introduction of gaps to maximize identity, dividing by the total number of residues of the sequence of interest or the second sequence (whichever is greater) that fall within the window, and multiplying by 100. When computing the number of identical residues needed to achieve a particular percent identity, fractions are to be rounded to the nearest whole number. Percent identity can be calculated with the use of a variety of computer programs known in the art. For example, computer programs such as BLAST2, BLASTN, BLASTP, Gapped BLAST, etc., generate alignments and provide percent identity between sequences of interest. The algorithm of Karlin and Altschul (Karlin and Altschul, Proc. Natl. Acad. Sci. USA 87:22264-2268, 1990) modified as in Karlin and Altschul, Proc. Natl. Acad. Sci. USA 90:5873-5877, 1993 is incorporated into the NBLAST and XBLAST programs of Altschul et al. (Altschul, et al., J. Mol. Biol. 215:403-410, 1990). To obtain gapped alignments for comparison purposes, Gapped BLAST is utilized as described in Altschul et al. (Altschul, et al. Nucleic Acids Res. 25: 3389-3402, 1997). When utilizing BLAST and Gapped BLAST programs, the default parameters of the respective programs may be used. A PAM250 or BLOSUM62 matrix may be used. Software for performing BLAST analyses is publicly available through the National Center for Biotechnology Information (NCBI). See the Web site having URL www.ncbi.nlm.nih.gov for these programs. In a specific embodiment, percent identity is calculated using BLAST2 with default parameters as provided by the NCBI. In some embodiments, a nucleic acid or amino acid sequence has at least 80%, or at least about 85%, or at least about 90%, or at least about 95%, or at least about 98% or at least about 99% sequence identity to the nucleic acid or amino acid sequence.

The term “isolated” or “partially purified” as used herein refers, in the case of a nucleic acid or polypeptide, to a nucleic acid or polypeptide separated from at least one other component (e.g., nucleic acid or polypeptide) that is present with the nucleic acid or polypeptide as found in its natural source and/or that would be present with the nucleic acid or polypeptide when expressed by a cell, or secreted in the case of secreted polypeptides. A chemically synthesized nucleic acid or polypeptide or one synthesized using in vitro transcription/translation is considered “isolated”. An “isolated cell” is a cell that has been removed from an organism in which it was originally found or is a descendant of such a cell. Optionally the cell has been cultured in vitro, e.g., in the presence of other cells. Optionally the cell is later introduced into a second organism or re-introduced into the organism from which it (or the cell from which it is descended) was isolated.

The term “isolated population” with respect to an isolated population of cells as used herein refers to a population of cells that has been removed and separated from a mixed or heterogeneous population of cells. In some embodiments, an isolated population is a substantially pure population of cells as compared to the heterogeneous population from which the cells were isolated or enriched from.

The term “substantially pure”, with respect to a particular cell population, refers to a population of cells that is at least about 75%, preferably at least about 85%, more preferably at least about 90%, and most preferably at least about 95% pure, with respect to the cells making up a total cell population. Recast, the terms “substantially pure” or “essentially purified”, with regard to a population of definitive endoderm cells, refers to a population of cells that contain fewer than about 20%, more preferably fewer than about 15%, 10%, 8%, 7%, most preferably fewer than about 5%, 4%, 3%, 2%, 1%, or less than 1%, of cells that are not definitive endoderm cells or their progeny as defined by the terms herein. In some embodiments, the present disclosure encompasses methods to expand a population of definitive endoderm cells, wherein the expanded population of definitive endoderm cells is a substantially pure population of definitive endoderm cells. Similarly, with regard to a “substantially pure” or “essentially purified” population of SCNT-derived stem cells or pluripotent stem cells, refers to a population of cells that contain fewer than about 20%, more preferably fewer than about 15%, 10%, 8%, 7%, most preferably fewer than about 5%, 4%, 3%, 2%, 1%, or less than 1%, of cells that are not stem cell or their progeny as defined by the terms herein.

As used herein, the term “xenogeneic” refers to cells that are derived from a different species.

The terms “polypeptide” as used herein refers to a polymer of amino acids. The terms “protein” and “polypeptide” are used interchangeably herein. A peptide is a relatively short polypeptide, typically between about 2 and 60 amino acids in length. Polypeptides used herein typically contain amino acids such as the 20 L-amino acids that are most commonly found in proteins. However, other amino acids and/or amino acid analogs known in the art can be used. One or more of the amino acids in a polypeptide may be modified, for example, by the addition of a chemical entity such as a carbohydrate group, a phosphate group, a fatty acid group, a linker for conjugation, functionalization, etc. A polypeptide that has a non-polypeptide moiety covalently or non-covalently associated therewith is still considered a “polypeptide”. Exemplary modifications include glycosylation and palmitoylation.

Polypeptides may be purified from natural sources, produced using recombinant DNA technology, synthesized through chemical means such as conventional solid phase peptide synthesis, etc. The term “polypeptide sequence” or “amino acid sequence” as used herein can refer to the polypeptide material itself and/or to the sequence information (i.e., the succession of letters or three letter codes used as abbreviations for amino acid names) that biochemically characterizes a polypeptide. A polypeptide sequence presented herein is presented in an N-terminal to C-terminal direction unless otherwise indicated.

The term “functional fragment” or “biologically active fragment” as used herein with respect to a nucleic acid sequence refers to a nucleic acid sequence which is smaller in size than the nucleic acid sequence which it is a fragment of, where the nucleic acid sequence has about at least 50%, or 60% or 70% or at 80% or 90% or 100% or greater than 100%, for example 1.5-fold, 2-fold, 3-fold, 4-fold or greater than 4-fold the same biological action as the biologically active fragment from which it is a fragment of. Without being limited to theory, an exemplary example of a functional fragment of the nucleic acid sequence of the DUXC protein comprises a fragment of (e.g., wherein the fragment is at least 50%, 60%, 70%, 80%, 85%, 90%, 95%, 98%, or 99% as long as a sequence described herein) which has about at least 50%, or 60% or 70% or at 80% or 90% or 100% or greater than 100%, for example 1.5-fold, 2-fold, 3-fold, 4-fold or greater than 4-fold the ability to increase the efficiency of SCNT or reprogramming as compared to a control using the same method and under the same conditions.

The terms “treat”, “treating”, “treatment”, etc., as applied to an isolated cell, include subjecting the cell to any kind of process or condition or performing any kind of manipulation or procedure on the cell. As applied to a subject, the terms refer to providing medical or surgical attention, care, or management to an individual. The individual is usually ill (suffers from a disease or other condition warranting medical/surgical attention) or injured, or at increased risk of becoming ill relative to an average member of the population and in need of such attention, care, or management.

“Individual” is used interchangeably with “subject” herein. In any of the embodiments of the disclosure, the “individual” may be a human, e.g., one who suffers or is at risk of a disease for which cell therapy is of use (“indicated”).

The term “substantially similar” as used herein in reference to nuclear DNA sequences refers to two nuclear DNA sequences that are nearly identical. The two sequences may differ by copy error differences that normally occur during the replication of a nuclear DNA. Substantially similar DNA sequences are preferably greater than 97% identical, more-preferably greater than 98% identical, and most preferably greater than 99% identical. Identity is measured by dividing the number of identical residues in the two sequences by the total number of residues and multiplying the product by 100. Thus, two copies of exactly the same sequence have 100% identity, while sequences that are less highly conserved and have deletions, additions, or replacements have a lower degree of identity. Those of ordinary skill in the art will recognize that several computer programs are available for performing sequence comparisons and determining sequence identity.

The terms “lower”,“reduced”,“reduction” or “decrease” or “inhibit” are all used herein generally to mean a decrease by a statistically significant amount. However, for avoidance of doubt, “lower”,“reduced”,“reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

The terms “increased” ,“increase” or “enhance” or “activate” are all used herein to generally mean an increase by a statically significant amount; for the avoidance of any doubt, the terms “increased”,“increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “statistically significant” or “significantly” refers to statistical significance and generally means a two standard deviation (2SD) below normal, or lower, concentration of the marker. The term refers to statistical evidence that there is a difference. It is defined as the probability of making a decision to reject the null hypothesis when the null hypothesis is actually true. The decision is often made using the p-value.

The term “xeno-free (XF)” or “animal component-free (ACF)” or “animal free,” when used in relation to a medium, an extracellular matrix, or a culture condition, refers to a medium, an extracellular matrix, or a culture condition which is essentially free from heterogeneous animal-derived components. For culturing human cells, any proteins of a non-human animal, such as mouse, would be xeno components. In certain aspects, the xeno-free matrix may be essentially free of any non-human animal-derived components, therefore excluding mouse feeder cells or Matrigel™. Matrigel™ is a solubilized basement membrane preparation extracted from the Engelbreth-Holm-Swarm (EHS) mouse sarcoma, a tumor rich in extracellular matrix proteins to include laminin (a major component), collagen IV, heparin sulfate proteoglycans, and entactin/nidogen.

Cells are “substantially free” of certain reagents or elements, such as serum, signaling inhibitors, animal components or feeder cells, exogenous genetic elements or vector elements, as used herein, when they have less than 10% of the element(s), and are “essentially free” of certain reagents or elements when they have less than 1% of the element(s). However, even more desirable are cell populations wherein less than 0.5% or less than 0.1% of the total cell population comprise exogenous genetic elements or vector elements.

A “vector ” or “construct” (sometimes referred to as gene delivery or gene transfer “vehicle”) refers to a macromolecule, complex of molecules, or viral particle, comprising a polynucleotide to be delivered to a host cell, either in vitro or in vivo. The polynucleotide can be a linear or a circular molecule.

A “plasmid”, a common type of a vector, is an extra-chromosomal DNA molecule separate from the chromosomal DNA which is capable of replicating independently of the chromosomal DNA. In certain cases, it is circular and double-stranded.

By “expression construct” or “expression cassette” is meant a nucleic acid molecule that is capable of directing transcription. An expression construct includes, at the least, a promoter or a structure functionally equivalent to a promoter. Additional elements, such as an enhancer, and/or a transcription termination signal, may also be included.

The term “corresponds to” is used herein to mean that a polynucleotide sequence is homologous (i.e., is identical, not strictly evolutionarily related) to all or a portion of a reference polynucleotide sequence, or that a polypeptide sequence is identical to a reference polypeptide sequence. In contradistinction, the term “complementary to” is used herein to mean that the complementary sequence is homologous to all or a portion of a reference polynucleotide sequence. For illustration, the nucleotide sequence “TATAC” corresponds to a reference sequence “TATAC” and is complementary to a reference sequence “GTATA”.

A “gene,” “polynucleotide,” “coding region,” “sequence,” “segment,” “fragment,” or “transgene” which “encodes” a particular protein, is a nucleic acid molecule which is transcribed and optionally also translated into a gene product, e.g., a polypeptide, in vitro or in vivo when placed under the control of appropriate regulatory sequences. The coding region may be present in either a cDNA, genomic DNA, or RNA form. When present in a DNA form, the nucleic acid molecule may be single-stranded (i.e., the sense strand) or double-stranded. The boundaries of a coding region are determined by a start codon at the 5′ (amino) terminus and a translation stop codon at the 3′ (carboxy) terminus. A gene can include, but is not limited to, cDNA from prokaryotic or eukaryotic mRNA, genomic DNA sequences from prokaryotic or eukaryotic DNA, and synthetic DNA sequences. A transcription termination sequence will usually be located 3′ to the gene sequence.

The term “cell” is herein used in its broadest sense in the art and refers to a living body which is a structural unit of tissue of a multicellular organism, is surrounded by a membrane structure which isolates it from the outside, has the capability of self-replicating, and has genetic information and a mechanism for expressing it. Cells used herein may be naturally-occurring cells or artificially modified cells (e.g., fusion cells, genetically modified cells, etc.).

As used herein, the term “stem cell” refers to a cell capable of self-replication and pluripotency or multipotency. Typically, stem cells can regenerate an injured tissue. Stem cells herein may be, but are not limited to, embryonic stem (ES) cells, induced pluripotent stem cells or tissue stem cells (also called tissue-specific stem cell, or somatic stem cell). ES cells refers to pluripotent cells derived from the inner cell mass of blastocysts or morulae that have been serially passaged as cell lines. The ES cells may be derived from fertilization of an egg cell with sperm or DNA, nuclear transfer, e.g., SCNT, parthenogenesis etc. The term “human embryonic stem cells” (hES cells) refers to human ES cells. The term “ntESC” refers to embryonic stem cells obtained from the inner cell mass of blastocysts or morulae produced from SCNT. The generation of ESC is disclosed in U.S. Pat. Nos. 5,843,780, 6,200,806, and ESC obtained from the inner cell mass of blastocysts derived from somatic cell nuclear transfer are described in U.S. Pat. Nos. 5,945,577, 5,994,619, 6,235,970, which are incorporated herein in their entirety by reference. The distinguishing characteristics of an embryonic stem cell define an embryonic stem cell phenotype. Accordingly, a cell has the phenotype of an embryonic stem cell if it possesses one or more of the unique characteristics of an embryonic stem cell such that that cell can be distinguished from other cells. Exemplary distinguishing embryonic stem cell characteristics include, without limitation, gene expression profile, proliferative capacity, differentiation capacity, karyotype, responsiveness to particular culture conditions, and the like.

Unlike ES cells, tissue stem cells have a limited differentiation potential. Tissue stem cells are present at particular locations in tissues and have an undifferentiated intracellular structure. Therefore, the pluripotency of tissue stem cells is typically low. Tissue stem cells have a higher nucleus/cytoplasm ratio and have few intracellular organelles. Most tissue stem cells have low pluripotency, a long cell cycle, and proliferative ability beyond the life of the individual. Tissue stem cells are separated into categories, based on the sites from which the cells are derived, such as the dermal system, the digestive system, the bone marrow system, the nervous system, and the like. Tissue stem cells in the dermal system include epidermal stem cells, hair follicle stem cells, and the like. Tissue stem cells in the digestive system include pancreatic (common) stem cells, liver stem cells, and the like. Tissue stem cells in the bone marrow system include hematopoietic stem cells, mesenchymal stem cells, and the like. Tissue stem cells in the nervous system include neural stem cells, retinal stem cells, and the like.

“Induced pluripotent stem cells,” commonly abbreviated as iPS cells or iPSCs, refer to a type of pluripotent stem cell artificially prepared from a non-pluripotent cell, typically an adult somatic cell, or terminally differentiated cell, such as fibroblast, a hematopoietic cell, a myocyte, a neuron, an epidermal cell, or the like, by introducing certain factors, referred to as reprogramming factors.

The term “pluripotent” as used herein refers to a cell with the capacity, under different conditions, to differentiate to more than one differentiated cell type, and preferably to differentiate to cell types characteristic of all three germ cell layers in the embryo proper. Pluripotent cells are characterized primarily by their ability to differentiate to more than one cell type, preferably to all three germ layers, using, for example, a nude mouse teratoma formation assay. Such cells include hES cells, human embryo-derived cells (hEDCs), human SCNT-embryo derived stem cells and adult-derived stem cells. Pluripotent stem cells may be genetically modified or not genetically modified. Genetically modified cells may include markers such as fluorescent proteins to facilitate their identification. Pluripotency is also evidenced by the expression of embryonic stem (ES) cell markers, although the preferred test for pluripotency is the demonstration of the capacity to differentiate into cells of each of the three germ layers. It should be noted that simply culturing such cells does not, on its own, render them pluripotent. Reprogrammed pluripotent cells (e.g. iPS cells as that term is defined herein) also have the characteristic of the capacity of extended passaging without loss of growth potential, relative to primary cell parents, which generally have capacity for only a limited number of divisions in culture.

The term “totipotent” as used herein in reference to SCNT embryos refers to SCNT embryos that can develop into a live born animal and also in reference to the reprogramming methods refers to a cell that retains the ability to become any embryonic or extraembryonic cell type. Totipotent cells are also cells that are in a 2-cell or 4-cell, early cleavage state.

By “operably linked” with reference to nucleic acid molecules is meant that two or more nucleic acid molecules (e.g., a nucleic acid molecule to be transcribed, a promoter, and an enhancer element) are connected in such a way as to permit transcription of the nucleic acid molecule. “Operably linked” with reference to peptide and/or polypeptide molecules is meant that two or more peptide and/or polypeptide molecules are connected in such a way as to yield a single polypeptide chain, i.e., a fusion polypeptide, having at least one property of each peptide and/or polypeptide component of the fusion. The fusion polypeptide is particularly chimeric, i.e., composed of heterologous molecules.

The terms “naïve” and “primed” as used herein with respect to stem cells relate to terms known in the art and describe distinct stem cell phentoypes. For example, the following table from Weinberger et al., Nature Reviews Molecular Cell Biology. (2016), 17, 55-169, which is herein incorporated by reference, describes differential characteristics of primed or naïve stem cells:

| | Naïve | Primed |
|---|---|---|
| | pluripotent | pluripotent |
| Pluripotent cell property | cell | cell |
| MEK-ERK dependence | No | Yes |
| Long-term dependence on | No | Yes |
| FGF2 signaling | | |
| Long-term dependence on | No | Yes |
| TGF-gena or Activin A | | |
| signalling | | |
| Dominant OCT4 enhancer | Distal | Proximal |
| H3K27me3 on developmental | Low | High |
| regulators | | |
| Global DNA hypomethylation | Yes | No |
| X chromosome inactivation | No | Yes |
| Dependence on DNMT1, | No | Yes |
| DICER, METTL3, MBD3 | | |
| Priming markers (OTX2, | decreased | increased |
| ZIC2) | | |
| Pluripotency markers | increased | decreased |
| (NANOG, KLFs, ESRR-beta) | | |
| CD24/MHC class 1 | Low/low | High/mod |
| Expressed adhesion molecules | E-cadherin | N-cadherin |
| Promotion of pluripotency | Yes | No |
| maintenance by NANOG or | | |
| PRDM14 | | |
| Metabolism | OxPhos, Glycolytic | Glycolytic |
| Competence as initial starting | High | Low |
| cells for PGCLC induction | | |
| Capacity for colonization of | High | Low |
| host pre-implantation ICM and | | |
| contribution to advanced | | |
| embryonic chimeras | | |
| Hypomethylation of promoter | Yes | No |
| and enhancer regions | | |
| KIT | Yes | No |
| Tolerance for absence of | Yes | No |
| exogenous L-glutamine | | |
| Mitochondrial membrane | High | Low |
| activity and depolarization | | |
| Competence as initial starting | High | Low |
| cell for TSC induction | | |

As used herein the term “comprising” or “comprises” is used in reference to compositions, methods, and respective component(s) thereof, that are essential to the disclosure, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the disclosure.

The term “consisting of” refers to compositions, methods, and respective components thereof as described herein, which are exclusive of any element not recited in that description of the embodiment.

As used in this specification and the appended claims, the singular forms “a”, “an”, and “the” include plural references unless the context clearly dictates otherwise. Thus for example, references to “the method” includes one or more methods, and/or steps of the type described herein and/or which will become apparent to those persons skilled in the art upon reading this disclosure and so forth.

### II. DUXC Double Homeodomain Proteins

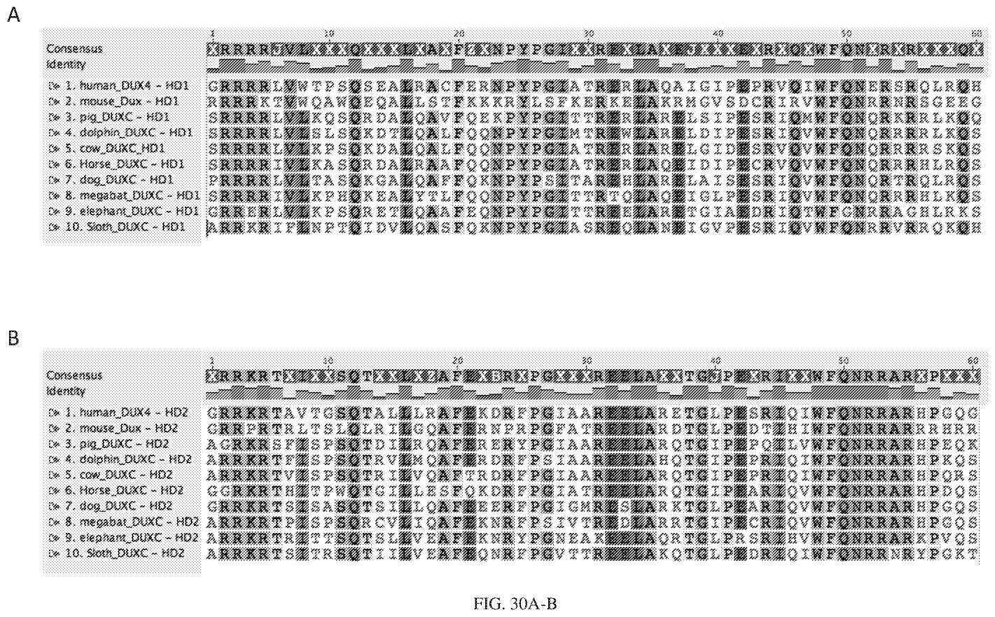

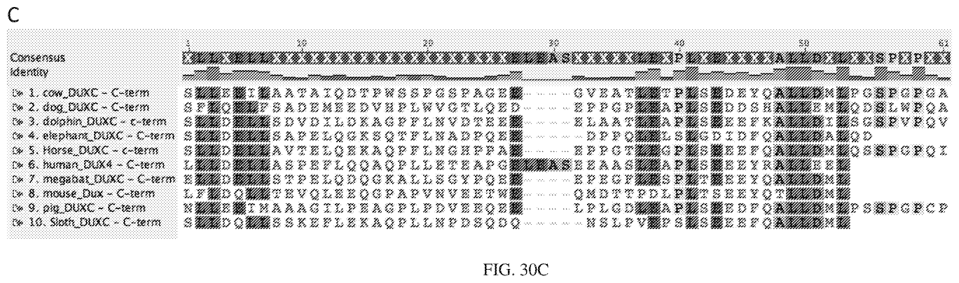

DUXC double homeodomain proteins are transcription factors. In humans, DUX4 is a DUX double homeodomain gene located within a D4Z4 repeat array in the subtelomeric region of chromosome 4q35. The D4Z4 repeat is polymorphic in length; a similar D4Z4 repeat array has been identified on chromosome 10. Each D4Z4 repeat unit has an open reading frame (named DUX4) that contains two homeodomains. DUX4 is a retrogene that arose from the retroposition of the parental DUXC gene. Each eutherian mammal has a DUXC ortholog, either as an intact gene or as a retrogene. Mice have a retroposed DUXC gene named Dux. Dogs, cows, horses and pigs have a DUXC gene that has not undergone retroposition. Alignments of homeodomain 1 and homeodomain 2 from various species is shown in FIG. 30A-B. Also shown is a consensus homeodomain. In some embodiments, the DUXC protein comprises a polypeptide comprising the consensus sequence shown for homeodomain 1 (FIG. 30A) and homeodomain 2 (FIG. 30B).

The common function of the DUXC-family in activating transcription of the early cleavage gene signature in different species is not obvious because of divergence of the DNA sequence encoding family members among eutherians. As shown in FIG. 30, a consensus sequence can be generated for the first (HD1) and second (HD2) homeodomains for DUXC-family members in representative eutherian species. FIG. 30 and the table below shows that there is at least 28% identity to this consensus sequence in the first homeodomain and at least 48% identity in the second homeodomain. A similar comparison performing pairwise alignments among representative DUXC-family members in eutherians shown in Table 2 (“Defining DUX4/C family using pairwise identity cutoff”) that does not rely on generating a consensus sequence, shows that there is at least 35% identity in the first homeodomain and 55% identify in the second homeodomain. As shown in FIG. 30C, the DUXC-family also contains one or more regions encoding the amino acid sequence LLxxL, where L represents leucine and X represents any amino acid. This region can occur in an exon that is alternatively used in different RNA transcripts from the DUXC-family gene locus and does not need to be present in all transcripts isoforms.

The percent identity to the consensus homeodomain 1 and 2 are shown in the Table 1 below:

TABLE 1

| Species | % Identity to Consensus HD1 | % Identity to Consensus HD2 |
|---|---|---|
| human | 28 (46.7%) | 35 (58.3%) |
| mouse | 17 (28.3%) | 29 (48.3%) |
| pig | 29 (48.3%) | 33 (55.0%) |
| dolphin | 30 (50.0%) | 35 (58.3%) |
| cow | 30 (50.0%) | 35 (58.3%) |
| horse | 30 (50.0%) | 32 (53.3%) |
| dog | 29 (48.3%) | 35 (58.3%) |
| megabat | 28 (46.7%) | 33 (55.0%) |
| elephant | 27 (45.0%) | 34 (56.7%) |
| sloth | 29 (48.3%) | 35 (58.3%) |

A similar comparison performing pairwise alignments among representative DUXC-family members in eutherians shown in the tables below, that does not rely on generating a consensus sequence, shows that there is at least 35% identity in the first homeodomain and 55% identify in the second homeodomain.

TABLE 2

| | Human | Mouse | Canine | cow | sloth |
|---|---|---|---|---|---|
| HD1 | DUX4 | DUX | DUXC | DUXC | DUXC |
| Human | 100% | x | x | x | x |
| DUX4 | | | | | |
| Mouse DUX | 35% | 100% | x | x | x |
| Canine | 60% | 41.7% | 100% | x | x |
| DUXC | | | | | |
| cow DUXC | 65% | 35% | 71.7% | 100% | x |
| sloth DUXC | 55% | 36.7% | 56.7% | 65% | 100% |
| | Human | Mouse | Canine | cow | sloth |
| HD2 | DUX4 | DUX | DUXC | DUXC | DUXC |
| Human | 100% | x | x | x | x |
| DUX4 | | | | | |
| Mouse DUX | 58.3% | 100% | x | x | x |
| Canine | 78.3% | 55% | 100% | x | x |
| DUXC | | | | | |
| cow DUXC | 71.7% | 60% | 71.7% | 100% | x |
| sloth DUXC | 66.7% | 58.3% | 66.7% | 68.3% | 100% |

In some embodiments, the DUXC protein comprises at least, at most, or exactly 10, 20, 25, 30, 35, 40, 45, 50, 55, 60, 65, 70, 75, 80, 81, 82, 83, 84, 85, 86, 87, 88, 89, 90, 91, 92, 93, 94, 95, 96, 97, 98, 99, or 100% identity (or any derivable range therein) to a polypeptide sequence of the disclosure or to a nucleic acid encoding a polypeptide as described herein. In some embodiments, the DUXC protein comprises a homeodomain 1 comprising at least, at most, or exactly 10, 20, 25, 30, 35, 40, 45, 50, 55, 60, 65, 70, 75, 80, 81, 82, 83, 84, 85, 86, 87, 88, 89, 90, 91, 92, 93, 94, 95, 96, 97, 98, 99, or 100% identity (or any derivable range therein) to a homeodomain 1 sequence of the disclosure or the consensus of FIG. 30A. In some embodiments, the DUXC protein comprises a homeodomain 2 comprising at least, at most, or exactly 10, 20, 25, 30, 35, 40, 45, 50, 55, 60, 65, 70, 75, 80, 81, 82, 83, 84, 85, 86, 87, 88, 89, 90, 91, 92, 93, 94, 95, 96, 97, 98, 99, or 100% identity (or any derivable range therein) to a homeodomain 2 sequence of the disclosure or the consensus of FIG. 30B. In some embodiments, the DUXC protein comprises a LLxxL motif at the C-terminus. In some embodiments, the DUXC protein comprises at least 25% identity to the homeodomain 1 consensus sequence of FIG. 30A. In some embodiments, the DUXC protein comprises at least 45% identity to the homeodomain 2 consensus sequence of FIG. 30B.

Below are exemplary DUXC double homeodomain proteins from different animals. An exemplary human DUXC ortholog, the DUX4 double homeodomain protein (DUX4; NCBI Reference Sequence: NC_000004.12) may be encoded by a nucleic acid comprising the following sequence (SEQ ID NO:81):

ATGGCCCTCCCGACACCCTCGGACAGCACCCTCCCCGCGGAAGCCCGGGG
ACGAGGACGGCGACGGAGACTCGTTTGGACCCCGAGCCAAAGCGAGGCCC
TGCGAGCCTGCTTTGAGCGGAACCCGTACCCGGGCATCGCCACCAGAGAA
CGGCTGGCCCAGGCCATCGGCATTCCGGAGCCCAGGGTCCAGATTTGGTT
TCAGAATGAGAGGTCACGCCAGCTGAGGCAGCACCGGCGGGAATCTCGGC
CCTGGCCCGGGAGACGCGGCCCGCCAGAAGGCCGGCGAAAGCGGACCGCC
GTCACCGGATCCCAGACCGCCCTGCTCCTCCGAGCCTTTGAGAAGGATCG
CTTTCCAGGCATCGCCGCCCGGGAGGAGCTGGCCAGAGAGACGGGCCTCC
CGGAGTCCAGGATTCAGATCTGGTTTCAGAATCGAAGGGCCAGGCACCCG
GGACAGGGTGGCAGGGCGCCCGCGCAGGCAGGCGGCCTGTGCAGCGCGGC
CCCCGGCGGGGGTCACCCTGCTCCCTCGTGGGTCGCCTTCGCCCACACCG
GCGCGTGGGGAACGGGGCTTCCCGCACCCCACGTGCCCTGCGCGCCTGGG
GCTCTCCCACAGGGGGCTTTCGTGAGCCAGGCAGCGAGGGCCGCCCCCGC
GCTGCAGCCCAGCCAGGCCGCGCCGGCAGAGGGGATCTCCCAACCTGCCC
CGGCGCGCGGGGATTTCGCCTACGCCGCCCCGGCTCCTCCGGACGGGGCG
CTCTCCCACCCTCAGGCTCCTCGCTGGCCTCCGCACCCGGGCAAAAGCCG
GGAGGACCGGGACCCGCAGCGCGACGGCCTGCCGGGCCCCTGCGCGGTGG
CACAGCCTGGGCCCGCTCAAGCGGGGCCGCAGGGCCAAGGGGTGCTTGCG
CCACCCACGTCCCAGGGGAGTCCGTGGTGGGGCTGGGGCCGGGGTCCCCA
GGTCGCCGGGGCGGCGTGGGAACCCCAAGCCGGGGCAGCTCCACCTCCCC
AGCCCGCGCCCCCGGACGCCTCCGCCTCCGCGCGGCAGGGGCAGATGCAA
GGCATCCCGGCGCCCTCCCAGGCGCTCCAGGAGCCGGCGCCCTGGTCTGC
ACTCCCCTGCGGCCTGCTGCTGGATGAGCTCCTGGCGAGCCCGGAGTTTC
TGCAGCAGGCGCAACCTCTCCTAGAAACGGAGGCCCCGGGGGAGCTGGAG
GCCTCGGAAGAGGCCGCCTCGCTGGAAGCACCCCTCAGCGAGGAAGAATA
CCGGGCTCTGCTGGAGGAGCTTTAG

A human DUX4 double homeodomain protein may also be encoded by a nucleic acid comprising the following sequence (SEQ ID NO:82):

ATGGCATTGCCTACACCTTCAGACTCTACGCTGCCTGCAGAGGCTAGGGG
AAGAGGTAGACGGCGGCGATTGGTGTGGACTCCATCACAATCCGAAGCTC
TTCGCGCATGCTTCGAGCGCAATCCCTATCCGGGGATTGCCACAAGGGAG
AGGCTTGCACAGGCTATCGGAATCCCGGAACCGAGAGTGCAGATCTGGTT
CCAAAATGAACGCTCTCGGCAGCTCAGACAGCATCGCAGGGAGTCCCGCC
CGTGGCCAGGAAGAAGGGGACCACCTGAAGGAAGAAGAAAACGCACAGCG
GTGACTGGCAGCCAAACGGCTCTGCTGCTCCGCGCTTTCGAGAAAGATCG
GTTCCCCGGAATTGCCGCACGCGAAGAACTCGCCAGAGAAACTGGGCTCC
CAGAATCACGAATACAGATTTGGTTCCAGAACCGCAGAGCAAGACACCCA
GGCCAGGGGGGACGGGCACCTGCTCAGGCCGGTGGACTCTGCTCTGCTGC
CCCTGGGGGCGGCCATCCAGCACCTTCCTGGGTGGCTTTCGCTCATACTG
GCGCTTGGGGTACCGGGCTGCCTGCTCCGCATGTTCCCTGTGCTCCAGGG
GCCCTCCCGCAGGGAGCGTTTGTTTCCCAGGCAGCTAGGGCTGCACCTGC
CCTGCAACCATCACAGGCAGCGCCAGCTGAAGGCATCAGCCAACCCGCCC
CAGCCCGCGGAGATTTTGCTTATGCAGCGCCAGCACCTCCAGACGGTGCC
CTGAGCCACCCCCAAGCCCCCAGATGGCCCCCTCACCCTGGTAAGTCCCG
GGAAGACCGCGATCCCCAACGAGATGGACTGCCCGGTCCTTGCGCTGTGG
CCCAGCCAGGACCTGCTCAAGCCGGCCCTCAGGGGCAAGGAGTGCTGGCC
CCACCTACAAGCCAGGGATCTCCCTGGTGGGGTTGGGGACGCGGACCTCA
GGTTGCTGGAGCCGCTTGGGAGCCTCAGGCCGGAGCTGCACCGCCGCCAC
AACCGGCCCCTCCCGACGCGTCAGCGTCCGCCCGACAAGGCCAGATGCAG
GGAATCCCAGCACCTAGCCAAGCTCTTCAAGAGCCTGCCCCTTGGAGCGC
ACTGCCGTGTGGGCTGCTCCTGGATGAACTCCTGGCTAGCCCAGAATTTC
TCCAGCAGGCACAGCCACTCCTGGAAACAGAAGCTCCGGGAGAGCTCGAA
GCCTCCGAAGAAGCAGCAAGCCTGGAGGCACCTCTTTCCGAGGAGGAGTA
TAGAGCCCTTCTGGAAGAACTTTGA

The amino acid sequence of the human DUX4 (NCBI Reference Sequence: NC_000004.12) may comprise the following (SEQ ID NO:83):

MALPTPSDSTLPAEARGRGRRRRLVWTPSQSEALRACFERNPYPGIATRE
RLAQAIGIPEPRVQIWFQNERSRQLRQHRRESRPWPGRRGPPEGRRKRTA
VTGSQTALLLRAFEKDRFPGIAAREELARETGLPESRIQIWFQNRRARHP
GQGGRAPAQAGGLCSAAPGGGHPAPSWVAFAHTGAWGTGLPAPHVPCAPG
ALPQGAFVSQAARAAPALQPSQAAPAEGISQPAPARGDFAYAAPAPPDGA
LSHPQAPRWPPHPGKSREDRDPQRDGLPGPCAVAQPGPAQAGPQGQGVLA
PPTSQGSPWWGWGRGPQVAGAAWEPQAGAAPPPQPAPPDASASARQGQMQ
GIPAPSQALQEPAPWSALPCGLLLDELLASPEFLQQAQPLLETEAPGELE
ASEEAASLEAPLSEEEYRALLEEL*

The amino acid sequence of the hDUX4 homeodomain 1 comprises: GRRRRLVWTPSQSEALRACFERNPYPGIATRERLAQAIGIPEPRVQIWFQNERSRQLR QH (SEQ ID NO:84). The amino acid sequence of the hDUX4 homeodomain 2 comprises: GRRKRTAVTGSQTALLLRAFEKDRFPGIAAREELARETGLPESRIQIWFQNRRARHPG QG (SEQ ID NO:85). The amino acid sequence of the hDUX4 Conserved C-terminal domain comprises LLLDELLASPEFLQQAQPLLETEAPGELEASEEAASLEAPLSEEEYRALLEEL (SEQ ID NO:86).

An exemplary mouse DUXC orhtolog, the mouse DUX double homeodomain containing protein (DUX; NCBI Reference Sequence: NM_001081954.1) may be encoded by a nucleic acid comprising the following sequence (SEQ ID NO:87):

ATGGCAGAAGCTGGCAGCCCTGTTGGTGGCAGTGGTGTGGCACGGGAATC
CCGGCGGCGCAGGAAGACGGTTTGGCAGGCCTGGCAAGAGCAGGCCCTGC
TATCAACTTTCAAGAAGAAGAGATACCTGAGCTTCAAGGAGAGGAAGGAG
CTGGCCAAGCGAATGGGGGTCTCAGATTGCCGCATCCGCGTGTGGTTTCA
GAACCGCAGGAATCGCAGTGGAGAGGAGGGGCATGCCTCAAAGAGGTCCA
TCAGAGGCTCCAGGCGGCTAGCCTCGCCACAGCTCCAGGAAGAGCTTGGA
TCCAGGCCACAGGGTAGAGGCATGCGCTCATCTGGCAGAAGGCCTCGCAC
TCGACTCACCTCGCTACAGCTCAGGATCCTAGGGCAAGCCTTTGAGAGGA
ACCCACGACCAGGCTTTGCTACCAGGGAGGAGCTGGCGCGTGACACAGGG
TTGCCCGAGGACACGATCCACATATGGTTTCAAAACCGAAGAGCTCGGCG
GCGCCACAGGAGGGGCAGGCCCACAGCTCAAGATCAAGACTTGCTGGCGT
CACAAGGGTCGGATGGGGCCCCTGCAGGTCCGGAAGGCAGAGAGCGTGAA
GGTGCCCAGGAGAACTTGTTGCCACAGGAAGAAGCAGGAAGTACGGGCAT
GGATACCTCGAGCCCTAGCGACTTGCCCTCCTTCTGCGGAGAGTCCCAGC
CTTTCCAAGTGGCACAGCCCCGTGGAGCAGGCCAACAAGAGGCCCCCACT
CGAGCAGGCAACGCAGGCTCTCTGGAACCCCTCCTTGATCAGCTGCTGGA
TGAAGTCCAAGTAGAAGAGCCTGCTCCAGCCCCTCTGAATTTGGATGGAG
ACCCTGGTGGCAGGGTGCATGAAGGTTCCCAGGAGAGCTTTTGGCCACAG
GAAGAAGCAGGAAGTACAGGCATGGATACTTCTAGCCCCAGCGACTCAAA
CTCCTTCTGCAGAGAGTCCCAGCCTTCCCAAGTGGCACAGCCCTGTGGAG
CGGGCCAAGAAGATGCCCGCACTCAAGCAGACAGCACAGGCCCTCTGGAA
CTCCTCCTCCTTGATCAACTGCTGGACGAAGTCCAAAAGGAAGAGCATGT
GCCAGTCCCACTGGATTGGGGTAGAAATCCTGGCAGCAGGGAGCATGAAG
GTTCCCAGGACAGCTTACTGCCCCTGGAGGAAGCAGTAAATTCGGGCATG
GATACCTCGATCCCTAGCATCTGGCCAACCTTCTGCAGAGAATCCCAGCC
TCCCCAAGTGGCACAGCCCTCTGGACCAGGCCAAGCACAGGCCCCCACTC
AAGGTGGGAACACGGACCCCCTGGAGCTCTTCCTCTATCAACTGTTGGAT
GAAGTCCAAGTAGAAGAGCATGCTCCAGCCCCTCTGAATTGGGATGTAGA
TCCTGGTGGCAGGGTGCATGAAGGTTCGTGGGAGAGCTTTTGGCCACAGG
AAGAAGCAGGAAGTACAGGCCTGGATACTTCAAGCCCCAGCGACTCAAAC
TCCTTCTTCAGAGAGTCCAAGCCTTCCCAAGTGGCACAGCGCCGTGGAGC
GGGCCAAGAAGATGCCCGCACTCAAGCAGACAGCACAGGCCCTCTGGAAC
TCCTCCTCTTTGATCAACTGCTGGACGAAGTCCAAAAGGAAGAGCATGTG
CCAGCCCCACTGGATTGGGGTAGAAATCCTGGCAGCATGGAGCATGAAGG
TTCCCAGGACAGCTTACTGCCCCTGGAGGAAGCAGCAAATTCGGGCAGGG
ATACCTCGATCCCTAGCATCTGGCCAGCCTTCTGCAGAAAATCCCAGCCT
CCCCAAGTGGCACAGCCCTCTGGACCAGGCCAAGCACAGGCCCCCATTCA
AGGTGGGAACACGGACCCCCTGGAGCTCTTCCTTGATCAACTGCTGACCG
AAGTCCAACTTGAGGAGCAGGGGCCTGCCCCTGTGAATGTGGAGGAAACA
TGGGAGCAAATGGACACAACACCTGATCTGCCTCTCACTTCAGAAGAATA
TCAGACTCTTCTAGATATGCTCTGA

An exemplary mouse DUX double homeodomain containing protein (DUX; NCBI Reference Sequence: NM_001081954.1) may also be encoded by a nucleic acid comprising the following sequence (SEQ ID NO:88):

ATGGCTGAGGCTGGCTCTCCAGTGGGAGGATCTGGAGTGGCCAGAGAATC
AAGGAGAAGGAGGAAAACTGTCTGGCAAGCTTGGCAGGAACAGGCACTCC
TGAGCACATTTAAGAAAAAAAGGTATCTGTCCTTTAAAGAAAGAAAGGAA
CTGGCAAAAAGGATGGGAGTTTCTGATTGCAGGATCAGAGTCTGGTTCCA
GAATAGGAGAAATAGGTCTGGGGAGGAAGGACATGCAAGCAAGAGAAGCA
TAAGAGGTTCCAGGAGGCTGGCATCCCCTCAACTTCAGGAGGAACTGGGA
AGTAGGCCCCAAGGCAGGGGCATGAGGTCCTCAGGGAGGAGACCCAGAAC
CAGGCTGACAAGTCTGCAGCTGAGAATCCTTGGTCAGGCTTTTGAAAGGA
ATCCAAGGCCAGGATTTGCCACCAGAGAGGAACTGGCCAGGGATACAGGC
CTTCCTGAGGATACTATCCATATCTGGTTCCAGAACAGGAGGGCCAGGAG
AAGGCACAGAAGGGGAAGACCTACAGCCCAGGACCAGGACCTCCTGGCTT
CCCAGGGTTCTGATGGAGCACCTGCTGGGCCTGAAGGTAGAGAGAGAGAA
GGAGCACAGGAAAATTTGCTGCCCCAGGAGGAGGCAGGATCAACAGGGAT
GGACACCTCAAGCCCTTCTGACCTCCCTTCATTCTGTGGTGAATCACAGC
CCTTTCAGGTGGCCCAGCCCAGGGGAGCTGGACAGCAGGAGGCTCCCACA
AGGGCAGGGAATGCTGGATCATTGGAGCCACTGTTGGACCAGCTCTTGGA
TGAGGTCCAGGTGGAGGAACCTGCCCCAGCTCCACTCAACCTGGATGGTG
ATCCTGGGGGGAGGGTTCATGAGGGTAGTCAGGAGTCCTTCTGGCCCCAG
GAGGAGGCTGGTTCTACTGGAATGGACACTTCTTCACCCTCTGACAGCAA
TAGCTTTTGCAGGGAGAGTCAACCCTCTCAGGTAGCTCAGCCTTGTGGGG
CTGGCCAGGAGGATGCTAGGACCCAGGCTGACTCAACAGGGCCCTTGGAG
CTGTTGCTGCTGGACCAGCTCCTGGATGAGGTACAGAAGGAGGAACATGT
ACCAGTGCCCCTGGACTGGGGGAGGAACCCTGGAAGCAGAGAACATGAGG
GTAGTCAGGATTCTCTCCTTCCTCTGGAAGAGGCTGTGAATTCTGGAATG
GACACTAGTATACCAAGTATTTGGCCTACATTTTGCAGGGAGTCACAACC
CCCACAGGTGGCTCAGCCTTCAGGACCTGGGCAGGCCCAGGCTCCTACCC
AAGGGGGTAATACAGACCCACTGGAACTCTTTCTGTATCAGCTGCTGGAT
GAGGTCCAGGTGGAGGAACATGCCCCAGCTCCACTCAACTGGGATGTGGA
TCCAGGGGGCAGAGTCCATGAGGGTTCCTGGGAGTCATTCTGGCCCCAGG
AGGAGGCAGGCTCTACAGGACTGGACACAAGCTCCCCTAGTGACAGCAAC
TCATTCTTTAGGGAGAGTAAGCCCTCTCAGGTTGCTCAAAGGAGGGGAGC
TGGGCAAGAGGATGCCAGGACTCAGGCTGACAGTACAGGACCCCTGGAGC
TGCTGTTGTTTGACCAGCTCCTGGATGAAGTGCAGAAGGAGGAACATGTT
CCAGCTCCCCTGGACTGGGGAAGGAACCCTGGTTCTATGGAACATGAGGG
CTCTCAGGACTCTCTCTTGCCTCTGGAAGAAGCTGCTAATAGTGGCAGAG
ATACAAGTATCCCAAGCATTTGGCCTGCCTTTTGCAGGAAAAGCCAGCCA
CCCCAGGTAGCCCAGCCTAGTGGACCTGGACAGGCTCAGGCACCTATACA
AGGAGGCAACACTGACCCATTGGAGTTGTTTCTGGACCAGCTGCTCACTG
AGGTGCAACTGGAGGAACAAGGGCCAGCACCTGTCAATGTTGAAGAGACC
TGGGAACAGATGGATACCACTCCAGACTTGCCACTGACTTCTGAAGAGTA
CCAGACCCTTCTTGACATGCTGTAA

The amino acid sequence of the mouse DUX (NCBI Reference Sequence: NM_001081954.1) may comprise the following (SEQ ID NO:89):

MAEAGSPVGGSGVARESRRRRKTVWQAWQEQALLSTFKKKRYLSFKERKE
LAKRMGVSDCRIRVWFQNRRNRSGEEGHASKRSIRGSRRLASPQLQEELG
SRPQGRGMRSSGRRPRTRLTSLQLRILGQAFERNPRPGFATREELARDTG
LPEDTIHIWFQNRRARRRHRRGRPTAQDQDLLASQGSDGAPAGPEGRERE
GAQENLLPQEEAGSTGMDTSSPSDLPSFCGESQPFQVAQPRGAGQQEAPT
RAGNAGSLEPLLDQLLDEVQVEEPAPAPLNLDGDPGGRVHEGSQESFWPQ
EEAGSTGMDTSSPSDSNSFCRESQPSQVAQPCGAGQEDARTQADSTGPLE
LLLLDQLLDEVQKEEHVPVPLDWGRNPGSREHEGSQDSLLPLEEAVNSGM
DTSIPSIWPTFCRESQPPQVAQPSGPGQAQAPTQGGNTDPLELFLYQLLD
EVQVEEHAPAPLNWDVDPGGRVHEGSWESFWPQEEAGSTGLDTSSPSDSN
SFFRESKPSQVAQRRGAGQEDARTQADSTGPLELLLFDQLLDEVQKEEHV
PAPLDWGRNPGSMEHEGSQDSLLPLEEAANSGRDTSIPSIWPAFCRKSQP
PQVAQPSGPGQAQAPIQGGNTDPLELFLDQLLTEVQLEEQGPAPVNVEET
WEQMDTTPDLPLTSEEYQTLLDML*

The amino acid sequence of the mDux homeodomain 1 comprises: RRRRKTVWQAWQEQALLSTFKKKRYLSFKERKELAKRMGVSDCRIRVWFQNRRNR SGEEG (SEQ ID NO:90). The amino acid sequence of the mDux homeodomain 2 comprises:
GRRPRTRLTS LQLRILGQAFERNPRPGFATREELARDTGLPEDTIHIWFQNRRARRRH RR (SEQ ID NO:91). The amino acid sequence of the mDux Conserved C-terminal domain comprises LFLDQLLTEVQLEEQGPAPVNVEETWEQMDTTPDLPLTSEEYQTLLDML (SEQ ID NO:92).

An exemplary canine (domesticated dog) DUXC double homeodomain protein may be encoded by a nucleic acid comprising the following sequence (SEQ ID NO:93):

ATGGCCTCCAGCAGCACCCCCGGCGGCCCACTCCCTCGAGCACCCCGACG
AAGGAGGCTCGTGTTGACGGCAAGCCAGAAGGGGGCCCTGCAGGCATTCT
TCCAGAAGAACCCTTACCCCAGCATCACTGCCAGAGAACACCTGGCCCGA
GAGCTGGCCATCTCCGAGTCTAGAATCCAGGTCTGGTTCCAAAACCAGAG
AACGAGACAGCTAAGGCAGAGCCGCCGACTGGACTCCAGAATTCCCCAAG
GAGAAGGGCCACCGAATGGAAAGGCACAGCCTCCAGGTCGAGTCCCGAAG
GAAGGCAGGAGAAAACGGACATCCATTTCTGCATCCCAAACCAGTATCCT
CCTTCAAGCCTTTGAGGAGGAGCGGTTTCCTGGCATTGGTATGAGGGAAA
GCCTGGCCAGAAAAACAGGCCTTCCAGAAGCCAGAATTCAGGTTTGGTTT
CAGAACAGAAGAGCTCGGCACCCAGGGCAGAGCCCAAGTGGGCCCGAGAA
TGCTTTGGCGGCAAACCACAAACCCAGTCCTCGCGGGACGGTCCCATTGG
ACCAAAGCCACCTGTCAAGGGTCCCCAGGAGCTCTCCAAATCTGGCTCCC
TTCGATCCCTTGGGAAGCATGCAGACGCAGGCTGCAGGGACACCTCCTGT
CTCCTCCGTGGTTGTTGTCCCTCCAGTTTCTTGTGGGGGCTTTGGGCGCC
TGATTCCGGGGGCCTGCCTGGTCACACCAACCTTAGGTGGGCAAGGAGGA
ATCGCTGCTGCTCCCAGAGTCCTGGGGAGCCGATGCTGCCCAGAACTGAC
TCCAGGAGGGGGCCTCTCACCAGGTCATGCTGACCTTGGCCTCCCCTCCC
CTGGGAGATGCCAGCAGCCGAAAGAGCACCCCAGCAAGGCGCCCCTGCCC
TCGCAAGTTGGCCCGCGGCCTCCGCCTGTTGATCCTCCTCAACACTGGGG
TCATGCAGGTCCCCCGGGCACCGGTCAGGCCACGCCGAGGAGGGGCCAAA
GTTCCCAGGCAGTCATGGGCACAGCAGGGTCCCAGGATGGGACAGGGCAG
CAGCCCGCCCCCGGGGAGAGCCCCGCTTGGTGGCAACAGCCTCCCCCTCC
TGCAGGGCCATGTGTCCCGCTGCCCCCACAACACCAGCTGTGTGCGGACA
CCTCCAGTTTCCTACAAGAGCTTTTCTCAGCCGATGAGATGGAAGAAGAT
GTCCACCCCTTGTGGGTGGGGACTCTGCAGGAGGACGAACCTCCAGGACC
CCTGGAAGCACCCCTCAGCGAGGACGATTCTCACGCTCTGCTGGAAATGC
TACAGGACTCCTTGTGGCCTCAGGCCTAG

The amino acid sequence of the canine DUXC may comprise the following (SEQ ID NO:94)

MASSSTPGGPLPRAPRRRRLVLTASQKGALQAFFQKNPYPSITAREHLAR
ELAISESRIQVWFQNQRTRQLRQSRRLDSRIPQGEGPPNGKAQPPGRVPK
EGRRKRTSISASQTSILLQAFEEERFPGIGMRESLARKTGLPEARIQVWF
QNRRARHPGQSPSGPENALAANHKPSPRGTVPLDQSHLSRVPRSSPNLAP
FDPLGSMQTQAAGTPPVSSVVVVPPVSCGGFGRLIPGACLVTPTLGGQGG
IAAAPRVLGSRCCPELTPGGGLSPGHADLGLPSPGRCQQPKEHPSKAPLP
SQVGPRPPPVDPPQHWGHAGPPGTGQATPRRGQSSQAVMGTAGSQDGTGQ
QPAPGESPAWWQQPPPPAGPCVPLPPQHQLCADTSSFLQELFSADEMEED
VHPLWVGTLQEDEPPGPLEAPLSEDDSHALLEMLQDSLWPQA*

The amino acid sequence of the canine DUXC homeodomain 1 comprises: PRRRRLVLTASQKGALQAFFQKNPYPSITAREHLARELAISESRIQVWFQNQRTRQLR QS (SEQ ID NO:95). The amino acid sequence of the canine DUXC homeodomain 2 comprises
GRRKRTSISASQTSILLQAFEEERFPGIGMRESLARKTGLPEARIQVWFQNRRARHPG QS (SEQ ID NO:96). The amino acid sequence of the canine DUXC conserved C-terminal domain comprises:

(SEQ ID NO: 97)

SFLQELFSADEMEEDVHPLWVGTLQEDEPPGPLEAPLSEDDSHALLEMLQ
DSLWPQA.

A chimera comprising mouse DUX (mDUX) homeodomains and human DUX4 (hDUX4) carboxy terminus (abbreviated as MMH in the examples) comprises the following sequence (SEQ ID NO:98):

ATGGCTGAGGCTGGCTCTCCAGTGGGAGGATCTGGAGTGGCCAGAGAATC
AAGGAGAAGGAGGAAAACTGTCTGGCAAGCTTGGCAGGAACAGGCACTCC
TGAGCACATTTAAGAAAAAAAGGTATCTGTCCTTTAAAGAAAGAAAGGAA
CTGGCAAAAAGGATGGGAGTTTCTGATTGCAGGATCAGAGTCTGGTTCCA
GAATAGGAGAAATAGGTCTGGGGAGGAAGGACATGCAAGCAAGAGAAGCA
TAAGAGGTTCCAGGAGGCTGGCATCCCCTCAACTTCAGGAGGAACTGGGA
AGTAGGCCCCAAGGCAGGGGCATGAGGTCCTCAGGGAGGAGACCCAGAAC
CAGGCTGACAAGTCTGCAGCTGAGAATCCTTGGTCAGGCTTTTGAAAGGA
ATCCAAGGCCAGGATTTGCCACCAGAGAGGAACTGGCCAGGGATACAGGC
CTTCCTGAGGATACTATCCATATCTGGTTCCAGAACAGGAGGGCCAGGAG
AAGGCACAGAAGGGGAAGACCTCCTGCTCAGGCCGGTGGACTCTGCTCTG
CTGCCCCTGGGGGCGGCCATCCAGCACCTTCCTGGGTGGCTTTCGCTCAT
ACTGGCGCTTGGGGTACCGGGCTGCCTGCTCCGCATGTTCCCTGTGCTCC
AGGGGCCCTCCCGCAGGGAGCGTTTGTTTCCCAGGCAGCTAGGGCTGCAC
CTGCCCTGCAACCATCACAGGCAGCGCCAGCTGAAGGCATCAGCCAACCC
GCCCCAGCCCGCGGAGATTTTGCTTATGCAGCGCCAGCACCTCCAGACGG
TGCCCTGAGCCACCCCCAAGCCCCCAGATGGCCCCCTCACCCTGGTAAGT
CCCGGGAAGACCGCGATCCCCAACGAGATGGACTGCCCGGTCCTTGCGCT
GTGGCCCAGCCAGGACCTGCTCAAGCCGGCCCTCAGGGGCAAGGAGTGCT
GGCCCCACCTACAAGCCAGGGATCTCCCTGGTGGGGTTGGGGACGCGGAC
CTCAGGTTGCTGGAGCCGCTTGGGAGCCTCAGGCCGGAGCTGCACCGCCG
CCACAACCGGCCCCTCCCGACGCGTCAGCGTCCGCCCGACAAGGCCAGAT
GCAGGGAATCCCAGCACCTAGCCAAGCTCTTCAAGAGCCTGCCCCTTGGA
GCGCACTGCCGTGTGGGCTGCTCCTGGATGAACTCCTGGCTAGCCCAGAA
TTTCTCCAGCAGGCACAGCCACTCCTGGAAACAGAAGCTCCGGGAGAGCT
CGAAGCCTCCGAAGAAGCAGCAAGCCTGGAGGCACCTCTTTCCGAGGAGG
AGTATAGAGCCCTTCTGGAAGAACTTTGA

The MMH comprises a polypeptide comprising the following amino acid sequence (SEQ ID NO: 99):

MAEAGSPVGGSGVARESRRRRKTVWQAWQEQALLSTFKKKRYLSFKERKE
LAKRMGVSDCRIRVWFQNRRNRSGEEGHASKRSIRGSRRLASPQLQEELG
SRPQGRGMRSSGRRPRTRLTSLQLRILGQAFERNPRPGFATREELARDTG
LPEDTIHIWFQNRRARRRHRRGRPPAQAGGLCSAAPGGGHPAPSWVAFAH
TGAWGTGLPAPHVPCAPGALPQGAFVSQAARAAPALQPSQAAPAEGISQP
APARGDFAYAAPAPPDGALSHPQAPRWPPHPGKSREDRDPQRDGLPGPCA
VAQPGPAQAGPQGQGVLAPPTSQGSPWWGWGRGPQVAGAAWEPQAGAAPP
PQPAPPDASASARQGQMQGIPAPSQALQEPAPWSALPCGLLLDELLASPE
FLQQAQPLLETEAPGELEASEEAASLEAPLSEEEYRALLEEL*

A chimera comprising the second hDUX4 homeodomain introduced into mDUX in place of the mDUX second homeodomain (abbreviated as MHM in the examples) comprises the following sequence (SEQ ID NO: 100):

ATGGCTGAGGCTGGCTCTCCAGTGGGAGGATCTGGAGTGGCCAGAGAATC
AAGGAGAAGGAGGAAAACTGTCTGGCAAGCTTGGCAGGAACAGGCACTCC
TGAGCACATTTAAGAAAAAAAGGTATCTGTCCTTTAAAGAAAGAAAGGAA
CTGGCAAAAAGGATGGGAGTTTCTGATTGCAGGATCAGAGTCTGGTTCCA
GAATAGGAGAAATAGGTCTGGGGAGGAAGGACATGCAAGCAAGAGAAGCA
TAAGAGGTTCCAGGAGGCTGGCATCCCCTCAACTTCAGGAGGAACTGGGA
AGTAGGCCCCAAGGCAGGGGCATGAGGTCCTCAGGAAGAAGAAAACGCAC
AGCGGTGACTGGCAGCCAAACGGCTCTGCTGCTCCGCGCTTTCGAGAAAG
ATCGGTTCCCCGGAATTGCCGCACGCGAAGAACTCGCCAGAGAAACTGGG
CTCCCAGAATCACGAATACAGATTTGGTTCCAGAACCGCAGAGCAAGACA
CCCAGGCCAGGGGGGAAGACCTACAGCCCAGGACCAGGACCTCCTGGCTT
CCCAGGGTTCTGATGGAGCACCTGCTGGGCCTGAAGGTAGAGAGAGAGAA
GGAGCACAGGAAAATTTGCTGCCCCAGGAGGAGGCAGGATCAACAGGGAT
GGACACCTCAAGCCCTTCTGACCTCCCTTCATTCTGTGGTGAATCACAGC
CCTTTCAGGTGGCCCAGCCCAGGGGAGCTGGACAGCAGGAGGCTCCCACA
AGGGCAGGGAATGCTGGATCATTGGAGCCACTGTTGGACCAGCTCTTGGA
TGAGGTCCAGGTGGAGGAACCTGCCCCAGCTCCACTCAACCTGGATGGTG
ATCCTGGGGGGAGGGTTCATGAGGGTAGTCAGGAGTCCTTCTGGCCCCAG
GAGGAGGCTGGTTCTACTGGAATGGACACTTCTTCACCCTCTGACAGCAA
TAGCTTTTGCAGGGAGAGTCAACCCTCTCAGGTAGCTCAGCCTTGTGGGG
CTGGCCAGGAGGATGCTAGGACCCAGGCTGACTCAACAGGGCCCTTGGAG
CTGTTGCTGCTGGACCAGCTCCTGGATGAGGTACAGAAGGAGGAACATGT
ACCAGTGCCCCTGGACTGGGGGAGGAACCCTGGAAGCAGAGAACATGAGG
GTAGTCAGGATTCTCTCCTTCCTCTGGAAGAGGCTGTGAATTCTGGAATG
GACACTAGTATACCAAGTATTTGGCCTACATTTTGCAGGGAGTCACAACC
CCCACAGGTGGCTCAGCCTTCAGGACCTGGGCAGGCCCAGGCTCCTACCC
AAGGGGGTAATACAGACCCACTGGAACTCTTTCTGTATCAGCTGCTGGAT
GAGGTCCAGGTGGAGGAACATGCCCCAGCTCCACTCAACTGGGATGTGGA
TCCAGGGGGCAGAGTCCATGAGGGTTCCTGGGAGTCATTCTGGCCCCAGG
AGGAGGCAGGCTCTACAGGACTGGACACAAGCTCCCCTAGTGACAGCAAC
TCATTCTTTAGGGAGAGTAAGCCCTCTCAGGTTGCTCAAAGGAGGGGAGC
TGGGCAAGAGGATGCCAGGACTCAGGCTGACAGTACAGGACCCCTGGAGC
TGCTGTTGTTTGACCAGCTCCTGGATGAAGTGCAGAAGGAGGAACATGTT
CCAGCTCCCCTGGACTGGGGAAGGAACCCTGGTTCTATGGAACATGAGGG
CTCTCAGGACTCTCTCTTGCCTCTGGAAGAAGCTGCTAATAGTGGCAGAG
ATACAAGTATCCCAAGCATTTGGCCTGCCTTTTGCAGGAAAAGCCAGCCA
CCCCAGGTAGCCCAGCCTAGTGGACCTGGACAGGCTCAGGCACCTATACA
AGGAGGCAACACTGACCCATTGGAGTTGTTTCTGGACCAGCTGCTCACTG
AGGTGCAACTGGAGGAACAAGGGCCAGCACCTGTCAATGTTGAAGAGACC
TGGGAACAGATGGATACCACTCCAGACTTGCCACTGACTTCTGAAGAGTA
CCAGACCCTTCTTGACATGCTGTAA

The MHM comprises a polypeptide comprising the following amino acid sequence (SEQ ID NO: 101):

MAEAGSPVGGSGVARESRRRRKTVWQAWQEQALLSTFKKKRYLSFKERKE
LAKRMGVSDCRIRVWFQNRRNRSGEEGHASKRSIRGSRRLASPQLQEELG
SRPQGRGMRSSGRRKRTAVTGSQTALLLRAFEKDRFPGIAAREELARETG
LPESRIQIWFQNRRARHPGQGGRPTAQDQDLLASQGSDGAPAGPEGRERE
GAQENLLPQEEAGSTGMDTSSPSDLPSFCGESQPFQVAQPRGAGQQEAPT
RAGNAGSLEPLLDQLLDEVQVEEPAPAPLNLDGDPGGRVHEGSQESFWPQ
EEAGSTGMDTSSPSDSNSFCRESQPSQVAQPCGAGQEDARTQADSTGPLE
LLLLDQLLDEVQKEEHVPVPLDWGRNPGSREHEGSQDSLLPLEEAVNSGM
DTSIPSIWPTFCRESQPPQVAQPSGPGQAQAPTQGGNTDPLELFLYQLLD
EVQVEEHAPAPLNWDVDPGGRVHEGSWESFWPQEEAGSTGLDTSSPSDSN
SFFRESKPSQVAQRRGAGQEDARTQADSTGPLELLLFDQLLDEVQKEEHV
PAPLDWGRNPGSMEHEGSQDSLLPLEEAANSGRDTSIPSIWPAFCRKSQP
PQVAQPSGPGQAQAPIQGGNTDPLELFLDQLLTEVQLEEQGPAPVNVEET
WEQMDTTPDLPLTSEEYQTLLDML*

A chimera comprising the first hDUX4 homeodomain introduced into mDUX in place of the mDUX first homeodomain (abbreviated as HMM in the examples) comprises the following sequence (SEQ ID NO: 102):

ATGGCTGAGGCTGGCTCTCCAGTGGGAGGATCTGGAGTGGCCAGAGAATC
AGGTAGACGGCGGCGATTGGTGTGGACTCCATCACAATCCGAAGCTCTTC
GCGCATGCTTCGAGCGCAATCCCTATCCGGGGATTGCCACAAGGGAGAGG
CTTGCACAGGCTATCGGAATCCCGGAACCGAGAGTGCAGATCTGGTTCCA
AAATGAACGCTCTCGGCAGCTCAGACAGCATCATGCAAGCAAGAGAAGCA
TAAGAGGTTCCAGGAGGCTGGCATCCCCTCAACTTCAGGAGGAACTGGGA
AGTAGGCCCCAAGGCAGGGGCATGAGGTCCTCAGGGAGGAGACCCAGAAC
CAGGCTGACAAGTCTGCAGCTGAGAATCCTTGGTCAGGCTTTTGAAAGGA
ATCCAAGGCCAGGATTTGCCACCAGAGAGGAACTGGCCAGGGATACAGGC
CTTCCTGAGGATACTATCCATATCTGGTTCCAGAACAGGAGGGCCAGGAG
AAGGCACAGAAGGGGAAGACCTACAGCCCAGGACCAGGACCTCCTGGCTT
CCCAGGGTTCTGATGGAGCACCTGCTGGGCCTGAAGGTAGAGAGAGAGAA
GGAGCACAGGAAAATTTGCTGCCCCAGGAGGAGGCAGGATCAACAGGGAT
GGACACCTCAAGCCCTTCTGACCTCCCTTCATTCTGTGGTGAATCACAGC
CCTTTCAGGTGGCCCAGCCCAGGGGAGCTGGACAGCAGGAGGCTCCCACA
AGGGCAGGGAATGCTGGATCATTGGAGCCACTGTTGGACCAGCTCTTGGA
TGAGGTCCAGGTGGAGGAACCTGCCCCAGCTCCACTCAACCTGGATGGTG
ATCCTGGGGGGAGGGTTCATGAGGGTAGTCAGGAGTCCTTCTGGCCCCAG
GAGGAGGCTGGTTCTACTGGAATGGACACTTCTTCACCCTCTGACAGCAA
TAGCTTTTGCAGGGAGAGTCAACCCTCTCAGGTAGCTCAGCCTTGTGGGG
CTGGCCAGGAGGATGCTAGGACCCAGGCTGACTCAACAGGGCCCTTGGAG
CTGTTGCTGCTGGACCAGCTCCTGGATGAGGTACAGAAGGAGGAACATGT
ACCAGTGCCCCTGGACTGGGGGAGGAACCCTGGAAGCAGAGAACATGAGG
GTAGTCAGGATTCTCTCCTTCCTCTGGAAGAGGCTGTGAATTCTGGAATG
GACACTAGTATACCAAGTATTTGGCCTACATTTTGCAGGGAGTCACAACC
CCCACAGGTGGCTCAGCCTTCAGGACCTGGGCAGGCCCAGGCTCCTACCC
AAGGGGGTAATACAGACCCACTGGAACTCTTTCTGTATCAGCTGCTGGAT
GAGGTCCAGGTGGAGGAACATGCCCCAGCTCCACTCAACTGGGATGTGGA
TCCAGGGGGCAGAGTCCATGAGGGTTCCTGGGAGTCATTCTGGCCCCAGG
AGGAGGCAGGCTCTACAGGACTGGACACAAGCTCCCCTAGTGACAGCAAC
TCATTCTTTAGGGAGAGTAAGCCCTCTCAGGTTGCTCAAAGGAGGGGAGC
TGGGCAAGAGGATGCCAGGACTCAGGCTGACAGTACAGGACCCCTGGAGC
TGCTGTTGTTTGACCAGCTCCTGGATGAAGTGCAGAAGGAGGAACATGTT
CCAGCTCCCCTGGACTGGGGAAGGAACCCTGGTTCTATGGAACATGAGGG
CTCTCAGGACTCTCTCTTGCCTCTGGAAGAAGCTGCTAATAGTGGCAGAG
ATACAAGTATCCCAAGCATTTGGCCTGCCTTTTGCAGGAAAAGCCAGCCA
CCCCAGGTAGCCCAGCCTAGTGGACCTGGACAGGCTCAGGCACCTATACA
AGGAGGCAACACTGACCCATTGGAGTTGTTTCTGGACCAGCTGCTCACTG
AGGTGCAACTGGAGGAACAAGGGCCAGCACCTGTCAATGTTGAAGAGACC
TGGGAACAGATGGATACCACTCCAGACTTGCCACTGACTTCTGAAGAGTA
CCAGACCCTTCTTGACATGCTGTAA

The HMM comprises a polypeptide comprising the following amino acid sequence (SEQ ID NO:103):

MAEAGSPVGGSGVARESGRRRRLVWTPSQSEALRACFERNPYPGIATRER
LAQAIGIPEPRVQIWFQNERSRQLRQHHASKRSIRGSRRLASPQLQEELG
SRPQGRGMRSSGRRPRTRLTSLQLRILGQAFERNPRPGFATREELARDTG
LPEDTIHIWFQNRRARRRHRRGRPTAQDQDLLASQGSDGAPAGPEGRERE
GAQENLLPQEEAGSTGMDTSSPSDLPSFCGESQPFQVAQPRGAGQQEAPT
RAGNAGSLEPLLDQLLDEVQVEEPAPAPLNLDGDPGGRVHEGSQESFWPQ
EEAGSTGMDTSSPSDSNSFCRESQPSQVAQPCGAGQEDARTQADSTGPLE
LLLLDQLLDEVQKEEHVPVPLDWGRNPGSREHEGSQDSLLPLEEAVNSGM
DTSIPSIWPTFCRESQPPQVAQPSGPGQAQAPTQGGNTDPLELFLYQLLD
EVQVEEHAPAPLNWDVDPGGRVHEGSWESFWPQEEAGSTGLDTSSPSDSN
SFFRESKPSQVAQRRGAGQEDARTQADSTGPLELLLFDQLLDEVQKEEHV
PAPLDWGRNPGSMEHEGSQDSLLPLEEAANSGRDTSIPSIWPAFCRKSQP
PQVAQPSGPGQAQAPIQGGNTDPLELFLDQLLTEVQLEEQGPAPVNVEET
WEQMDTTPDLPLTSEEYQTLLDML*

A chimera comprising the the second mDUX homeodomain introduced into hDUX4 in place of the hDUX4 second homeodomain (abbreviated as HMH in the examples) comprises the following sequence (SEQ ID NO: 104):

ATGGCATTGCCTACACCTTCAGACTCTACGCTGCCTGCAGAGGCTAGGGG
AAGAGGTAGACGGCGGCGATTGGTGTGGACTCCATCACAATCCGAAGCTC
TTCGCGCATGCTTCGAGCGCAATCCCTATCCGGGGATTGCCACAAGGGAG
AGGCTTGCACAGGCTATCGGAATCCCGGAACCGAGAGTGCAGATCTGGTT
CCAAAATGAACGCTCTCGGCAGCTCAGACAGCATCGCAGGGAGTCCCGCC
CGTGGCCAGGAAGAAGGGGACCACCTGAAGGGAGGAGACCCAGAACCAGG
CTGACAAGTCTGCAGCTGAGAATCCTTGGTCAGGCTTTTGAAAGGAATCC
AAGGCCAGGATTTGCCACCAGAGAGGAACTGGCCAGGGATACAGGCCTTC
CTGAGGATACTATCCATATCTGGTTCCAGAACAGGAGGGCCAGGAGAAGG
CACAGAAGGGGACGGGCACCTGCTCAGGCCGGTGGACTCTGCTCTGCTGC
CCCTGGGGGCGGCCATCCAGCACCTTCCTGGGTGGCTTTCGCTCATACTG
GCGCTTGGGGTACCGGGCTGCCTGCTCCGCATGTTCCCTGTGCTCCAGGG
GCCCTCCCGCAGGGAGCGTTTGTTTCCCAGGCAGCTAGGGCTGCACCTGC
CCTGCAACCATCACAGGCAGCGCCAGCTGAAGGCATCAGCCAACCCGCCC
CAGCCCGCGGAGATTTTGCTTATGCAGCGCCAGCACCTCCAGACGGTGCC
CTGAGCCACCCCCAAGCCCCCAGATGGCCCCCTCACCCTGGTAAGTCCCG
GGAAGACCGCGATCCCCAACGAGATGGACTGCCCGGTCCTTGCGCTGTGG
CCCAGCCAGGACCTGCTCAAGCCGGCCCTCAGGGGCAAGGAGTGCTGGCC
CCACCTACAAGCCAGGGATCTCCCTGGTGGGGTTGGGGACGCGGACCTCA
GGTTGCTGGAGCCGCTTGGGAGCCTCAGGCCGGAGCTGCACCGCCGCCAC
AACCGGCCCCTCCCGACGCGTCAGCGTCCGCCCGACAAGGCCAGATGCAG
GGAATCCCAGCACCTAGCCAAGCTCTTCAAGAGCCTGCCCCTTGGAGCGC
ACTGCCGTGTGGGCTGCTCCTGGATGAACTCCTGGCTAGCCCAGAATTTC
TCCAGCAGGCACAGCCACTCCTGGAAACAGAAGCTCCGGGAGAGCTCGAA
GCCTCCGAAGAAGCAGCAAGCCTGGAGGCACCTCTTTCCGAGGAGGAGTA
TAGAGCCCTTCTGGAAGAACTTTGA

The HMH comprises a polypeptide comprising the following amino acid sequence (SEQ ID NO:105):

MALPTPSDSTLPAEARGRGRRRRLVWTPSQSEALRACFERNPYPGIATRE
RLAQAIGIPEPRVQIWFQNERSRQLRQHRRESRPWPGRRGPPEGRRPRTR
LTSLQLRILGQAFERNPRPGFATREELARDTGLPEDTIHIWFQNRRARRR
HRRGRAPAQAGGLCSAAPGGGHPAPSWVAFAHTGAWGTGLPAPHVPCAPG
ALPQGAFVSQAARAAPALQPSQAAPAEGISQPAPARGDFAYAAPAPPDGA
LSHPQAPRWPPHPGKSREDRDPQRDGLPGPCAVAQPGPAQAGPQGQGVLA
PPTSQGSPWWGWGRGPQVAGAAWEPQAGAAPPPQPAPPDASASARQGQMQ
GIPAPSQALQEPAPWSALPCGLLLDELLASPEFLQQAQPLLETEAPGELE
ASEEAASLEAPLSEEEYRALLEEL*

A chimera comprising the first mDUX homeodomain introduced into hDUX4 in place of the hDUX4 first homeodomain (abbreviated as MHH in the examples) comprises the following sequence (SEQ ID NO:106):

ATGGCATTGCCTACACCTTCAGACTCTACGCTGCCTGCAGAGGCTAGGGG
AAGAAGGAGAAGGAGGAAAACTGTCTGGCAAGCTTGGCAGGAACAGGCAC
TCCTGAGCACATTTAAGAAAAAAAGGTATCTGTCCTTTAAAGAAAGAAAG
GAACTGGCAAAAAGGATGGGAGTTTCTGATTGCAGGATCAGAGTCTGGTT
CCAGAATAGGAGAAATAGGTCTGGGGAGGAAGGACGCAGGGAGTCCCGCC
CGTGGCCAGGAAGAAGGGGACCACCTGAAGGAAGAAGAAAACGCACAGCG
GTGACTGGCAGCCAAACGGCTCTGCTGCTCCGCGCTTTCGAGAAAGATCG
GTTCCCCGGAATTGCCGCACGCGAAGAACTCGCCAGAGAAACTGGGCTCC
CAGAATCACGAATACAGATTTGGTTCCAGAACCGCAGAGCAAGACACCCA
GGCCAGGGGGGACGGGCACCTGCTCAGGCCGGTGGACTCTGCTCTGCTGC
CCCTGGGGGCGGCCATCCAGCACCTTCCTGGGTGGCTTTCGCTCATACTG
GCGCTTGGGGTACCGGGCTGCCTGCTCCGCATGTTCCCTGTGCTCCAGGG
GCCCTCCCGCAGGGAGCGTTTGTTTCCCAGGCAGCTAGGGCTGCACCTGC
CCTGCAACCATCACAGGCAGCGCCAGCTGAAGGCATCAGCCAACCCGCCC
CAGCCCGCGGAGATTTTGCTTATGCAGCGCCAGCACCTCCAGACGGTGCC
CTGAGCCACCCCCAAGCCCCCAGATGGCCCCCTCACCCTGGTAAGTCCCG
GGAAGACCGCGATCCCCAACGAGATGGACTGCCCGGTCCTTGCGCTGTGG
CCCAGCCAGGACCTGCTCAAGCCGGCCCTCAGGGGCAAGGAGTGCTGGCC
CCACCTACAAGCCAGGGATCTCCCTGGTGGGGTTGGGGACGCGGACCTCA
GGTTGCTGGAGCCGCTTGGGAGCCTCAGGCCGGAGCTGCACCGCCGCCAC
AACCGGCCCCTCCCGACGCGTCAGCGTCCGCCCGACAAGGCCAGATGCAG
GGAATCCCAGCACCTAGCCAAGCTCTTCAAGAGCCTGCCCCTTGGAGCGC
ACTGCCGTGTGGGCTGCTCCTGGATGAACTCCTGGCTAGCCCAGAATTTC
TCCAGCAGGCACAGCCACTCCTGGAAACAGAAGCTCCGGGAGAGCTCGAA
GCCTCCGAAGAAGCAGCAAGCCTGGAGGCACCTCTTTCCGAGGAGGAGTA
TAGAGCCCTTCTGGAAGAACTTTGA

The MHH comprises a polypeptide comprising the following amino acid sequence (SEQ ID NO:107):

MALPTPSDSTLPAEARGRRRRRKTVWQAWQEQALLSTFKKKRYLSFKERK
ELAKRMGVSDCRIRVWFQNRRNRSGEEGRRESRPWPGRRGPPEGRRKRTA
VTGSQTALLLRAFEKDRFPGIAAREELARETGLPESRIQIWFQNRRARHP
GQGGRAPAQAGGLCSAAPGGGHPAPSWVAFAHTGAWGTGLPAPHVPCAPG
ALPQGAFVSQAARAAPALQPSQAAPAEGISQPAPARGDFAYAAPAPPDGA
LSHPQAPRWPPHPGKSREDRDPQRDGLPGPCAVAQPGPAQAGPQGQGVLA
PPTSQGSPWWGWGRGPQVAGAAWEPQAGAAPPPQPAPPDASASARQGQMQ
GIPAPSQALQEPAPWSALPCGLLLDELLASPEFLQQAQPLLETEAPGELE
ASEEAASLEAPLSEEEYRALLEEL*

An exemplary cow DUXC double homeodomain containing protein may be encoded by a nucleic acid comprising the following sequence (SEQ ID NO:108):

ACCATGGTGA GCAAGGGCGA GGAGCTGTTC ACCGGGGTGG
TGCCCATCCT GGTCGAGCTG GACGGCGACG TAAACGGCCA
CAAGTTCAGC GTGTCCGGCG AGGGCGAGGG CGATGCCACC
TACGGCAAGC TGACCCTGAA GTTCATCTGC ACCACCGGCA
AGCTGCCCGT GCCCTGGCCC ACCCTCGTGA CCACCCTGAC
CTACGGCGTG CAGTGCTTCA GCCGCTACCC CGACCACATG
AAGCAGCACG ACTTCTTCAA GTCCGCCATG CCCGAAGGCT
ACGTCCAGGA GCGCACCATC TTCTTCAAGG ACGACGGCAA
CTACAAGACC CGCGCCGAGG TGAAGTTCGA GGGCGACACC
CTGGTGAACC GCATCGAGCT GAAGGGCATC GACTTCAAGG
AGGACGGCAA CATCCTGGGG CACAAGCTGG AGTACAACTA
CAACAGCCAC AACGTCTATA TCATGGCCGA CAAGCAGAAG
AACGGCATCA AGGTGAACTT CAAGATCCGC CACAACATCG
AGGACGGCAG CGTGCAGCTC CCGACCACT ACCAGCAGAA
CACCCCCATC GGCGACGGCC CCGTGCTGCT GCCCGACAAC
CACTACCTGA GCACCCAGTC CGCCCTGAGC AAAGACCCCA
ACGAGAAGCG CGATCACATG GTCCTGCTGG AGTTCGTGAC
CGCCGCCGGG ATCACTCTCG GCATGGACGA GCTGTAcAcc
GGAAGCGGTG CAAGCAGCGG ATCATCCAGT ACCAGCCGGG
GTCCTATTGC AACGGGCTCA AGGCGGCGGC GCTTGGTTCT
GAAACCTAGC CAAAAAGATG CTCTTCAAGC CTTGTTTCAA
CAGAATCCAT ACCCAGGCAT AGCGACCCGC GAGAGATTGG
CTCGAGAGTT GGGTATCGAC GAGAGTAGGG TGCAAGTCTG
GTTTCAAAAT CAGCGACGGA GGAGAAGTAA GCAGAGTAGG
CCGCCTTCTG AACATGTGCG ACAGGAGGGA GAGGGGGGGC
CTACTTCTAC ACCTCGCCCC CCAAGCCCGC CTCCGAGGCC
ACAGAGTAGC AGTCAAGGCA AACTCGCGAG CGTCCTCTCA
AAAGGGAAGG AGGCACGGAG GAAACGAACC GTGATCTCAC
CGAGCCAAAC ACGCATACTC GTGCAAGCTT TCACGAGGGA
CAGATTCCCG GGGATAGCCG CTCGCGAGGA ATTGGCACGA
CAAACCGGCA TTCCGGAACC GCGCATACAG ATTTGGTTTC
AAAATAGACG AGCCCGCCAC CCGCAACGCT CCCCGTCTGG
TCCTGGGAAT GGCAGAGCAC AGGGGCCCGG TGGTGCTCCC
GCAACGACCA CTACACCAGC CCCCGAAGAT CGCCGAGCTC
CACCCGCTGT TCAATCAACA AGTCCTCCGC TTAGGCCATC
CCAACCACAG GAAAGCATGC CCCCTCTTGC AGCAGCGGCC
CCTTTTGGAG CACCTACCTT CTGGGTGCTA GGAGCTGCAA
GCGGAGTCTG TGTGGGTCAA CCCTTGATGA TCTTTGTGGT
GCAGCCCAGC CCAGCCGCGT TGCAACCGTC TGGGAGGCCT
CCTCCTCCTC CTCAGGGTGC CGCACCATGG GCGGCTTGCA
GCCCCGCGGT GACAGCGCCC GGTCTGCCAG GTCAGGGCGC
GATTCTGCCA CCGGGACAAC CGGAGACTCA CATCCCCCGA
TGGCCgGAAT CCCCCTCCGG TGAAGGGACC GCACCCCCTC
TCGAGCCCCA ACCACAAGCC CCGAGTCTCC CCAGCTCCAC
TTCTCTTCTC GATGAGCTCC TGGCCGCTAC TGGGGTTCCC
GACACCCAAG CGCCCAGTCC TGGGGCAGCT GCGGATGAAG
GTGTTGGGCC CGCTCTCCCG GGAGCTCCGA GCTTCTTGGA
TGAGCTGCTG GCTGCAACGG GAACGCCGGA TACTCCCGGG
CCGTCCCTTG GGCCAAGCGC AGACGAGCGA GCCCACCTGG
CGCTCCCCGG CGAATTGCTC GCGGCCGCTG GACTTCCTGG
TTCACCCGGC CCAAGTCCTG GCTCATCTCC TGTCGTCGCT
GGCCCTCACC CTGCGCTGCC TGGTCCCCCA TCTCTTCTGG
AAGAGATACT TGCTGCAACC GCTATACAGG ACACACCATG
GAGCAGCCCG GGAAGTCCCG CCGGGGAAGA AGGTGTTGAA
GCGACCTTGG AAACTCCATT GAGTGAAGAT GAATACCAAG
CTCTGCTCGA CATGCTGCCC GGCTCTCCAG GGCCCGGTGC
G.

An exemplary cow DUXC double homeodomain protein may comprise a polypeptide comprising the following sequence (SEQ ID NO:109):

MSGASSGSSSTSRGPIATGSRRRRLVLKPSQKDALQALFQQNPYPGIATR
ERLARELGIDESRVQVWFQNQRRRRSKQSRPPSEHVRQEGEGGPTSTPRP
PSPPPRPQSSSQGKLASVLSKGKEARRKRTVISPSQTRILVQAFTRDRFP
GIAAREELARQTGIPEPRIQIWFQNRRARHPQRSPSGPGNGRAQGPGGAP
ATTTTPAPEDRRAPPAVQSTSPPLRPSQPQESMPPLAAAAPFGAPTFWVL
GAASGVCVGQPLMIFVVQPSPAALQPSGRPPPPPQGAAPWAACSPAVTAP
GLPGQGAILPPGQPETHIPRWPESPSGEGTAPPLEPQPQAPSLPSSTSLL
DELLAATGVPDTQAPSPGAAADEGVGPALPGAPSFLDELLAATGTPDTPG
PSLGPSADERAHLALPGELLAAAGLPGSPGPSPGSSPVVAGPHPALPGPP
SLLEEILAATAIQDTPWSSPGSPAGEEGVEATLETPLSEDEYQALLDMLP
GSPGPGA.

The cow DUXC homeodomain #1 comprises the following polypeptide sequence: SRRRRLVLKPSQKDALQALFQQNPYPGIATRERLARELGIDESRVQVWFQNQRRRRS KQS (SEQ ID NO:110). The cow DUXC homeodomain #2 comprises the following polypeptide sequenc:
ARRKRTVISPSQTRILVQAFTRDRFPGIAAREELARQTGIPEPRIQIWFQNRRARHPQRS (SEQ ID NO:111). The cow DUXC conserved C-terminal activation domain comprises the following polypeptide sequence:

(SEQ ID NO: 112)

SLLEEILAATAIQDTPWSSPGSPAGEEGVEATLETPLSEDEYQALLDMLP
GSPGPGA

An exemplary horse DUXC double homeodomain containing protein may be encoded by a nucleic acid comprising the following sequence (SEQ ID NO: 113):

ATGGCCTGTGCGGAGACGGTCCTGGGCGCTGTCAAGAGGCCCTGGCTGTC
GTGCCCGCAGACGGCGGCTGCCGCTCAGGGAAACCACCTGCAGACGAGGC
GTCCTGGTGGCAGCGGTGGAGGCGTGGCAGCTGGCCCGCATCAGAGAGGA
TCCCGACGCAGGAGGATTGTTTTGAAGGCGAGTCAGAGGGACGCTCTGCG
AGCAGCGTTTCAACAGAACCCTTACCCTGGGATCGCCACCAGAGAACGCC
TGGCCCAAGAGATTGACATTCCGGAATGCAGAGTCCAGGTTTGGTTTCAA
AACCAACGCAGAAGACATCTAAGGCAGAGCCGGTCGGGCTCGGCGAGCTC
CGTGGGAGAAGGGCAATCGCCTGGAGAGGAGCAGCCCCAAGCTCGGGCCG
CAGAAGGCGGAAGAAAGCGGACACACATCACTCCGTGGCAAACCGGGATC
CTCCTTGAGAGCTTCCAGAAGGACCGATTTCCTGGCATCGCTACCAGGGA
AGAACTGGCCAGACAAACGGGCATCCCAGAGGCGAGAATTCAGGTGTGGT
TTCAGAACCGAAGAGCTCGGCACCCAGACCAGAGTGGAAGCGGCCCGGTG
AATGCCTTGGCGGAAGGCCCCAGTCCCAGGGCTCCCCTGACTGCCCTCCA
GGACCAAGCCAACCTGTCCTCTGTCCCCAGCAGCTCTCCGCATCTGCCTC
CCTGGAACCCTCCTGGGCTCTTGCCATCGCCCGCGACAGCCGCTCCTCCA
CTCTGCCCGGTGTTCTTCGTTCCTTGGGTTCCCTCTGGGGCCTGTGTGGG
CCGGCCACCGGAGCCCCTGGTGGTCATGACAGCCCAGCCTGTGCTGGGAA
AGGAGAACGTTCACCCTCCTTGGACACTTCTGTGTCCCTGCTCAACCGGG
CCGCCTCTGGCAGGCGGTCTCTCAGCGATGCAGCCTCCTCTCCGGCCCAC
GCCCGGAGGAAAATGCCAGGAGCACGACGGGCACGCTGGCGGGAGGGGGC
TGCCCTTCCCACACTCCCCTCAGCCTCACCCTGACCGTCCTCAGCAACAG
TGGCAGCACCTGGGTGGGCCAGGAGCCTTCCCCGCTATGCAGCCTTGGGG
CGAGTGGCCTCAGGTCCTCCCGGCCCCAGAGGAGCCTCAGGGAAGGGCGG
TTCAGCAGTCTGCGCACCCTGACACACACGTGTGGCCATGGGAGGAGCCA
TCAGCCGGAGAGCCCTCTGCTCAGCCGGGCCCACAGCAGCAGCACTCTGC
GCAAACCCCCAGCCTCCTAGATGAGCTGCTCGCAGTCACAGAGCTGCAGG
AAAAGGCACAGCCGTTCCTGAACGGGCATCCGCCGGCAGAGGAGCCTCCG
GGAACACTGGAAGGTCCCCTCAGCGAGGAGGAATTTCAGGCTCTGCTCGA
CATGCTGCAAAGCTCACCAGGGCCTCAGATTTAG.

An exemplary horse DUXC double homeodomain protein may comprise a polypeptide comprising the following sequence (SEQ ID NO:114):

MACAETVLGAVKRPWLSCPQTAAAAQGNHLQTRRPGGSGGGVAAGPHQRG
SRRRRIVLKASQRDALRAAFQQNPYPGIATRERLAQEIDIPECRVQVWFQ
NQRRRHLRQSRSGSASSVGEGQSPGEEQPQARAAEGGRKRTHITPWQTGI
LLESFQKDRFPGIATREELARQTGIPEARIQVWFQNRRARHPDQSGSGPV
NALAEGPSPRAPLTALQDQANLSSVPSSSPHLPPWNPPGLLPSPATAAPP
LCPVFFVPWVPSGACVGRPPEPLVVMTAQPVLGKENVHPPWTLLCPCSTG
PPLAGGLSAMQPPLRPTPGGKCQEHDGHAGGRGLPFPHSPQPHPDRPQQQ
WQHLGGPGAFPAMQPWGEWPQVLPAPEEPQGRAVQQSAHPDTHVWPWEEP
SAGEPSAQPGPQQQHSAQTPSLLDELLAVTELQEKAQPFLNGHPPAEEPP
GTLEGPLSEEEFQALLDMLQSSPGPQI*.

The horse DUXC homeodomain 1 polypeptide comprises the following amino acid sequence:
SRRRRIVLKASQRDALRAAFQQNPYPGIATRERLAQEIDIPECRVQVWFQNQRRRHL RQS (SEQ ID NO:115). The horse DUXC homeodomain 2 polypeptide comprises the following amino acid sequence:
GGRKRTHITPWQTGILLES FQKDRFPGIATREELARQTGIPEARIQVWFQNRRARHPD QS (SEQ ID NO:116). The horse domain DUXC conserved C-terminal domain comprises the following amino acid sequence:

(SEQ ID NO: 117)

SLLDELLAVTELQEKAQPFLNGHPPAEEPPGTLEGPLSEEEFQALLDMLQ
SSPGPQI.

An exemplary pig DUXC double homeodomain containing protein may be encoded by a nucleic acid comprising the following sequence (SEQ ID NO:118):

ATGCCCCTCAAGTTGGCAGTGTTGGCTCTTTGCTTGGCCTCATGCCAGCA
ATCATTTTTCCTAATGGGCTCACTTTCTAGAGGATCACGGAGAAGGAGGC
TTGTTCTGAAACAGAGTCAGCGGGATGCTCTGCAAGCAGTCTTTCAAGAG
AAGCCCTACCCTGGTATAACGACCAGAGAACGACTGGCCAGAGAACTTAG
CATCCCAGAAAGCCGAATTCAGATGTGGTTCCAAAACCAAAGAAAACGAC
GTCTCAAGCAGCAGAGCAGAGGGCCACCTGAGACTATCCCCCAACCAGGG
CCACCACAGCGGGAGCAACAGCTTCAGACTTCTCCCACTCCTGCAATCCC
AAAAGAGGCTGGGAGAAAGCGGTCATTCATCTCTCCCTCACAAACAGACA
TCCTTCGGCAAGCCTTTGAGCGGGAACGATACCCAGGCATTGCCGCCAGG
GAAGAACTGGCACGTCAAACAGGGATTCCAGAACCTCAGATTCTGGTGTG
GTTTCAGAACCGACGAGCTCGGCACCCAGAGCAGAAGGGAAGTGGGTCTG
CCAATGTGCCCGGAGTAGACCCCAATTCTGCAAAAGGCCTACCACTTCCA
TCGGACCAGGGCATGCCAACCACTGCCCACAGCAGCCCTACTCACAGTGC
TCCTCCTCCTCCCTCTAACCCACCAAGGGAGAACATGCTGTCCATCACCC
CCATGGTGGCCACTGCTGCGATCGCCCCCAAATTCATAGTTCCTGGGGCT
CCCACAGCAGGCTGTGAGGGCCAGAGCCTGCCCATGATCTTCATCATGGC
CCAGCCAAGTCCAGTTCTGCAGGCAATAGTGAACCCTCCCATGCTTTGGA
CGCTTCCTCTGACTCAGTCCTCACCAGGGCCAATGCCCATTCCTGCAGGG
GGTCTCACACCTATTCACACAGGGCTCTGGCCAACATCCCAAGAAGGACC
ATGGCAGGAGAACAATCTGCACACTATGCCAGCAGAAAAATGCCTCCCAC
ACATCCCTCAGCCACCCCTTGCCAGTCGTGCAGAGCCCCTGCCACTGCTG
GACCCAGTGAAGACCTGCACTTATGCCAGGCCAGAATGGGCCCAGGCATC
CTCAGCTCAAGTCACCAGTGGGAAGCCTGTGCATGGGGCCATGCTGCAGC
CTGCACAGGCTGACACACTTATCTGCCCCTCTCATCTGGCCCCCTCAAAT
GAAGAGCTGTGCCCTCCCATTGACCTGCAGCAGAACAAGCCCTCAGCCTT
CCAGGGCTCATCAAACCTCCTTGAGGAAATTATGGCAGCTGCAGGCATTC
TGCCTGAGGCAGGGCCTCTTCCAGACGTGGAGGAACAGGAAGAGCTTCCC
CTAGGAGACCTGGAAGCACCCCTCAGTGAGGAAGATTTCCAGGCCCTCCT
CGACATGCTGCCAAGCTCCCCAGGTCCTTGTCCTTAG

An exemplary pig DUXC double homeodomain protein may comprise a polypeptide comprising the following sequence (SEQ ID NO:119):

MPLKLAVLALCLASCQQSFFLMGSLSRGSRRRRLVLKQSQRDALQAVFQE
KPYPGITTRERLARELSIPESRIQMWFQNQRKRRLKQQSRGPPETIPQPG
PPQREQQLQTSPTPAIPKEAGRKRSFISPSQTDILRQAFERERYPGIAAR
EELARQTGIPEPQILVWFQNRRARHPEQKGSGSANVPGVDPNSAKGLPLP
SDQGMPTTAHSSPTHSAPPPPSNPPRENMLSITPMVATAAIAPKFIVPGA
PTAGCEGQSLPMIFIMAQPSPVLQAIVNPPMLWTLPLTQSSPGPMPIPAG
GLTPIHTGLWPTSQEGPWQENNLHTMPAEKCLPHIPQPPLASRAEPLPLL
DPVKTCTYARPEWAQASSAQVTSGKPVHGAMLQPAQADTLICPSHLAPSN
EELCPPIDLQQNKPSAFQGSSNLLEEIMAAAGILPEAGPLPDVEEQEELP
LGDLEAPLSEEDFQALLDMLPSSPGPCP*.

The pig DUXC homeodomain 1 polypeptide comprises the following amino acid sequence:
SRRRRLVLKQSQRDALQAVFQEKPYPGITTRERLARELSIPESRIQMWFQNQRKRRLK QQ (SEQ ID NO:120). The pig DUXC homeodomain 2 polypeptide comprises the following amino acid sequence:
AGRKRSFISPSQTDILRQAFERERYPGIAAREELARQTGIPEPQILVWFQNRRARHPEQK (SEQ ID NO:121). The pig conserved C-terminal domain comprises the following amio acid sequence. domain:

(SEQ ID NO: 122)

NLLEEIMAAAGILPEAGPLPDVEEQEELPLGDLEAPLSEEDFQALLDMLP
SSPGPCP.

An exemplary elephant DUXC double homeodomain containing protein may be encoded by a nucleic acid comprising the following sequence (SEQ ID NO:123):

ATGGATCCGACCGGCGCTTCGAGTCGCTCTCAAAATCCACGAGGCCGACG
AGAGAGGTTGGTTTTGAAGCCCAGTCAAAGAGAGACCCTGCAAGCAGCGT
TTGAACAGAACCCCTACCCTGGTATAACTACCAGAGAAGAACTCGCCAGA
GAAACCGGCATCGCGGAGGATCGCATTCAGACTTGGTTTGGAAACCGCAG
AGCAGGTCACCTAAGGAAGAGCCGCTCGGCCTCTGGACAGGCCTCCGAAG
AAGAGCCGTCCCAGGGACAGGGAGAGCCTCAGCCTTGGTCTCCGGAAAAT
TTCCCCAAAGCGGCCAGACGAAAACGCACACGCATCACCACATCGCAAAC
GAGTCTCCTAGTCGAGGCCTTCGAGAAGAACCGGTACCCTGGTAACGAGG
CCAAGGAAGAACTGGCTCAACGAACTGGCCTTCCGCGATCCCGAATTCAC
GTATGGTTTCAGAACCGAAGAGCTCGGAAGCCGGTGCAGAGCGCGAGTGC
ACCGCCGAAGTCCTTGGCAGACAGCCCGACTCCTGCGGCCACGCTTCCAC
TCGACCAAAGCGACCTGTCCTCTGTACAGAGCACCTACCCTCTCGGCCCA
CCCTCCCATCCTTCTAGCAGCAACCAAGCCATCCTACCTGTTCTCACTGA
GTCCCGTACACCATTTCTTCCTTCGGAACCCACCCAGGGCTGTGCCGGCC
AAGCACCGGGTGCCGTGTTGGACCAGCCCGCCCTGATTGTGAAGAAGACA
GCAGAGACCTCTCACGCGCCGGGGACACACCTGAACCAATCGCCAACAGG
ACCCACTGTGGGAGACAGGCTGTCAGACCCTCAGGCTCCTTTCTGGCCCC
AATACCCAGGAAATTACCAGGATCGCGACCAACATGCTGTCTCGGCAGGG
TGGCTCGCCCAAGACCCTTCTCGGCCTGACAATTCAAAGACGCAAGGGCA
GGTTCCGGCTCAGCAAGTCACAGCTCCCTTCACGCAATGGGGCTGTGAGG
TGGCCCAGGGTGTGACCGCCCGATGGGAACCCAGCCAAGAGACACTCCAG
CAGCCCGGACACTCCGAGGCACACCTGTGGCCAGAGCCGGCACAATCGGC
TCAAGAGTCATCTCATCCACCAGACCAAGACTGCCAGGAAACCGAGAGCC
TTTTAGATGAACTCCTCTCCGCCCCAGAGTTGCAGGGAAAGTCCCAAACC
TTTCTGAACGCGGATCCACAGGAGGAGGACCCTCCACAACTCGAACTCTC
CCTCGGCGACATTGACTTTCAGGCTCTGCTTGACGCGCTGCAAGATTGA

An exemplary elephant DUXC double homeodomain protein may comprise a polypeptide comprising the following sequence (SEQ ID NO:124):

MDPTGASSRSQNPRGRRERLVLKPSQRETLQAAFEQNPYPGITTREELAR
ETGIAEDRIQTWFGNRRAGHLRKSRSASGQASEEEPSQGQGEPQPWSPEN
FPKAARRKRTRITTSQTSLLVEAFEKNRYPGNEAKEELAQRTGLPRSRIH
VWFQNRRARKPVQSASAPPKSLADSPTPAATLPLDQSDLSSVQSTYPLGP
PSHPSSSNQAILPVLTESRTPFLPSEPTQGCAGQAPGAVLDQPALIVKKT
AETSHAPGTHLNQSPTGPTVGDRLSDPQAPFWPQYPGNYQDRDQHAVSAG
WLAQDPSRPDNSKTQGQVPAQQVTAPFTQWGCEVAQGVTARWEPSQETLQ
QPGHSEAHLWPEPAQSAQESSHPPDQDCQETESLLDELLSAPELQGKSQT
FLNADPQEEDPPQLELSLGDIDFQALLDALQD*.

The elephant DUXC homeodomain 1 polypeptide comprises the following amino acid sequence:
GRRERLVLKPSQRETLQAAFEQNPYPGITTREELARETGIAEDRIQTWFGNRRAGHLR KS (SEQ ID NO:125). The elephant DUXC homeodomain 2 polypeptide comprises the following amino acid sequence:
ARRKRTRITTSQTSLLVEAFEKNRYPGNEAKEELAQRTGLPRSRIHVWFQNRRARKP VQS (SEQ ID NO:126). The elephant DUXC conserved C-terminal domain comprises the following amino acid sequence:

(SEQ ID NO: 127)

SLLDELLSAPELQGKSQTFLNADPQEEDPPQLELSLGDIDFQALLDALQ
D.

An exemplary sloth DUXC double homeodomain containing protein may be encoded by a nucleic acid comprising the following sequence (SEQ ID NO:128):

ATGCGGATGACCCGAATCGCCATCTCCCTGGTGTCCGCTGATGACAGCCT
TCCAAGTACCCTGAAAGGAGTGGCCCGAAGAAAGAGGATCTTTTTGAACC
CAACTCAAATTGATGTCCTGCAAGCATCGTTTCAAAAGAACCCCTACCCT
GGTATAGCTTCCAGGGAACAACTGGCTAATGAAATTGGTGTTCCAGAGTC
TCGAATTCAGGTTTGGTTTCAGAACCGGAGAGTAAGACGCCAAAAGCAGC
ATCAACCGCAGTCTGGATCCTGCTCAGAAGATTGTTTACCCAAAGAAGCC
CGTCGTAAGCGCACATCCATCACCAGATCCCAAACCATCATTCTGGTTGA
GGCCTTTGAGCAGAACCGATTCCCTGGTGTTACAACCAGAGAAGAACTTG
CTAAACAAACAGGCCTTCCAGAAGATAGAATTCAGATATGGTTTCAGAAT
CGGAGAAATCGGTACCCAGGGAAGACACCAAGCGGACACAGAAATTCCGC
GGCAGGTGCCCCAAATCGGAGGCCTCATCTGACCATTGGGCAGGAGAAAA
CTCACCTGATCACTGTCCCAAGAAGGCCCCATCATCTTGCTTCCTGCAAT
ATTTTCCACGAGACATGCATAATTCCCTCCACTATTCTTTTGTGCCTCAC
AACCTCTGCTCTTAAGGATTCAAATGTGAACTGCATGAGTCAGGCACCCC
ATTTCCTGGAGGCCCAGCCCACACTGACTGCACAGGCAGGGGCAAACGCT
TACCCCACACAGACTATTATCAGTCACTGCCCAGCAGAGCAACCTCTGGG
AATGGGGTTCTCAGATAAGCCAAATAATTTCAAGCTCCCTTTCCAGGGAA
AATGCCAGGATCAAGATGAATCCACTGGAAGGGGAGTGGTGCAGTTGAAA
GACAATCCCCTGACACAAACTGACAATGAAAAACAACAATTACATGATGT
TGGTCGGGCAGACACATCTCACAACATGCAGTGGTGCAGCGAGGAGTTGC
AAAGTGTGAATGCAGAAGGAGAAACTCCTGAAGGGAAACTTCATCAGCCT
AGACACTCTGAGATGCAGCCAGGGCAGCAGCAGGCAGAATCAGCTGAAGA
GCCATCACTTCCCCCTGCCCAGGAGCACCAGCAAGATCTGGAGTCCTGGA
GCCTTCTGGACCAACTGCTGTCGAGCAAAGAATTTCTGGAAAAGGCCCAA
CCTCTTCTCAATCCAGATTCCCAGGACCAGAATTCTCTACCAGTTGAACC
ATCCCTCAGTGAGGAAGAGTTTCAGGCTCTGCTTGACATGCTGTGA.

An exemplary sloth DUXC double homeodomain protein may comprise a polypeptide comprising the following sequence (SEQ ID NO:129):

MRMTRIAISLVSADDSLPSTLKGVARRKRIFLNPTQIDVLQASFQKNPYP
GIASREQLANEIGVPESRIQVWFQNRRVRRQKQHQPQSGSCSEDCLPKEA
RRKRTSITRSQTIILVEAFEQNRFPGVTTREELAKQTGLPEDRIQIWFQN
RRNRYPGKTPSGHRNSAAGAPNRRPHLTIGQEKTHLITVPRRPHHLASCN
IFHETCIIPSTILLCLTTSALKDSNVNCMSQAPHFLEAQPTLTAQAGANA
YPTQTIISHCPAEQPLGMGFSDKPNNFKLPFQGKCQDQDESTGRGVVQLK
DNPLTQTDNEKQQLHDVGRADTSHNMQWCSEELQSVNAEGETPEGKLHQP
RHSEMQPGQQQAESAEEPSLPPAQEHQQDLESWSLLDQLLSSKEFLEKAQ
PLLNPDSQDQNSLPVEPSLSEEEFQALLDML*.

The sloth DUXC homeodomain 1 polypeptide comprises the following amino acid sequence:
ARRKRIFLNPTQIDVLQASFQKNPYPGIASREQLANEIGVPESRIQVWFQNRRVRRQK QH (SEQ ID NO:130). The sloth DUXC homeodomain 2 polypeptide comprises the following amino acid sequence:
ARRKRTSITRSQTIILVEAFEQNRFPGVTTREELAKQTGLPEDRIQIWFQNRRNRYPGK T (SEQ ID NO:131). The sloth DUXC conserved C-terminal domain comprises the following amino acid sequence:

(SEQ ID NO: 132)

SLLDQLLSSKEFLEKAQPLLNPDSQDQNSLPVEPSLSEEEFQALLDML.

Embodiments of the disclosure include expressing a DUXC protein in a cell. In certain embodiments, the DUXC protein comprises an amino acid sequence of a DUXC protein described herein or is encoded by a nucleic acid comprising a nucleic acid sequence disclosed herein. Also contemplated are variants of the proteins described herein. Varaints may comprise conservative amino acid substitutions in the functional domains, such as the homeodomains and/or C-terminal activation domain. The additional portions of the polypeptide may have conservative or non-conservative variations and continue to retain its functional activity. Conservative substitutions are when one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Alternatively, substitutions may be non-conservative. Non-conservative changes typically involve substituting a residue with one that is chemically dissimilar, such as a polar or charged amino acid for a nonpolar or uncharged amino acid, and vice versa.

Proteins of the disclosure may be recombinant, or synthesized in vitro. Alternatively, a non-recombinant or recombinant protein may be isolated from bacteria. It is also contemplated that a bacteria containing such a variant may be implemented in compositions and methods of the disclosure. Consequently, a protein need not be isolated.

### III. Early Cleavage-Like State

Aspects of the disclosure relate to methods of reprogramming a cell into a totipotent cell and/or a cell that exhibits an early cleavage-like state. In some embodiments, the early cleavage-like state is one that comprises activation of 2 or more, such as at least, at most, or exactly 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, 50, 55, 60, 65, 70, 75, 80, 85, 90, 95, or 100 (or any derivable range therein) cleavage-stage genes and/or families. In some embodiments, the cleavage stage genes or families comprise ZSCAN gene or family and in particular embodiments the Zscan4 gene or gene family, PRAME (preferentially expressed antigen in melanoma) gene or family, TRIM gene family, and in particular embodiments the TRIM43 gene or family (tripartite motif containing 43), RFPL4 (ret finger protein-like 4) gene or family, UBTF (upstream binding transcription factor, RNA polymerase 1) gene or family, DPPA gene or family FGF (fibroblast growth factor) gene or family, USP17 (ubiquitin specific peptidase 17)/DUB gene or family, ALYREF(Aly/REF export factor)/Thoc4 gene, ALPP (alkaline phosphatase placental) gene, Klf17 (Kruppel like factor 17) gene, Klf18/Zfp352, KDM4E (lysine demthylase 4E, SLC34A2 (solute carrier family 34 member 2), SNAI1 (snail family transcriptional repressor 1), retroviral elements ERVL, ERVL-MaLR, and Major Satellite repeats, or combinations thereof, or homologs or orthologs thereof.

In some embodiments, the cleavage stage genes comprise 1, 2, 3, 4, 5, 6, 7, 8, or 9 (or any derivable range therein) Zscan4 family members such as Zscan4a, Zscan4b, Zscan4, Zscan4-ps 1, Zscan4d, Zscan4e, Zscan4f, Zscan4-ps2, Zscan4-ps3 or orthologs or homologs thereof. In some embodiments, the cleavage stage genes comprise 1, 2, 3, 4, 5, 6, 7, 8, 9, 10, 11, 12, 13, 14, 15, 16, 17, 18, 19, 20, 21, 22, 23, 24, 25, 26, 27 or 28 (or any derivable range therein) of PRAME family members such as PRAME, PRAMEF1, PRAMEF2, PRAMEF4, PRAMEF5, PRAMEF6, PRAMEF7, PRAMEF8, PRAMEF9, PRAFEF10, PRAMEF11, PRAMEF12, PRAMEF13, PRAMEF14, PRAMEF15, PRAMEF16, PRAMEF17, PRAMEF18, PRAMEF19, PRAMEF20, PRAMEF22, PRAMEF25, PRAMEF26, PRAMEF27, and/or PRAMENP or orthologs or homologs thereof. In some embodiments, the cleavage stage genes comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33 (or any derivable range therein) of TRIMfamily members such as TRIM4, TRIM5α, TRIM6, TRIM7, TRIM10, TRIM11, TRIM15, TRIM17, TRIM21, TRIM22, TRIM25, TRIM26, TRIM27, TRIM34, TRIM35, TRIM38, TRIM39, TRIM41, TRIM43, TRIM47, TRIM48, TRIM49, TRIM50, TRIM53, TRIM58, TRIM60, TRIM62, TRIM64, TRIM65, TRIM68, TRIM69, TRIM72, TRIM75 or homologs or orthologs thereof. In some embodiments, the cleavage stage genes comprise 1, 2, 3, or 4 (or any derivable range therein) RFPL family members such as RFPL1, RFPL2, RFPL3, or RFPL4 or orthologs or homologs thereof. In some embodiments, the cleavage stage genes comprise 1, 2, 3, 4, 5, 6, or 7 (or any derivable range therein) of USP17/DUB family members such as DUB3, USP17L3, USP17L4, USP1717, DUB4, USP17L5, and USP17 or homologs or orthologs thereof.

### IV. Donor Mammalian Cells

The methods, kits and compositions as disclosed herein comprise a donor mammalian cell, from which the nuclei is injected into an enucleated oocyte to generate a SCNT embryo or for which is used as the cell in the reprogramming methods of the disclosure. In some embodiments, the donor mammalian cell is a terminally differentiated somatic cell. In some embodiments, the donor mammalian cell is not an embryonic stem cell or an adult stem cell or an iPS cell. In some embodiments, the donor mammalian cell is a human or animal cell for use in the methods as disclosed herein as donor mammalian cells where the nuclei from the donor cell is transferred into an enucleated oocyte. In some embodiments, the donor somatic cell is obtained from a male mammalian subject, e.g., XY subject. In alternative embodiments, the donor of a somatic cell is obtained from a female subject, e.g., XX subject. In some embodiments, the donor of the somatic cell is obtained from a XXY subject.

Somatic dedifferentiated cells for use with the methods of the disclosure may be primary cells or immortalized cells. Such cells may be primary cells (non-immortalized cells), such as those freshly isolated from an animal, or may be derived from a cell line (immortalized cells). Human and animal/mammalian donor somatic cells useful in the methods of the disclosure include, by way of example, epithelial, neural cells, epidermal cells, keratinocytes, hematopoietic cells, melanocytes, chondrocytes, lymphocytes (B and T lymphocytes), other immune cells, erythrocytes, macrophages, melanocytes, monocytes, mononuclear cells, fibroblasts, cardiac muscle cells, cumulus cells and other muscle cells, etc. Moreover, the human cells used for nuclear transfer may be obtained from different organs, e.g., skin, lung, pancreas, liver, stomach, intestine, heart, reproductive organs, bladder, kidney, urethra and other urinary organs, etc. These are just some examples of suitable mammalian donor cells. Suitable donor cells, i.e., cells useful in the subject disclosure, may be obtained from any cell or organ of the body. This includes all somatic and in some embodiments, germ cells e.g., primordial germ cells, sperm cells. In some embodiments, the donor cell or nucleus (i.e., nuclear genetic material) from the donor cell is actively dividing, i.e., non-quiescent cells, as this has been reported to enhance cloning efficacy. Such donor somatic cells include those in the G1, G2 S or M cell phase. Alternatively, quiescent cells may be used. In some embodiments, such donor cells will be in the G1 cell cycle. In certain embodiments, donor and/or recipient cells of the application do not undergo a 2-cell block.

In some embodiments, the nuclear genetic material (i.e., the nucleus) of a mammalian donor somatic cell is obtained from a cumulus cell, Sertoli cells or from a embryonic fibroblast or adult fibroblast cell.

In some embodiments, the nuclear genetic material is genetically modified, e.g., to correct for a genetic mutation or abnormality, or to introduce a genetic modification, for example, to study the effect of the genetic modification in a disease model, e.g., in ntESCs obtained from the SCNT embryo or totipotent cells obtained from the repgrogramming methods. In some embodiments, the nuclear genetic material is genetically modified, e.g., to introduce a desired characteristic into the somatic donor cell. Methods to genetically modify a somatic cell are well known by persons of ordinary skill in the art and are encompassed for use in the methods and compositions as disclosed herein.

In some embodiments, a donor somatic cell is selected according to the methods as disclosed in US patent Application US2004/0025193, which is incorporated herein in its entirety by reference, which discloses introducing a desired transgene into the donor somatic cell and selecting the somatic cells having the transgene prior to obtaining the nucleus for injection into the recipient oocyte.

In certain embodiments, donor nuclei (e.g., the nuclear genetic material from the donor somatic cell) may be labeled. Cells may be genetically modified with a transgene encoding a easily visualized protein such as the Green Fluorescent protein (Yang, M., et al., 2000, Proc. Natl. Acad. Sci. USA, 97:1206-1211), or one of its derivatives, or modified with a transgene constructed from the Firefly (Photinus pyralis) luciferase gene (Fluc) (Sweeney, T. J., et al. 1999, Proc. Natl. Acad. Sci. USA, 96: 12044-12049), or with a transgene constructed from the Sea Pansey (Renilla reniformis) luciferase gene (Rluc) (Bhaumik, S., and Ghambhir, S. S., 2002, Proc. Natl. Acad. Sci. USA, 99:377-382).

One or more transgenes (such as a DUXC double homeodomain protein) introduced into the nuclear genetic material of the donor somatic cell may be constitutively expressed using a “house-keeping gene” promoter such that the transgene(s) are expressed in many or all cells at a high level, or the transgene(s) may be expressed using a tissue specific and/or specific developmental stage specific gene promoter, such that only specific cell lineages or cells that have located into particular niches and developed into specific tissues or cell types express the transgene(s) and visualized (if the transgene is a reporter gene), or the transgene(s) may be expressed using an inducible promoter, such that only in the presence of the inducing agent will the transgene be expressed, to permit a transient pulse of transgene expression. Additional reporter transgenes or labeling reagents include, but are not limited to, luminescently labeled macromolecules including fluorescent protein analogs and biosensors, luminescent macromolecular chimeras including those formed with the green fluorescent protein and mutants thereof, luminescently labeled primary or secondary antibodies that react with cellular antigens involved in a physiological response, luminescent stains, dyes, and other small molecules. Labeled cells from a mosaic blastocyst can be sorted for example by flow cytometry to isolate the cloned population.

In some embodiments, mammalian donor somatic cell can be from healthy donors, e.g., healty humans, or donors with pre-existing medical conditions (e.g., Parkinson's Disease (PD) and Age Related Macular Degeneration (AMD), diabetes, obesity, cystic fibrosis, an autoimmune disease, a neurodegenerative disease, any subject with a genetic or acquired disease) or any subject whom is in need to a regenerative therapy or a stem cell transplantation to treat an existing, or pre-existing or developing condition or disease. For example, in some embodiments, a donor mammalian somatic cell is obtained from a subject who is to be a recipient of a stem cell transplant of human ES cells derived from the SCTN or reprogramming methods of the disclosure, thereby allowing autologous transplantation of patient-specific hES cells. Accordingly, in some embodiments, the methods and compositions allow for the production of patient-specific isogenic embryonic stem cell lines.

In some embodiments, a DUXC double homeodomain protein is expressed in the cell by either administering the protein to the cell or by transferring a nucleic acid encoding the protein into the cell.

### V. Method of Nuclear Transfer

Aspects of the disclosure relate to increasing the efficiency of cloning of somatic cells. The methods and compositions of the disclosure may be used for cloning a mammal, e.g., a non-human mammal, for obtaining mammalian (e.g., human and non-human mammalian) pluripotent and totipotent cells, and for reprogramming a mammalian cell.

Nuclear transfer techniques or nuclear transplantation techniques are known in the literature. See, in particular, Campbell et al, Theriogenology, 43:181 (1995); Collas et al, Mol. Report Dev., 38:264-267 (1994); Keefer et al, Biol. Reprod., 50:935-939 (1994); Sims et al, Proc. Natl. Acad. Sci., USA, 90:6143-6147 (1993); WO 94/26884; WO 94/24274, and WO 90/03432, which are incorporated by reference in their entirety herein. Also, U.S. Pat. Nos. 4,944,384 and 5,057,420 describe procedures for bovine nuclear transplantation. See, also Cibelli et al, Science, Vol. 280:1256-1258 (1998).

Transferring the donor nucleus into a recipient fertilized embryo may be done with a microinjection device. In certain embodiments, minimal cytoplasm is transferred with the nucleus. Transfer of minimal cytoplasm is achievable when nuclei are transferred using microinjection, in contrast to transfer by cell fusion approaches. In one embodiment, the microinjection device includes a piezo unit. Typically, the piezo unit is operably attached to the needle to impart oscillations to the needle. However, any configuration of the piezo unit which can impart oscillations to the needle is included within the scope of the disclosure. In certain instances, the piezo unit can assist the needle in passing into the object. In certain embodiments, the piezo unit may be used to transfer minimal cytoplasm with the nucleus. Any piezo unit suitable for the purpose may be used. In certain embodiments a piezo unit is a Piezo micromanipulator controller PMM150 (PrimeTech, Japan).

In some embodiments, the method includes a step of fusing the donor nuclei with enucleated oocyte. Fusion of the cytoplasts with the nuclei is performed using a number of techniques known in the art, including polyethylene glycol (see Pontecorvo “Polyethylene Glycol (PEG) in the Production of Mammalian Somatic Cell Hybrids” Cytogenet Cell Genet. 16(1-5):399-400 (1976), the direct injection of nuclei, Sendai viral-mediated fusion (see U.S. Pat. No. 4,664,097 and Graham Wistar Inst. Symp. Monogr. 919 (1969)), or other techniques known in the art such as electrofusion. Electrofusion of cells involves bringing cells together in close proximity and exposing them to an alternating electric field. Under appropriate conditions, the cells are pushed together and there is a fusion of cell membranes and then the formation of fusate cells or hybrid cells. Electrofusion of cells and apparatus for performing same are described in, for example, U.S. Pat. Nos. 4,441,972, 4,578,168 and 5,283,194, International Patent Application No. PCT/AU92/00473 [published as WO1993/05166], Pohl, “Dielectrophoresis”, Cambridge University Press, 1978 and Zimmerman et al., Biochimica et Bioplzysica Acta 641: 160-165, 1981.

Methods of SCNT, and activation (i.e. fusion) of the donor nuclear genetic material with the cytoplasm of the recipient oocyte are disclosed in US application 2004/0148648, which is incorporated herein in its entirety by reference.

A. Oocyte Collection.

Oocyte donors can be synchronized and superovulated as previously described (Gavin W. G., 1996), and mated to vasectomized males over a 48-hour interval. After collection, oocytes can be cultured in equilibrated M199 with 10% FBS supplemented with 2 mM L-glutamine and 1% penicillin/streptomycin (10,000 IU each/ml). Nuclear transfer can also utilize oocytes that could have been matured in vivo or in vitro.

B. Cytoplast Preparation and Enucleation.

Oocytes with attached cumulus cells are typically discarded. Cumulus-free oocytes can be divided into two groups: arrested Metaphase-II (one polar body) and Telophase-II protocols (no clearly visible polar body or presence of a partially extruding second polar body). The oocytes allocated to the activated Telophase-II protocols can be prepared by culturing for 2 to 4 hours in M199/10% FBS. After this period, all activated oocytes (presence of a partially extruded second polar body) can be grouped as culture-induced, calcium-activated Telophase-II oocytes (Telophase-Il-Ca) and enucleated. Oocytes that are not activated during the culture period can be subsequently incubated 5 minutes in M199, 10% FBS containing 7% ethanol to induce activation and then and cultured in M199 with 10% FBS for an additional time period to reach Telophase-II (Telophase-II-EtOH protocol). Oocytes may be treated with cytochalasin-B prior to enucleation. Metaphase-II stage oocytes may be enucleated with a glass pipette by aspirating the first polar body and adjacent cytoplasm surrounding the polar body (˜30% of the cytoplasm) to remove the metaphase plate. Telophase-Il-Ca and Telophase-II-EtOH oocytes can be enucleated by removing the first polar body and the surrounding cytoplasm (10 to 30% of cytoplasm) containing the partially extruding second polar body. After enucleation, all oocytes can be immediately reconstructed.

C. Nuclear Transfer and Reconstruction

Donor cell injection can be conducted in the same medium used for oocyte enucleation. One donor cell can be placed between the zona pellucida and the ooplasmic membrane using a glass pipet. The cell-oocyte couplets can be incubated in M199 before electrofusion and activation procedures. Reconstructed oocytes can be equilibrated in fusion buffer (300 mM mannitol, 0.05 mM CaCl2, 0.1 mM MgSO₄, 1 mM K₂HPO₄, 0.1 mM glutathione, 0.1 mg/ml BSA). Electrofusion and activation can be conducted at room temperature, in a fusion chamber with 2 stainless steel electrodes fashioned into a “fusion slide” (500 μm gap; BTX-Genetronics, San Diego, Calif.) filled with fusion medium.

Fusion (e.g., activation) can be performed using a fusion slide. The fusion slide can be placed inside a fusion dish, and the dish may be flooded with a sufficient amount of fusion buffer to cover the electrodes of the fusion slide. Couplets can be removed from the culture incubator and washed through fusion buffer. Using a stereomicroscope, couplets can be placed equidistant between the electrodes, with the karyoplast/cytoplast junction parallel to the electrodes. It should be noted that the voltage range applied to the couplets to promote activation and fusion can be from 1.0 kV/cm to 10.0 kV/cm. In some embodiments, the initial single simultaneous fusion and activation electrical pulse has a voltage range of 2.0 to 3.0 kV/cm, or at 2.5 kV/cm, for at least 20 pec duration. This can be applied to the cell couplet using a BTX ECM 2001 Electrocell Manipulator. The duration of the micropulse can vary from 10 to 80 μsec. After the process the treated couplet is typically transferred to a drop of fresh fusion buffer. Fusion treated couplets can be washed through equilibrated SOF/FBS, then transferred to equilibrated SOF/FBS with or without cytochalasin-B. If cytocholasin-B is used its concentration can vary from 1 to 15 μg/ml, most preferably at 5 μg/ml. The couplets can be incubated at 37-39° C. in a humidified gas chamber containing approximately 5% CO₂ in air. It should be noted that mannitol may be used in the place of cytocholasin-B throughout any of the protocols provided in the current disclosure (HEPES-buffered mannitol (0.3 mm) based medium with Ca⁺² and BSA). Starting at between 10 to 90 minutes post-fusion, most preferably at 30 minutes post-fusion, the presence of an actual karyoplast/cytoplast fusion is determined for the development of a transgenic embryo for later implantation or use in additional rounds of nuclear transfer.

Following cycloheximide treatment, couplets can be washed extensively with equilibrated SOF medium supplemented with at least 0.1% bovine serum albumin, preferably at least 0.7%, preferably 0.8%, plus 100 U/ml penicillin and 100 μg/ml streptomycin (SOF/BSA). Couplets can be transferred to equilibrated SOF/BSA, and cultured undisturbed for 24-48 hours at 37-39° C. in a humidified modular incubation chamber containing approximately 6% O2, 5% CO2, balance Nitrogen. Nuclear transfer embryos with age appropriate development (1-cell up to 8-cell at 24 to 48 hours) can be transferred to surrogate synchronized recipients.

D. Culture of SCNT Embryos

It has been suggested that embryos derived by SCNT may benefit from, or even require culture conditions in vivo other than those in which embryos are usually cultured (at least in vivo). In routine multiplication of bovine embryos, reconstituted embryos (many of them at once) have been cultured in sheep oviducts for 5 to 6 days (as described by Willadsen, In Mammalian Egg Transfer (Adams, E. E., ed.) 185 CRC Press, Boca Raton, Fla. (1982)). In certain embodiments, the SCNT embryo may be embedded in a protective medium such as agar before transfer and then dissected from the agar after recovery from the temporary recipient. The function of the protective agar or other medium is twofold: first, it acts as a structural aid for the SCNT embryo by holding the zona pellucida together; and secondly it acts as barrier to cells of the recipient animal's immune system. Although this approach increases the proportion of embryos that form blastocysts, there is the disadvantage that a number of embryos may be lost. In some embodiments, SCNT embryos can be co-cultured on monolayers of feeder cells, e.g., primary goat oviduct epithelial cells, in 50 μl droplets. Embryo cultures can be maintained in a humidified 39° C. incubator with 5% CO₂ for 48 hours before transfer of the embryos to recipient surrogate mothers.

Prior SCNT expreiments showed that nuclei from adult differentiated somatic cells can be reprgrammed to a totipotent state. Accordingly, a SCNT embryo generated using the methods as disclosed herein can be cultured in a suitable in vitro culture medium for the generation of totipotent or embryonic stem cell or stem-like cells and cell colonies. Culture media suitable for culturing and maturation of embryos are well known in the art. Examples of known media, which may be used for bovine embryo culture and maintenance, include Ham's F-10+10% fetal calf serum (FCS), Tissue Culture Medium-199 (TCM-199)+10% fetal calf serum, Tyrodes-Albumin-Lactate-Pyruvate (TALP), Dulbecco's Phosphate Buffered Saline (PBS), Eagle's and Whitten's media. One of the most common media used for the collection and maturation of oocytes is TCM-199, and 1 to 20% serum supplement including fetal calf serum, newborn serum, estrual cow serum, lamb serum or steer serum. A preferred maintenance medium includes TCM-199 with Earl salts, 10% fetal calf serum, 0.2 Ma pyruvate and 50 ug/ml gentamicin sulphate. Any of the above may also involve co-culture with a variety of cell types such as granulosa cells, oviduct cells, BRL cells and uterine cells and STO cells.

In particular, human epithelial cells of the endometrium secrete leukemia inhibitory factor (LIF) during the preimplantation and implantation period. Therefore, in some embodiments, the addition of LIF to the culture medium is encompassed to enhancing the in vitro development of the SCNT-derived embryos. The use of LIF for embryonic or stem-like cell cultures has been described in U.S. Pat. No. 5,712,156, which is herein incorporated by reference. Another maintenance medium is described in U.S. Pat. No. 5,096,822, which is incorporated herein by reference. This embryo medium, named CR1, contains the nutritional substances necessary to support an embryo. CR1 contains hemicalcium L-lactate in amounts ranging from 1.0 mM to 10 mM, preferably 1 mM to 5 mM. Hemicalcium L-lactate is L-lactate with a hemicalcium salt incorporated thereon. Also, suitable culture medium for maintaining human embryonic stem cells in culture as discussed in Thomson et al., Science, 282:1145-1147 (1998) and Proc. Natl. Acad. Sci., USA, 92:7844-7848 (1995).

In some embodiments, the feeder cells will comprise mouse embryonic fibroblasts. Means for preparation of a suitable fibroblast feeder layer are described in the example which follows and is well within the skill of the ordinary artisan.

Methods of deriving ES cells (e.g., ntESCs) from blastocyst-stage SCNT embryos (or the equivalent thereof) are well known in the art. Such techniques can be used to derive ES cells from SCNT embryos. Additionally or alternatively, ES cells can be derived from cloned SCNT embryos during earlier stages of development.

### VI. Isolation of Reprogrammed Cells and Other Stem Cells

In some embodiments, the method further comprises isolation of reprogrammed cells. The cells may be isolated based on selection of any feature specific to reprogrammed cells such as induced pluripotent stem cells compared to other somatic differentiated cells.

In particular, depending on the type of somatic differentiated cells, reprogrammed cells can be identified and isolated by any one of means of: i) isolation according to stem cell or pluripotent cell specific cell surface markers; ii) isolation by flow cytometry based on side-population (SP) phenotype by DNA dye exclusion; iii) embryoid body formation, and iv) stem cell colony picking.

In method i), cells are isolated based on stem cell-specific cell surface markers. In this method, transduced differentiated somatic cells are stained using antibodies directed to one or more stem cell-specific cell surface markers, and cells having the desired surface marker phenotype are sorted. Those skilled in the art know how to implement such isolation based on surface cell markers. For instance, flow cytometry cell-sorting may be used, transduced somatic cells are directly or indirectly fluorescently stained with antibodies directed to one or more iPSC-specific cell surface markers and cells by detected by flow cytometer laser as having the desired surface marker phenotype are sorted. In another embodiment, magnetic separation may be used. In this case, antibody labelled transduced somatic cells (which correspond to reprogrammed cells if an antibody directed to a stem cell marker is used, or to non-stem cell if an antibody specifically not expressed by stem cells is used) are contacted with magnetic beads specifically binding to the antibody (for instance via avidin/biotin interaction, or via antibody-antigen binding) and separated from antibody non-labelled transduced somatic cells. Several rounds of magnetic purification may be used based on markers specifically expressed and non-expressed by stem cells. The most common surface markers used to distinguish stem cells or induced pluripotent stem cells (iPSCs) are SSEA3, SSEA4, TRA-1 -60, and TRA-1 -81. The expression of SSEA3 and SSEA4 by reprogramming cells usually precedes the expression of TRA-1 -60 and TRA-1 -81, which are detected only at later stages of reprogramming. It has been proposed that the antibodies specific for the TRA-1 -60 and TRA-1 -81 antigens recognize distinct and unique epitopes on the same large glycoprotein Podocalyxin (also called podocalyxin-like, PODXL)1. Other surface modifications including the presence of specific lectins have also been shown to distinguish stem cells or iPSCs from non-iPSCs. Several CD molecules have been associated with pluripotency such as CD30 (tumor necrosis factor receptor superfamily, member 8, TNFRSF8), CD9 (leukocyte antigen, MIC3), CD50 (intercellular adhesion molecule-3, ICAM3), CD200 (MRC OX-2 antigen, MOX2) and CD90 (Thy-1 cell surface antigen, THY1). It also possible to distinguish iPSC by negative selection with CD44. Furthermore iPSC may be selected by the expression of the Yamanaka transcription factors (Oct4, Sox2, cMyc and Nanog).

The skilled artisan knows how to adapt the selection protocol by using one or more of different surface markers of iPSC well known in the art.

In method ii), reprogrammed cells are isolated by flow cytometry cell-sorting based on DNA dye side population (SP) phenotype. This method is based on the passive uptake of cell-permeable DNA dyes by live cells and pumping out of such DNA dyes by a side population of stem cells via ATP-Binding Cassette (ABC) transporters allowing the observation of a side population that has a low DNA dye fluorescence at the appropriate wavelength. ABC pumps can be specifically inhibited by drugs such as verapamil (100 μM final concentration) or reserpine (5 μM final concentration), and these drugs may be used to generate control samples, in which no SP phenotype may be detected. Appropriate cell-permeable DNA dyes that may be used include Hoechst 33342 (the main used DNA dye for this purpose, see Golebiewska et al., 2011) and Vybrant® DyeCycle™ stains available in various fluorescences (violet, green, and orange; see Telford et al-2010).

In method iii), reprogrammed cells are isolated by embryoid body (EB) formation. Embryoid bodies (EB) are the three dimensional aggregates formed in suspension by stem cells and/or induced pluripotent stem cells. There are several protocols to generate embryoid bodies and those skilled in the art know how to implement such isolation based on embryoid body formation. Communally, the cell population containing the reprogrammed cells are cultured previously by the embryoid formation in appropriate culture medium. On the day of EB formation when the cells grow to 60-80% confluence, cells are washed and then incubated in EDTA/PBS for 3-15 minutes to dissociate colonies to cell clumps or single cells according to EB formation methods. Often, the aggregate formation is induced by using different reagents. According to used protocol it is possible to obtain different EB formation such as self-aggregated EBs, hanging drop EBs, EBs in AggreWells ect (Lin et a/., 2014).

### VII. Selectable or Screenable Markers

In certain embodiments, cells containing a heterologous genes and nucleic acid may be identified in vitro or in vivo by including a marker in the expression vector or the nucleic acid. Such markers would confer an identifiable change to the cell permitting easy identification of cells containing the expression vector. Generally, a selection marker may be one that confers a property that allows for selection. A positive selection marker may be one in which the presence of the marker allows for its selection, while a negative selection marker is one in which its presence prevents its selection. An example of a positive selection marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selection markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes as negative selection markers such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selection and screenable markers are well known to one of skill in the art.

Selectable markers may include a type of reporter gene used in laboratory microbiology, molecular biology, and genetic engineering to indicate the success of a transfection or other procedure meant to introduce foreign DNA into a cell. Selectable markers are often antibiotic resistance genes; cells that have been subjected to a procedure to introduce foreign DNA are grown on a medium containing an antibiotic, and those cells that can grow have successfully taken up and expressed the introduced genetic material. Examples of selectable markers include: the Abicr gene or Neo gene from Tn5, which confers antibiotic resistance to geneticin.

A screenable marker may comprise a reporter gene, which allows the researcher to distinguish between wanted and unwanted cells. Certain embodiments of the present disclosure utilize reporter genes to indicate specific cell lineages. For example, the reporter gene can be located within expression elements and under the control of the ventricular- or atrial-selective regulatory elements normally associated with the coding region of a ventricular- or atrial-selective gene for simultaneous expression. A reporter allows the cells of a specific lineage to be isolated without placing them under drug or other selective pressures or otherwise risking cell viability.

Examples of such reporters include genes encoding cell surface proteins (e.g., CD4, HA epitope), fluorescent proteins, antigenic determinants and enzymes (e.g., β-galactosidase). The vector containing cells may be isolated, e.g., by FACS using fluorescently-tagged antibodies to the cell surface protein or substrates that can be converted to fluorescent products by a vector encoded enzyme.

In specific embodiments, the reporter gene is a fluorescent protein. A broad range of fluorescent protein genetic variants have been developed that feature fluorescence emission spectral profiles spanning almost the entire visible light spectrum (see below table for non-limiting examples). Mutagenesis efforts in the original Aequorea victoria jellyfish green fluorescent protein have resulted in new fluorescent probes that range in color from blue to yellow, and are some of the most widely used in vivo reporter molecules in biological research. Longer wavelength fluorescent proteins, emitting in the orange and red spectral regions, have been developed from the marine anemone, Discosoma striata, and reef corals belonging to the class Anthozoa. Still other species have been mined to produce similar proteins having cyan, green, yellow, orange, and deep red fluorescence emission. Developmental research efforts are ongoing to improve the brightness and stability of fluorescent proteins, thus improving their overall usefulness.

| | | | | | | Relative |
|---|---|---|---|---|---|---|
| | Excitation | Emission | | | | Brightness |
| Protein | Maximum | Maximum | Molar Extinction | Quantum | in vivo | (% of |
| (Acronym) | (nm) | (nm) | Coefficient | Yield | Structure | EGFP) |
| GFP (wt) | 395/475 | 509 | 21,000 | 0.77 | Monomer* | 48 |

Green Fluorescent Proteins

| | | | | | | |
|---|---|---|---|---|---|---|
| EGFP | 484 | 507 | 56,000 | 0.60 | Monomer* | 100 |
| AcGFP | 480 | 505 | 50,000 | 0.55 | Monomer* | 82 |
| TurboGFP | 482 | 502 | 70,000 | 0.53 | Monomer* | 110 |
| Emerald | 487 | 509 | 57,500 | 0.68 | Monomer* | 116 |
| Azami | 492 | 505 | 55,000 | 0.74 | Monomer | 121 |
| Green | | | | | | |
| ZsGreen | 493 | 505 | 43,000 | 0.91 | Tetramer | 117 |

Blue Fluorescent Proteins

| | | | | | | |
|---|---|---|---|---|---|---|
| EBFP | 383 | 445 | 29,000 | 0.31 | Monomer* | 27 |
| Sapphire | 399 | 511 | 29,000 | 0.64 | Monomer* | 55 |
| T-Sapphire | 399 | 511 | 44,000 | 0.60 | Monomer* | 79 |

Cyan Fluorescent Proteins

| | | | | | | |
|---|---|---|---|---|---|---|
| ECFP | 439 | 476 | 32,500 | 0.40 | Monomer* | 39 |
| mCFP | 433 | 475 | 32,500 | 0.40 | Monomer | 39 |
| Cerulean | 433 | 475 | 43,000 | 0.62 | Monomer* | 79 |
| CyPet | 435 | 477 | 35,000 | 0.51 | Monomer* | 53 |
| AmCyan1 | 458 | 489 | 44,000 | 0.24 | Tetramer | 31 |
| Midori-Ishi | 472 | 495 | 27,300 | 0.90 | Dimer | 73 |
| Cyan | | | | | | |
| mTFP1 (Teal) | 462 | 492 | 64,000 | 0.85 | Monomer | 162 |

Yellow Fluorescent Proteins

| | | | | | | |
|---|---|---|---|---|---|---|
| EYFP | 514 | 527 | 83,400 | 0.61 | Monomer* | 151 |
| Topaz | 514 | 527 | 94,500 | 0.60 | Monomer* | 169 |
| Venus | 515 | 528 | 92,200 | 0.57 | Monomer* | 156 |
| mCitrine | 516 | 529 | 77,000 | 0.76 | Monomer | 174 |
| YPet | 517 | 530 | 104,000 | 0.77 | Monomer* | 238 |
| PhiYFP | 525 | 537 | 124,000 | 0.39 | Monomer* | 144 |
| ZsYellow1 | 529 | 539 | 20,200 | 0.42 | Tetramer | 25 |
| mBanana | 540 | 553 | 6,000 | 0.7 | Monomer | 13 |

Orange and Red Fluorescent Proteins

| | | | | | | |
|---|---|---|---|---|---|---|
| Kusabira | 548 | 559 | 51,600 | 0.60 | Monomer | 92 |
| Orange | | | | | | |
| mOrange | 548 | 562 | 71,000 | 0.69 | Monomer | 146 |
| dTomato | 554 | 581 | 69,000 | 0.69 | Dimer | 142 |
| dTomato- | 554 | 581 | 138,000 | 0.69 | Monomer | 283 |
| Tandem | | | | | | |
| DsRed | 558 | 583 | 75,000 | 0.79 | Tetramer | 176 |
| DsRed2 | 563 | 582 | 43,800 | 0.55 | Tetramer | 72 |
| DsRed- | 555 | 584 | 38,000 | 0.51 | Tetramer | 58 |
| Express (T1) | | | | | | |
| DsRed- | 556 | 586 | 35,000 | 0.10 | Monomer | 10 |
| Monomer | | | | | | |
| mTangerine | 568 | 585 | 38,000 | 0.30 | Monomer | 34 |
| mStrawberry | 574 | 596 | 90,000 | 0.29 | Monomer | 78 |
| AsRed2 | 576 | 592 | 56,200 | 0.05 | Tetramer | 8 |
| mRFP1 | 584 | 607 | 50,000 | 0.25 | Monomer | 37 |
| JRed | 584 | 610 | 44,000 | 0.20 | Dimer | 26 |
| mCherry | 587 | 610 | 72,000 | 0.22 | Monomer | 47 |
| HcRed1 | 588 | 618 | 20,000 | 0.015 | Dimer | 1 |
| mRaspberry | 598 | 625 | 86,000 | 0.15 | Monomer | 38 |
| HcRed-Tandem | 590 | 637 | 160,000 | 0.04 | Monomer | 19 |
| mPlum | 590 | 649 | 41,000 | 0.10 | Monomer | 12 |
| AQ143 | 595 | 655 | 90,000 | 0.04 | Tetramer | 11 |

* Weak Dimer

### VIII. Gene Editing

In certain embodiments, engineered nucleases may be used to introduce nucleic acid sequences for genetic modification of any cells used herein, particularly the starting cells, such as somatic cells or differentiated cells as described herein.

Genome editing, or genome editing with engineered nucleases (GEEN) is a type of genetic engineering in which DNA is inserted, replaced, or removed from a genome using artificially engineered nucleases, or “molecular scissors.” The nucleases create specific double-stranded break (DSBs) at desired locations in the genome, and harness the cell's endogenous mechanisms to repair the induced break by natural processes of homologous recombination (HR) and nonhomologous end-joining (NHEJ).

Non-limiting engineered nucleases include: Zinc finger nucleases (ZFNs), Transcription Activator-Like Effector Nucleases (TALENs), the CRISPR/Cas9 system, and engineered meganuclease re-engineered homing endonucleases. Any of the engineered nucleases known in the art can be used in certain aspects of the methods and compositions.

It is commonly practiced in genetic analysis that in order to understand the function of a gene or a protein function one interferes with it in a sequence-specific way and monitors its effects on the organism. However, in some organisms it is difficult or impossible to perform site-specific mutagenesis, and therefore more indirect methods have to be used, such as silencing the gene of interest by short RNA interference (siRNA). Yet gene disruption by siRNA can be variable and incomplete. Genome editing with nucleases such as ZFN is different from siRNA in that the engineered nuclease is able to modify DNA-binding specificity and therefore can in principle cut any targeted position in the genome, and introduce modification of the endogenous sequences for genes that are impossible to specifically target by conventional RNAi. Furthermore, the specificity of ZFNs and TALENs are enhanced as two ZFNs are required in the recognition of their portion of the target and subsequently direct to the neighboring sequences.

Meganucleases, found commonly in microbial species, have the unique property of having very long recognition sequences (>14 bp) thus making them naturally very specific. This can be exploited to make site-specific DSB in genome editing; however, the challenge is that not enough meganucleases are known, or may ever be known, to cover all possible target sequences. To overcome this challenge, mutagenesis and high throughput screening methods have been used to create meganuclease variants that recognize unique sequences. Others have been able to fuse various meganucleases and create hybrid enzymes that recognize a new sequence. Yet others have attempted to alter the DNA interacting aminoacids of the meganuclease to design sequence specific meganucelases in a method named rationally designed meganuclease (U.S. Pat. No. 8,021,867 B2, incorporated herein by reference).

Meganuclease have the benefit of causing less toxicity in cells compared to methods such as ZFNs likely because of more stringent DNA sequence recognition; however, the construction of sequence specific enzymes for all possible sequences is costly and time consuming as one is not benefiting from combinatorial possibilities that methods such as ZFNs and TALENs utilize. So there are both advantages and disadvantages.

As opposed to meganucleases, the concept behind ZFNs and TALENs is more based on a non-specific DNA cutting enzyme which would then be linked to specific DNA sequence recognizing peptides such as zinc fingers and transcription activator-like effectors (TALEs). One way was to find an endonuclease whose DNA recognition site and cleaving site were separate from each other, a situation that is not common among restriction enzymes. Once this enzyme was found, its cleaving portion could be separated which would be very non-specific as it would have no recognition ability. This portion could then be linked to sequence recognizing peptides that could lead to very high specificity. An example of a restriction enzyme with such properties is FokI. Additionally FokI has the advantage of requiring dimerization to have nuclease activity and this means the specificity increases dramatically as each nuclease partner would recognize a unique DNA sequence. To enhance this effect, FokI nucleases have been engineered that can only function as heterodimers and have increased catalytic activity. The heterodimer functioning nucleases would avoid the possibility of unwanted homodimer activity and thus increase specificity of the DSB.

Although the nuclease portion of both ZFNs and TALENs have similar properties, the difference between these engineered nucleases is in their DNA recognition peptide. ZFNs rely on Cys2-His2 zinc fingers and TALENs on TALEs. Both of these DNA recognizing peptide domains have the characteristic that they are naturally found in combinations in their proteins. Cys2-His2 Zinc fingers typically happen in repeats that are 3 bp apart and are found in diverse combinations in a variety of nucleic acid interacting proteins such as transcription factors. TALEs on the other hand are found in repeats with a one-to-one recognition ratio between the amino acids and the recognized nucleotide pairs. Because both zinc fingers and TALEs happen in repeated patterns, different combinations can be tried to create a wide variety of sequence specificities. Zinc fingers have been more established in these terms and approaches such as modular assembly (where Zinc fingers correlated with a triplet sequence are attached in a row to cover the required sequence), OPEN (low-stringency selection of peptide domains vs. triplet nucleotides followed by high-stringency selections of peptide combination vs. the final target in bacterial systems), and bacterial one-hybrid screening of zinc finger libraries among other methods have been used to make site specific nucleases.

### IX. Gene Delivery

In certain embodiments, vectors could be constructed to comprise nucleic acids encoding for a DUXC double homeodomain protein (or other genese, such as detectable markers) for genetic modification of any cells used herein, particularly the somatic cells or differentiated cells of the methods of the disclosure. Details of components of these vectors and delivery methods are disclosed below.

A. Vector

One of skill in the art would be well equipped to construct a vector through standard recombinant techniques (see, for example, Maniatis et al., 1988 and Ausubel et al., 1994, both incorporated herein by reference).

Vectors can also comprise other components or functionalities that further modulate gene delivery and/or gene expression, or that otherwise provide beneficial properties to the targeted cells. Such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide.

Such components also might include markers, such as detectable and/or selection markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. A large variety of such vectors are known in the art and are generally available. When a vector is maintained in a host cell, the vector can either be stably replicated by the cells during mitosis as an autonomous structure, incorporated within the genome of the host cell, or maintained in the host cell's nucleus or cytoplasm.

B. Regulatory Elements

Eukaryotic expression cassettes included in the vectors particularly contain (in a 5′-to-3′ direction) a eukaryotic transcriptional promoter operably linked to a protein-coding sequence, splice signals including intervening sequences, and a transcriptional termination/polyadenylation sequence.

### 1. Promoter/Enhancers

A “promoter” is a control sequence that is a region of a nucleic acid sequence at which initiation and rate of transcription are controlled. It may contain genetic elements at which regulatory proteins and molecules may bind, such as RNA polymerase and other transcription factors, to initiate the specific transcription a nucleic acid sequence. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence.

A promoter generally comprises a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region 30-110 bp upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include the β-lactamase (penicillinase), lactose and tryptophan (trp) promoter systems. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. Nos. 4,683,202 and 5,928,906, each incorporated herein by reference). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook et al. 1989, incorporated herein by reference). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Additionally any promoter/enhancer combination (as per, for example, the Eukaryotic Promoter Data Base EPDB, through world wide web at epd.isb-sib.ch/) could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

Non-limiting examples of promoters include early or late viral promoters, such as, SV40 early or late promoters, cytomegalovirus (CMV) immediate early promoters, Rous Sarcoma Virus (RSV) early promoters; eukaryotic cell promoters, such as, e. g., beta actin promoter (Ng, 1989; Quitsche et al., 1989), GADPH promoter (Alexander et al., 1988, Ercolani et al., 1988), metallothionein promoter (Karin et al., 1989; Richards et al., 1984); and concatenated response element promoters, such as cyclic AMP response element promoters (cre), serum response element promoter (sre), phorbol ester promoter (TPA) and response element promoters (tre) near a minimal TATA box. It is also possible to use human growth hormone promoter sequences (e.g., the human growth hormone minimal promoter described at Genbank, accession no. X05244, nucleotide 283-341) or a mouse mammary tumor promoter (available from the ATCC, Cat. No. ATCC 45007). A specific example could be a phosphoglycerate kinase (PGK) promoter.

### 2. Protease Cleavage Sites/Self-Cleaving Peptides and Internal Ribosome Binding Sites

Suitable protease cleavages sites and self-cleaving peptides are known to the skilled person (see, e.g., in Ryan et al., 1997; Scymczak et al., 2004). Examples of protease cleavage sites are the cleavage sites of potyvirus NIa proteases (e.g. tobacco etch virus protease), potyvirus HC proteases, potyvirus P1 (P35) proteases, byovirus NIa proteases, byovirus RNA-2-encoded proteases, aphthovirus L proteases, enterovirus 2A proteases, rhinovirus 2A proteases, picorna 3C proteases, comovirus 24K proteases, nepovirus 24K proteases, RTSV (rice tungro spherical virus) 3C-like protease, PY\IF (parsnip yellow fleck virus) 3C-like protease, thrombin, factor Xa and enterokinase. Due to its high cleavage stringency, TEV (tobacco etch virus) protease cleavage sites may be used.

Exemplary self-cleaving peptides (also called “cis-acting hydrolytic elements”, CHYSEL; see deFelipe (2002) are derived from potyvirus and cardiovirus 2A peptides. Particular self-cleaving peptides may be selected from 2A peptides derived from FMDV (foot-and-mouth disease virus), equine rhinitis A virus, Thoseà asigna virus and porcine teschovirus.

A specific initiation signal also may be used for efficient translation of coding sequences in a polycistronic message. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments, the use of internal ribosome entry sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, each herein incorporated by reference).

### 3. Multiple Cloning Sites

Vectors can include a multiple cloning site (MCS), which is a nucleic acid region that contains multiple restriction enzyme sites, any of which can be used in conjunction with standard recombinant technology to digest the vector (see, for example, Carbonelli et al., 1999, Levenson et al., 1998, and Cocea, 1997, incorporated herein by reference.) “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

### 4. Splicing Sites

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression (see, for example, Chandler et al., 1997, herein incorporated by reference.)

### 5. Termination Signals

The vectors or constructs may comprise at least one termination signal. A “termination signal” or “terminator ” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, in other embodiments involving eukaryotes, the terminator comprises a signal for the cleavage of the RNA, and the terminator signal promotes polyadenylation of the message. The terminator and/or polyadenylation site elements can serve to enhance message levels and to minimize read through from the cassette into other sequences.

Terminators contemplated include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be a lack of transcribable or translatable sequence, such as due to a sequence truncation.

### 6. Polyadenylation Signals

In expression, particularly eukaryotic expression, one will typically include a polyadenylation signal to effect proper polyadenylation of the transcript. The nature of the polyadenylation signal is not believed to be crucial to the successful practice, and any such sequence may be employed. Exemplary embodiments include the SV40 polyadenylation signal or the bovine growth hormone polyadenylation signal, convenient and known to function well in various target cells. Polyadenylation may increase the stability of the transcript or may facilitate cytoplasmic transport.

### 7. Origins of Replication

In order to propagate a vector in a host cell, it may contain one or more origins of replication sites (often termed “ori”), for example, a nucleic acid sequence corresponding to oriP of EBV as described above or a genetically engineered oriP with a similar or elevated function in differentiation programming, which is a specific nucleic acid sequence at which replication is initiated. Alternatively a replication origin of other extra-chromosomally replicating virus as described above or an autonomously replicating sequence (ARS) can be employed.

C. Vector Delivery

Genetic modification or introduction of nucleic acids into starting cells may use any suitable methods for nucleic acid delivery for transformation of a cell, as described herein or as would be known to one of ordinary skill in the art. Such methods include, but are not limited to, direct delivery of DNA or RNA such as by ex vivo transfection (Wilson et al., 1989, Nabel et al, 1989), by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference; Tur-Kaspa et al., 1986; Potter et al., 1984); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991) and receptor-mediated transfection (Wu and Wu, 1987; Wu and Wu, 1988); by microprojectile bombardment (PCT Application Nos. WO 94/09699 and 95/06128; U.S. Pat. Nos. 5,610,042; 5,322,783 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium-mediated transformation (U.S. Pat. Nos. 5,591,616 and 5,563,055, each incorporated herein by reference); by PEG-mediated transformation of protoplasts (Omirulleh et al., 1993; U.S. Pat. Nos. 4,684,611 and 4,952,500, each incorporated herein by reference); by desiccation/inhibition-mediated DNA uptake (Potrykus et al., 1985), and any combination of such methods. Through the application of techniques such as these, organelle(s), cell(s), tissue(s) or organism(s) may be stably or transiently transformed.

### 1. Liposome-Mediated Transfection

In a certain embodiment, a nucleic acid may be entrapped in a lipid complex such as, for example, a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated is an nucleic acid complexed with Lipofectamine (Gibco BRL) or Superfect (Qiagen). The amount of liposomes used may vary upon the nature of the liposome as well as the, cell used, for example, about 5 to about 20 μg vector DNA per 1 to 10 million of cells may be contemplated.

Liposome-mediated nucleic acid delivery and expression of foreign DNA in vitro has been very successful (Nicolau and Sene, 1982; Fraley et al., 1979; Nicolau et al., 1987). The feasibility of liposome-mediated delivery and expression of foreign DNA in cultured chick embryo, HeLa and hepatoma cells has also been demonstrated (Wong et al., 1980).

In certain embodiments, a liposome may be complexed with a hemagglutinating virus (HVJ). This has been shown to facilitate fusion with the cell membrane and promote cell entry of liposome-encapsulated DNA (Kaneda et al., 1989). In other embodiments, a liposome may be complexed or employed in conjunction with nuclear non-histone chromosomal proteins (HMG-1) (Kato et al., 1991). In yet further embodiments, a liposome may be complexed or employed in conjunction with both HVJ and HMG-1. In other embodiments, a delivery vehicle may comprise a ligand and a liposome.

### 2. Electroporation

In certain embodiments, a nucleic acid is introduced into a cell via electroporation. Electroporation involves the exposure of a suspension of cells and DNA to a high-voltage electric discharge. Recipient cells can be made more susceptible to transformation by mechanical wounding. Also the amount of vectors used may vary upon the nature of the cells used, for example, about 5 to about 20 μg vector DNA per 1 to 10 million of cells may be contemplated.

Transfection of eukaryotic cells using electroporation has been quite successful. Mouse pre-B lymphocytes have been transfected with human kappa-immunoglobulin genes (Potter et al., 1984), and rat hepatocytes have been transfected with the chloramphenicol acetyltransferase gene (Tur-Kaspa et al., 1986) in this manner.

### 3. Calcium Phosphate

In other embodiments, a nucleic acid is introduced to the cells using calcium phosphate precipitation. Human KB cells have been transfected with adenovirus 5 DNA (Graham and Van Der Eb, 1973) using this technique. Also in this manner, mouse L(A9), mouse C127, CHO, CV-1, BHK, NIH3T3 and HeLa cells were transfected with a neomycin marker gene (Chen and Okayama, 1987), and rat hepatocytes were transfected with a variety of marker genes (Rippe et al., 1990).

### 4. DEAE-Dextran

In another embodiment, a nucleic acid is delivered into a cell using DEAE-dextran followed by polyethylene glycol. In this manner, reporter plasmids were introduced into mouse myeloma and erythroleukemia cells (Gopal, 1985).

### X. Therapeutic Applications

A. Reprogrammed Cells

Certain aspects of the disclosure relate to methods for reprogramming cells and cells comprising a heterologous gene encoding for a protein containing a DUXC double homeodomain protein. In some embodiments, the methods do not require a step of expression of Yamanaka transcription factors (Oct4, Sox2, cMyc and Klf4) or a depletion of p53 or an expression of p53 mutated proteins, and the cells obtained by the reprogramming method of the disclosure are stable and non-cancerous and have better capacity to be re-differentiated in non-cancerous somatic multipotent, unipotent or differentiated somatic cells. In some embodiments, the method further comprises expression of Yamanaka transcription factors (Oct4, Sox2, cMyc and Klf4) or a depletion of p53 or an expression of p53 mutated proteins. In some embodiments, the method may comprise expression of a DNA methyltransferase such as DNMT3.

In some embodiments, the reprogrammed cells obtained from the methods described herein may be differentiated to hematopoietic stem cells.

In another aspect, the reprogrammed cells as produced by the reprogramming method of the disclosure are used in cell therapy. In some embodiments, the reprogrammed cells are used as therapeutic agent in the treatment of aging-associated and/or degenerative diseases. Examples of aging-associated diseases are diseases include atherosclerosis, cardiovascular disease, cancer, arthritis, cataracts, osteoporosis, type 2 diabetes, hypertension, Alzheimer's disease and Parkinson disease. Examples of degenerative diseases include diseases affecting the central nervous system (Alzheimer's disease and Parkinson disease, Huntington diseases), bones (Duchene and Becker muscular dystrophies), blood vessels or heart.

In some embodiments, the reprogrammed cells are used as therapeutic agent for the treatment of aging-associated and degenerative diseases; wherein the disease is cardiovascular diseases, diabetes, cancer, arthritis, hypertension, myocardial infection, strokes, amyotrophic lateral sclerosis, Alzheimer's disease and/or Parkinson disease.

In further aspects, the reprogrammed cells are used in vitro as model for studying diseases. The models may be for studying diseases such as amyotrophic lateral sclerosis, adenosine deaminase deficiency-related severe combined immunodeficiency, Shwachman-Bodian-Diamond syndrome, Gaucher disease type III, Duchene and Becker muscular dystrophies, Parkinson's disease, Huntington's disease, type 1 diabetes mellitus, Down syndrome and/or spinal muscular atrophy.

In some embodiments, the reprogrammed cells may be used in the SCNT methods described herein.

B. Obtaining Totipotent Cells

Totipotent cells may be obtained by the reprogramming and SCNT methods described herein. In certain embodiments, blastomeres generated from SCNT embryos may be dissociated using a glass pipette to obtain totipotent cells. In some embodiments, dissociation may occur in the presence of 0.25% trypsin (Collas and Robl, 43 BIOL. REPROD. 877-84, 1992; Stice and Robl, 39 BIOL. REPROD. 657-664, 1988; Kanka et al., 43 MOL. REPROD. DEV. 135-44, 1996).

In certain embodiments, the resultant blastocysts, or blastocyst-like clusters from the SCNT embryos can be used to obtain embryonic stem cell lines, eg., nuclear transfer ESC (ntESC) cell lines. Such lines can be obtained, for example, according to the culturing methods reported by Thomson et al., Science, 282:1145-1147 (1998) and Thomson et al., Proc. Natl. Acad. Sci., USA, 92:7544-7848 (1995), incorporated by reference in their entirety herein.

Pluripotent embryonic stem cells can also be generated from a single blastomere removed from a SCNT embryo without interfering with the embryo's normal development to birth. See PCT application no. PCT/US05/39776, filed Nov. 4, 2005, the disclosures of which are incorporated by reference in their entirety; see also Chung et al., Nature V. 439, pp. 216-219 (2006), the entire disclosure of each of which is incorporated by reference in its entirety.

In some embodiments, the method comprises the utilization of cells derived from the SCNT embryo or the progeny thereof in research and in therapy. Such pluripotent or totipotent cells may be differentiated into any of the cells in the body including, without limitation, skin, cartilage, bone, skeletal muscle, cardiac muscle, renal, hepatic, blood and blood forming, vascular precursor and vascular endothelial, pancreatic beta, neurons, glia, retinal, inner ear follicle, intestinal, lung, cells.

In another embodiment of the disclosure, the SCNT embryo, or blastocyst, or pluripotent or totipotent cells obtained from a SCNT embryo (e.g., ntESCs) or the reprogramming methods of the disclosure can be exposed to one or more inducers of differentiation to yield other therapeutically-useful cells such as retinal pigment epithelium, hematopoietic precursors and hemangioblastic progenitors as well as many other useful cell types of the ectoderm, mesoderm, and endoderm. Such inducers include but are not limited to: cytokines such as interleukin-alpha A, interferon-alpha A/D, interferon-beta, interferon-gamma, interferon-gamma-inducible protein-10, interleukin-1-17, keratinocyte growth factor, leptin, leukemia inhibitory factor, macrophage colony-stimulating factor, and macrophage inflammatory protein-1 alpha, 1-beta, 2,3 alpha, 3 beta, and monocyte chemotactic protein 1-3,6kine, activin A, amphiregulin, angiogenin, B-endothelial cell growth factor, beta cellulin, brain-derived neurotrophic factor, C10, cardiotrophin-1, ciliary neurotrophic factor, cytokine-induced neutrophil chemoattractant-1, eotaxin, epidermal growth factor, epithelial neutrophil activating peptide-78, erythropoietin, estrogen receptor-alpha, estrogen receptor-beta, fibroblast growth factor (acidic and basic), heparin, FLT-3/FLK-2 ligand, glial cell line-derived neurotrophic factor, Gly-His-Lys, granulocyte colony stimulating factor, granulocytemacrophage colony stimulating factor, GRO-alpha/MGSA, GRO-beta, GRO-gamma, HCC-1, heparin-binding epidermal growth factor, hepatocyte growth factor, heregulin-alpha, insulin, insulin growth factor binding protein-1, insulin-like growth factor binding protein-1, insulin-like growth factor, insulin-like growth factor II, nerve growth factor, neurotophin-3,4, oncostatin M, placenta growth factor, pleiotrophin, rantes, stem cell factor, stromal cell-derived factor 1B, thromopoietin, transforming growth factor—(alpha, beta 1,2,3,4,5), tumor necrosis factor (alpha and beta), vascular endothelial growth factors, and bone morphogenic proteins, enzymes that alter the expression of hormones and hormone antagonists such as 17β-estradiol, adrenocorticotropic hormone, adrenomedullin, alpha-melanocyte stimulating hormone, chorionic gonadotropin, corticosteroid-binding globulin, corticosterone, dexamethasone, estriol, follicle stimulating hormone, gastrin 1, glucagons, gonadotropin, L-3,3′,5′-triiodothyronine, leutinizing hormone, L-thyroxine, melatonin, MZ-4, oxytocin, parathyroid hormone, PEC-60, pituitary growth hormone, progesterone, prolactin, secretin, sex hormone binding globulin, thyroid stimulating hormone, thyrotropin releasing factor, thyroxin-binding globulin, and vasopres sin, extracellular matrix components such as fibronectin, proteolytic fragments of fibronectin, laminin, tenascin, thrombospondin, and proteoglycans such as aggrecan, heparan sulphate proteoglycan, chontroitin sulphate proteoglycan, and syndecan. Other inducers include cells or components derived from cells from defined tissues used to provide inductive signals to the differentiating cells derived from the reprogrammed cells of the present disclosure. Such inducer cells may derive from human, non-human mammal, or avian, such as specific pathogen-free (SPF) embryonic or adult cells.

In certain embodiments of the disclosure, pluripotent, or totipotent cells obtained from a SCNT embryo (e.g., ntESCs) or a reprogramming method of the disclosure can be optionally differentiated, and introduced into the tissues in which they normally reside in order to exhibit therapeutic utility. For example, pluripotent or totipotent cells obtained from a SCNT embryo can be introduced into the tissues. In certain other embodiments, pluripotent or totipotent cells obtained from a SCNT embryo or reprogramming method can be introduced systemically or at a distance from a site at which therapeutic utility is desired. In such embodiments, the pluripotent or totipotent cells obtained from a SCNT embryo or reprogramming method can act at a distance or may hone to the desired site.

In certain embodiments of the disclosure, cloned cells, pluripotent or totipotent obtained from a SCNT embryo or reprogramming method can be utilized in inducing the differentiation of other pluripotent stem cells. The generation of single cell-derived populations of cells capable of being propagated in vitro while maintaining an embryonic pattern of gene expression is useful in inducing the differentiation of other pluripotent stem cells. Cell-cell induction is a common means of directing differentiation in the early embryo. Many potentially medically-useful cell types are influenced by inductive signals during normal embryonic development including spinal cord neurons, cardiac cells, pancreatic beta cells, and definitive hematopoietic cells. Single cell-derived populations of cells capable of being propagated in vitro while maintaining an embryonic pattern of gene expression can be cultured in a variety of in vitro, in ovo, or in vivo culture conditions to induce the differentiation of other pluripotent stem cells to become desired cell or tissue types.

The pluripotent or totipotent cells obtained from a SCNT embryo (e.g., ntESCs) or reprogramming method can be used to obtain any desired differentiated cell type. Therapeutic usages of such differentiated human cells are unparalleled. For example, human hematopoietic stem cells may be used in medical treatments requiring bone marrow transplantation. Such procedures are used to treat many diseases, e.g., late stage cancers such as ovarian cancer and leukemia, as well as diseases that compromise the immune system, such as AIDS. Hematopoietic stem cells can be obtained, e.g., by fusing an donor adult terminally differentiated somatic cells of a cancer or AIDS patient, e.g., epithelial cells or lymphocytes with a recipient enucleated oocyte, e.g., but not limited to bovine oocyte, obtaining a SCNT embryo according to the methods as disclosed herein which can then be used to obtain pluripotent or totipotent cells or stem-like cells as described above, and culturing such cells under conditions which favor differentiation, until hematopoietic stem cells are obtained. Such hematopoietic cells may be used in the treatment of diseases including cancer and AIDS. As discussed herein, the adult donor cell, or the recipient oocyte or SCNT embryo can be treated with other factors described herein.

Alternatively, the donor mammalian cells used in the SCNT methods or reprogramming methods can be adult somatic cells from a patient with a neurological disorder, and the generated SCNT embryos or totipotent cells can be used to produce pluripotent or totipotent cells which can be cultured under differentiation conditions to produce neural cell lines. Specific diseases treatable by transplantation of such human neural cells include, by way of example, Parkinson's disease, Alzheimer's disease, ALS and cerebral palsy, among others. In the specific case of Parkinson's disease, it has been demonstrated that transplanted fetal brain neural cells make the proper connections with surrounding cells and produce dopamine. This can result in long-term reversal of Parkinson's disease symptoms.

In some embodiments, the pluripotent or totipotent cells obtained from the SCNT embryo (e.g., ntESCs) or reprogramming method can be differentiated into cells with a dermatological prenatal pattern of gene expression that is highly elastogenic or capable of regeneration without causing scar formation. Dermal fibroblasts of mammalian fetal skin, especially corresponding to areas where the integument benefits from a high level of elasticity, such as in regions surrounding the joints, are responsible for synthesizing de novo the intricate architecture of elastic fibrils that function for many years without turnover. In addition, early embryonic skin is capable of regenerating without scar formation. Cells from this point in embryonic development from pluripotent or totipotent cells obtained from the SCNT embryo or reprogramming methods are useful in promoting scarless regeneration of the skin including forming normal elastin architecture. This is particularly useful in treating the symptoms of the course of normal human aging, or in actinic skin damage, where there can be a profound elastolysis of the skin resulting in an aged appearance including sagging and wrinkling of the skin.

To allow for specific selection of differentiated cells, in some embodiments, donor mammalian cells may be transfected with selectable markers expressed via inducible promoters, thereby permitting selection or enrichment of particular cell lineages when differentiation is induced. For example, CD34-neo may be used for selection of hematopoietic cells, Pw1-neo for muscle cells, Mash-1-neo for sympathetic neurons, Ma1-neo for human CNS neurons of the grey matter of the cerebral cortex, etc.

The current disclosure describes a method of using DUXC expression to make SCNT more efficient than previous methods and also the ability to make totipotent cells from differentiated donor cells. Therefore, the methods described herein provide for an essentially limitless supply of isogenic or synegenic human cells, particularly pluripotent that are not induced pluripotent stem cells, which are suitable for transplantation. In some embodiments, these are patient-specific pluripotent cells obtained from SCNT embryos or reprogramming methods, where the donor mammalian cell was obtained from a subject to be treated with the pluripotent stem cells or differentiated progeny thereof. Therefore, it will obviate the significant problem associated with current transplantation methods, i.e., rejection of the transplanted tissue which may occur because of host-vs-graft or graft-vs-host rejection. Conventionally, rejection is prevented or reduced by the administration of anti-rejection drugs such as cyclosporin. However, such drugs have significant adverse side-effects, e.g., immunosuppression, carcinogenic properties, as well as being very expensive. The present disclosure should eliminate, or at least greatly reduce, the need for anti-rejection drugs, such as cyclosporine, imulan, FK-506, glucocorticoids, and rapamycin, and derivatives thereof.

Other diseases and conditions treatable by isogenic cell therapy include, by way of example, spinal cord injuries, multiple sclerosis, muscular dystrophy, diabetes, liver diseases, i.e., hypercholesterolemia, heart diseases, cartilage replacement, burns, foot ulcers, gastrointestinal diseases, vascular diseases, kidney disease, urinary tract disease, and aging related diseases and conditions.

C. Reproductive Cloning of Non-Human Animals

In some embodiments, the methods and compositions can be used to increase the efficiency of production of SCNT embryos for cloning a non-human mammal. Methods for cloning a non-human mammal from a SCNT embryo derived from the methods and compositions as disclosed herein are well known in the art. The two main procedures used for cloning mammals are the Roslin method and the Honolulu method. These procedures were named after the generation of Dolly the sheep at the Roslin Institute in Scotland in 1996 (Campbell, K. H. et al. (1996) Nature 380:64-66) and of Cumulina the mouse at the University of Hawaii in Honolulu in 1998 (Wakayama, T. et al. (1998) Nature 394:369-374).

In other embodiments, the methods of the disclosure can be used to produce cloned cleavage stage embryos or morula stage embryos that can be used as parental embryos. Such parental embryos can be used to generate ES cells. For example, a blastomere (1, 2, 3, 4 blastomeres) can be removed or biopsied from such parental embryos and such blastomeres can be used to derive ES cells.

In particular, the present disclosure is applicable to use SCNT to generate non-human mammals having certain desired traits or characteristics, such as increased weight, milk content, milk production volume, length of lactation interval and disease resistance have long been desired. Traditional breeding processes are capable of producing animals with some specifically desired traits, but often these traits these are often accompanied by a number of undesired characteristics, are time-consuming, costly and unreliable. Moreover, these processes are completely incapable of allowing a specific animal line from producing gene products, such as desirable protein therapeutics that are otherwise entirely absent from the genetic complement of the species in question (i.e., spider silk proteins in bovine milk).

In some embodiments, the methods and compositon as disclosed herein can be used to generate transgenic non-human mammals, e.g., with an introduced desired characteristic, or absent or lacking (e.g., by gene knockout) of a particular undesirable characteristic. The development of technology capable of generating transgenic animals provides a means for exceptional precision in the production of animals that are engineered to carry specific traits or are designed to express certain proteins or other molecular compounds. That is, transgenic animals are animals that carry a gene that has been deliberately introduced into somatic and/or germline cells at an early stage of development. As the animals develop and grow the protein product or specific developmental change engineered into the animal becomes apparent.

Alternatively, the methods and compositions can be used to clone non-human mammals, e.g., produce genetically identical offspring of a particular non-human mammal. Such methods are useful in cloning of, for example, industrial or commercial animal with desirable characteristics (e.g. a cow/cattle with quality milk production and/or muscle for meat production), or cloning or producing genetically identical companion animals, e.g., pets or animals near extinction.

Briefly stated, one advantage of the methods of the discosure allows the increased efficiency of the production of transgenic non-human mammals homozygous for a selected trait. In some embodiments, where a non-human donor somatic cell has been genetically modified by transfecting the non-human mammalian cell-line with a given transgene construct containing at least one DNA encoding a desired gene; selecting a cell line(s) in which the desired gene has been inserted into the genome of that cell or cell-line; performing a nuclear transfer procedure to generate a transgenic animal heterozygous for the desired gene; characterizing the genetic composition of the heterozygous transgenic animal; selecting cells homozygous for the desired transgene through the use of selective agents; characterizing surviving cells using known molecular biology methods; picking surviving cells or cell colonies cells for use in a second round of nuclear transfer or embryo transfer; and producing a homozygous animal for a desired transgene.

An additional step that may performed according to the disclosure is to expand the cell-line obtained from the heterozygous animal in cell and/or cell-line in culture. An additional step that may performed according to the disclosure is to biopsy the heterozygous transgenic animal.

Alternatively a nuclear transfer procedure can be conducted to generate a mass of transgenic cells useful for research, serial cloning, or in vitro use. In some embodiments of the current disclosure, surviving SCNT embryos are characterized by one of several known molecular biology methods including without limitation FISH, Southern Blot, PCR. The methods provided above will allow for the accelerated production of herd homozygous for desired transgene(s) and thereby the more efficient production of a desired biopharmaceutical.

In some embodiments, the methods of the disclosure allow for the production of genetically desirable livestock or non-human mammals. For instance, in some embodiments, one or more multiple proteins can be integrated into the genome of the donor somatic cell used in the SCNT process to produce a transgenic cell line. Successive rounds of transfection with additional DNA transgenes for additional genes/molecules of interest (e.g., molecules that could be so produced, without limitation, include antibodies, biopharmaceuticals). In some embodiments, these molecules could utilize different promoters that would be actuated under different physiological conditions or would lead to production in different cell types. The beta casein promoter is one such promoter turned on during lactation in mammary epithelial cells, while other promoters could be turned on under different conditions in other cellular tissues.

In addition, the methods of the current disclosure will allow the accelerated development of one or more homozygous animals that carry a particularly beneficial or valuable gene, enabling herd scale-up and potentially increasing herd yield of a desired protein much more quickly than previous methods Likewise, the methods of the current disclosure will also provide for the replacement of specific transgenic animals lost through disease or their own mortality. It will also facilitate and accelerate the production of transgenic animals constructed with a variety of DNA constructs so as to optimize the production and lower the cost of a desirable biopharmaceutical. In another embodiment, homozygous transgenic animals are more quickly developed for xenotransplantation purposes or developed with humanized Ig loci.

D. Blastomere Culturing.

In one embodiment, the SCNT embryos can be used to generate blastomeres and utilize in vitro techniques related to those currently used in pre-implantation genetic diagnosis (PGD) to isolate single blastomeres from a SCNT embryo, generated by the methods as disclosed herein, without destroying the SCNT embryos or otherwise significantly altering their viability. As demonstrated herein, pluripotent human embryonic stem (hES) cells and cell lines can be generated from a single blastomere removed from a SCNT embryo as disclosed herein without interfering with the embryo's normal development to birth.

E. Therapeutic Cloning

The discoveries of Wilmut et al. (Wilmut, et al, Nature 385, 810 (1997) in sheep cloning of “Dolly”, together with those of Thomson et al. (Thomson et al., Science 282, 1145 (1998)) in deriving hESCs, have generated considerable enthusiasm for regenerative cell transplantation based on the establishment of patient-specific hESCs derived from SCNT-embryos or SCNT-engineered cell masses generated from a patient's own nuclei. This strategy, aimed at avoiding immune rejection through autologous transplantation, is perhaps the strongest clinical rationale for SCNT. By the same token, derivations of complex disease-specific SCNT-hESCs may accelerate discoveries of disease mechanisms. For cell transplantations, innovative treatments of murine SCID and PD models with the individual mouse's own SCNT-derived mESCs are encouraging (Rideout et al, Cell 109, 17 (2002); Barberi, Nat. Biotechnol. 21, 1200 (2003)). Ultimately, the ability to create banks of SCNT-derived stem cells with broad tissue compatibility would reduce the need for an ongoing supply of new oocytes.

The methods and composition as described herein for increasing the efficiency of SCNT and/or for producing multipotpent cells through the reprogramming methods of the disclosure have numerous important uses that will advance the field of stem cell research and developmental biology. For example, the SCNT embryos or totipotent cells can be used to generate ES cells, ES cell lines, totipotent stem (TS) cells and cell lines, and cells differentiated therefrom can be used to study basic developmental biology, and can be used therapeutically in the treatment of numerous diseases and conditions. Additionally, these cells can be used in screening assays to identify factors and conditions that can be used to modulate the growth, differentiation, survival, or migration of these cells. Identified agents can be used to regulate cell behavior in vitro and in vivo, and may form the basis of cellular or cell-free therapies.

The isolation of pluripotent human embryonic stem cells and breakthroughs in SCNT and cell reprogramming in mammals have raised the possibility of performing human SCNT or cell reprogramming to generate potentially unlimited sources of undifferentiated cells for use in research, with potential applications in tissue repair and transplantation medicine.

In the process of SCNT, the oocyte's cytoplasm would reprogram the transferred nucleus by silencing all of the somatic cell genes and activating the embryonic ones. ES cells (i.e., ntESCs) can be isolated from the inner cell mass (ICM) of the cloned pre-implantation stage embryos. With totipotent cells derived from the reprogramming methods of the disclosure, no nuclear transfer of the embryo is required. Instead, the cells are reprogrammed by expression of a DUXC protein and optionally other factors known in the art and described herein. When applied in a therapeutic setting, these cells would carry the nuclear genome of the patient; therefore, it is proposed that after directed cell differentiation, the cells could be transplanted without immune rejection to treat degenerative disorders such as diabetes, osteoarthritis, and Parkinson's disease (among others). Previous reports have described the generation of bovine ES-like cells (Cibelli et al., Nature Biotechnol. 16, 642 (1998)), and mouse ES cells from the ICMs of cloned blastocysts (Munsie et al., Curro Bio! 10, 989 (2000); Kawase, et al., Genesis 28, 156 (2000); Wakayama et al., Science 292, 740 (2001)) and the development of cloned human embryos to the 8- to 10-cell stage and blastocysts (Cibelli et al., Regen. Med. 26, 25 (2001); Shu, et al., Fertil. Steril. 78, S286 (2002)). Here, the methods and compositions of the disclosure can be used to generate human, patient-specific ES cells from SCNT-engineered cell masses or from reprogrammed cells generated by the methods as disclosed herein. Such ES cells generated from SCNTs are referred to herein as“ntESCs,” and the ntESCs as well as the totipotent cells derived from the reprogramming methods and can include patient-specific isogenic embryonic stem cell lines.

The present technique for producing human lines of hESCs utilizes excess IVF clinic embryos, and does not yield patient-specific ES cells. Patient-specific, immune-matched hESCs are anticipated to be of great biomedical importance for studies of disease and development and to advance methods of therapeutic stem cell transplantation. Accordingly, the methods of the disclosure can be used to establish hESC lines from SCNT and/or totipotent generated from human donor skin cells, human donor cumulus cells, or other human donor somatic cells from informed donors. These lines of SCNT-derived hESCs or totipotent cells derived from the reprogramming methods of the disclosure can be grown on animal protein-free culture media.

The major histocompatibility complex identity of each SCNT-derived hESCs or totipotent cell can be compared to the patient's own to show immunological compatibility, which is important for eventual transplantation. With the generation of these SCNT or totipotent cell-derived hESCs, evaluations of genetic and epigenetic stability can be made.

Many human injuries and diseases result from defects in a single cell type. If defective cells could be replaced with appropriate stem cells, progenitor cells, or cells differentiated in vitro, and if immune rejection of transplanted cells could be avoided, it might be possible to treat disease and injury at the cellular level in the clinic (Thomson et al., Science 282, 1145 (1998)). By generating hESCs from human SCNT embryos, SCNT-engineered cell masses, or totipotent reprogrammed cells, in which the somatic cell nucleus comes from the individual patient—a situation where the nuclear (though not mitochondrial DNA (mtDNA) genome is identical to that of the donor—the possibility of immune rejection might be eliminated if these cells were to be used for human treatment (Jaenisch, N. Engl. Med. 351, 2787 (2004); Drukker, Benvenisty, Trends Biotechnol. 22, 136 (2004)). Recently, mouse models of severe combined immunodeficiency (SCID) and Parkinson's disease (PD) (Barberi et al., Nat. Biotechnol. 21, 1200 (2003) have been successfully treated through the transplantation of autologous differentiated mouse embryonic stem cells (mESCs) derived from NT blastocysts, a process also referred to as therapeutic cloning.

Generating hESCs from human SCNT embryos, SCNT-engineered cell masses, or totipotent reprogrammed cells generated using the methods as disclosed herein can be assessed for the expression of hESC pluripotency markers, including alkaline phosphatase (AP), stage-specific embryonic antigen 4 (SSEA-4), SSEA-3, tumor rejection antigen 1-81 (Tra-I-81), Tra-I-60, and octamer-4 (Oct-4). DNA fingerprinting with human short tandem-repeat probes can also be used to show with high certainty that every NT-hESC line derived originated from the respective donor of the somatic mammalian cell and that these lines were not the result of enucleation failures and subsequent parthenogenetic activation. Stem cells are defined by their ability to self-renew as well as differentiate into somatic cells from all three embryonic germ layers: ectoderm, mesoderm, and endoderm. Differentiation will be analyzed in terms of teratoma formation and embryoid body (EB) formation as demonstrated by IM injection into appropriate animal models.

In summary, the present method to increase the efficiency of SCNT and for cell reprogramming provides an alternative to the current methods for deriving ES cells. However, unlike current approaches, the methods of the disclosure can be used to generate ES cell lines histocompatible with donor tissue. As such, SCNT embryos and/or reprogrammed cells produced by the methods as disclosed herein may provide the opportunity in the future to develop cellular therapies histocompatible with particular patients in need of treatment.

In some embodiments, the methods, systems, kits and devices as disclosed herein can be performed by a service provider, for example, where an investigator can request a service provider to provide a SCNT embryo, or repgrorammed totipotent cells, or pluripotent stem cells, or totipotent stem cells derived from using the methods as disclosed herein in a laboratory operated by the service provider. In such an embodiment, after obtaining a donor cell, the service provider performs the method as disclosed herein to produce the reprogrammed totipotent cell, SCNT embryo, or blastocysts derived from such a SCNT-embryo and provide the investigator with the material. In some embodiments, the investigator can send the donor cell samples to the service provider via any means, e.g., via mail, express mail, etc., or alternatively, the service provider can provide a service to collect the donor mammalian cell samples from the investigator and transport them to the diagnostic laboratories of the service provider. In some embodiments, the investigator can deposit the donor mammalian cell samples to be used in the methods of the disclosure at the location of the service provider laboratories. In alternative embodiments, the service provider provides a stop-by service, where the service provider send personnel to the laboratories of the investigator and also provides the kits, apparatus, and reagents for performing the methods and systems of the disclosure as disclosed herein of the investigators desired donor mammalian cell in the investigators laboratories. Such a service is useful for reproductive cloning of non-human mammals, e.g., for companion pets and animals as disclosed herein, or for therapeutic cloning, e.g., for obtaining pluripotent stem cells from blastocyst from the SCNT-embryos, e.g., for patient-specific pluripotent stem cells for transplantation into a subject in need of regenerative cell or tissue therapy.

### XI. Compositions and Kits

Another aspect of the disclosure relates to a population of ntESCs and/or totipotent cells (or derivatives thereof) obtained by the methods as disclosed herein. In some embodiments, the cells are human cells, for example patient-specific ntESC or totipotent cells (or derivatives), and/or patient-specific isogenic ntESCs or totipotent cells (or derivatives). In some embodiments, the cells are present in culture medium, such as a culture medium which maintains the cells in a desired state, such as in a totipotent or pluripotent state. In some embodiments, the culture medium is a medium suitable for cryopreservation. In some embodiments, the population of nt ESC are cryopreserved.

Cryogenic preservation is useful, for example, to store the cells for future use, e.g., for therapeutic use of for other uses, e.g., research use. The cells may be amplified and a portion of the amplified cells may be used and another portion may be cryogenically preserved. The ability to amplify and preserve cells allows considerable flexibility, for example, production of multiple patient-specific human cells as well in the choice of donor somatic cells for use in the methods of the disclosure. For example, cells from a histocompatible donor, may be amplified and used in more than one recipient. Cryogenic preservation of cells can be provided by a tissue bank. Cells may be cryopreserved along with histocompatibility data. ntESC produced using the methods as disclosed herein can be cryopreserved according to routine procedures. For example, cryopreservation can be carried out on from about one to ten million cells in “freeze” medium which can include a suitable proliferation medium, 10% BSA and 7.5% dimethylsulfoxide. Cells are centrifuged. Growth medium is aspirated and replaced with freeze culture medium. Ccells are resuspended as spheres. Cells are slowly frozen, by, e.g., placing in a container at −80° C. Frozen ntESCs are thawed by swirling in a 37° C. bath, resuspended in fresh stem cell medium, and grown as described above.

In some embodiments, ntESC are generated from a SCNT embryo that was generated from injection of nuclear genetic material from a donor somatic cell into the cytoplasm of a recipient oocyte, where the recipient oocyte comprises mtDNA from a third donor subject.

The current disclosure also relates to a SCNT embryo or totipotent cell produced by the methods as disclosed herein. In some embodiments, the SCNT embryo is a human embryo, and in some embodiments, the SCNT embryo is a non-human mammalian embryo. In some embodiments, the totipotent cell is a human cell or the totipotent cell is a non-human cell. In some embodiments, the non-human mammalian SCNT embryo or totipotent cell is genetically modified, e.g., at least one transgene was modified (e.g., introduced or deleted or changed) in the genetic material of the donor nucleus prior to the SCNT procedure (i.e., prior to collecting the donor nucleus and fusing with the cytoplasm of the recipient oocyte) or reprogramming procedure. In some embodiments, the SCNT embryo comprises nuclear DNA from the donor somatic cell, cytoplasm from the recipient oocyte, and mtDNA from a third donor subject.

The current disclosure also relates to a viable or living offspring of a mammal, e.g., a non-human mammal, where the living offspring is developed from an SCNT embryo produced by the methods as disclosed herein.

In another embodiment, this disclosure provides kits for the practice of the methods of this disclosure. Another aspect of the current disclosure relates to a kit, including one or more containers comprising a nucleic acid encoding for a DUXC double homeodomain protein and/or a polypeptide comprising a DUXC double homeodomain protein. In some embodiments, the kits may comprise a mammalian oocyte. The kit may optionally comprise culture medium for the recipient oocyte, the SCNT embryo, or for totipotent cells. The kit may also comprise one or more regaents for activation (e.g., fusion) of the donor nuclear genetic material with the cytoplasm of the recipient oocyte. In some embodiments, the mammalian oocyte is an enucleated oocyte. In some embodiments, the mammalian oocyte is a non-human oocyte or a human oocyte. In some embodiments, the oocyte is frozen and/or present in a cryopreservation freezing medium. In some embodiments, the oocyte is obtained from a donor female subject that has a mitochondrial disease or has a mutation or abnormality in a mtDNA. In some embodiments, the oocyte is obtained from a donor female subject that does not has a mitochondrial disease, or does not have a mutation in mtDNA. In some embodiments, the oocyte comprises mtDNA from a third subject.

### XII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Conservation and Innovation in the DUXC-Family Gene Network with Specific Reference to Human DUX4, Mouse DUX, and Canine DUXC

### I. Introduction

Facioscapulohumeral dystrophy (FSHD) is caused by the mis-expression of the DUX4 transcription factor in skeletal muscle. Animal models of FSHD have been hampered by incomplete knowledge of the conservation of the DUX4 transcriptional program in other species. This example demonstrates that both mouse Dux and human DUX4 activate genes associated with cleavage-stage embryos, including MERV-L and ERVL-MaLR retrotransposons, in mouse and human muscle cells respectively, despite divergence of their binding motifs. When expressed in mouse cells, human DUX4 maintained modest activation of genes driven by conventional promoters, but did not activate MERV-L-promoted genes. These and additional findings indicate that the ancestral DUX4-factor regulated an early cleavage-stage program driven by conventional promoters, whereas divergence of the DUX4/Dux homeodomains correlates with their retrotransposon specificity. These results provide insight into how species balance conservation of a core developmental program with innovation at retrotransposon promoters and provide a basis for developing crucial animal models of FSHD.

### II. Results

While the human DUX4 (hDUX4) transcriptome is known, the mouse DUX (mDUX) transcriptome remains largely unknown and there is not yet consensus on whether hDUX4 and mDUX are true orthologs. Both were derived by retroposition of DUXC mRNA but have diverged significantly at the sequence level. Beyond understanding their evolutionary relationship, functional comparisons critically inform improvements to murine models of FSHD, a disease which still lacks treatment options.

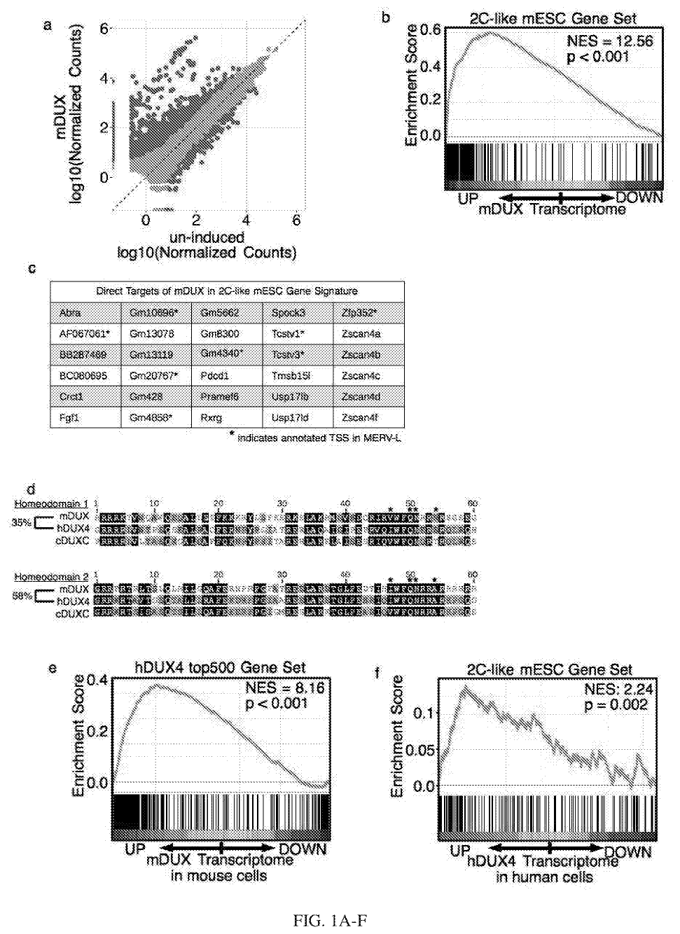

Therefore, to compare the mDUX transcriptome with the previously published hDUX4 transcriptome in FSHD muscle cells, RNA-seq and ChIP-seq datasets were generated for mDUX in mouse skeletal muscle cells. Increased expression of 962 genes and decreased expression of 204 genes were observed (FIG. 1A). In these data, the most upregulated genes were normally expressed in the mouse 2-cell embryo (e.g. Zscan4a-e, Tcstvl/3), therefore gene set enrichment analysis was used to compare the inventors' data to 2-cell-like embryonic stem cells (GSEA; 2C-like). The mDUX transcriptome was significantly enriched for the 2C-like gene signature (NES=12.56, p-value <0.001; FIG. 1B). In addition, direct targets of mDUX were enriched in the 2C-like gene signature based on hypergeometric testing (20-fold more direct targets in the 2C-like gene signature than the 1.47 genes expected by chance, p=7.8E-31), including Zscan4a-f, Tcstv1/3, Usp171b/d and Zfp352 (FIG. 1C, 7A-B). Importantly, the published 2C-like transcriptome included mDUX itself and mDUX RNA is expressed in mESC (data not shown). Impartial gene ontology analysis also identified “embryo development” among significantly enriched terms. Together, these results demonstrated that mDUX directly regulates a large portion of the 2C-like transcriptome in myoblasts.

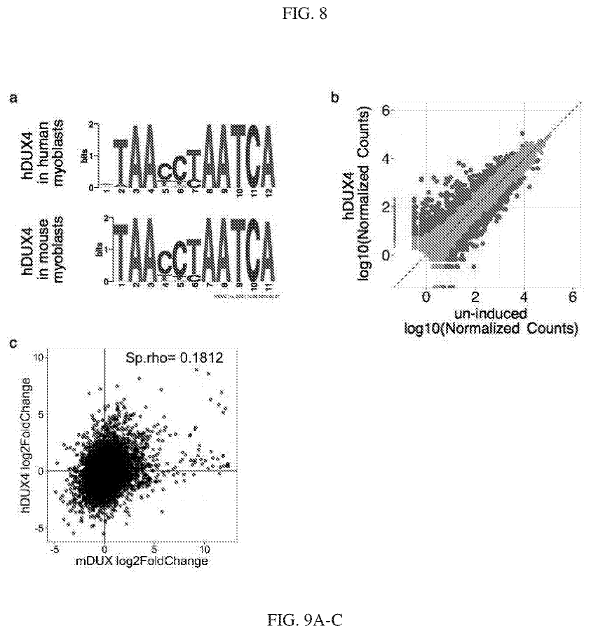

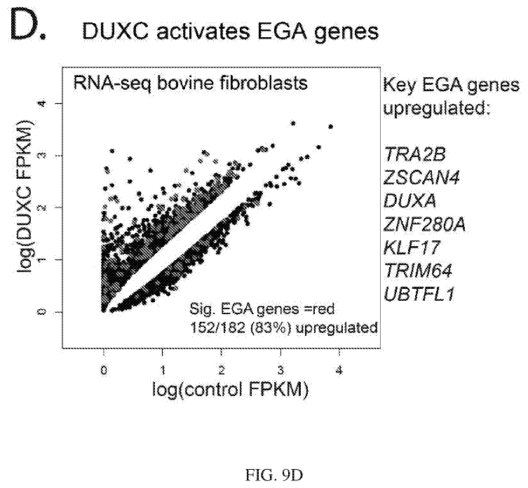

Despite considerable sequence divergence in their two DNA-binding homeodomain regions (FIG. 1D), it was found that mDUX and hDUX4 activated orthologous genes in myoblasts of their respective species, including genes in the mouse 2C-like gene signature. For this analysis only genes with simple 1:1 mouse-to-human orthology according to HomoloGene were considered. GSEA determined that the 500 genes most upregulated by hDUX4 were significantly enriched in the genes most upregulated by mDUX (NES=8.16, p-value<0.001; FIG. 1E) and vice versa (NES=6.01, p-value<0.001; FIG. 8). GSEA also demonstrated that hDUX4 activated the human orthologs of the mouse 2C-like gene signature (NES=2.24, p-value=0.002, FIG. 1F). It should be noted, however, that these analyses of similarity using the HomoloGene method was conservative. Complex gene families, such as the ZSCAN4, PRAME, THOC4/ALYREF, and USP17 families, were excluded from the HomoloGene dataset because 1:1 orthology cannot be reliably established, but members of each of these families were upregulated in both species. Together, these data demonstrate a strong functional conservation for mDUX and hDUX4 in regulation of this 2C-like network in their respective species. The inventors also confirmed that the bovine orthologue DUXC, activated many of the same key EGA genes in bovine fibroblast (FIG. 9D).

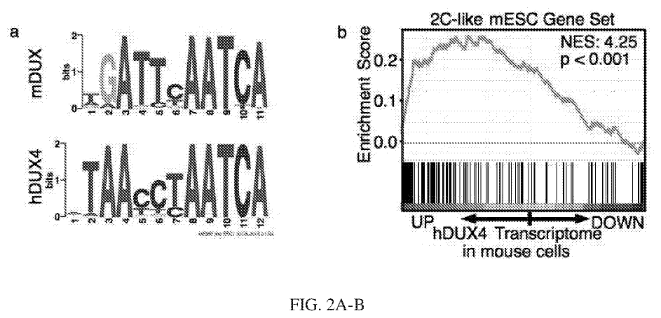

Despite this functional conservation, a de novo motif-finding algorithm identified a mDUX binding motif in the ChIP-seq data that diverged from the published hDUX4 binding motif in the first half of the motif but not the second (FIG. 2A), perhaps reflecting that the four predicted DNA-binding-specificity residues are identical between hDUX4 and mDUX in the second homeodomain but not the first (FIG. 1E).

Because of the apparent paradox of the functional conservation of their transcriptomes and the partial divergence of their binding motifs, RNA-seq and ChIP-seq datasets for hDUX4 were next generated in mouse muscle cells to better understand their conservation and divergence. In this context, hDUX4 showed the same binding motif as in human cells (FIG. 9A), increased expression of 582 genes and decreased expression of 428 genes (FIG. 9B). Although hDUX4 regulated many genes that were not orthologous to mDUX regulated genes and overall showed little similarity to the mDUX transcriptome (FIG. 9C), GSEA showed significant enrichment of the 2C-like gene signature activated by hDUX4 in mouse cells (NES=4.25, p-value<0.001; FIG. 2B). The activation of this signature, however, was not as robust by log2 fold-change compared to mDUX in mouse cells. For example, Tcstv3 and Zscan4d had log2 fold-changes of only 0.92 and 0.66, respectively, compared to 10.1 and 12.4 by mDUX, indicating that hDUX4 activates the 2C-like gene signature through moderate induction of many members. We also confirmed that the bovine orthologue DUXC, activated many of the same key EGA genes in bovine fibroblast (FIG. 9D).

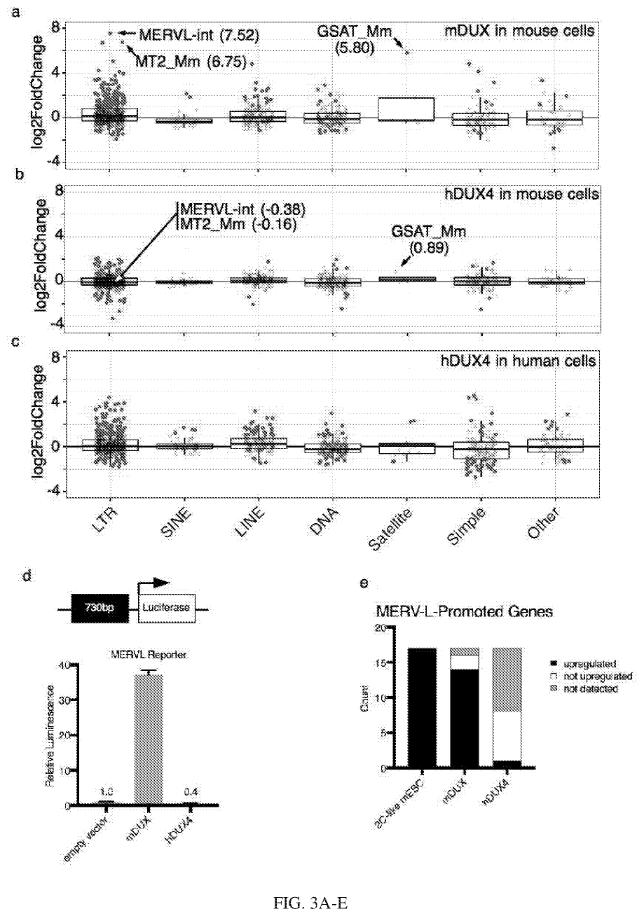

In contrast to the moderate conservation of hDUX4's activation of the 2C-like program in mouse cells, hDUX4's activation of retrotransposons completely diverged. Transcription of repetitive elements has been reported in 2C-like mouse ES cells and it was found that mDUX, but not hDUX4, induced expression of MERV-L elements by 100-fold and pericentromeric satellite DNA by 50-fold (FIG. 3A-C, 10A-C). ChIP-seq data indicated that MERV-L elements were a direct target of mDUX, but not hDUX4 (FIG. 11A-B). Consistently, mDUX, but not hDUX4, activated a reporter driven by a MERVL element (FIG. 3D). MERV-L elements have been reported to function as alternative promoters in 2C-embryos, which was observed in mDUX-expressing, but not hDUX4-expressing, mouse cells (FIG. 3E). These results indicate that hDUX4 activated a portion of the 2C-like gene signature in mouse cells, but it did not activate repetitive elements characteristic of the 2C mouse embryo.

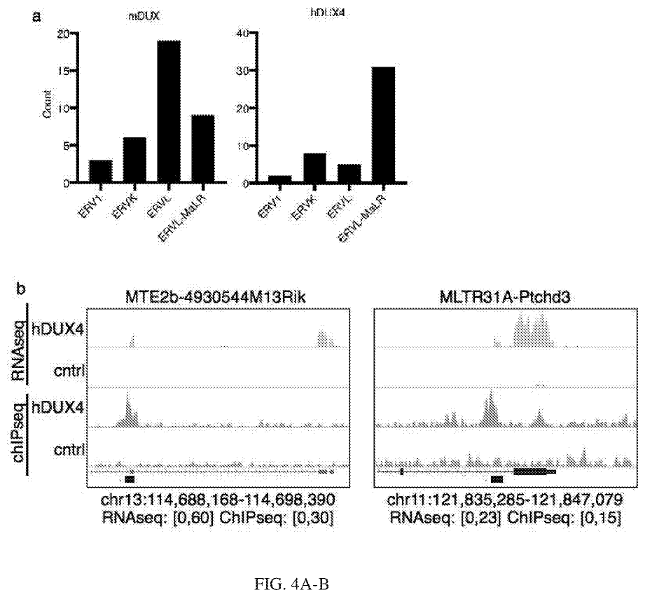

Notably, although hDUX4 did not bind MERV-L elements, hDUX4 bound ERVL-MaLR elements in mouse cells (FIG. 11B) and in at least 30 cases used them as alternative promoters (FIG. 4A). In some cases, hDUX4 binding to an ERVL-MaLR retroelement caused robust expression of the adjacent gene (FIG. 4B), consistent with the inventors' previous finding that hDUX4 binds ERVL-MaLRs when expressed in human cells and uses them as alternative promoters.

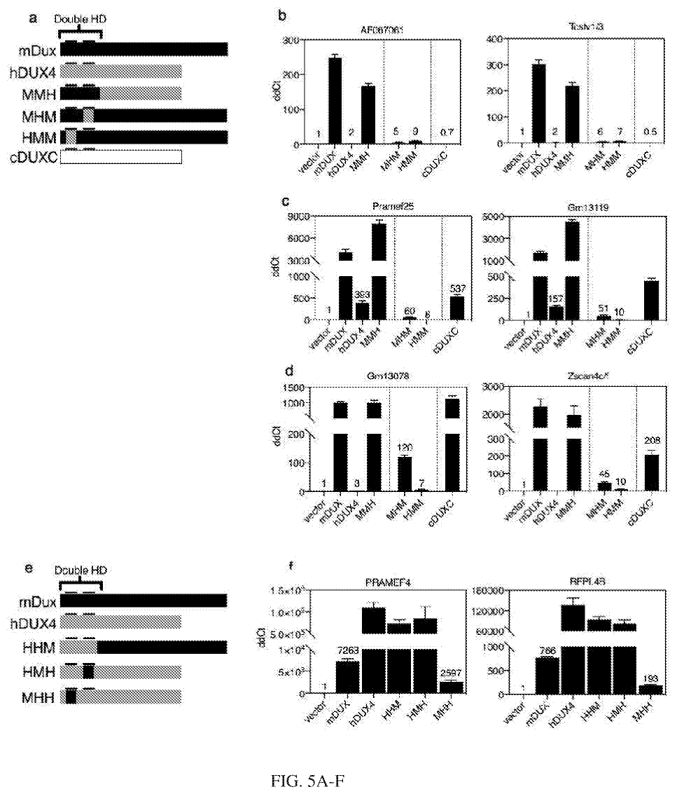

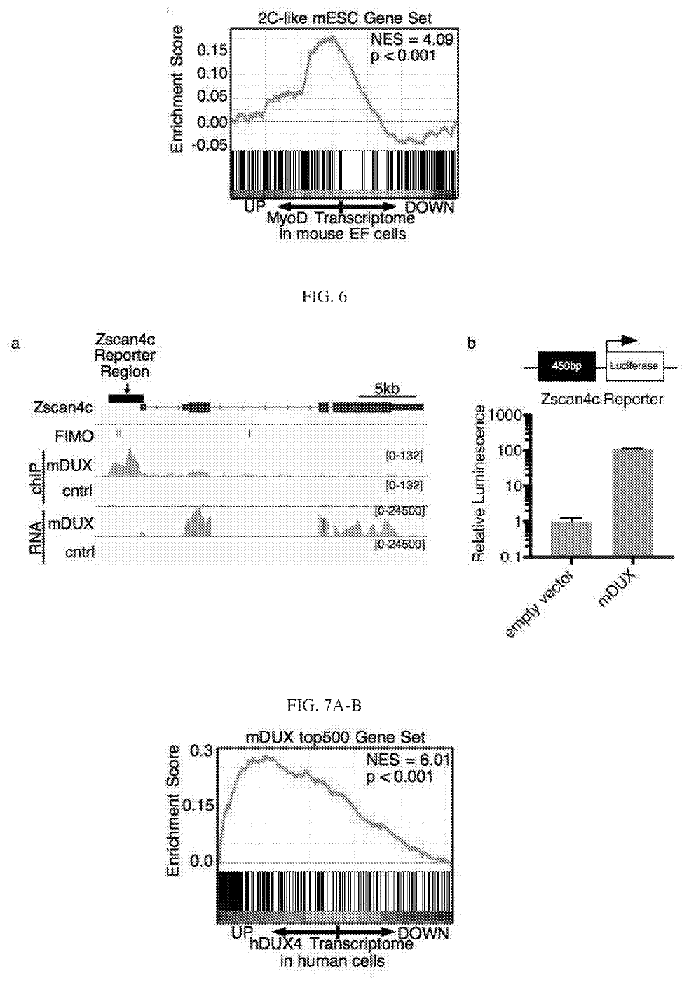

The above results indicate that mDUX and hDUX4 have maintained the ability to regulate a set of 2C-like genes in mouse cells despite considerable divergence of their homeodomains; however, conservation does not extend to the retrotransposons activated by each. Chimeric proteins were used to identify the regions of mDUX and hDUX4 responsible for this partial conservation of function (FIG. 5A). An initial chimera with the mDUX homeodomains and the hDUX4 carboxy-terminus (MMH) matched the transcriptional activity of mDUX (FIG. 5A-C), indicating that the transcriptional divergence between mDUX and hDUX4 mapped to the region containing the two homeodomains.

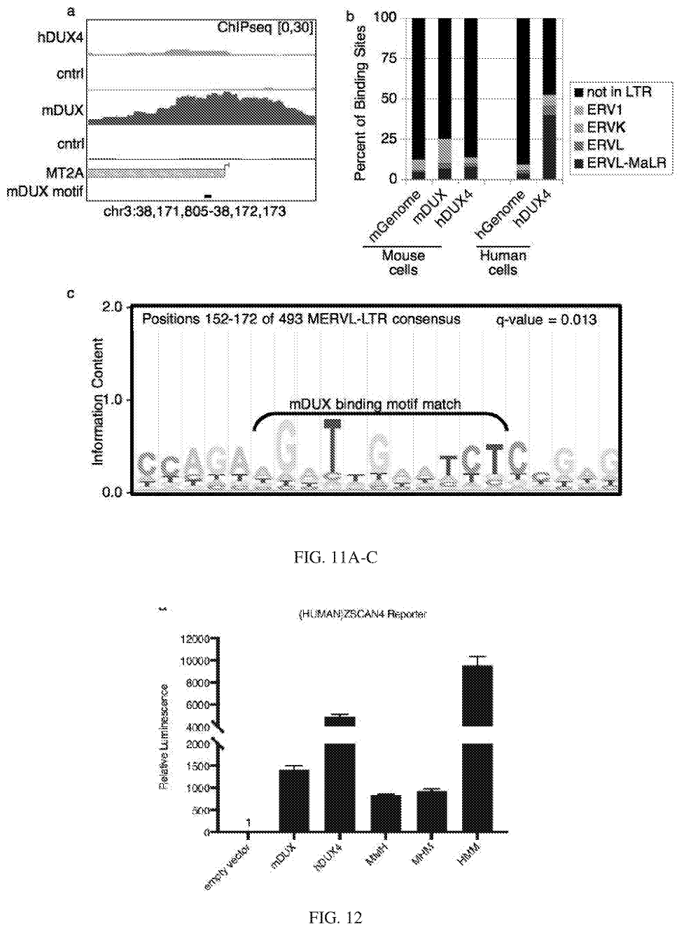

To determine the relative contribution of each homeodomain, each human homeodomain was introduced individually into mDUX to create the MHM and HMM chimeras (FIG. 5A). Neither MHM nor HMM activated transcription of MERV-L-promoted genes (FIG. 5B); whereas for 2C-like genes with conventional promoters, the individual hDUX4 homeodomains showed different capacities to substitute for the corresponding mDUX homeodomain, with MHM consistently showing stronger activation of the target genes compared to HMM (FIG. 5C-D). MHM and HMM expression and stability was confirmed using a reporter assay (FIG. 12). Reciprocal experiments in human cells were also performed and again, it was observed that the second homeodomains were more equivalent than the first homeodomains (FIG. 5E-F), indicating that the similarity of the second homeodomain was important to maintain the functional conservation of the 2C-like gene signature at conventional promoters.

To further explore the evolutionary conservation of the DUX4-family to activate an early embryo gene signature, the canine DUXC gene (cDUXC) was accessed. Both mDUX and hDUX4 are retroposed copies of ancestral DUXC mRNA and neither mice nor humans have retained DUXC (FIG. 1D). When expressed in mouse muscle cells, cDUXC did not activate MERV-L-promoted genes (FIG. 5B), but did activate transcription of 2C-like genes with conventional promoters (FIG. 5C-D), again indicating that the ancestral DUX4-like gene activated an early embryonic developmental program that was independent of retrotransposon-promoted genes.

Unlike many developmental processes that are strictly conserved between species, the homeodomain sequences and binding sites of mDUX and hDUX4 have diverged. Nevertheless, these factors have maintained the ability to activate a core developmental program, but diverged in their ability to activate subsets of retrotransposons. Genes regulated by all DUX4-family factors likely represent the core ancestral network, while retrotransposon-promoted genes likely contribute species-specific additions. Such comparisons are particularly relevant to FSHD where it remains unclear how to model this disease in non-primate animals. The fact that both hDUX4 and mDUX expression leads to apoptosis in mouse muscle cells supported the use of hDUX4 in mice as a model of FSHD. However, this study shows that homeodomain divergence will require using mDUX to best reproduce the FSHD transcriptional program in murine models of FSHD, which is lacking in current models and would facilitate evaluation of candidate FSHD therapies, none of which currently exist. This study also provides a model for studying genome evolution especially in regards to the critical balance between conservation of a key developmental program with the innovation driven by binding to mobile retrotransposon promoters.

### III. Methods

A. Whole Genome RNA-Sequencing (RNA-Seq)

C2C12, mouse myoblasts, were grown in DMEM (Gibco/Life Technologies) supplemented with 10% fetal bovine serum (Thermo Scientific) and 1% penicillin/streptomycin (Life Technologies). mDUX transgene was cloned into the pCW57.1 lentiviral vector, a gift from David Root (Addgene plasmid #41393), which has a doxyclycline-inducible promoter. mDUX and hDUX4 transgenes were codon-altered to decrease overall CpG content because this was shown to enhance transgene expression of the inducible hDUX4 vector. To create monoclonal cell lines, pCW57.1-mDUX was transduced into 293T cells, along with the packaging and envelope plasmids pMD2.G and psPAX2 using lipofectamine 2000 reagent (ThermoFisher). Viral-like-particles containing pCW57.1-hDUX4 was prepared in a similar manner. C2C12 were plated at low density and transduced with lentivirus at a low multiplicity of infection (MOI <1) in the presence of polybrene. Cells were selected and maintained in 2.6 ug/ml puromycin. Individual clones were isolated using cloning cylinders about 7 days after transfection and chosen for analysis based on robust transgene expression following 2 ug/ml doxycycline treatment for 36 hours.

Biological triplicates were prepared and total RNA was extracted from whole cells using NucleoSpin RNA kit (Macherey-Nagel) following the manufacturer's instructions. Total RNA integrity was checked using an Agilent 2200 TapeStation (Agilent Technologies, Inc., Santa Clara, Calif.) and quantified using a Trinean DropSense96 spectrophotometer (Caliper Life Sciences, Hopkinton, Mass.). RNA-seq libraries were prepared from total RNA using the TruSeq RNA Sample Prep v2 Kit (Illumina, Inc., San Diego, Calif., USA) and a Sciclone NGSx Workstation (PerkinElmer, Waltham, Mass., USA). Library size distributions were validated using an Agilent 2200 TapeStation (Agilent Technologies, Santa Clara, Calif., USA). Additional library QC, blending of pooled indexed libraries, and cluster optimization were performed using Life Technologies' Invitrogen Qubit® 2.0 Fluorometer (Life Technologies-Invitrogen, Carlsbad, Calif., USA). RNA-seq libraries were pooled (14-plex) and clustered onto two flow cell lanes. Sequencing was performed using an Illumina HiSeq 2500 in “rapid run” mode employing a single-read, 100 base read length (SR100) sequencing strategy. Image analysis and base calling was performed using Illumina's Real Time Analysis v1.18 software, followed by ‘demultiplexing’ of indexed reads and generation of FASTQ files, using Illumina's bcl2fastq Conversion Software v1.8.4 (http://support.illumina.com/downloads/bc12fastq_conversion_software_184.html).

B. RNA-seq Data Analysis

Reads of low quality were filtered prior to alignment to the reference genome (mm10 assembly) using R (development version 3.4.0) and Bioconductor (3.3.0) to call TopHat v2.1.0²², Bowtie and GenomicAlignments. Reads were allowed to map up to 20 locations. Reads overlapping UCSC known genes were counted using summerizeOverlaps and differential gene expression was determined using DESeq2. Gene Set Enrichment Analysis (GSEA) was performed using the GSEApreranked module of the Broad Institute's GenePattern²³ algorithm, using 1000 permutations and the classic scoring scheme. Gene Ontology analysis (GO) analysis was done using Gene List Analysis tool of the PANTHER Classification System (version: 10.0). Repeat element analysis was accomplished using repStats (version: 0.99.0; which will be deposited on GitHub pending publication: Link XXX), which uses summerizeOverlaps to count reads that overlap RepeatMasker-annotated repeat elements. Note, reads counts based on reads that mapped to multiple locations were divided by the number of mapped locations. Reads that support repeats used as alternative promoters or alternative first exons were identified and activation scores were calculated according to methods known in the art (see, for example, Young, J. M. et al. PLoS Genet 9, e1003947 (2013)), with the one exception that reads that linked chlPseq peaks to annotated exons were retained regardless of whether they spliced across an intron or not.

C. Whole Genome Sequencing after Chromatin Immunoprecipitation (ChIP-Seq)

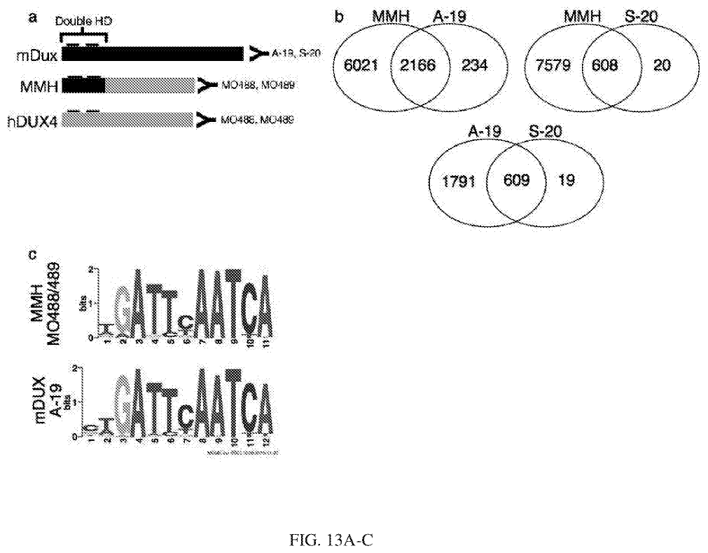

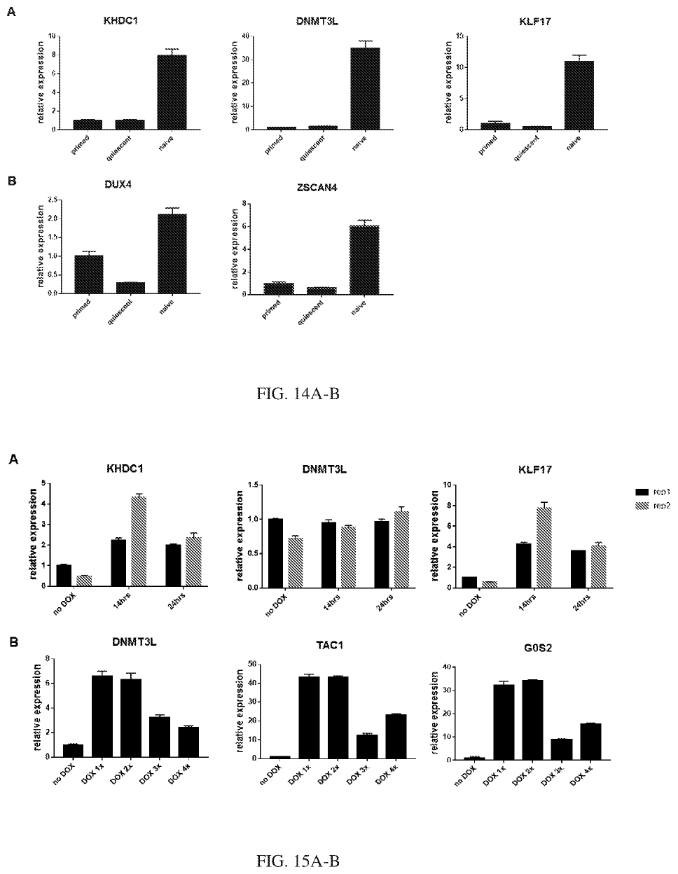

hDUX4 ChIP-seq datasets were based on monoclonal cell lines described above and were straight-forward given the availability of polyclonal antibodies to hDUX4: MO488 and MO489 were used in this study. ChIP-seq for mDUX was performed using two complementary approaches. First, two commercially available mDUX antibodies were used on a mDUX-indcucible C2C12 clonal cell line prepared as described for RNA-seq. Second, a polyclonal population of cells with the doxycycline inducible vector expressing a chimeric protein that fuses the codon-altered mDUX homeodomains with the codon-altered hDUX4 carboxyterminus (MMH) was created. The MMH-chimera maintains the DNA binding domain of mDUX and the carboxy-terminal epitopes of hDUX4, permitting us to use the same hDUX4 antisera to IP the MMH-chimera and hDUX4 (FIG. 13A). It was confirmed that the MMH-chimera retained the mDUX DNA-binding specificity by comparing the ChIP-seq peaks of the chimera to those of mDUX. Although the mDUX antibodies had a lower signal-to-noise ratio, and thus identified fewer peaks, the vast majority of the peaks identified by the mDUX-antibody were a subset of the chimera-identified peaks (FIG. 13B). ChIP-seq with one mDUX antibody, A-19, found 2,400 peaks, 90% of these peaks overlap a peak in the MMH-chimera dataset (8,187 peaks). Similarly, ChIP-seq with a second mDUX antibody, S-20, found 628 peaks, 97% of these peaks overlap with a peak in the MMH-chimera dataset. Furthermore, the MEME motif predication algorithm predicted nearly identical motifs for A-19 peaks and MMH peaks (FIG. 13C). The ChIP-seq data set from the MMH-chimera was used for all the analyses described in this example because of the superior signal-to-noise compared to the commercially available antisera to mDUX.

Cross-linked ChIP was performed similar to previous reports for other transcription factors. Briefly, ˜10⁸ cells were fixed in 1% formaldehyde for 11 minutes, quenched with glycine, lysed, and then sonicated to generate final DNA fragments of 150-600 bp. The soluble chromatin was diluted 1:10 and pre-cleared with protein A:G beads for 2 hours. Remaining chromatin was incubated with primary antibody overnight, then protein A:G beads were added for an additional 2 hours. Beads were washed and then de-crosslinked overnight. ChIP samples were validated by RT-qPCR and then prepared for sequencing per the Nugen Ovation Ultralow library system protocol with direct read barcodes. ChIP-seq libraries were prepared from IP samples using an Ovation Ultralow Library System kit (NuGEN Technologies., San Carlos, Calif., USA). Library size distributions were validated using an Agilent 2200 TapeStation (Agilent Technologies, Santa Clara, Calif., USA). Additional library QC, blending of pooled indexed libraries, and cluster optimization were performed using Life Technologies' Invitrogen Qubit® 2.0 Fluorometer (Life Technologies-Invitrogen, Carlsbad, Calif., USA). ChIP-seq libraries were pooled (12-plex) and clustered onto two flow cell lanes. Sequencing was performed using an Illumina HiSeq 2500 in Rapid Mode employing a single-read, 100-base read length (SR100) sequencing strategy. hDUX4 ChIP-seq was performed separately from mDUX and MMH.

D. ChIP-Seq Data Analysis

Image analysis and base calling were performed using Illumina's Real Time Analysis v1.18 software, followed by ‘demultiplexing’ of indexed reads and generation of FASTQ files, using Illumina's bcl2fastq Conversion Software v1.8.4 (http://support.illumina.com/downloads/bc12fastq_conversion_software_184.html). Reads of low quality were filtered out prior to alignment to mm10, using BWA 0.7.10²⁷. Further ChlPseq computational analyses were performed using R (development version 3.4.0) and Bioconductor (3.3.0). Raw reads were aligned to mm10 using Rsamtools, ShortRead, and Rsubread. Peak calling was done with MACS2 (macs2 2.1.0.20151222). Motif prediction was done with MEME-ChIP 4.11.2¹⁸, which includes FIMO analysis.

E. Transient Transfection and RT-qPCR

Transient DNA transfections of C2C12 cells were performed using SuperFect (QIAGEN) according to manufacturer specifications. Briefly, 80,000 cells were seeded per well of a 6-well plate the day prior to transfection, 2 ug DNA/well and 10 ul SuperFect/well. 24 hrs post-transfection, total RNA was extracted from whole cells using NucleoSpin RNA kit (Macherey-Nagel) following the manufacturer's instructions. One microgram of total RNA was digested with DNAseI (Invitrogen) and then reverse transcribed into first strand cDNA in a 20 uL reaction using SuperScript III (Invitrogen) and oligo(dT) (Invitrogen). cDNA was diluted and used for RT-qPCR with iTaq Universal SYBR Green Supermix (Bio-Rad). Primer efficiency was determined by standard curve and all primer sets used were >90% efficient. Relative expression levels were normalized to the endogenous control locus Timml7b and empty vector by DeltaDeltaCT.

F. Transient Transfection and Dual Luciferase Assay

Transient DNA transfections of C2C12 cells were performed using SuperFect (QIAGEN) according to manufacturer specifications. Briefly, 16,000 cells were seeded per well of a 24-well plate the day prior to transfection, 1 μg total DNA/well and 5 μl SuperFect/well. Cells to be analyzed via RT-qPCR were transfected with the expression plasmid indicated and RNA was harvested 24 hours post-transfection, then RT-qPCR proceeded as described above. Cells to be analyzed via dual luciferase assay were co-transfected with a pCS2 expression vector carrying the affector construct indicated (500 ng/well), a pCS2 expression vector carrying renilla luciferase (20 ng/well) and a pGL3-basic reporter vector (500 ng/well) carrying test promoter fragment upstream of the firefly luciferase gene. Cells were lysed 24 hours post-transfection in Passive Lysis Buffer (Promega). Luciferase activities were quantified using reagents from the Dual-Luciferase Reporter Assay System (Promega) following manufacturer's instructions. Light emission was measured using BioTek Synergy2 luminometer. Luciferase data are given as the averages±SEM of at least triplicates.

### Example 2

### DUX4 and Naïve Marker Expression in Human iPS Cells Cultured in Naïve State

### I. Results

A. FSHD2 iPS Cells Cultured in Naïve State Show Induction of Naïve Markers, DUX4, and DUX4 Target ZSCAN4

The FSHD2 iPS cell line was converted from primed state to naïve state by using the protocol from UW ES cell core (Ware et al., 2014). To check induction of known naïve markers, KHDC1, DNMT3L, and KLF17 using qRT-PCR. All of three makers were induced in iPS cells cultured in naïve state, compared to primed and quiescent state (FIG. 14A). To test whether DUX4 and its targets are also increased in the naïve state, DUX4 and ZSCAN4 expression was measured by qRT-PCR. DUX4 was induced by about 2 fold and ZSCAN4 was induced by ˜6 folds in naïve iPS cells (FIG. 14B).

B. DUX4 Expression in Control iPS Cells Increase Naïve Marker Gene Expression in Primed Control iPS Cell

To study potential roles of DUX4 in maintenance of pluripotency or reprogramming, Doxycycline (DOX) inducible codon altered DUX4 (DUX4CA) control iPS cell line was used. Cells were treated with DOX for either 14 hrs or 24 hrs and expression of three naïve markers, KHDC1, DNMT3L, and KLF17 was measured. It was observed that KHDC1 and KLF17 were induced at 14 hrs and 24 hrs DOX treated samples. (FIG. 15A).

Although the inventors did not observe distinct cell death within 24 hrs after Doxycycline (DOX) treatment on DOX-inducible DUX4CA control iPS line, some nuclei seemed to be fragmented in IF experiment, suggesting potential cell death caused by DUX4 overexpression. Thus, DOX was administered for only 8 hrs per day and up to 4 DOX treatments to test whether DUX4 expression in primed control iPS cells may induce naïve state. Four naïve markers, DNMT3L, TAC1, GOS2, and ATF5 were measured using qRT-PCR. Three out of four tested genes (except ATF5, data not shown) were induced in control iPS cells following each 8 hrs DUX4 pulse (FIG. 15B). The decreased induction following subsequent pulses is likely due to cell death from prolonged exposure to DUX4 after several pulses because it was also found that Ct values of human GAPDH in samples pulsed for three or four times were higher than after one or two times pulsed samples, suggesting greater than 8 hrs exposure to DUX4 may affect cell viability. Therefore, strategies to induce the naive state would entail one or more pulses of DUX4 expression limiting the overall time to an amount shorter than the time needed to kill the cells, approximately 16 hrs of induction.

### II. Methods

A. Human iPS Lines

FSHD2 iPS line was the gift of Dr. Daniel Miller at the University of Washington. These cell line were generated by transducing retroviral vectors expressing human OCT4, SOX2, and KLF4 (pMXs-hOCT4, pMXshSOX2, and pMXs-hKLF4) on keratinocyte from unaffected individual and fibroblast from FSHD2 patient, respectively.

eMHF2 iPS cell line was obtained from UW ES cell core. eMHF2 iPS cell line generated through transfection of episomal reprogramming vectors, pSIN4-EF2-N2L (addgene ID: 21163) and pSIN4-EF2-02S (addgene ID:21162) on human lung fibroblast (the current control iPS cell line).

B. Naïve Cell Culture

Primed iPS cells were treated with HDAC inhibitors, Sodium butyrate (0.1 mM) and SAHA (50 nM) and passaged with dispase. HDAC inhibitors were treated for at least 3 passages (quiescent state). Then, quiescent iPS cells were treated with MEK inhibitor (Selleck #S 1036: 1 μM), GSK3 inhibitor (Selleck #263: 1 μM), human LIF (10 ng/ml), IGF1 (5 ng/ml), and FGF (10 ng/ml) for at least 3 passages (naïve state). While inhibitors and growth factors were treated to iPS cells, trypsin was used to passage them.

### Example 3

### Conserved Roles for Murine DUX and Human DUX4 in Activating Cleavage Stage Genes and MERVL/HERVL Retrotransposons

The inventors sought to define the changes in transcription/transcript abundance that accompany human egg and pre-implantation embryo development. Analysis of the results revealed the cleavage stage as highly unique, similar to observations made in mouse, and the in silico analyses suggested upstream regulatory involvement of a cleavage-specific homeodomain transcription factor called human DUX4 (hereinafter hDUX4). hDUX4 has been characterized previously for its causal role in the disease facioscapulohumeral muscular dystrophy (FSHD), whereby its improper expression in muscle cells activates genes and retrotransposons normally expressed in human embryos, inciting apoptosis. This example provides multiple lines of evidence that hDUX4 and its mouse ortholog, mDUX, likely share central roles in driving cleavage-specific gene transcription (including Zscan4, Kdm4e, Zfp352, MERVL, etc.) and chromatin remodeling, and eliciting key cleavage-specific processes. Taken together, hDUX4 and mDUX appear to reside at the top of a transcriptional hierarchy initiated at EGA that helps define and drive the unique cleavage stage in mammalian embryogenesis.

### I. Results

A. RNA Transcriptomes from Developing human Oocytes and Early Embryos

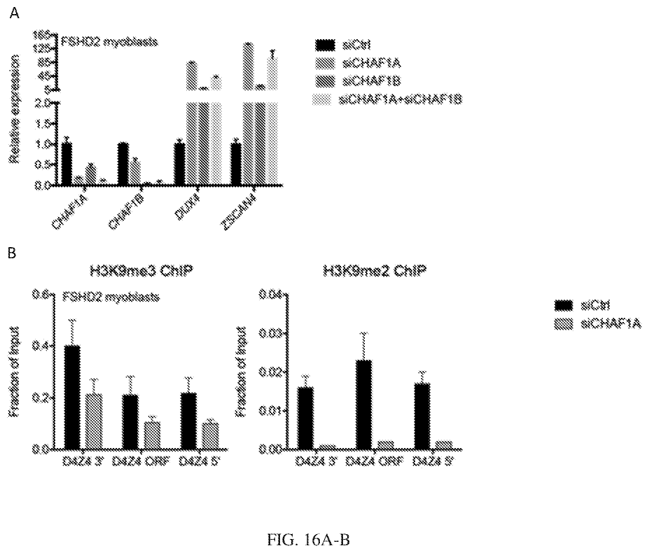

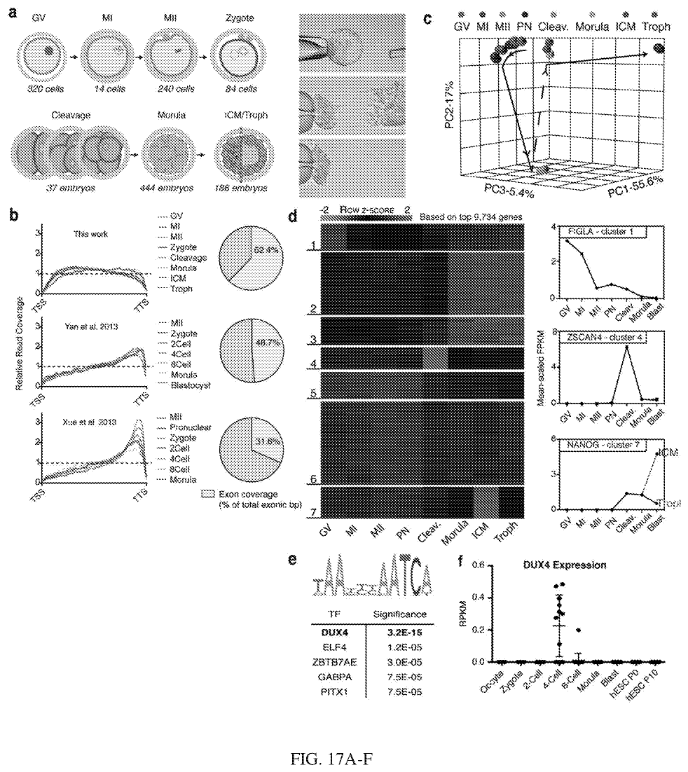

Samples from seven stages of human oogenesis and early embryogenesis were donated from consented patients undergoing in vitro fertilization (IVF) in accordance with Institutional Review Board (IRB) guidelines and approval, using standard IVF culture condition. Through laser dissection, blastocyst samples were separated into ICM (with minimal contaminating polar trophectoderm) and mural trophectoderm (FIG. 16A). To minimize variation, all samples were processed together. For each, total RNA was divided (providing two technical replicates) and processed in parallel using a transposase-based library method involving random hexamer priming to sequence total RNA without 3′ biases. To maximize dataset utility, deep RNA sequencing (RNAseq) was performed using a paired-end 101 bp sequencing format on a HiSeq2000. Only unique reads were used for the analyses, enabled by the long reads and paired-end formats. Each developmental stage replicate yielded an average of ˜76 million mappable unique reads and technical replicates were highly similar (r>0.92). Importantly, read coverage from the transcription start site (TSS) to the transcription termination site was well-balanced compared to prior work (FIG. 16B, 24A), providing deeper exonic coverage.

B. PCA and Clustering Analyses Reveal a Unique Cleavage-Stage Transcriptome

Collectively, 19,534 (33.3%) of the 58,721 genes annotated by Ensembl were expressed across the sample series (count>10). Remarkably, 17,335 (88.7%) were differentially expressed (fold change>2; FDR<0.01) in at least one stage by adjacent stage pairwise analyses. To examine developmental order, principal component analysis (PCA) was performed using all genes of moderate-to-high expression (9,734; Fragments Per Kilobase Per Million [FPKM] >1). The top three principal components effectively separated the sampled stages, while replicates of the same stage remained closely associated (FIG. 16C). Here, separation distances within the PCA map represent the extent to which developmental transitions are accompanied by major changes in transcription (or transcript abundance). Notably, the stages of oocyte development (along with the pronuclear stage) co-localize along a short temporal arc, consistent with progressive but moderate changes in transcript abundance. In contrast, the cleavage replicates were clearly distinct, consistent with major new transcription at embryonic-genome activation (EGA). An additional major change involves transition to the morula stage, which appears markedly similar to trophectoderm replicates—whereas the ICM replicates formed a distinct separate group. Thus, PCA and pair wise analyses of transcription indicate three major developmental phases: pre-EGA, EGA, and post-EGA.

K-means clustering (FIG. 16D) likewise partitioned transcription into three clear phases: pre-EGA (Clusters 1-3), EGA, (Cluster 4) and post-EGA (Clusters 5-7). Here, cluster 1 transcripts are those highest at GV stage (e.g. FIGLA), Cluster 4 transcripts are enriched in known cleavage-specific factors (e.g. ZSCAN4), and Cluster 7 transcripts in known ICM factors (e.g. NANOG).

C. Examination of Alternative Splicing and Novel Transcription

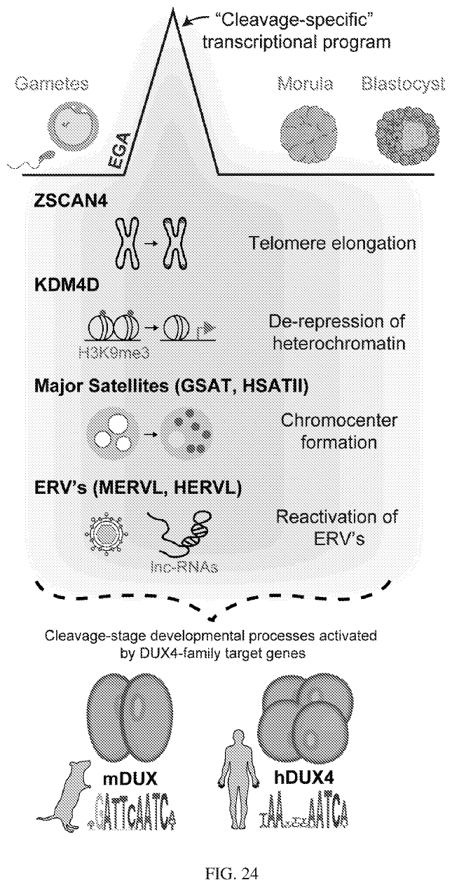

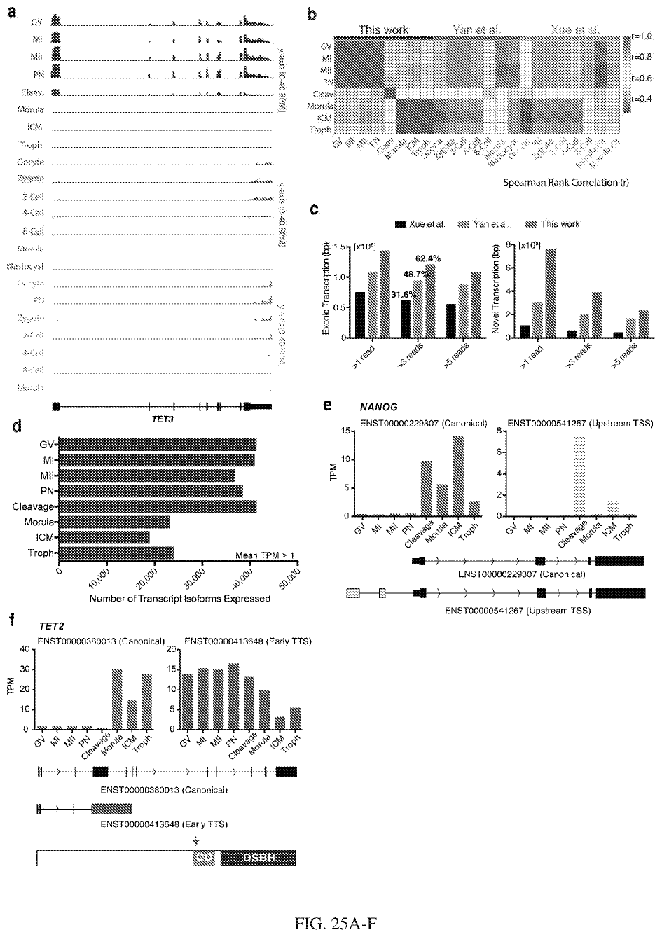

Overall, the transcription profiles were consistent with prior single cell datasets (FIG. 16B). However the improvements in read coverage balance enabled improved analyses of novel transcription and alternative splicing (FIG. 24C-D). For example, thousands of non-canonical splice isoforms expressed dynamically during pre-implantation development were identified, including prominent transcription factors (e.g. NANOG, TET2; FIG. 24E-F). Furthermore, this approach yielded considerably more novel transcribed regions during these developmental stages than prior datasets, by multiple measures. Taken together, the combined approaches yielded datasets with extensive information on differential gene expression, novel transcription, and alternative splicing, providing a major resource for future studies.

D. A hDUX4 Binding Motif is Enriched Upstream of Cleavage-Specific Genes

The inventors then addressed a key question in pre-implantation embryo development—which transcription factors define and drive the distinctive cleavage stage/EGA transcriptome? The inventors identified above a set of genes strongly and transiently transcribed in the human cleavage embryo (FIG. 16D [Cluster 4]). To identify candidate transcription factor(s) responsible for the selective activation of endogenous genes and/or retrotransposons during human EGA, Cluster 4 gene promoter regions (5 kb) were analyzed for enriched transcription factor binding motif(s). Notably, only one motif provided striking enrichment: DUX4 (p=3.2E-15)(FIG. 16E), a member of the double homeobox (DUX) family of transcription factors, whose improper expression causes a human muscular dystrophy, facioscapulohumeral muscular dystrophy (FSHD). hDUX4 is one of three coding DUX genes in humans, which also includes DUXA and DUXB. This family belongs to the larger ‘paired’ (PRD) class of homeodomains which further includes a set of diverging tandem duplicates of the CRX gene; ARGFX, LEUTX, DPRX, and TPRX1 (FIG. 25A). Their temporal expression is remarkable; hDUX4 mRNA is restricted to the 4-cell cleavage stage (early EGA) (FIG. 16F), which precedes the expression of the other PRD-family members DUXA, DUXB, ARGFX, LEUTX, DPRX, and TPRX1—all of which display strong and transient expression solely during late cleavage and/or morula stages (FIG. 25B) and at no other reported time in development.

E. hDUX4 Potently Activates Cleavage-Specific Genes and Retroviral Elements

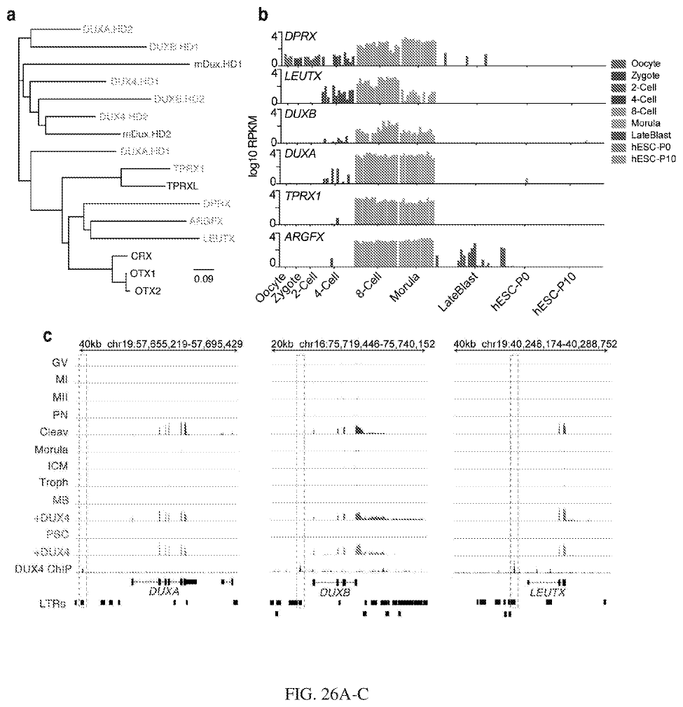

To provide functional tests of hDUX4 in defining and driving cleavage stage-specific transcription, hDUX4 transcriptional targets were identified by introducing a doxycycline—inducible hDUX4 expression cassette (or luciferase control) into a human induced pluripotent stem cell line (iPSC), induced expression via doxycycline (dox) for 14 or 24 hr, and performed RNAseq. This yielded 305 and 324 differentially expressed genes (FC>2; FDR<0.01), respectively (FIG. 26A) with the vast majority being upregulated (97.4% and 80.1%, respectively). Remarkably, these upregulated genes overlapped greatly with genes transiently and specifically expressed in cleavage embryos (FIG. 17A, FIG. 26B), including some of the related PRD class members DUXA, DUXB, and LEUTX (FIG. 25C).

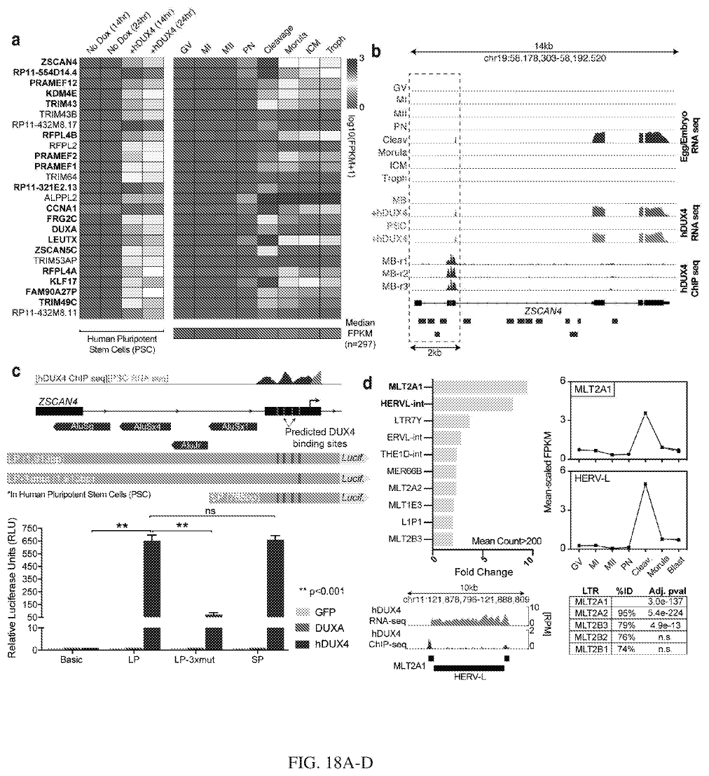

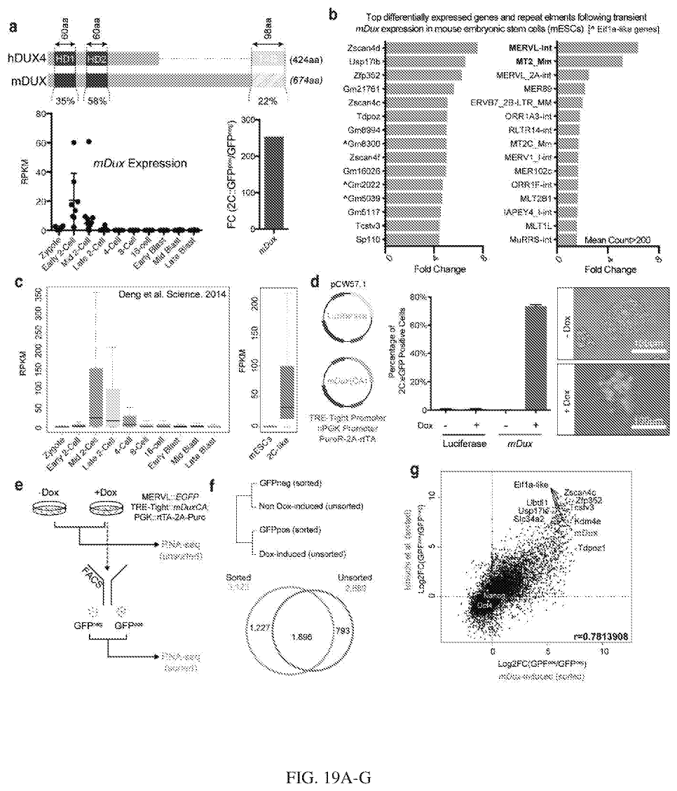

Notably, the marquee cleavage-specific transcription factor ZSCAN4 was the single most highly upregulated gene. A key question is whether hDUX4 activates ZSCAN4 directly in the embryo through its identified binding sites. Here, the inventors examined the ability of hDUX4 to activate transcription from a construct bearing the 2 kb region flanking the TSS of ZSCAN4 (which contains four predicted hDUX4 binding sites; FIG. 18B) fused to the SV40 promoter driving luciferase. Ectopic hDUX4 expression in human embryonic stem cells greatly induced luciferase activity, which could be eliminated by mutating three of the four predicted hDUX4 binding sites. Prior work claimed DUXA as the key transcription factor for ZSCAN4 activation, through DUXA binding to a 36 bp motif (including multiple HOX consensus sites) residing in the proximal upstream Alu elements. However, it was found that co-transfection with DUXA had no effect on luciferase activation, nor did the removal of upstream Alu elements affect activation by hDUX4 (FIG. 18C).

DUX4 expression also activated particular repetitive elements, including ACRO1 and HSATII satellite repeats, which normally peak in cleavage stage (FIG. 26C). However, the most striking induction was of HERVL retrotransposons (FIG. 18D) which along with their flanking LTR elements (most frequently, MLT2A1) are selectively transcribed during cleavage. In keeping with endogenous targets like ZSCAN4, the hDUX4 consensus binding site was significantly enriched in MLT2A1 and MLT2A2 LTR elements (FIG. 17D, table inset). Taken together, these results strongly implicate hDUX4 as the direct activator of ZSCAN4 and HERVL in human cleavage embryos, consistent with prior results following hDUX4 expression in muscle cells.

F. Functional Conservation of DUX Proteins in Defining the Cleavage Stage Transcriptome in Mammals

As genetic tools and genomic datasets involving cleavage stage transcription and chromatin have been developed primarily in murine cells and embryos, the inventors turned to the murine system to test whether DUX4-related proteins likewise display conserved and central roles in cleavage-stage transcription. The inventors' analysis of prior RNAseq datasets revealed cleavage-specific transcription of a mouse DUX4 homolog, mouse Dux, hereinafter referred to as mDux for clarity, which is only moderately conserved at the sequence level (FIG. 18A, 27A). Notably, mDux is transiently and specifically expressed in the early 2-cell mouse embryo, and also in ‘2C-like’ cells, a rare subpopulation of mESCs identified/characterized by the spontaneous reactivation of a MERVL::GFP reporter.

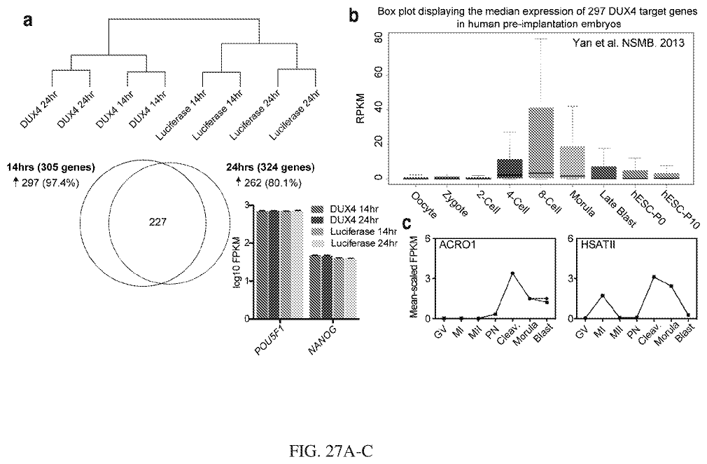

To test whether mDux expression can drive a cleavage-specific transcriptional program, the inventors initially expressed mDux in myoblasts (to link to prior work on hDUX4 in myoblasts) and performed qRT-PCR, which revealed strong upregulation of key cleavage-specific genes such as Zscan4, Zfp352, and Tcstv] (FIG. 27G). To extend these findings transcriptome-wide in a developmentally relevant cell-type, the inventors then transfected mESCs with a dox-inducible lentivirus encoding mDux (codon altered to reduce CpG content). Dox addition for 36 hr followed by RNAseq revealed the upregulation of 123 genes (FC>2, FDR<0.01) (FIG. 18B), with no genes significantly downregulated at the RNA level. This mDux-upregulated cohort of genes is transiently and specifically expressed in the mouse cleavage stage embryo and, in keeping, is re-activated in ‘2C-like’ cells (FIG. 3c). Interestingly, many of the genes activated by mDUX (e.g. Zscan4, Pramef, Zfp352, Ubtfl1, Kdm4e) have orthologs in human that are likewise transiently expressed in the human cleavage stage embryo and re-activated in human pluripotent stem cells upon ectopic DUX4 protein expression. While these genes likely have important and conserved roles in transcriptional and translational processes during the mammalian cleavage stage, hDUX4 and mDUX also have many unique targets (e.g. KHDC1L, LEUTX, Tcstv1-3, Tdpoz1-5, etc.) that may serve species-specific functions requiring further investigation (FIG. 27B).

Regarding repeat elements, in mice hundreds of cleavage-specific genes are activated through the co-option of MERVL repetitive elements (a murine-specific endogenous retrovirus), using MERVL-associated LTRs as either promoters or enhancers. Importantly, it was found that MERVL elements were strongly induced by mDux expression, with MERVL elements representing the most upregulated repetitive element class (FIG. 18B).

G. Conversion of mESCs to ‘2C-like’ Cells by mDux Expression

Prior work has revealed the ability of mESCs to naturally fluctuate between two states: >99% reside as conventional pluripotent stem cells whereas <1% reside in a ‘2C/cleavage-like’ state, characterized by the transcriptional re-activation of MERVL elements and cleavage-stage genes, the downregulation of pluripotency factors (e.g. OCT4/POU5F1) and the dissolution of chromocenters. The inventors' initial expression studies with mDux in mESCs suggested that it was only capable of turning on a fraction of the ‘2C-like’ transcription signature. In principle, however, as the inventors were relying on population average in a non-clonal cell line, the expression of mDux could be weak and heterogenous. To more accurately gauge the effects of mDUX, the inventors next integrated the dox-inducible mDux construct (or luciferase control) into mESCs bearing an integrated MERVL::GFP reporter, isolated clones that yielded high expression of mDux following doxycycline administration, and tested how efficiently they converted to a GFP-positive (GFP^{pos}) ‘2C-like’ state. Remarkably, in the selected cell line, ˜74% of cells activated the reporter within 24 hr of dox induction, whereas only ˜0.14% cells were GFP^{pos} in the absence of dox (>500 fold induction), demonstrating high potency and penetrance (FIG. 18D). Importantly, induction with dox correlates with mDux transgene RNA levels (FIG. 27C) and the relative number of GFP-positive cells (FIG. 27D). Dox-induced cells were then either sorted by FACS into GFP^{neg} and GFP^{pos} populations, or left unsorted, and subjected to RNAseq (FIG. 18E). Here, the unsorted (versus no dox-induction) and sorted GFP^{pos} cells (versus sorted GFP^{neg} cells) yielded a high number of overlapping upregulated genes (FIG. 18F). Notably, the transcriptional profile of mDux-induced cells was strongly correlated (r=0.78) with naturally fluctuating ‘2C-like’ cells (FIG. 18G, 27E), even at repetitive elements (e.g. MERVL, GSAT), strongly suggesting that mDUX regulates ‘2C-like’ conversion.

To examine whether mDux expression elicits additional known molecular features of cleavage embryos and ‘2C-like’ cells, the status of OCT4 was also examined, and chromocenters. Here, the IHC results demonstrated a complete loss of OCT4 protein(despite no change in Oct4 mRNA) in GFP^{pos} cells, and staining with DAPI revealed an absence of chromocenters in the same cells that contain GFP and lack OCT4 (FIG. 19A). Thus, mDux expression elicits in mESCs multiple molecular/biological characteristics of 2-cell embryos (FIG. 19B), and also supports changes in translational control and a reorganization of pericentric heterochromatin.

H. mDux is Necessary for Chaf1a-Mediated Induction of 2C-like Cells

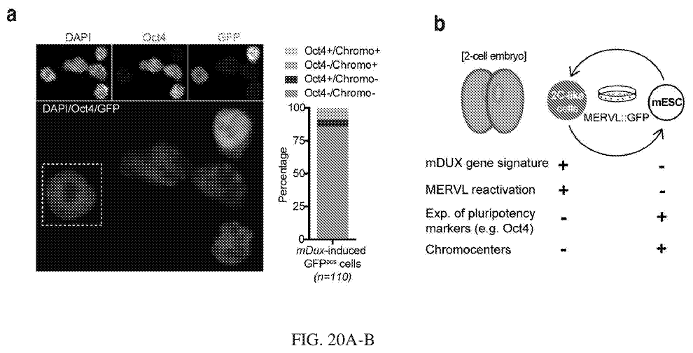

Interestingly, depletion of Chaf1a (the p150 subunit of the Chromatin assembly factor 1 complex; CAF-1) also induces the conversion of mESCs to a ‘2C-like’ state, prompting an examination of the relationship between CAF-1 and mDux. First, genes upregulated following mDux induction both overlap with, and also compose the most highly upregulated genes in Chaf1a-depleted mESCs (FIG. 20A-B). Of particular relevance, it was found that mDux itself upregulated 11-18 fold in the CAF-1 subunit-depleted datasets. To determine whether mDux expression was necessary for entry into a ‘2C-like’ state, mESCs containing the integrated MERVL::GFP reporter were co-transfected with siRNA against Chaf1a, and siRNA pools targeting mDux mRNA. First, prior results showing an increase in ‘2C-like’ cells following knockdown of CAF-1 (FIG. 20C) were confirmed. Interestingly, however, simultaneous knockdown of mDux mRNA strongly mitigated this affect, showing that mDux plays a major role in mediating the impact of CAF-1 subunit depletion on enabling ‘2C-like’ conversion.

I. mDux Expression Coverts the Chromatin Landscape of mESCs to One Strongly Resembling Early 2-Cell Mouse Embryos

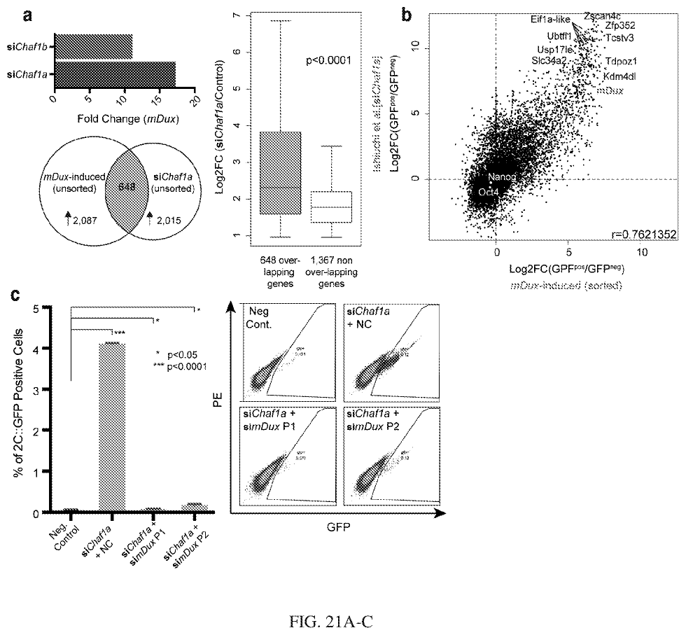

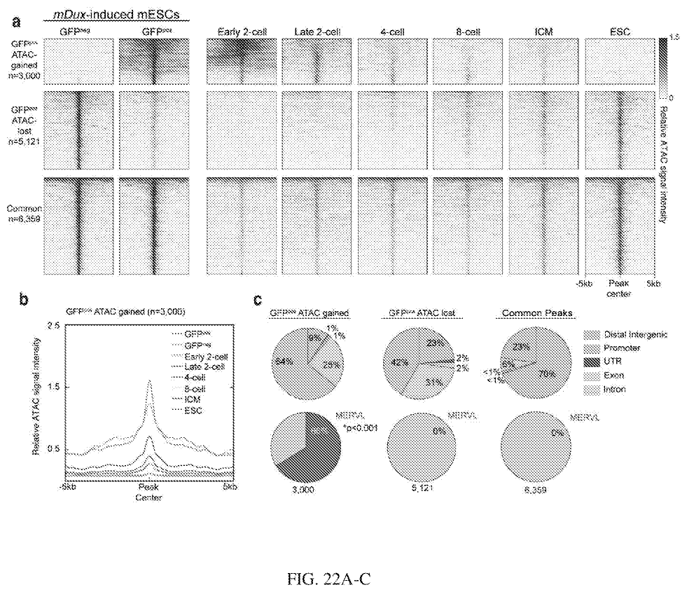

New genomics methodologies, namely ATAC-seq, enable the determination of open versus closed chromatin genome-wide. Cleavage stage chromatin undergoes extensive reorganization to facilitate EGA and the conversion of gametes into totipotent embryos, supported by the distinctive ATAC/chromatin profiles recently revealed in 2-cell cleavage embryos. The inventors therefore tested whether mDUX can convert the chromatin landscape of mESCs to that of 2-cell cleavage embryos, by conducting ATAC-seq analyses on sorted MERVL:: GFP^{pos} and MERVL:: GFP^{neg} cells following mDux expression (24 hr). Using statistical thresholds (FDR<0.05), 3,000 regions shared across two independent replicates that gain ATAC signal in GFP^{pos} cells compared to GFP^{neg} cells were identified (FIG. 21A, 28A). Likewise, 5,121 regions that lose ATAC signal were identified. Fascinatingly, the chromatin state in both gained and lost regions strongly resembles those of 2-cell embryos rather than mESCs. Moreover, the GFP-gained ATAC regions were unique for their breadth—with many extending over 10 kb in length (FIG. 21B). Notably, these broad ATAC-gained regions largely overlap with intergenic regions and, more specifically, with repetitive elements of the MERVL subfamily (FIG. 21C), whereas the more compact regions lost in GFP^{pos} cells (or common to GFP^{pos} and GFP^{neg}) overlap better with gene promoters (FIG. 21C). Importantly, regions that gain ATAC signal are linked to genes highly and significantly activated in ‘2C-like’ cells, whereas regions that lose ATAC signal generally link to genes displaying moderate but significant downregulation (FIG. 20B). Taken together, the ATAC-seq approaches demonstrate that mDux-induced ‘2C-like’ cells largely convert to the chromatin patterns of 2-cell mouse embryos and away from the patterns of mESCs, and include changes at many of the marquee genes and retrotransposons expressed specifically during cleavage.

J. mDux Occupancy is Strongly Correlated with Dynamic Chromatin Sites

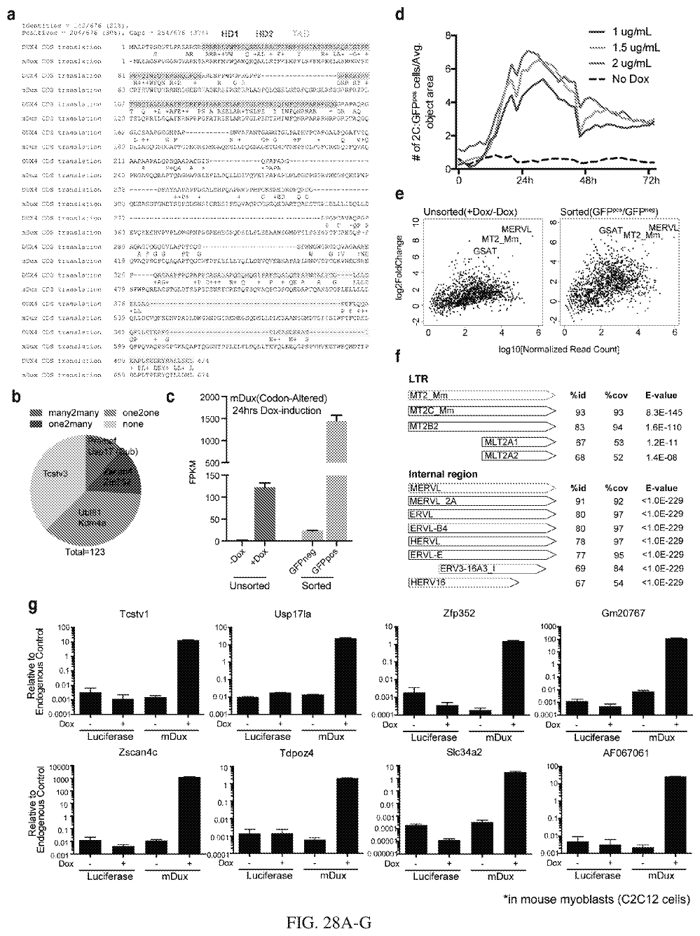

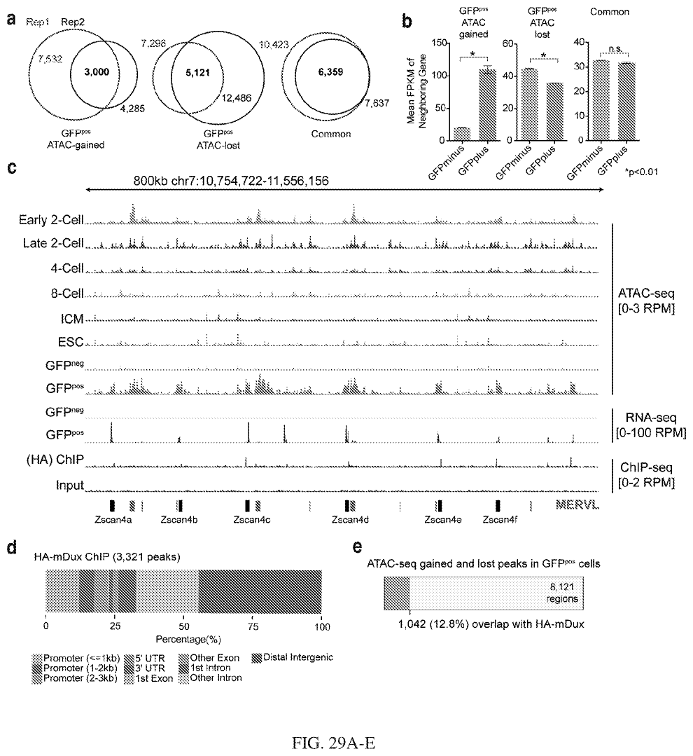

To determine which ATAC-seq changes are due directly to mDUX binding, and to test whether the mDUX affect in the earlier transcription data was truly direct, chromatin immunoprecipitation followed by sequencing (ChIP-seq) was performed on unsorted mESCs following 24 hrs of dox-induction, this time expressing an mDux transgene containing an N-terminal human influenza hemagglutinin (HA)-epitope tag. First, the ineventors found clear peaks at many mDUX-induced genes (e.g. Zscan4a-f, Usp171d, Tdpozl, and Gm20767), as well as many intergenic locations overlapping with MERVL-associated LTRs (FIG. 28C-D).

Importantly, using the top 1500 ChIP-seq peak summits based on enrichment score, a consensus mDUX binding motif (FIG. 22A) was identified. As expected, this motif is highly enriched in MT2_Mm elements (the canonical LTR for MERVL), but is not enriched in related, unaffected LTR variants like MT2C_Mm. Finally, the HA-mDUX peaks overlapped greatly with genes and repetitive elements (e.g. MT2_Mm/MERVL) that are silenced in mESCs but gain ATAC signal following mDux expression (FIG. 22B, FIG. 28E), suggesting a role in targeting chromatin opening. Taken together, this work provides multiple lines of evidence that mDUX plays a major role in driving and defining the chromatin landscape and transcriptional program of ‘2C-like’ cells.

### II. Discussion

Using RNAseq, improved transcriptional profiles of human oocytes and embryos during pre-implantation development were generated. The invenors then focused on cleavage stage embryogenesis, during which the embryonic genome becomes transcriptionally activated, gametic constitutive heterochromatin is reduced and subsequently re-established (resulting in the formation of chromocenters), and maternal telomeres (which are inherited unusually short) are lengthened. All three events are critical for progression beyond cleavage—but whether and how each is interconnected and ultimately initiated are key unanswered questions.

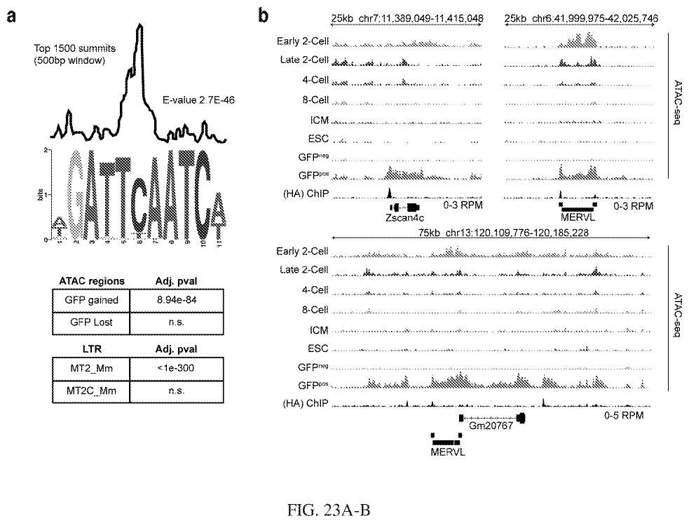

In human and mouse, a unique transcriptional program is robustly activated at EGA and firmly restricted to the cleavage stages of preimplantation embryonic development. Here, the inventors have shown that many cleavage-specific genes are targets of a functionally conserved double homeobox retrogene called hDUX4 in humans, and mDux in mice (collectively referred to here as the DUXC-family), which is transiently expressed at the outset of EGA in both species (FIG. 23A). Based on temporal dynamics, the DUXC-family driven transcriptional program does not intrinsically activate the embryonic genome per se. Instead, our data supports a central role for the DUXC-family in regulating vital EGA-coupled molecular events through the transcriptional activation/targeting of their key mediator(s). For example, ZSCAN4 directs the telomerase-independent, recombination-based telomere elongation mechanism that operates in the cleavage-stage mammalian embryo, and KDM4E, a probable H3K9me3 demethylase enzyme, may function to reprogram the genome towards totipotency. Thus, the DUXC-family helps couple EGA to several major reprogramming events. Remarkably, this link (at least in the mouse) relies on the reactivation of retrotransposons (e.g. MERVL) which have been exapted to wire the cleavage-specific transcriptional program together. Previously, activation of MERVL elements during this developmental widow was considered a consequence of the permissive chromatin landscape. The ATAC-seq data instead show that mDUX selectively binds and creates open chromatin at MERVL elements, a result that aligns with reported interactions of hDUX4 with p300—an enhancer-associated histone acetyltransferase—and the notion of hDUX4 as a pioneer transcription factor.

Despite clear functional conservation, hDUX4 and mDUX bear only modest sequence conservation, though both are intron-less and can be found in tandem arrays on multiple chromosomes. One leading hypothesis suggests derivation through independent retrotransposition events involving the ancient, intron-containing, DUXC gene, which has since been lost in both species. Both DUX4-family retrogenes have subsequently undergone multiple rounds of duplication and considerable change, including the creation of multiple paralogs (which greatly complicate genetic loss-of-function approaches). Until now, the biological relevance of hDUX4 outside of FSHD pathology was unclear, but its maintenance and expansion strongly suggests important fitness contributions. Notably, the DUX-family (e.g. DUXA, DUXB, DUXC) origination aligns with trophectoderm/placental development; they are specific to placental animals, they are expressed prior to the first lineage decision, and they are rapidly expanding/evolving—features common in genes driving placentation.

In many species and systems, endogenous retroviruses (ERVs) have shaped specific transcriptional programs through the provision of cis-regulatory elements. This firstly relies on viral co-option of host cell transcription factors to achieve expression/amplify, and subsequently the exaptation of those viral elements by the host. Accordingly, with the context of previous evolutionary analysis, this work suggests that an ancient DUXC ortholog arose in the common ancestor of placental mammals to regulate embryonic reprogramming by activating the expression of specific genes (e.g. ZSCAN4) during cleavage. This early eutherian species was likely infected by an ERV-L foamy retrovirus that then integrated into its genome. In primate and rodent lineages, the inventors speculate that endogenous ERVL acquired a DUX binding site that allowed it to expand and give rise to HERVL and MERVL elements respectively (FIG. 27F). Whether or not it was the expansion of ERVLs that accelerated DUX4-family diversification in these species is not clear, but of interest.

It remains unknown how the genes encoding DUXC-family transcription factors are themselves briefly activated during early cleavage. One possibility is that genome-wide DNA demethylation in the zygote coupled with a maternally loaded transcription factor enables their transcription at EGA. Alternatively, Ishiuchi et al. report a transient uncoupling of CAF-1 mediated chromatin assembly with DNA synthesis in the early 2-cell embryo, which may reduce nucleosome occupancy in the genome (and/or generally de-repress heterochromatin) and allow a burst of mDux expression. A similar sequence of events, potentially in response to extended cell cycle times, may occur in rare mESCs to repair shortened telomeres.

Taken together, this work may have significant implications for early embryo lineage decision-making (impacting human infertility and recurrent pregnancy loss), the reprogramming field, cancer biology, and FSHD. This data supports a role for DUX4-family proteins in establishing the cleavage stage transcriptome, a stage which holds broad developmental potential. Notably, the ability of mDux expression to drive the vast majority of mESCs into a ‘2C-like’ state raises the possibility of deriving cell lines with cleavage-like developmental potential for mechanistic studies. Here, although this data supports a major role for DUXC-family proteins, the inventors expect that maternally-derived/inherited transcription factors likely collaborate to achieve full cleavage stage potential, and speculate that factor combinations may lead to the highest success of reprogramming. Regarding FSHD, as cleavage embryos resist the apoptosis conferred by DUX4 expression in muscle cells, ‘2C-like’ cell lines might provide mechanistic or therapeutic insights. Finally, DUX4 fusion proteins (that omit the C-terminus of DUX4) driven by the IGH enhancer have recently emerged as the leading cause of acute leukemias in adolescents and young adults, prompting need for a greater understanding of DUX4 biochemically and molecularly in normal and oncogenic circumstances.

### III. Methods

No statistical methods were used to predetermine sample size. All experiments were performed at least twice with multiple replicates and consistent results.

A. Sample Collection

Germinal Vesicle (GV) stage oocytes were collected from IVF patients at the University of Utah and the Minnesota Center for Reproductive Medicine from October 2011 to February 2013. Enrollment was limited to patients who were undergoing IVF with Intra Cytoplasmic Sperm Injection (ICSI) procedures of their own accord. Metaphase I and metaphase II oocytes were collected from fifteen healthy women, aged 21-28, who were voluntarily enrolled for this study. Donors underwent an ovarian stimulation cycle-using a long agonist protocol -followed by oocyte retrieval. Pre-implantation embryos were donated to IRB-approved research by consenting patients at the Utah Center for Reproductive Medicine and the Minnesota Center for Reproductive Medicine. Each patient's informed consent was reviewed and documented by two clinical investigators prior to their use in the study. No embryos were created for research purposes. In all cases, embryos were donated by patients ending their fertility treatments, and therefore the remaining embryos would otherwise have been discarded.

B. Sample Preparation

Within 3 hours of collection, GV, MI, and MII oocytes were completely denuded of their cumulous cells. Denuded oocytes were then stored in 10 uL of protein free media in slow freeze 250 uL straws and kept at −80C. until RNA preparation Likewise, embryos used for this study were cryopreserved according to standard IVF protocols. Prior to RNA preparation, the embryos were thawed and pooled according to developmental stage. Embryos that failed to survive the freeze-thaw procedures were discarded. Blastocyst stage embryos were hatched and, using laser microdissection, were manually separated into Inner Cell mass (ICM) and mural trophectoderm (Troph). RNA extraction from pooled oocytes and embryos was preformed using the Qiagen AllPrep kit®. All sample handling of embryonic stages, from retrieval through nucleic acid isolation, was conducted in clinical facilities by clinically-funded staff, separate from NIH/NCl/HCl funded facilities and personnel.

C. Plasmid Construction and Generation of Stable Mouse Cell Lines

DUX4-family gene coding sequences were codon altered (to aid in synthesis and expression) and synthesized as gBlocks from IDT. Fragments were then cloned into a dox-on lentiviral backbone containing a puromycin selectable marker; pCW57.1 (a gift from David Root, Addgene plasmid # 41393). Stable 2C::EGEP mESCs, containing the MERVL::EGEP reporter and a 6418 selectable marker, were generously gifted by Maria-Elena Torres-Padilla. Plasmids were transfected using Lipofectamine 2000 (ThermoFischer) and several stable cell lines were generated through antibiotic selection and subsequent clonal expansion in 2i media.

D. Mouse ES Cell Culture

E14 mESCs were cultured on gelatin in PluriQ™ ES-DMEM medium containing non-essential amino acids, B-mercaptoethanol, and dipeptide glutamine and supplemented with 15% ES-grade FBS, Primocin™, and leukemia inhibitory factor (ThermoFischer cat. PMC9484). For 2i culture, media was supplemented with 1mM PD0325901 (Sigma-Aldrich cat. PZ0162) and3 mM CHIR99021 (Sigma-Aldrich cat. SML1046). For selection, media was supplemented with Geneticin® (G418 Sulfate, ThermoFischer cat. 10131027) and/or Puromycin Dihydrochloride (ThermoFischer cat. A11138-03)

E. Human iPS Cell Culture and Generation of Stable Cell Lines

Human induced pluripotent stem cells were grown on Matrigel in mTeSR1 with ROCK inhibitor. To create stable lines, cells were incubated with DUX4CA or luciferase lentivirus (MOI=5) for 16 hrs. After 2 days of recovery, cells were split and plated on MEFs and cultured for 3 passages in the presence of Puromycin Dihydrochloride (ThermoFischer cat. A11138-03). Resistant cells were split again with dispase (to remove MEFs) and re-plated on Matrigel prior to dox-induction.

F. Myoblast Cell Culture and Real-Time RT-qPCR

C2C12 mouse myoblast cells (ATCC) were grown in 10% fetal bovine serum and 1% penicillin/streptomycin at 37° C., 5% CO2. Cells were transduced with lentivirus carrying either pCW57.1-Luciferase or—mouse Dux (mDux) and selected with 2.6 ug/ml puromycin. Individual colonies were isolated and chosen for analysis based on robust transgene expression following 2 ug/ml doxycycline treatment. Biological triplicates were prepared by plating 1.5×10⁵ cells into six-well dishes with 2.6 ug/ml puromycin and induced with 2 ug/ml doxycycline for 36 hours, as indicated in graphs. RNA was isolated using Clontech RNA Isolation kit. One microgram of total RNA was digested with DNAseI (Invitrogen) and then reverse transcribed into first strand cDNA in a 20 uL reaction using SuperScript III (Invitrogen) and oligo(dT) (Invitrogen). cDNA was diluted and used for RT-qPCR with iTaq Universal SYBR Green Supermix (Bio-Rad). Relative expression levels were normalized to the endogenous control locus Timml7b by DeltaCT.

G. Luciferase Assay

A 1.9 kb region containing the putative enhancer and promoter of ZSCAN4 was cloned into a PGL3-basic reporter vector (LP; long promoter). Two variants, one containing mutations in three of the four DUX4 binding sites (LP-3xmut) and another in which three of the four upstream ALU elements had been removed (SP; short promoter) were also created. Each reporter was separately and transiently co-transfected into human ES cells with a GFP, GFP-DUXA, or GFP-DUX4 expression construct and induced with doxycycline for 24 h. Following induction, nuclear expression was verified using the EVOS imaging system. Then the cells were lysed in Passive Lysis Buffer and luciferase intensity was measured using the Dual-luciferase™ Reporter Assay from Promega.

H. Egg and Embryo Library Preparation and RNA Sequencing

High-quality RNA (RIN>7) was extracted from all stages. Using the TotalScript RNA-Seq kit (Epicentre ; Cat. num. TSRNA1296), two stranded libraries were prepared for each stage. This approach enabled low inputs (5 ng of total RNA/reaction), and random hexamer priming facilitated transcript coverage balance. Each cDNA library was then split and amplified for 12 or 14 PCR cycles, resulting in four technical replicates per developmental stage. All libraries were sequenced on the Illumina HiSeq 2000 platform.

I. Cell Line Library Preparation and RNA Sequencing

The RNA seq libraries generated from cultured cells were prepared using the Illumina TruSeq kit. Briefly, cells were lysed in Trizol and RNA extracted using the Direct-zol™ RNA MiniPrep kit by Zymo Research. Intact poly(A) RNA was purified from total RNA samples (100-500 ng) with oligo(dT) magnetic beads and stranded mRNA sequencing libraries were prepared as described using the Illumina TruSeq Stranded mRNA Library Preparation Kit (RS-122-2101, RS-122-2102). Purified libraries were qualified on an Agilent Technologies 2200 TapeStation using a D1000 ScreenTape assay (cat# 5067-5582 and 5067-5583). The molarity of adapter-modified molecules was defined by quantitative PCR using the Kapa Biosystems Kapa Library Quant Kit (cat#KK4824). Individual libraries were normalized to 10 nM and equal volumes were pooled in preparation for Illumina sequence analysis. Sequencing libraries (25 pM) were chemically denatured and applied to an Illumina HiSeq v4 single- or paired-end flow cell using an Illumina cBot. Hybridized molecules were clonally amplified and annealed to sequencing primers with reagents from an Illumina HiSeq SR Cluster Kit v4-cBot (GD-401-4001) or PE Cluster Kit v4-cBot (PE-401-4001). Following transfer of the flowcell to an Illumina HiSeq 2500 instrument (HCS v2.2.38 and RTA v1.18.61), a 50 cycle single-read or a 125 cycle paired-end sequence run was performed using HiSeq SBS Kit v4 sequencing reagents (FC-401-4003).

J. RNA-Seq Data Processing

Raw sequencing reads were aligned with Novoalign (Novocraft, Inc.) to hg19 or mm10 [−r All 50]. Splice junction alignments were converted to genomic coordinates and low quality and non-unique reads were further parsed using Sam Transcriptome Parser (USeq; v8.8.8). Stranded differential expression analysis was calculated with DESeq2 using a reference hg19 or mm10 Ensembl gene table downloaded from UCSC. mDux transgene levels were measured by aligning each dataset to an index file of the codon-altered (CA) sequence. Splice isoform quantification was determined using Sailfish V0.10.0¹⁴. Principal Component Analysis and Partition Clustering (using the Davies-Bouldin statistic) were performed using the Partek Genomics Suite (Partek Inc.) based on FPKM values. Heatmaps were produced in R using the ‘pheatmap’ package and various graphical analyses in R and GraphPad Prisim (V6). Genome snapshots were generated from IGV (Integrated Genomics Viewer; Broad Institute).

K. Comparative Analysis

RNA sequencing reads from Yan et al (GSE36552) and Xue et al (GSE44183) were downloaded from GEO and processed as described above. Single cell data for each developmental stage was merged. Relative read coverage graphs were generated using the CollectRnaSeqMetrics application from Picard tools (http://broadinstitute.github.io/picard/). Exonic and novel transcription was estimated using the Sam2USeq application (USeq; v8.8.8) on the alignments from each stage. Regions of >1, >3, or >5 non-stranded read coverage were output to a BED file that was subsequently intersected with a BED file containing all known Ensembl, UCSC, and NONCODE v4 exons plus 500 bp in both directions. Intersecting regions are reported as exonic transcription in base pairs. Non-intersecting regions are reported as novel transcription.

L. Novel Transcription

Novel transcription was evaluated using the same novo-alignments used for the gene expression analysis. In short, the non-annotated genome was scanned for enriched or reduced regions of expression. Using MultipleReplicaScanSeq (USeq; v8.8.8) 27,419 non-overlapping regions of novel expression were identified, with 2,875 displaying differential expression between adjacent developmental stages (fold change>2; FDR <0.01). Coding potential scores calculated using the Coding Potential Calculator known in the art.

M. Repetitive Element Read Coverage

Repeat masker (rmsk-hg19, rmsk-mm10) files were downloaded from UCSC table browser. Each instance of a particular repeat subfamily (RepName) was given a unique identifier and annotated with repeat type (RepType) and repeat family (RepFamily) information. This modified repeat table was then appended to an exon table and reads were counted over all repeat/exon instances using DefinedRegionDifferentialSeq (USeq; v8.8.8). As before, only reads that mapped uniquely to the genome were considered. Using a custom perl script, reads were summed by subfamily or gene annotation. Differential expression of repeat subfamilies between stages was calculated using DESeq2.

N. Motif Identification

To identify potential transcriptional regulators of the genes enriched in each cluster, the Motif Enrichment Tool (MET)(found on the world wide web at veda.cs.uiuc.edu/cgi-bin/MET/interface.pl) was used to query the regulatory regions 5 kb and 20 kb upstream of each gene set for enrichment of the known TF motifs in the HT SELEX and JASPAR collections.

O. Phylogeny

To create the phylogenetic tree diagram, the homeodomain amino acid sequences for all human PRD-class transcription factors of interest (and mouse; mDUX) were downloaded from the homeobox database (http://homeodb.zoo.ox.ac.uk). The phylogenetic tree was created using Geneious Tree Builder (Geneious; v 8.1.5) with the neighbor-joining method and Juke-Cantor model.

P. Fluorescence-Activated Cell Sorting

Quantification of GFP-positive cells was done using a Cytek DxP Analyzer and data was processed in Flow Jo. For sorted RNA sequencing and ATAC-sequencing, an Avalon Cell Sorter (Propel Labs) and FACSAris Cell Sorter (BD Biosciences) was used to sort GFP-positive and negative cells prior to library preparation.

Q. Immunofluorescence and Imaging

Cells were plated on gelatin coated coverslips and allowed to adhere for 3-5 hours before fixing in 4% paraformaldehyde in PBS for 10 minutes at room temperature. Subsequently, the cells were permeabilized in 0.1% Triton-X-100 in PBS for 10 minutes at room temperature and then blocked in 3% BSA in PBS for 1 hour at room temperature. Primary antibodies (see below) were diluted in 3% BSA and the cells were incubated for 1 hour at room temperature. Cells were then washed and incubated in diluted Alexa-conjugated secondary antibodies plus DAPI (4′,6-diamidino-2-phenylindole) for 1 hour at room temperature before mounting. Imagining was done on a Nikon A1 confocal microscope. Simple fluorescence images of 2C:EGFP cells were collected on the EVOS™ FL cell imaging system and quantitative live-cell capture and analysis using the IncuCyte® ZOOM system. Primary antibodies to the following proteins were used: Anti-GFP (abcam, ab13970), Anti-Oct3/4 (Santa Cruz Biotechnology, sc-5279). Secondary antibodies included an Alexa 488 Goat Anti-Chicken (Thermo Scientific, A11039) and an Alexa 594 Donkey Anti-Mouse (Life Technologies, A21203).

R. siRNA Generation and Transfection

Chaf1a (s77588) and negative control-Silencer Select siRNA were purchased from LifeTechnologies. mDux siRNA pools were generated using Giardia Dicer. Briefly, primers were designed to amplify two ˜400 bp fragments of the endogenous mDux locus from genomic mouse DNA and add T7 handles (see below). Purified PCR products were then used as template for in vitro transcription using the MEGAscript® T7 Transcription Kit (ThermoFischer, AM1334). Template DNA was then degraded and the ssRNA allowed to anneal before dicing. Diced siRNAs were purified using the PureLink™ Micro-to-Midi Total RNA purification Kit (Invitrogen, 12183-018) with modifications. siRNA concentration was measured with the Qubit® RNA HS Assay Kit (ThermoFisher, Q32852). mESCs were transfected with 20 pmol (10 pmol of each) of total siRNA using RNAiMax (Life Technologies). All transfections were performed twice (on back to back days) to ensure knockdown before measuring the effects by FACS.

simDuxP1-

(SEQ ID NO: 133)

1049F(AACTCCTCCTCCTTGATCAACTG),

(SEQ ID NO: 134)

1456R(CTTCTCTCTGTGGCCAAAAGC)
simDuxP2-

(SEQ ID NO: 135)

1503F(CTTCTGCAGAGAGTCCCAGAC),

(SEQ ID NO: 136)

1982R(GGCAGATCAGGTGTTGTGTC)

S. ATAC-Seq Library Preparation and Sequencing

The ATAC-seq libraries were prepared as previously described (ref) on ˜30k sorted (GFP^{pos} or GFP^{neg}) mESCs after 24 hours of dox-induction (mDuxCA expression). Immediately following FACS, the cells were lysed in cold lysis buffer (10 mM Tris-HCl. pH 7.4, 10 mM NaCl, 3 mM MgCl2 and 0.1% IGEPAL CA-630) and the nuclei were pelleted and resuspended in Transposase buffer. The Tn5 enzyme was made in house (Picelli, et al. Genome Research 2014) and the transposition reaction was carried out for 30 minutes at 37° C. Following purification, the Nextera libraries were amplified for 12 cycles using the NEBnext PCR master mix and purified using the Qiagen PCR cleanup kit. All libraries were sequenced on the Illumina HiSeq 2500 platform.

T. ChIP-Seq Library Preparation and Sequencing

To investigate mDUX binding, an N-terminal HA-epitope tag fused to the integrated mDuxCA transgene was utilized to perform Chromatin Immunoprecipitation and sequencing (ChIP-seq). Briefly, mESCs were induced with doxycycline for 24 hrs and then cross-linked with 1% formaldehyde for 10 minutes. Cells were lysed and chromatin was sonicated using the BioRuptor® system (Diagenode). Cellular debris was pelleted and the DNA was precipitated overnight at 4° C. using a CUP Grade Anti-HA tag antibody (Abeam, ab9110). After reversing crosslinks, libraries were prepped using the NEBnext DNA Library Prep Kit (NEB, E7370L). Adapter ligated DNA was size selected and purified using AMPure XP beads (Beckman Coulter, A63881) before sequencing on the Illumina HiSeq 2500 platform.

U. ATAC-Seq and ChIP-Seq Data Processing

Paired-end, raw read files were first processed by Trim Galore (Babraham Institute) to trim low quality reds and remove adapters. Processed reads were then aligned to mm10 using Bowtie2 (v2.2.6) with the following parameters: (-t -q -N1-L 25-X 2000 -no-mixed-no-discordant). ATAC-seq peaks were called on each replicate with MACS2 callpeak (-B-nomodel-nolambda-shift -100 -extsize 200). Subsequently, the ‘bdgdiff’ subcommand was used to call “differential peaks” between each replicate of sorted GFP^{pos} versus GFP^{neg} cells. Only the gained, lost, and common peaks identified in both replicates were considered further. For comparisons to pre-impination embryo, data was downloaded from GEO (GSE66390) and re-processed as described above. Biological replicates were aligned independently and merged in MACS2. ChIP-seq peaks were called above input on each replicate with MACS2 callpeak (-B -SPMR-nomodel-extsize 200). Downstream analyses with ChlPseeker and Galaxy deepTools. Motif discovery and enrichment analyses performed using the MEME suite tools.

V. Code Availability

All newly developed code used in the bioinformatic analyses described above is available through the USeq package. USeq is a collection of open source software tools that is under continuous development at the Huntsman Cancer Institute.

W. Data Accession

All sequencing data has been deposited to GEO and can be found under the series accession number GSE85632, which is herein incorporated by reference for all purposes.

### Example 4

### Chimera Contribution of Control or DUX-Expressing Mouse Embryonic Stem Cells

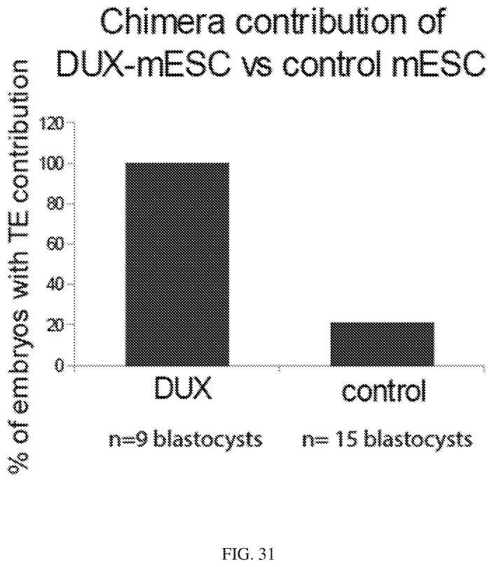

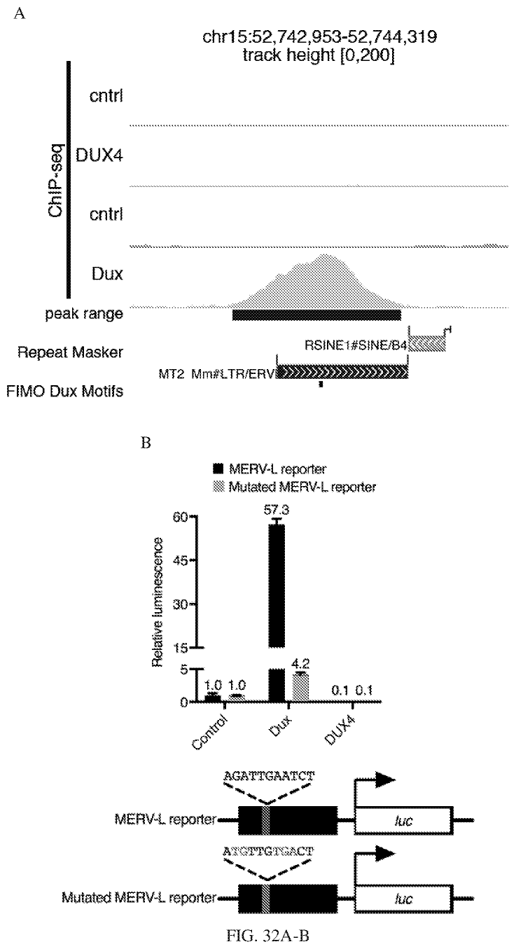

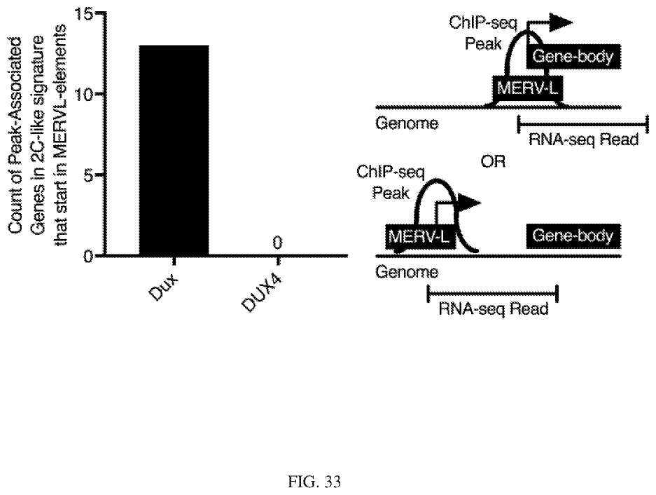

To test the chimera contribution of DUX-expressing mESCs, embryos were arranged in drops of culture media under oil. A Narashige micromaninpulator and piezo drill was used to make a hole in the zona pelucida. Then 3-4 mESC (control cells or DUX-expressing mESC) were transferred into E3.0 morulas with a 12 micron inner-diameter 25 degree angle transfer pipette. Then injected morulas were then returned to KSOM (mouse embryo cell culture media) and incubated in at 37° C. until E4.5 blastocysts developed. Next, the contribution to blastocyst lineages (inner cell mass or trophectoderm) was quantified (FIG. 31) at E4.5. mCherry-transgene was used to mark mESC and DUX-mESC. Using this chimera assay in which the cells incorporate into both the trophectoderm and the inner-cell mass, it was found that DUX-expressing mESC can regain totipotency. This indicates that DUX contributes to the acquisition of totipotency, and this cellular state is a better SCNT nucleus donor.

Since, DUX-expressing cells provide a superior donor cell for SCNT experiments, it is believed that DUX expression will improve the cloning efficiency for mammalian embryos.

### Example 5

### Conserved Roles for Murine DUX and Human DUX4 in Activating Cleavage Stage Genes and MERVL/HERVL Retrotransposons

Examples 5 and 3 may have duplicative text, which is not necessarily indicative of different or the same experiments.

### 1. RNA Transcriptomes from Developing Human Oocytes and Early Embryos

Samples from seven stages of human oogenesis and early embryogenesis were donated from consented patients undergoing in vitro fertilization (IVF) in accordance with Institutional Review Board (IRB) guidelines and approval, using standard IVF culture conditions (FIG. 17A, left panel). Blastocyst embryos were manually separated into ICM and mural trophectoderm by laser dissection (FIG. 17A, right panel). To minimize variation, all samples were processed together. For each, total RNA was divided (providing two technical replicates) and processed in parallel using a transposase-based library method to sequence total RNA without 3′ bias. To maximize dataset utility, the inventors performed deep RNA sequencing (RNA-seq) using a paired-end 101 bp sequencing format. Replicates were highly concordant (spearman correlation, r>0.92), and yielded on average ˜76 million unique, stranded, mappable reads. Importantly, read coverage from the transcription start site (TSS) to the transcription termination site (TTS) was exceptionally well-balanced compared to prior work (FIG. 17B, FIG. 25A), making these new datasets the most comprehensive transcriptomes of human oocyte and pre-implantation embryonic development to date.

### 2. PCA and Clustering Analyses Reveal a Unique Cleavage-Stage Transcriptome

Collectively, 19,534 (33.3%) of the 58,721 genes annotated by Ensembl were expressed across our sample series (count>10). Remarkably, 17,335 (88.7%) were differentially expressed (fold change>2; FDR<0.01) in at least one stage by adjacent stage pairwise analyses. To examine developmental order, the inventors performed principal component analysis (PCA) using all genes of moderate-to-high expression (9,734; Fragments Per Kilobase Per Million [FPKM] >1). The top three principal components effectively separated the sampled stages, while replicates of the same stage remained closely associated (FIG. 17C). Here, separation distances within the PCA map represent the extent to which developmental transitions are accompanied by major changes in transcript abundance. Notably, the stages of oocyte development (along with the pronuclear stage) co-localize along a short temporal arc, consistent with progressive but moderate changes in transcript abundance. In contrast, the cleavage-stage replicates were clearly distinct, consistent with new transcription after embryonic genome activation (EGA). An additional major change involves transition to the morula stage, which appears strikingly similar to trophectoderm replicates, whereas the ICM replicates form a distinct separate group. K-means algorithims were used to cluster genes based on their temporal expression and enrichment (FIG. 17D). Stage-specific gene sets pertaining to the immature egg (Cluster 1), cleavage (Cluster 4), and ICM (Cluster 7) stages were identified and contained genes of both known (e.g. FIGLA, ZSCAN4, and NANOG) and unknown specificity and developmental function.

### 3. Examination of Alternative Splicing and Novel Transcription

Overall, our transcription profiles were consistent with prior single cell datasets (FIG. 25B). However, improvements in read coverage balance and directionality enabled the discovery of new novel transcription (FIG. 25C) and splice isoform expression during pre-implantation development (FIG. 25D-F). Together, these datasets yield extensive new information providing a major resource for future studies.

### 4. A hDUX4 Binding Motif is Enriched Upstream of Cleavage-Specific Genes

The inventors then addressed a key question in pre-implantation embryo development—what transcription factors drive stage-specific gene expression? To identify candidates, the inventors performed de novo motif calling on the promoters of genes in clusters 1, 4, and 7 (data not shown). The most highly enriched motif was associated with cluster 4 genes and matched the predicted binding site of a well-studied transcription factor called hDUX4 (p=1c-11)(FIG. 17E). hDUX4 is one of three coding DUX (double homeobox) genes in humans, which also includes DUXA and DUXB. The DUX family is notable for its relatedness to the paried (PRD)-like homedomains, ARGFX, LEUTX, DPRX, and TPRX1, all of which show signs of rapid evolution/divergence and an involvement in human EGA.

### 5. hDUX4 Potently Activates Cleavage-Specific Genes and Repetitive Elements

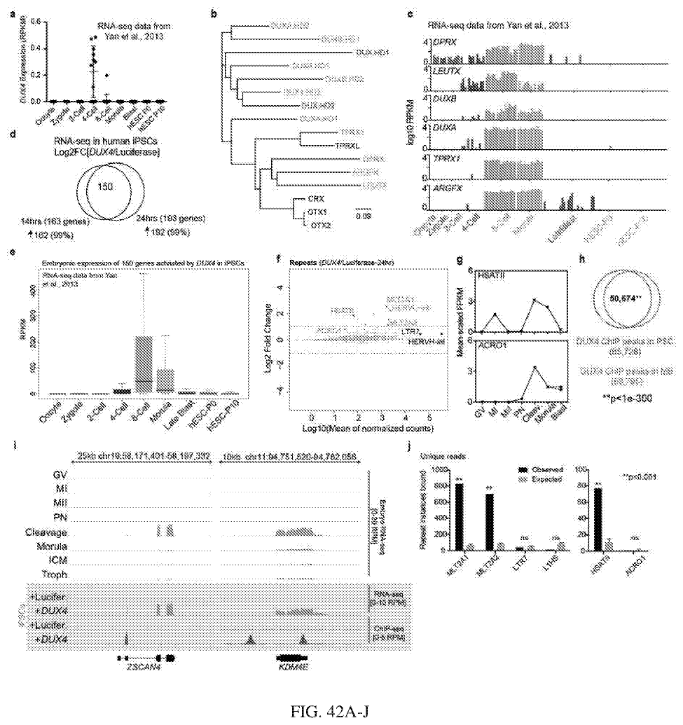

hDUX4 mRNA and protein are restricted to the 4-cell stage (early EGA) (data not shown, FIG. 42A) preceding the transient expression/enrichment of the other ‘PRD-like’ genes during the 8-cell and morula stages (FIG. 42B, C). To identify hDUX4 transcriptional targets the inventors overexpressed it in human induced pluripotent stem cells (iPSC) and performed RNA sequencing (RNA-seq). Compared to luciferase controls, induction of hDUX4 for 14 or 24 hrs via dox administration led to significant differential expression (FC>2; FDR<0.01) of 163 and 193 genes, respectively (FIG. 42D)—all of which were upregulated except one (ZNF208). Remarkably, as a group this gene set (which included notable DUX/PRD factors listed above) showed robust and transient expression in the cleavage stage embryo (FIG. 18A, FIG. 42E).

The most highly activated gene was ZSCAN4, a defining cleavage-stage gene in both human and mouse. Based on previous ChIP-sequencing data from human myoblasts (MB), ZSCAN4 is directly bound by hDUX4 and contains four distinct hDUX4 binding sites. To test for direct hDUX4 activity in embryonic stem cells (hESCs) the inventors developed a luciferase reporter using the ˜2 kb promoter (LP) sequence for ZSCAN4 (FIG. 18C). Transient co-transfection with hDUX4 induced luciferase expression >2,000-fold. However, in contrast to prior work, transient co-transfection with DUXA had no effect. Omitting three of the four hDUX4 binding sites (LP-3xmut) greatly reduced activation, whereas eliminating the proximal Alu elements (SP), previously implicated in ZSCAN4 activation via DUXA, had no affect. Thus, ZSCAN4 activation is specifically controlled by the direct binding of hDUX4 to its predicted binding sites.

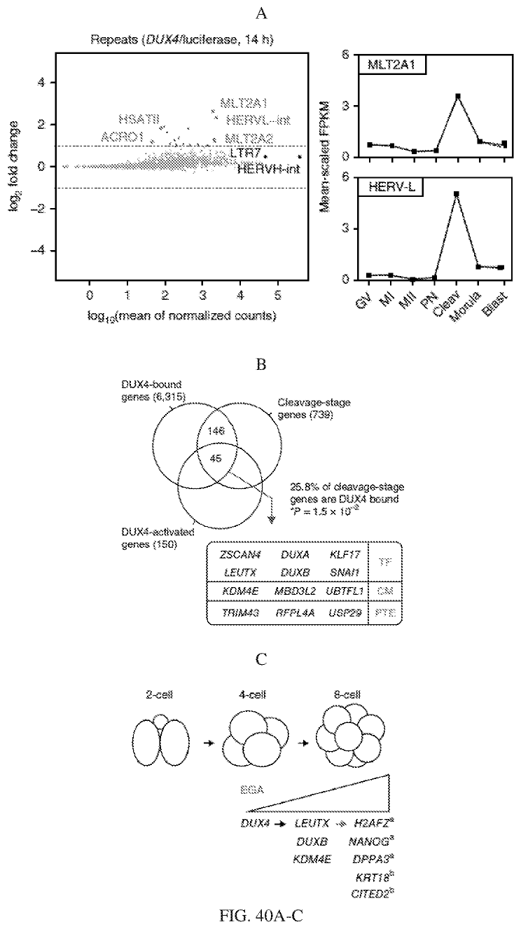

In addition to activating gene expression, hDUX4 also activated specific repetitive elements, including ACRO1 and HSATII satellite repeats, which are also enriched in cleavage-stage embryos (FIG. 42F, G). Most striking, however, was the strong induction of HERVL retrotransposons (FIG. 40A) which are selectively transcribed in the cleavage stage, consistent with previous findings. In keeping with endogenous targets like ZSCAN4, hDUX4 ChIP-sequencing (ChIP-seq) peaks in myoblasts are highly enriched in activated LTR and satellites repeats suggesting that the observed effects are direct. To confirm and extend, the inventors repeated the hDUX4 ChIP-seq experiment in human iPSCs post 24 hr hDUX4 (or luciferase) expression. At standard statistical thresholds (qval<0.01), the inventors observed more than 200,000 peaks (vs. control) shared between two technical replicates. At high thresholds (qval<10⁻²⁰) the inventors observed 65,728 shared peaks-50,674 (77%, p<le-300) of which overlap with the 63,795 peaks previously identified in myoblasts (FIG. 42H). Using GREAT, the inventors next determined direct hDUX4 targets. Of the 739 cleavage-stages genes the inventors identified, at least 25% (191, pval=0.01) were directly occupied by hDUX4 in iPSCs; including prominent cleavage-stage transcription factors (TF), chromatin modifiers (CM), and post-translational modification enzymes (PTE) many of which are also markedly upregualted by hDUX4 expression in iPSCs (FIG. 18C, FIG. 42I). Unique reads also reveal significant hDUX4 enrichment at activated LTR elements (e.g. MLT2A1, MLT2A2) and HSATII satellites (FIG. 42J), consistent with prior findings and the notion of direct repeat element activation. Taken together, the inventors speculate that hDUX4 directly activates a transcriptional program at EGA which helps de-repress germ cell heterochromatin and coordinate gene expression for ensuing lineage decisions (FIG. 40C).

### 6. Functional Conservation of DUX Proteins in Defining the Cleavage Stage Transcriptome in Mammals

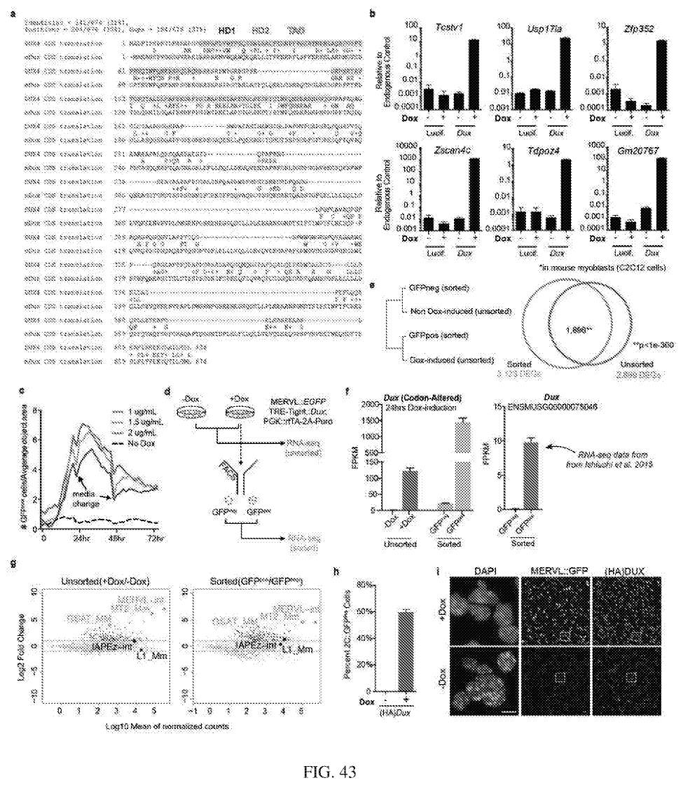

As genetic tools and genomic datasets involving cleavage stage transcription and chromatin dynamics are really only available for mouse, the inventors turned here to test whether DUX4 displays conserved and central roles in mammalian embryogenesis. Our analysis of prior RNA-seq datasets revealed cleavage-stage specific transcription of a weakly conserved DUX4 homolog in mouse, called Dux, hereinafter referred to as mDux for clarity (FIG. 19A, FIG. 43A). Notably, mDux is transiently and specifically expressed in early 2-cell stage mouse embryos (FIG. 19A), one cell cycle earlier than hDUX4 expression in human embryos but consistent with the onset of EGA.

To test whether mDux expression can function as an early embryonic transcriptional activator, the inventors initially expressed it in myoblasts and performed qRT-PCR. Like hDUX4, mDux robustly activated the expression of key cleavage-specific genes such as Zscan4, Zfp352, and Testv1 (FIG. 43B). To extend these findings transcriptome-wide in a developmentally relevant cell-type, the inventors next transfected mESCs with a dox-inducible mDux expression construct (codon altered to facilitate robust expression). RNA-seq on a non-clonal population revealed the upregulation of 123 genes (FC>2, FDR<0.01) (FIG. 19B), including notable retrotransposons (e.g. MERVL and its LTR, MT2) with no genes being significantly downregulated. This cohort of differentially expressed genes is transiently and specifically expressed in the mouse cleavage-stage embryo (FIG. 19C) and contains several orthologs (e.g. Zscan4, Pramef, Ubtfl1, Kdm4e) of genes enriched in human cleavage stage, and directly activated by hDUX4 in iPSCs. Thus, mDux appears to operate as a functional ortholog of hDUX4 in mouse, regulating gene expression during EGA.

### 7. Conversion of mESCs to ‘2C-like’ Cells by mDux Expression

The inventors next tested whether mDux could convert mESCs to a state that resembles the 2-cell mouse embryo (‘2C-like’). ‘2C-like’ cells are a rare metastable subpopulation of mESCs previously identified and isolated by their spontaneous reactivation of MERVL, a murine-specific retrotransposon otherwise only expressed in the 2-cell stage mouse embryo (data not shown). Remarkably, MERVL reactivation in mESCs, revealed by the expression of a MERVL-linked fluorescent protein (MERVL::tdTomato or MERVL::GFP) is linked to the acquisition of molecular and functional features that are specific to the totipotent cleavage embryo, including the expression of early embryonic (2C) genes, the loss of OCT4 protein, and the disaggregation and reformation of constitutive heterochromatin into chromocenters.

Accordingly, the inventors find mDux (data not shown) and mDux-induced genes strongly upregulated in MERVL-expressing cells (FIG. 3C). To evaluate whether mDux could drive conversion of mESCs to the ‘2C-like’ state, the inventors then stably integrated our dox-inducible mDux construct (or luciferase control) into MERVL::GFP reporter mESCs and expanded clonal cell lines (FIG. 19D, left panel). Using flow cytometry to count the number of GFP-positive (GFP^{pos}) cells post dox-induction (24 hrs), the inventors observed conversion efficiencies in mDux-expressing clones ranging from 10-74% GFP^{pos}, with the most efficient clone exhibiting a >500-fold increase compared to controls (FIG. 19D, middle panel). Live imaging fluorescent microscopy confirmed this observation (FIG. 19D, right panel) and further revealed dose-dependency (FIG. 43C).

Dox-induced cells were then either sorted by FACS into GFP^{neg} and GFP^{pos} populations, or left unsorted (versus ‘no dox’ control), and subjected to RNA-seq (FIG. 43D). These two approaches yielded a highly significant overlap (p<le-300) of differentially expressed genes (DEGs) resulting in the unbiased clustering of sorted and unsorted mDux-expressing cells (FIG. 43E). Notably, mDux transgene RNA levels correlated with dox induction and with conversion to a GFP^{pos} state, Although transgene expression in the induced cells exceeded the expression of endogenous mDux RNA in spontaneously fluctuating ‘2C-like’ cells (FIG. 43F), the transcriptional profiles were highly similar (r=0.78) (FIG. 19G). Together, these data indicate mDUX as a potent transcriptional activator of ‘2C-like’ genes and retrotransposons (FIG. 43G). To further determine whether mDux expression imposed other attributes of the ‘2C-like’ state, the inventors examined the status of POU5F1 (OCT4) protein and chromocenters. Here, our IHC results demonstrated a complete loss of OCT4 (despite no change in mRNA) in GFP^{pos} cells, coinciding with the loss of chromocenters (FIG. 19B). Thus, mDux expression appears to elicit in mESCs multiple molecular/biological features of ‘2C-like’ cells, implicating mDUX as the driver of ‘2C-like’ conversion.

### 8. mDux is Necessary for Induction of ‘2C-like’ Cells

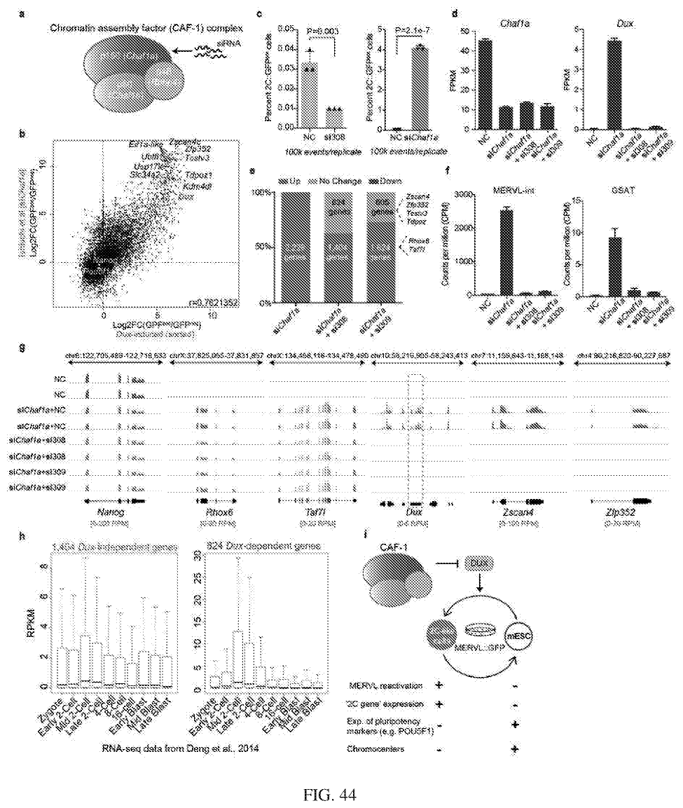

Depletion of Chaf1a, the p150 subunit of the Chromatin assembly factor 1 complex (CAF-1) (FIG. 44A) also induces the conversion of mESCs to a ‘2C-like’ state, prompting an examination of the relationship between CAF-1 and mDux in this process. To begin, the inventors examined prior RNA-seq datasets of mESCs following CAF-1 depletion; this revealed striking mDux upregulation (11-18 fold) in CAF-1 depleted mESCs (FIG. 21A, top panel). Moreover, the downstream targets of mDux (determine in our mDux overexpression studies) composed the most highly activated genes in the CAF-1 depleted datasets (FIG. 21A, bottom and right panel; FIG. 44B).

The inventors next determined whether mDux was necessary for Chaf1a knockdown-mediated entry into a ‘2C-like’ state. To test, the inventors transfected mESCs containing the MERVL::GFP reporter with siRNA pools targeting mDux mRNA (si308 and si309) and/or a previously validated siRNA against Chaf1a. First, depletion of mDux alone (si308) was sufficient to reduce the spontaneous conversion of mESCs to a ‘2C-like’ state (FIG. 44C, left panel), and the inventors confirm prior results showing that depletion of Chaf1a alone leads to a >20-fold increase (FIG. 44C, right panel). Interestingly, co-transfection of mESCs with siRNA against mDux and Chaf1a nearly abolished the inductive effect of Chaf1a knockdown alone (FIG. 21C). To examine the extent to which entry into the ‘2C-like’ state was inhibited, the inventors repeated the knockdowns (two replicates per condition) and isolated RNA for sequencing. First, knockdown of Chaf1a alone greatly altered gene expression, resulting in the upregulation of 2,229 genes (FC>2, FDR<0.01) including mDux and other prominent ‘2C-like’ genes and repetitive elements (FIG. 44D). Moreover, co-depletion of Chaf1a and mDux prevented the activation of 605-824 (27-36%, with si309 or si308, respectively) of the original 2,229 upregulated genes including 123/422 (˜29%; hypergeometric probability p=2.1e-65) of the previously defined ‘2C’ genes induced by Chaf1a knockdown and notable ‘2C-like’ genes and repetitive elements: Zscan4, Zfp352, Tcstv3, and MERVL (FIG. 44E, G). Based on this data, the inventors defined the 824-gene cohort as ‘mnDux-dependent’ and the remaining 1404-gene cohort as ‘mDux-independent’. Remarkably, while the ‘mDux-independent’ cohort lacks developmental stage enrichment, the ‘mDux-dependent’ cohort is predominantly expressed in the 2-cell stage embryo (FIG. 44F). Thus, conversion of mESCs to a ‘2C-like’ state—either spontaneous or through CAF-1 knockdown—is dependent on mDux (FIG. 44H).

### 9. mDux Expression Coverts the Chromatin Landscape of mESCs to One Strongly Resembling Early 2-Cell Mouse Embryos

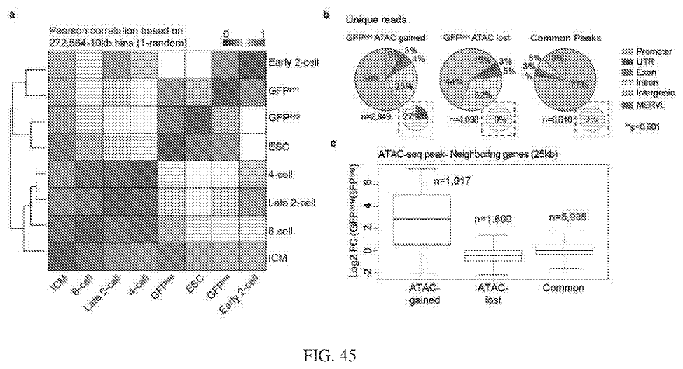

New genomics methodologies, namely ATAC-seq, enable the determination of open versus closed chromatin genome-wide. Cleavage stage chromatin undergoes extensive reorganization to facilitate EGA and the conversion of gametes into totipotent embryos, supported by the distinctive ATAC/chromatin profiles recently revealed in early 2-cell stage embryos. To further characterize mDux function, the inventors next tested whether its expression could convert the chromatin in mESCs to a landscape resembling that of an early 2-cell stage embryo. Accordingly, the inventors performed ATAC-seq on sorted MERVL:: GFP^{pos} and MERVL:: GFP^{neg} cells post 24 hrs dox-induced mDux expression. After calling peaks in each condition, regions of significantly different ATAC-sensitivity (log10 likelihood ratio >3) were identified. Here, the inventors identified 6,071 regions (>500 bp in length) that gained ATAC signal in GFP^{pos} cells compared to GFP^{neg} cells (ATAC-gained) and 4,231 regions that lost ATAC signal (ATAC-lost) (FIG. 22A). Remarkably, not only did the ATAC signal in these regions resemble that seen in early embryos, but unbiased correlation clustering based on genome-wide ATAC-signal clustered the ‘2C-like’ cells with early 2-cell stage (FIG. 45A). In contrast to the 9,131 common peaks found primarily at gene promoters, the ATAC-gained regions were mostly in intergenic space (FIG. 22C), with the majority (64.5%, P<0.001) directly overlapping a MERVL element. Using metagene analysis, the inventors show that mDux-induced ‘2C-like’ cells exhibit extensive and specific opening of chromatin at MERVL-instances, mimicking that of an early 2-cell stage embryo (data not shown). To determine the number and precise location of the MERVL instances that become open following mDux expression, the inventors re-analysed our ATAC-seq analysis using only unique reads. Here, although the number of called ATAC-gained regions was severely reduced, a still significant fraction (27%, p<0.001) overlapped a MERVL element (FIG. 45B). Furthermore, while the ATAC-gained regions were located near genes highly and significantly expressed in ‘2C-like’ cells, the regions of lost ATAC sensitivity were generally located near genes displaying moderate downregulation (FIG. 45C). Taken together, these data demonstrate that mDux-induced ‘2C-like’ cells acquire chromatin accessibility at MERVL elements, which are used specifically in 2-cell stage embryos to regulate the gene expression program at EGA.

### 10. mDux Occupancy is Strongly Correlated with Dynamic Chromatin Sites

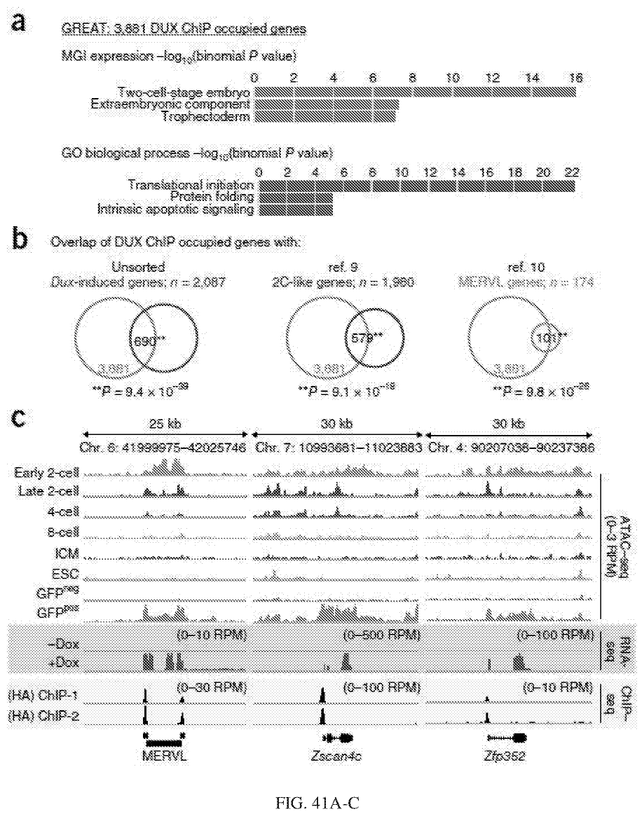

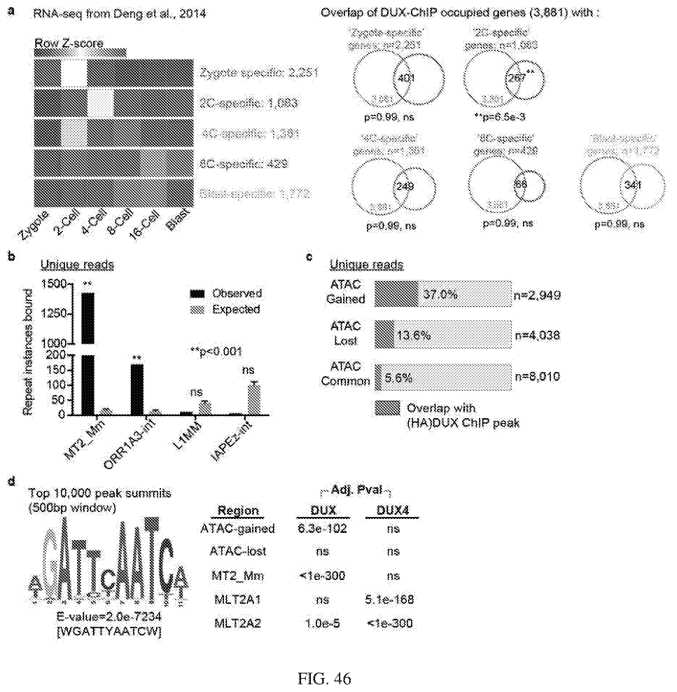

To determine if the observed changes in gene expression and chromatin architecture in ‘2C-like’ cells is due to direct mDUX binding, the inventors localized mDUX in mESCs by CUP sequencing. As no ChIP-grade antibody for mDUX is available, here the inventors created a 3×HA-tagged mDux expression construct and isolated a new clonal MERVL::GFP mESC line. As with earlier clones, our HA-tagged clone displayed high conversion efficiency (60% GFP^{pos} 24 hrs post dox-induction) and expression of HA-mDux coincided with the acquisition of key ‘2C-like’ features (FIG. 43H, I). The HA ChIP-seq (two biological replicates) yielded ˜19,000 shared peaks over input (FDR>0.05), occupying 3,881 genes enriched in a gene expression signature that specifically defines the ‘Two-cell stage embryo” (FIG. 41A, FIG. 46A). Importantly, many of the 3,881 mDUX-occupied genes (-20%) were also activated following mDux overexpression in mESCs and were identified by prior studies as markers of the ‘2C’ and ‘2C-like’ state (FIG. 41B, C). Conservative analyses using unique reads revealed at least 53% of MERVL-LTRs (MT2) and at least 37% of the regions that gain ATAC-sensitivity in ‘2C-like’ cells are directly bound by mDUX (FIG. 46B, C)

Using the top 10,000 peak summits based on enrichment score, the inventors further identified a consensus mDUX binding motif (FIG. 46D), with the top hit (WGATTYAATCW) (SEQ ID NO:137) scoring an E-value of 2.0e-7234. Notably, this motif was highly enriched (adj. pvalue=6.3e-102) in regions of gained ATAC-sensitivity following mDux-overexpression. Finally, the inventors note a lack of hDUX4 motif enrichment within MERVL-LTRs (MT2), and a minimal enrichment for an mDUX motif within HERVL-LTRs (MLT2A1/2). This suggests that DUX4 orthologs, although functionally conserved, have evolved to be species-specific, perhaps in response to ERVs.

### Example 6

### Conservation and Innovation in the DUX4-Family Gene Network

Examples 6 and 1 may have duplicative text, which is not necessarily indicative of different or the same experiments.

While the transcriptome of human DUX4 expressed in human cells is known, the transcriptome of mouse Dux in mouse cells has been largely unknown. Both proteins are encoded by retrogenes derived by the retroposition of DUXC mRNA and both proteins induce apoptosis when expressed in cultured human and mouse muscle cells. Recent studies expressing Dux in human muscle cells or DUX4 in mouse cells showed a partial overlap of regulated genes and a similar consensus binding site; however, these two proteins have diverged significantly at the sequence level, including their homeodomains. Determination of the degree of similarity in their transcriptional programs might help us understand the rapid evolutionary divergence of Dux and DUX4 and inform murine models of FSHD, a disease which still lacks treatment options.

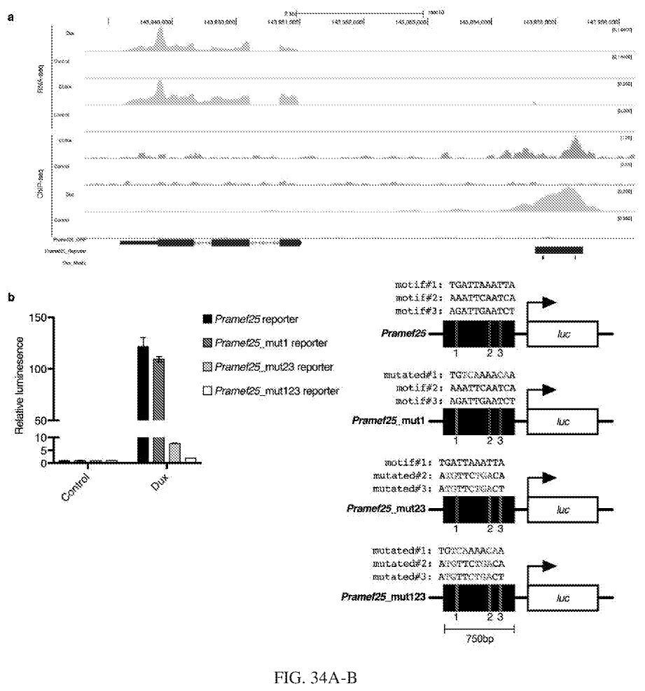

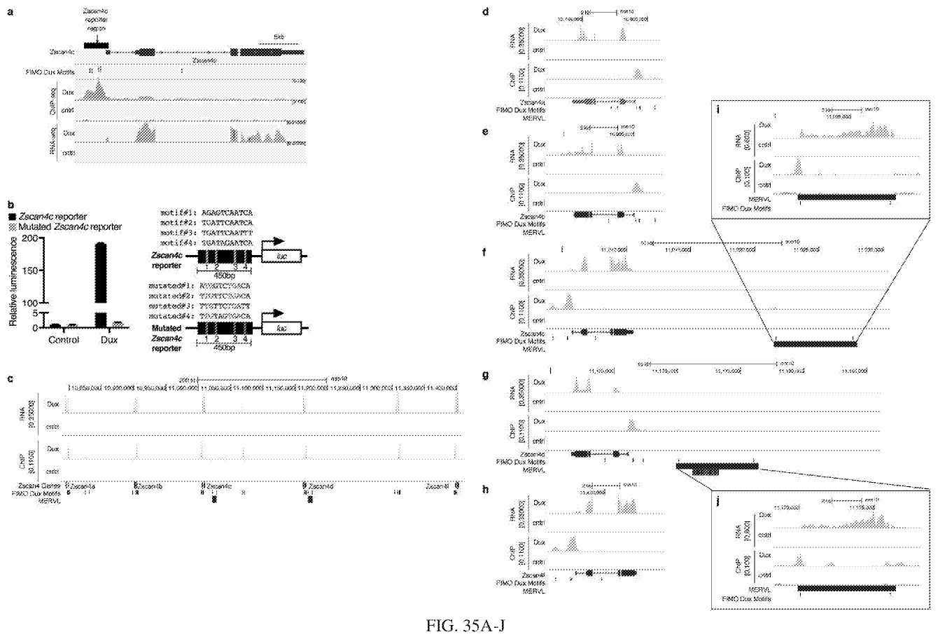

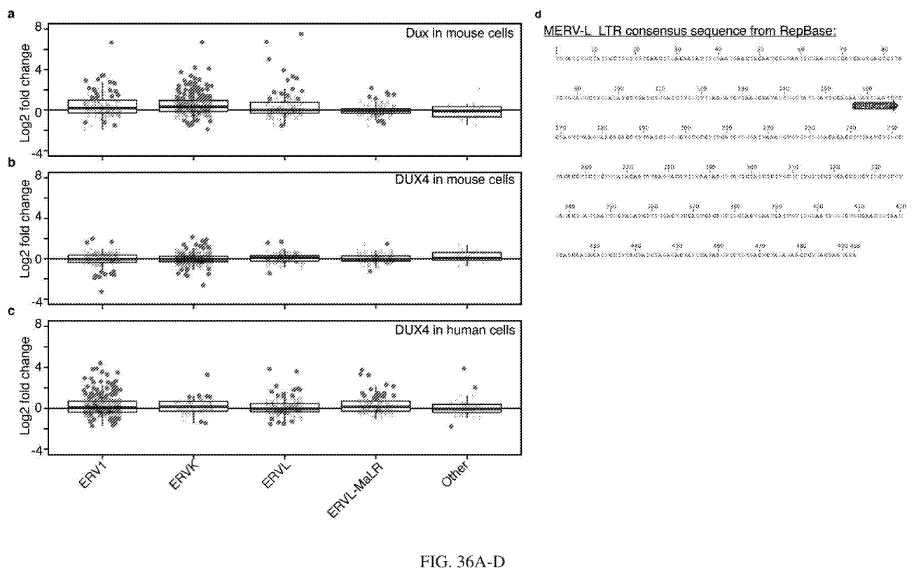

To compare the Dux transcriptome with the previously published DUX4 transcriptome in FSHD muscle cells, the inventors generated RNA-seq and ChIP-seq datasets for Dux expressed in mouse skeletal muscle cells (see Online Methods). The inventors observed increased expression of 962 genes and decreased expression of 204 genes (FIG. 1A). In these data, the most upregulated genes were normally expressed in the mouse 2-cell embryo (e.g. Zscan4a-e, Tcstv1/3), therefore the inventors used gene set enrichment analysis to compare our data to 2-cell-like embryonic stem cells (GSEA; 2C-like). The top of the Dux transcriptome was significantly enriched for the 2C-like gene signature (258/469 genes in the 2C-like gene signature contributed to the GSEA core enrichment, NES=12.56, p-value <0.001; FIG. 1B). In addition, direct targets of Dux (i.e. genes whose RNA increased expression 4-fold or more and have a ChIP-seq peak within 1 kb of the annotated transcriptional start site (TSS)) were enriched in the 2C-like gene signature based on hypergeometric testing (60 direct targets in 2C-like signature/189 total direct targets; 16-fold more direct targets in the 2C-like gene signature than the 3.7 genes expected by chance, p=9.1E-56), including Zscan4a-f, Tcstv1/3, Usp171b/d, Pramef25 and Zfp352. The inventors further confirmed that robust induction of both Pramef25 and Zscan4c reporter constructs depended on intact Dux binding sites (FIG. 34A-B, FIG. 35A-B). ChIP-seq peaks at the TSS of each of the five Zscan4-cluster genes supports the hypothesis that Dux directly binds and activates each Zscan4-cluster gene (FIG. 35C-H). Although there are two MERV-L elements in the Zscan4 locus, the inventors did not observe RNA-seq reads that spliced from these MERV-Ls to any Zscan4 gene (FIG. 35I-J). Importantly, the published 2C-like signature included Dux itself and Dux RNA is expressed in mESC (J. Whiddon, unpublished data). Impartial gene ontology analysis also identified “embryo development” among significantly enriched terms. Together, these results demonstrated that Dux directly regulates many genes in the 2C-like transcriptome in myoblasts.

Despite considerable sequence divergence in their two DNA-binding homeodomain regions (FIG. 1D), the inventors found that Dux and DUX4 activated orthologous genes in myoblasts of their respective species, including genes in the mouse 2C-like gene signature. For this analysis the inventors only considered genes with simple 1:1 mouse-to-human orthology according to HomoloGene. GSEA determined that the 500 genes most upregulated by DUX4 were significantly enriched in the genes most upregulated by Dux (NES=8.16, p-value<0.001; FIG. 1E) and vice versa (NES=6.01, p-value<0.001; FIG. 8). GSEA also demonstrated that DUX4 activated the human orthologs of the mouse 2C-like gene signature (NES=2.24, p-value=0.002, FIG. 1F). It should be noted, however, that these analyses of similarity using the HomoloGene method were conservative. Complex gene families, such as the ZSCAN4, PRAME, THOC4/ALYREF, and USP17 families, were excluded from the HomoloGene dataset because 1:1 orthology cannot be established reliably, but members of each of these families were upregulated in both species. Together, these data demonstrate a strong functional conservation for Dux and DUX4 in the regulation of this 2C-like network in their respective species.

Despite this functional conservation, a de novo motif-finding algorithm identified a Dux binding motif in our ChIP-seq data that diverged from the published DUX4 binding motif in the first half of the motif but not the second (FIG. 2A), perhaps reflecting that the four predicted DNA-binding-specificity residues are identical between DUX4 and Dux in the second homeodomain but not the first (FIG. 1D). The motif identified in this analysis is similar to the recently published motif for Dux in human muscle cells, supporting the notion that the Dux binding motif is cell type independent.

Because of the apparent paradox of the functional conservation of Dux and DUX4 transcriptomes and the partial divergence of their binding motifs, the inventors next generated RNA-seq and ChIP-seq datasets for DUX4 in mouse muscle cells to better understand their conservation and divergence. In this context, DUX4 showed the same binding motif as in human cells (FIG. 9A), increased expression of 582 genes and decreased expression of 428 genes (FIG. 9A). Although DUX4 regulated many genes that were not orthologous to Dux-regulated genes and overall showed little similarity to the Dux transcriptome (FIG. 9C), the genes that were upregulated in both the Dux and DUX4 transcriptomes were enriched for 2C-like genes by hypergeometric testing (p=1.07e-11) and GSEA showed significant enrichment of the 2C-like gene signature activated by DUX4 in mouse cells (NES=4.25, p-value<0.001; FIG. 2B). The activation of this signature, however, was not as robust compared to Dux in mouse cells. For example, Tcstv3 and Zscan4d had 1og2 fold-changes of only 0.92 and 0.66, respectively, compared to 10.1 and 12.4 by Dux, indicating that the top of the DUX4 transcriptome is enriched for the 2C-like gene signature through moderate induction of many members.

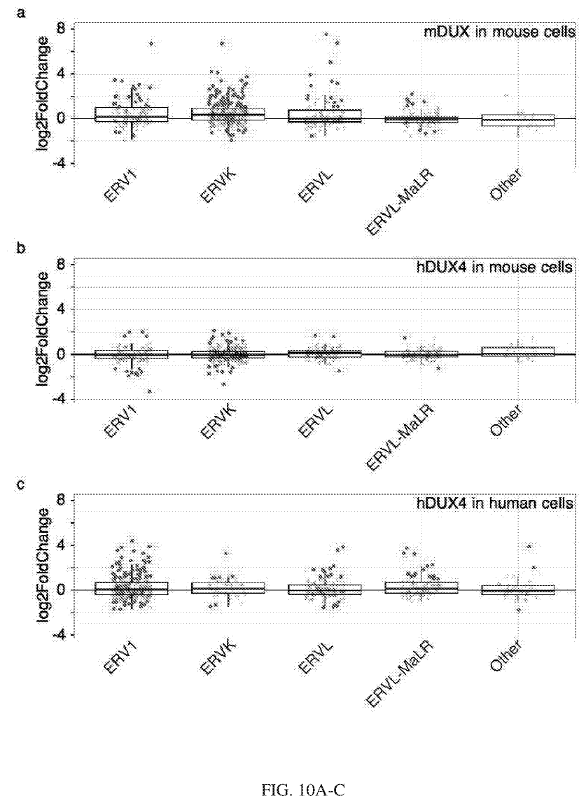

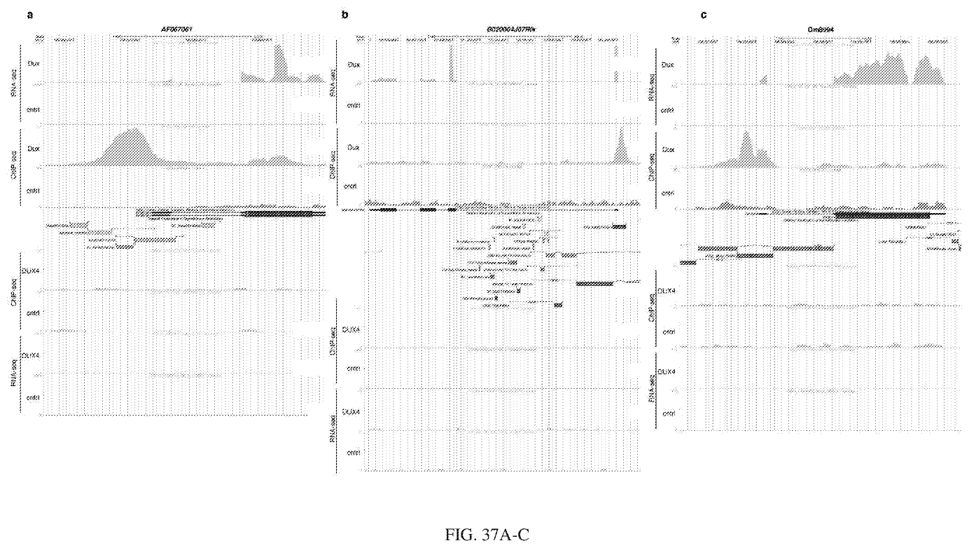

In contrast to the moderate conservation of DUX4's activation of the conventionally-promoted 2C-like program in mouse cells, activation of 2C-like repetitive elements was specific to Dux. Transcription of certain repetitive elements has been reported in 2C-like mouse ES cells and the inventors found that Dux, but not DUX4, induced expression of MERV-L elements by 100-fold and pericentromeric satellite DNA by 50-fold (FIG. 3A-C, FIG. 10A-C). ChIP-seq data indicated that MERV-L elements were a direct target of Dux, but not DUX4 (FIG. 32A), and the MERV-L consensus sequence carries a Dux binding site (FIG. 37D). Consistently, Dux, but not DUX4, activated a reporter driven by a MERVL element and this activation was lost when the inventors mutated the predicted Dux binding site (FIG. 32B). MERV-L elements have been reported to function as alternative promoters in 2C-embryos, which the inventors observed in Dux-expressing, but not DUX4-expressing, mouse cells using two complementary approaches (FIG. 3E, 36D, 37A-C). These results indicate that DUX4 activated a portion of the 2C-like gene signature in mouse cells, but it did not activate repetitive elements characteristic of the 2C mouse embryo.

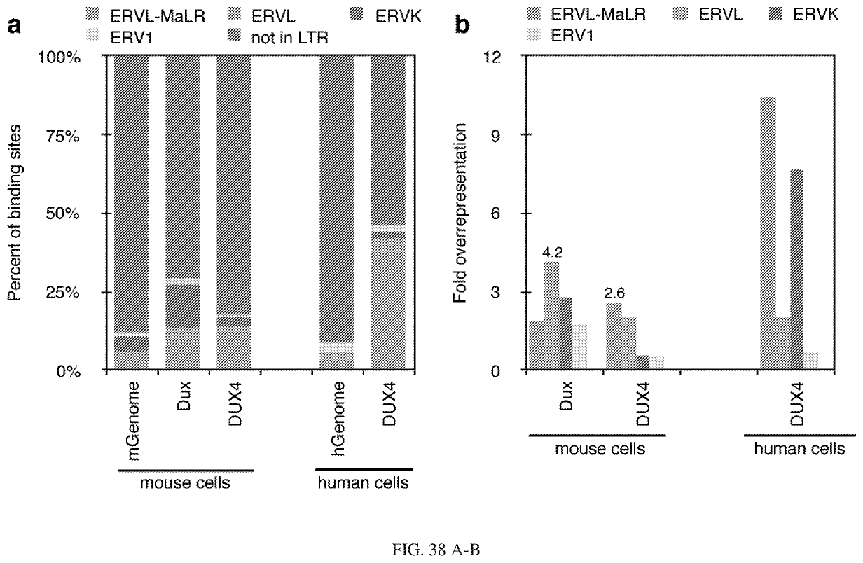

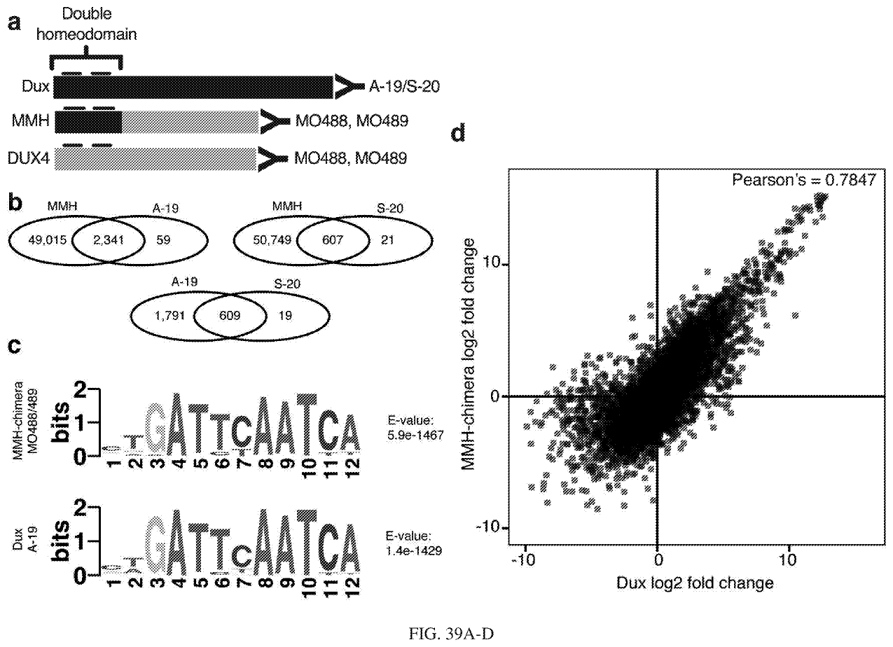

Notably, although DUX4 did not bind nor activate MERV-L elements, DUX4 ChIP-seq peaks were 2.6-fold overrepresented in ERVL-MaLR elements in mouse cells (FIG. 38A-B) and in at least 30 cases used them as alternative promoters (FIG. 4A). It is important to note, however, that Dux and DUX4 bound to mostly distinct sets of ERVL-MaLR elements with less than 4% of all the bound ERVL-MaLR sites in common and only 1 shared alternative promoter. In some cases, DUX4 binding to an ERVL-MaLR retroelement caused robust expression of the adjacent gene (FIG. 4B), consistent with our previous finding that DUX4 bound ERVL-MaLRs when expressed in human cells and used them as alternative promoters. That DUX4 bound and activated transcription of specific endogenous retrotransposon elements in the mouse genome that were not activated by Dux, suggests that homeodomain divergence can selectively activate pre-existing subsets of endogenous retrotransposons and induce the expression of adjacent genes.

The above results indicate that Dux and DUX4 have maintained the ability to regulate a set of 2C-like genes in mouse cells despite considerable divergence of their homeodomains; however, conservation does not extend to the retrotransposons activated by each. The inventors used chimeric proteins to identify the regions of Dux and DUX4 responsible for this partial conservation of function (FIG. 5A). The chimera with the Dux homeodomains and the DUX4 carboxy-terminus (MMH) matched the transcriptional activity of Dux (FIG. 5B), indicating that the transcriptional divergence between Dux and DUX4 mapped to the region containing the two homeodomains.

To determine the relative contribution of each homeodomain, the inventors introduced each human homeodomain individually into Dux to create the MHM and HMM chimeras (FIG. 5A). Neither MHM nor HMM activated transcription of MERV-L-promoted genes (FIG. 5B); whereas for 2C-like genes with conventional promoters, the individual DUX4 homeodomains showed different capacities to substitute for the corresponding Dux homeodomain, with MHM consistently showing stronger activation of the target genes compared to HMM (FIG. 5C-D). The inventors confirmed MHM and HMM expression and stability using a reporter assay (FIG. 12A). The inventors also performed reciprocal experiments in human cells and again observed the second homeodomains were more equivalent than the first homeodomains (FIG. 5E-F), indicating that the similarity of the second homeodomain was important to maintain the functional conservation of the 2C-like gene signature at conventional promoters.

To further explore the evolutionary conservation of the DUX4-family to activate an early embryo gene signature, the inventors assessed the canine DUXC gene. Both Dux and DUX4 are retroposed copies of an ancestral DUXC mRNA and neither mice nor humans have retained DUXC (FIG. 1D). When expressed in mouse muscle cells, canine DUXC did not activate MERV-L-promoted genes (FIG. 5B), but did activate transcription of 2C-like genes with conventional promoters (FIG. 5C-D), again indicating that the ancestral DUX4-like gene activated genes characteristic of early cleavage-stage embryos that was independent of retrotransposon-promoted genes.

Our current study shows that Dux and DUX4 activate genes associated with an early 2C-like program when expressed in muscle cells, consistent with a recent study showing Dux and DUX4 regulate the 2C-like program in early embryos. Despite the divergence of their homeodomains and binding sequences, these factors have maintained the ability to activate the 2C-like gene signature within their own species, but diverged in their ability to activate subsets of retrotransposons, suggesting evolutionary pressure to maintain activation of endogenous genes and a subset of beneficial retrotransposon driven genes, but diverge away from the activation of retrotransposons driving deleterious genes. Genes regulated by all DUX4-family factors likely represent the core ancestral network, while retrotransposon-promoted genes likely contribute species-specific additions. Such comparisons are particularly relevant to FSHD where it remains unclear how to model this disease in non-primate animals. The fact that both DUX4 and Dux expression leads to apoptosis in mouse muscle cells supported the use of DUX4 in mice as a model of FSHD. The cellular toxicity exhibited by cross-species expression might be due to the few classes of genes robustly activated, such as members of the PRAME family, the aggregate action of the larger number of genes moderately activated, such as the 2C/cleavage-stage signature, or the fact that each factor activates classes of retrotransposons and repetitive elements, albeit different classes in different species. Nonetheless, because the pathophysiologic mechanisms of FSHD remain poorly understood, our study suggests that homeodomain divergence might require using Dux to best reproduce the FSHD transcriptional program in murine models of FSHD, although therapies targeting DUX4 RNA or protein would necessarily rely on expression of DUX4. Our study also provides a model for studying genome evolution especially in regards to the critical balance between conservation of a key transcriptional program with the innovation driven by binding to mobile retrotransposon promoters.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

Tawil, R., van der Maarel, S. M. & Tapscott, S. J. Facioscapulohumeral dystrophy: the path to consensus on pathophysiology. Skelet Muscle 4, 12 (2014).

Lek, A., Rahimov, F., Jones, P. L. & Kunkel, L. M. Emerging preclinical animal models for FSHD. Trends Mol Med 21, 295-306 (2015).

Wallace, L. M. et al. DUX4, a candidate gene for facioscapulohumeral muscular dystrophy, causes p53-dependent myopathy in vivo. Ann Neurol 69, 540-52 (2011).

Krom, Y. D. et al. Intrinsic epigenetic regulation of the D4Z4 macrosatellite repeat in a transgenic mouse model for FSHD. PLoS Genet 9, e1003415 (2013).

Dandapat, A. et al. Dominant lethal pathologies in male mice engineered to contain an X-linked DUX4 transgene. Cell Rep 8, 1484-96 (2014).

Geng, L. N. et al. DUX4 activates germline genes, retroelements, and immune mediators: implications for facioscapulohumeral dystrophy. Dev Cell 22, 38-51 (2012).

Young, J. M. et al. DUX4 binding to retroelements creates promoters that are active in FSHD muscle and testis. PLoS Genet 9, e1003947 (2013).

Bosnakovski, D., Daughters, R. S., Xu, Z., Slack, J. M. & Kyba, M. Biphasic myopathic phenotype of mouse DUX, an ORF within conserved FSHD-related repeats. PLoS One 4, e7003 (2009).

Clapp, J. et al. Evolutionary conservation of a coding function for D4Z4, the tandem DNA repeat mutated in facioscapulohumeral muscular dystrophy. Am J Hum Genet 81, 264-79 (2007).

Leidenroth, A. & Hewitt, J. E. A family history of DUX4: phylogenetic analysis of DUXA, B, C and Duxbl reveals the ancestral DUX gene. BMC Evol Biol 10, 364 (2010).

Leidenroth, A. et al. Evolution of DUX gene macrosatellites in placental mammals. Chromosoma 121, 489-97 (2012).

Falco, G. et al. Zscan4: a novel gene expressed exclusively in late 2-cell embryos and embryonic stem cells. Dev Biol 307, 539-50 (2007).

Zhang, W. et al. Zfp206 regulates ES cell gene expression and differentiation. Nucleic Acids Res 34, 4780-90 (2006).

Macfarlan, T. S. et al. Embryonic stem cell potency fluctuates with endogenous retrovirus activity. Nature 487, 57-63 (2012).

Akiyama, T. et al. Transient bursts of Zscan4 expression are accompanied by the rapid derepression of heterochromatin in mouse embryonic stem cells. DNA Res 22, 307-18 (2015).

Jagannathan, S. et al. Model systems of DUX4 expression recapitulate the transcriptional profile of FSHD cells. Hum Mol Genet (2016).

Coordinators, N. R. Database resources of the National Center for Biotechnology Information. Nucleic Acids Res 44, D7-19 (2016).

Bailey, T. L. et al. MEME SUITE: tools for motif discovery and searching. Nucleic Acids Res 37, W202-8 (2009).

Noyes, M. B. et al. Analysis of homeodomain specificities allows the family-wide prediction of preferred recognition sites. Cell 133, 1277-89 (2008).

Peaston, A. E. et al. Retrotransposons regulate host genes in mouse oocytes and preimplantation embryos. Dev Cell 7, 597-606 (2004).

Bosnakovski, D. et al. An isogenetic myoblast expression screen identifies DUX4-mediated FSHD-associated molecular pathologies. EMBO J 27, 2766-79 (2008).

Trapnell, C., Pachter, L. & Salzberg, S. L. TopHat: discovering splice junctions with RNA-Seq. Bioinformatics 25, 1105-11 (2009).

Reich, M. et al. GenePattern 2.0. Nat Genet 38, 500-1 (2006).

Mi, H., Poudel, S., Muruganujan, A., Casagrande, J. T. & Thomas, P. D. PANTHER version 10: expanded protein families and functions, and analysis tools. Nucleic Acids Res 44, D336-42 (2016).

Conerly, M. L., Yao, Z., Zhong, J. W., Groudine, M. & Tapscott, S. J. Distinct Activities of Myf5 and MyoD Indicate Separate Roles in Skeletal Muscle Lineage Specification and Differentiation. Dev Cell 36, 375-85 (2016).

Cao, Y. et al. Genome-wide MyoD binding in skeletal muscle cells: a potential for broad cellular reprogramming. Dev Cell 18, 662-74 (2010).

Li, H. & Durbin, R. Fast and accurate short read alignment with Burrows-Wheeler transform. Bioinformatics 25, 1754-60 (2009).

Choi, J. et al. MyoD converts primary dermal fibroblasts, chondroblasts, smooth muscle, and retinal pigmented epithelial cells into striated mononucleated myoblasts and multinucleated myotubes. Proc Natl Acad Sci U S A 87, 7988-92 (1990).

Davis, R. L., Weintraub, H. & Lassar, A.B. Expression of a single transfected cDNA converts fibroblasts to myoblasts. Cell 51,987-1000 (1987).

Weintraub, H. et al. Activation of muscle-specific genes in pigment, nerve, fat, liver, and fibroblast cell lines by forced expression of MyoD. Proc Natl Acad Sci U S A 86,5434-8 (1989).

Robinson, J. T. et al. Integrative genomics viewer. Nat Biotechnol 29,24-6 (2011).

Thorvaldsdottir, H., Robinson, J. T. & Mesirov, J. P. Integrative Genomics Viewer (IGV): high-performance genomics data visualization and exploration. Brief Bioinform 14, 178-92 (2013).

Zhou, L.-Q. & Dean, J. Reprogramming the genome to totipotency in mouse embryos. Trends Cell Biol. 25, 82-91 (2015).

Liu, L. et al. Telomere lengthening early in development. Nat Cell Biol 9, 1436-1441 (2007).

Matoba, S. et al. Embryonic development following somatic cell nuclear transfer impeded by persisting histone methylation. Cell 159, 884-895 (2014).

Chung, Y. G. et al. Histone Demethylase Expression Enhances Human Somatic Cell Nuclear Transfer Efficiency and Promotes Derivation of Pluripotent Stem Cells. Cell Stem Cell 17, 758-766 (2015).

Zalzman, M. et al. Zscan4 regulates telomere elongation and genomic stability in ES cells. Nature 464, 858-863 (2010).

Schlesinger, S. & Goff, S. P. Retroviral transcriptional regulation and embryonic stem cells: war and peace. Mol. Cell. Biol. 35, 770-777 (2015).

Macfarlan, T. S. et al. Embryonic stem cell potency fluctuates with endogenous retrovirus activity. Nature 487, 57-63 (2012).

Ishiuchi, T. et al. Early embryonic-like cells are induced by downregulating replication-dependent chromatin assembly. Nat. Struct. Mol. Biol. (2015). doi:10.1038/nsmb.3066

Geng, L. N. et al. DUX4 Activates Germline Genes, Retroelements, and Immune Mediators: Implications for Facioscapulohumeral Dystrophy. Dev Cell 22, 38-51 (2012).

Young, J. M. et al. DUX4 Binding to Retroelements Creates Promoters That Are Active in FSHD Muscle and Testis. PLoS Genet 9, e1003947 (2013).

Gertz, J. et al. Transposase mediated construction of RNA-seq libraries. Genome Res. 22, 134-141 (2012).

Xue, Z. et al. Genetic programs in human and mouse early embryos revealed by single-cell RNA sequencing. Nature (2013). doi:10.1038/nature12364

Yan, L. et al. Single-cell RNA-Seq profiling of human preimplantation embryos and embryonic stem cells. Nat. Struct. Mol. Biol. (2013). doi:10.1038/nsmb.2660

Patro, R., Mount, S. M. & Kingsford, C. Sailfish enables alignment-free isoform quantification from RNA-seq reads using lightweight algorithms. Nat Biotechnol 32, 462-464 (2014).

Rickard, A. M., Petek, L. M. & Miller, D. G. Endogenous DUX4 expression in FSHD myotubes is sufficient to cause cell death and disrupts RNA splicing and cell migration pathways. Hum. Mol. Genet. 24, 5901-5914 (2015).

Jagannathan, S. et al. Model systems of DUX4 expression recapitulate the transcriptional profile of FSHD cells. Hum. Mol. Genet. ddw271 (2016). doi:10.1093/hmg/ddw271

Leidenroth, A. & Hewitt, J. E. A family history of DUX4: phylogenetic analysis of DUXA, B, C and Duxbl reveals the ancestral DUX gene. BMC Evol. Biol. 10, 364 (2010).

Holland, P. W. H., Booth, H. A. F. & Bruford, E. A. Classification and nomenclature of all human homeobox genes. BMC Biol. 5, 47 (2007).

Burglin, T. R. & Affolter, M. Homeodomain proteins: an update. Chromosoma 125, 497-521 (2016).

Tohonen, V. et al. Novel PRD-like homeodomain transcription factors and retrotransposon elements in early human development. Nat Commun 6, 8207 (2015).

Madissoon, E. et al. Characterization and target genes of nine human PRD-like homeobox domain genes expressed exclusively in early embryos. Sci Rep 6, 28995 (2016).

Göke, J. et al. Dynamic Transcription of Distinct Classes of Endogenous Retroviral Elements Marks Specific Populations of Early Human Embryonic Cells. Cell Stem Cell 16, 135-141 (2015).

Young, J. M. et al. DUX4 binding to retroelements creates promoters that are active in FSHD muscle and testis. PLoS Genet 9, e1003947 (2013).

Leidenroth, A. et al. Evolution of DUX gene macrosatellites in placental mammals. Chromosoma 121, 489-497 (2012).

Macfarlan, T. S. et al. Endogenous retroviruses and neighboring genes are coordinately repressed by LSD1/KDM1A. Genes Dev. 25, 594-607 (2011).

Schoorlemmer, J., Perez-Palacios, R., Climent, M., Guallar, D. & Muniesa, P. Regulation of Mouse Retroelement MuERV-L/MERVL Expression by REX1 and Epigenetic Control of Stem Cell Potency. Front. Oncol. 4, (2014).

Probst, A. V. et al. A strand-specific burst in transcription of pericentric satellites is required for chromocenter formation and early mouse development. Dev Cell 19, 625-638 (2010).

Casanova, M. et al. Heterochromatin reorganization during early mouse development requires a single-stranded noncoding transcript. Cell Rep 4, 1156-1167 (2013).

Buenrostro, J. D., Giresi, P. G., Zaba, L. C., Chang, H. Y. & Greenleaf, W. J. Transposition of native chromatin for fast and sensitive epigenomic profiling of open chromatin, DNA-binding proteins and nucleosome position. Nat Meth 10, 1213-1218 (2013).

Wu, J. et al. The landscape of accessible chromatin in mammalian preimplantation embryos. Nature 534, 652-657 (2016).

Borsos, M. & Torres-Padilla, M.-E. Building up the nucleus: nuclear organization in the establishment of totipotency and pluripotency during mammalian development. Genes Dev. 30, 611-621 (2016).

Falco, G. et al. Zscan4: A novel gene expressed exclusively in late 2-cell embryos and embryonic stem cells. Dev Biol 307, 539-550 (2007).

Ishiuchi, T. & Torres-Padilla, M.-E. Towards an understanding of the regulatory mechanisms of totipotency. Curr Opin Genet Dev 23, 512-518 (2013).

Choi, S. H. et al. DUX4 recruits p300/CBP through its C-terminus and induces global H3K27 acetylation changes. Nucleic Acids Res. gkw141 (2016). doi:10.1093/nar/gkw141

Rawn, S. M. & Cross, J. C. The evolution, regulation, and function of placenta-specific genes. Annu. Rev. Cell Dev. Biol. 24, 159-181 (2008).

Feschotte, C. Transposable elements and the evolution of regulatory networks. Nat Rev Genet (2008).

Gifford, W. D., Pfaff, S. L. & Macfarlan, T. S. Transposable elements as genetic regulatory substrates in early development. Trends Cell Biol. 23, 218-226 (2013).

Thompson, P. J., Macfarlan, T. S. & Lorincz, M. C. Long Terminal Repeats: From Parasitic Elements to Building Blocks of the Transcriptional Regulatory Repertoire. Mol. Cell 62, 766-776 (2016).

Bénit, L., Lallemand, J. B., Casella, J. F., Philippe, H. & Heidmann, T. ERV-L elements: a family of endogenous retrovirus-like elements active throughout the evolution of mammals. Journal of Virology 73, 3301-3308 (1999).

Cordonnier, A., Casella, J. F. & Heidmann, T. Isolation of novel human endogenous retrovirus-like elements with foamy virus-related pol sequence. Journal of Virology 69, 5890-5897 (1995).

Bénit, L. et al. Cloning of a new murine endogenous retrovirus, MuERV-L, with strong similarity to the human HERV-L element and with a gag coding sequence closely related to the Fv1 restriction gene. Journal of Virology 71, 5652-5657 (1997).

Nakai-Futatsugi, Y. & Niwa, H. Zscan4 Is Activated after Telomere Shortening in Mouse Embryonic Stem Cells. Stem Cell Reports 6, 483-495 (2016).

De Paepe, C., Krivega, M., Cauffman, G., Geens, M. & Van de Velde, H. Totipotency and lineage segregation in the human embryo. Molecular Human Reproduction 20, 599-618 (2014).

Yasuda, T. et al. Recurrent DUX4 fusions in B cell acute lymphoblastic leukemia of adolescents and young adults. Nat Genet 48, 569-574 (2016).

1. A method for reprogramming a cell into a totipotent state, the method comprising expressing a DUXC family protein in the cell.

2. The method of claim 1, wherein the cell is a differentiated somatic cell or iPSC.

3-4. (canceled)

5. The method of claim 1, wherein the totipotent state is an early cleavage-like state.

6-14. (canceled)

15. The method of claim 1, wherein the method further comprises expressing one or more of OCT3/4, Sox2, Klf4, or c-Myc.

16. The method of claim 1, wherein the method further comprises expressing a DNA methyltransferase protein in the cell or administering a DNA methyltransferase (DNMT), a histone dimethylase activator, and/or a H3K9 methyltransferase inhibitor to the cell.

17-18. (canceled)

19. The method of claim 1, wherein the cell is a human, non-human primate, mouse, dog, cow, sheep, or horse cell.

20-22. (canceled)

23. The method of claim 1, wherein expressing a protein comprises transferring a DUXC polypeptide or nucleic acid encoding a DUXC polypeptide into the cell.

24. The method of claim 23, wherein the method comprises transferring a DUXC RNA into the cell.

25. (canceled)

26. The method of claim 1, wherein the method further comprises differentiating the cell into an extraembryonic cell, an embryonic cell, or a derivative thereof.

27. (canceled)

28. The method of claim 26, wherein the method comprises differentiating the cell into an extraembryonic cell and wherein the extraembryonic cell comprises a placental cell, yolk sac cell, extraembryonic endoderm cell, or a derivative thereof.

29. The method of claim 26, wherein the method comprises differentiating the cell into an embryonic cell and wherein the embryonic cell comprises a mesoderm cell, ectoderm cell, endoderm cell, or a derivative thereof.

30. The method of claim 26, wherein the method comprises differentiating the cell into a blood cell, a neural cell, a bone cell, or a skin cell.

31. A method for making a somatic cell nuclear transfer (SCNT) embryo comprising expressing a DUXC protein in a somatic cell and transferring the nucleus of the somatic cell to an enucleated oocyte, thereby making a SCNT embryo.

32-47. (canceled)

48. An animal clone prepared by the method of claim 1.

49. A method for inducing a naïve cell from a primed cell, the method comprising expressing a protein containing a DUXC double homeodomain in the primed cell.

50. (canceled)

51. An isolated totipotent cell prepared by the method of claim 1.

52. (canceled)

53. A method for treating a disease in a subject, the method comprising administering the totipotent cell of claim 1, or a progeny thereof to the subject.

54-60. (canceled)

61. The method of claim 53, wherein the disease is selected from an autoimmune disease, a neurodegenerative disease, or cancer.

62. (canceled)

63. An SCNT embryo prepared by the method of claim 31.

64. (canceled)

65. A method for generating human extraembryonic tissue in vitro, the method comprising differentiating the cell of claim 1 into extraembryonic cells.

66. (canceled)


SEQUENCE LISTING (137 sequences)

SEQ ID NO: 1 - protein, 60 aa, Mus musculus
RRRRKTVWQAWQEQALLSTFKKKRYLSFKERKELAKRMGVSDCRIRVWFQNRRNRSGEEG

SEQ ID NO: 2 - protein, 60 aa, Homo sapiens
GRRRRLVWTPSQSEALRACFERNPYPGIATRERLAWAIGIPEPRVQIWFQNERSRQLRQH

SEQ ID NO: 3 - protein, 60 aa, Canis familiaris
PRRRRLVLTASQKGALQAFFQKNPYPSITAREHLARELAISESRIQVWFQNQRTRQLRQS

SEQ ID NO: 4 - protein, 60 aa, Mus musculus
GRRPRTRLTSLQLRILGQAFERNPRPGFATREELARDTGLPEDTIHIWFQNRRARRRHRR

SEQ ID NO: 5 - protein, 60 aa, Homo sapiens
GRRKRTAVTGSQTALLLRAFEKDRFPGIAAREELARETGLPESRIQIWFQNRRARHPGQG

SEQ ID NO: 6 - protein, 60 aa, Canis familiaris
GRRKRTSISASQTSILLQAFEEERFPGIGMRESLARKTGLPEARIQVWFQNRRARHPGQS

SEQ ID NO: 7 - DNA, 11 bp, Artificial Sequence
  Synthetic Nucleotide
  misc_feature (1)..(1): N is T, A or G
  misc_feature (2)..(2): N is G or A
  misc_feature (4)..(4): N is T, G, or A
  misc_feature (5)..(5): N is T or C
  misc_feature (6)..(6): N is C, T, or G
  misc_feature (10)..(10): N is C or T
  misc_feature (11)..(11): N is A or T
nnannnaatnn

SEQ ID NO: 8 - DNA, 12 bp, Artificial Sequence
  Synthetic Nucleotide
  misc_feature (1)..(1): N is A or T
  misc_feature (2)..(2): N is A or T
  misc_feature (5)..(5): N is C, T, or A
  misc_feature (6)..(6): N is C, T, or G
  misc_feature (7)..(7): N is C or T
nnaannnaatca

SEQ ID NO: 9 - DNA, 12 bp, Artificial Sequence
  Synthetic Nucleotide
  misc_feature (1)..(1): N is A or T
  misc_feature (2)..(2): N is A or T
  misc_feature (5)..(5): N is T, C, or A
  misc_feature (6)..(6): N is C, T, or G
  misc_feature (7)..(7): N is T or C
nnaannnaatca

SEQ ID NO: 10 - DNA, 11 bp, Artificial Sequence
  Synthetic Primer
  misc_feature (1)..(1): N is T or A
  misc_feature (4)..(4): N is C, T, or A
  misc_feature (5)..(5): N is C, T, or G
  misc_feature (6)..(6): N is T or C
naannnaatca

SEQ ID NO: 11 - DNA, 21 bp, Artificial Sequence
  Synthetic Nucleotide
  misc_feature (1)..(21): n is a, c, g, or t
nnnnnnnnnnnnnnnnnnnnn

SEQ ID NO: 12 - DNA, 11 bp, Artificial Sequence
  Synthetic Nucleotide
  misc_feature (1)..(1): N is T, A, or G
  misc_feature (2)..(2): N is G or A
  misc_feature (4)..(4): N is T, G, or A
  misc_feature (5)..(5): N is T or C
  misc_feature (6)..(6): N is C, T, or G
  misc_feature (10)..(10): N is C or T
  misc_feature (11)..(11): N is A or T
nnannnaatnn

SEQ ID NO: 13 - DNA, 12 bp, Artificial Sequence
  Synthetic Nucleotide
  misc_feature (1)..(1): N is C, G, or T
  misc_feature (2)..(2): N is T, A, or G
  misc_feature (3)..(3): N is G or A
  misc_feature (5)..(5): N is T, A, or G
  misc_feature (6)..(6): N is T or C
  misc_feature (7)..(7): N is C, T, or G
  misc_feature (9)..(9): N is A or C
  misc_feature (11)..(11): N is C or T
  misc_feature (12)..(12): N A, T, or C
nnnannnantnn

SEQ ID NO: 14 - DNA, 11 bp, Artificial Sequence
  Synthetic Nucleotide
  misc_feature (1)..(1): N is T, A, or C
  misc_feature (2)..(2): N is A or G
  misc_feature (4)..(4): N is T, C, or A
  misc_feature (5)..(5): N is T, C, or G
  misc_feature (6)..(6): N is T, C, or G
  misc_feature (7)..(7): N is A or C
  misc_feature (10)..(10): N is C or T
  misc_feature (11)..(11): N is A or T
nnannnnatnn

SEQ ID NO: 15 - DNA, 11 bp, Artificial Sequence
  Synthetic Nucleotide
  misc_feature (1)..(1): N is A, T, or G
  misc_feature (4)..(4): N is T or G
  misc_feature (5)..(5): N is T or G
  misc_feature (6)..(6): N is C, T, or G
  misc_feature (7)..(7): N is A or T
  misc_feature (11)..(11): N is A or T
ngannnnatcn

SEQ ID NO: 16 - DNA, 11 bp, Mus musculus
  misc_feature (1)..(1): N is A, T, or G
  misc_feature (4)..(4): N is T or G
  misc_feature (5)..(5): N is T or G
  misc_feature (6)..(6): N is C, T, or G
  misc_feature (7)..(7): N is A or T
  misc_feature (11)..(11): N is A or T
ngannnnatcn

SEQ ID NO: 17 - DNA, 11 bp, Homo sapiens
  misc_feature (1)..(1): N is T, A, or C
  misc_feature (2)..(2): N is A or G
  misc_feature (4)..(4): N is T, C or A
  misc_feature (5)..(5): N is T, C, or G
  misc_feature (6)..(6): N is T, C, or G
  misc_feature (7)..(7): N is A or C
  misc_feature (10)..(10): N is C or T
  misc_feature (11)..(11): N is A or T
nnannnnatnn

SEQ ID NO: 18 - protein, 424 aa, Homo sapiens
MALPTPSDSTLPAEARGRGRRRRLVWTPSQSEALRACFERNPYPGIATRERLAQAIGIPEPRVQIWFQNERSRQLRQHRRESRPWPGRRGPPEGRRKRTAVTGSQTALLLRAFEKDRFPGIAAREELARETGLPESRIQIWFQNRRARHPGQGGRAPAQAGGLCSAAPGGGHPAPSWVAFAHTGAWGTGLPAPHVPCAPGALPQGAFVSQAARAAPALQPSQAAPAEGISQPAPARGDFAYAAPAPPDGALSHPQAPRWPPHPGKSREDRDPQRDGLPGPCAVAQPGPAQAGPQGQGVLAPPTSQGSPWWGWGRGPQVAGAAWEPQAGAAPPPQPAPPDASASARQGQMQGIPAPSQALQEPAPWSALPCGLLLDELLASPEFLQQAQPLLETEAPGELEASEEAASLEAPLSEEEYRALLEEL

SEQ ID NO: 19 - protein, 673 aa, Mus musculus
MAEAGSPVGGSGVARESRRRRKMVWQAWQEQALLSTFKEKRYLSFKERKELAKRMGVSDCRIRVWFQNRRNRSGEEGHASKRSIRGSRRLASPQLQEELGSRPQGRGLRSSGRRPRTRLTSLQRRILGQAFERNPRPGFATREELARDTGLPEDTIHIWFQNRRARRRHRRGRPTAQDQDLLASQGSDGAPTGPEGREREGAQESLLPQEEAGSTGMDTSSPSDLPSFCRESQPFQVAQPRGAGQQEAPTQAGNAGSLEPLLDQLLDEVDVEEPAPAPLNLDGDPGGRVHEGSQKSFWPQEEAGSTGMDTSSPSDSNSFCRESFPSQVAQPCGSGQEDARTQADSTGPLELLLLDQLLDEVQKEEHVPVPLDWGRNPGSREHEGSQDSLLPLEEAANSGMDTSIPSIWPTFCRESQPPQVAQPSGPGQAQAPTQGGNTDPLELLLYQLLDEVQVEEHAPAPLNWDVDPGGRVHEGSWESFWPQREAGSTGLDTSSPSDSNSFCRESQTSQVAQPCGSGQEDARTQANSTGPLELLLLDQLLDEVQKEEHVPAPLDQGRNPGSREHEGSQDSLLPLEEAANSGMDTSIPIWPTFCRESQPPQVAQPSGPGKAQAPTQGGNTDPLELFLDQLLTEVQLEEQGPSPVNVEETWEQMDTTPDLPLTPEEYQTLLDML

SEQ ID NO: 20 - protein, 60 aa, Artificial Sequence
  Synthetic Polypeptide
  MISC_FEATURE (1)..(1): X is G, R, A, S or P
  MISC_FEATURE (6)..(6): X is L, T, or I
  MISC_FEATURE (9)..(9): X is T, Q, K, S, T, or N
  MISC_FEATURE (10)..(10): X is P, A, Q, or L
  MISC_FEATURE (11)..(11): X is S, Q, H, or T
  MISC_FEATURE (13)..(13): X is S, E, R, K, or I
  MISC_FEATURE (14)..(14): X is E, Q, D, or G
  MISC_FEATURE (15)..(15): X is A, T or V
  MISC_FEATURE (17)..(17): X is R, L, Q, or Y
  MISC_FEATURE (19)..(19): X is C, T, V, L, A, F, or S
  MISC_FEATURE (21)..(21): X is E, K, or Q
  MISC_FEATURE (22)..(22): X is R, K, E, or Q
  MISC_FEATURE (29)..(29): X is A, K, T, or M
  MISC_FEATURE (30)..(30): X is T, E, A, or S
  MISC_FEATURE (33)..(33): X is R, E, Q, G, or W
  MISC_FEATURE (36)..(36): X is Q, K, R, or N
  MISC_FEATURE (38)..(38): X is I, M, L or T
  MISC_FEATURE (39)..(39): X is I, V, or L
  MISC_FEATURE (40)..(40): X is I, V, or L
  MISC_FEATURE (41)..(41): X is P, S, or A
  MISC_FEATURE (43)..(43): X is P, C, S, or D
  MISC_FEATURE (45)..(45): X is V or I
  MISC_FEATURE (47)..(47): X is I, V, M, or T
  MISC_FEATURE (52)..(52): X is E, R, or Q
  MISC_FEATURE (54)..(54): X is S, N, R, K, T, A or V
  MISC_FEATURE (56)..(56): X is Q, S, R, or H
  MISC_FEATURE (57)..(57): X is L, G, or S
  MISC_FEATURE (58)..(58): X is R, E, or K
  MISC_FEATURE (60)..(60): X is H, G, Q, or S
XRRRRXVLXXXQXXXLXAXFZXNPYPGIXXREXLAXEXXXXEXRXQXWFQNXRXRXXXQX

SEQ ID NO: 21 - protein, 60 aa, Homo sapiens
GRRRRLVWTPSQSEALRACFERNPYPGIATRERLAQAIGIPEPRVQIWFQNERSRQLRQH

SEQ ID NO: 22 - protein, 60 aa, Mus musculus; identical to SEQ ID NO: 1

SEQ ID NO: 23 - protein, 60 aa, Sus scrofa
SRRRRLVLKQSQRDALQAVFQEKPYPGITTRERLARELSIPESRIQMWFQNQRKRRLKQQ

SEQ ID NO: 24 - protein, 60 aa, Delphinus delphis
SRRRRLVLSLSQKDTLQALFQQNPYPGIMTREWLARELDIPESRIQVWFQNQRRRRLKQS

SEQ ID NO: 25 - protein, 60 aa, Bos taurus
SRRRRLVLKPSQKDALQALFQQNPYPGIATRERLARELGIDESRVQVWFQNQRRRRSKQS

SEQ ID NO: 26 - protein, 60 aa, Equus caballus
SRRRRIVLKASQRDALRAAFQQNPYPGIATRERLAQEIDIPECRVQVWFQNQRRRHLRQS

SEQ ID NO: 27 - protein, 60 aa, Canis familiaris; identical to SEQ ID NO: 3

SEQ ID NO: 28 - protein, 60 aa, Unknown
  Megachiroptera
SRRRRIVLKPHQKEALYTLFQQNPYPGITTRTQLAQEIGLPESRIQVWFQNQRRRHLKQS

SEQ ID NO: 29 - protein, 60 aa, Loxodonta africana
GRRERLVLKPSQRETLQAAFEQNPYPGITTREELARETGIAEDRIQTWFGNRRAGHLRKS

SEQ ID NO: 30 - protein, 60 aa, Unknown
  Folivora
ARRKRIFLNPTQIDVLQASFQKNPYPGIASREQLANEIGVPESRIQVWFQNRRVRRQKQH

SEQ ID NO: 31 - protein, 60 aa, Artificial Sequence
  Synthetic Polypeptide
  MISC_FEATURE (1)..(1): X is G or A
  MISC_FEATURE (7)..(7): X is A, R, F, B, H, S, or P
  MISC_FEATURE (9)..(9): X is T or S
  MISC_FEATURE (10)..(10): X is G, S, P, A, T, or R
  MISC_FEATURE (14)..(14): X is A, R, D, G, S, C, or I
  MISC_FEATURE (15)..(15): X is L, I,  or V
  MISC_FEATURE (17)..(17): X is L, G, R, M, V, or I
  MISC_FEATURE (18)..(18): X is R, Q, or E
  MISC_FEATURE (22)..(22): X is K, R, or Q
  MISC_FEATURE (25)..(25): X is F, R, or Y
  MISC_FEATURE (28)..(28): X is I, F, N, or V
  MISC_FEATURE (29)..(29): X is A, V, G, E or T
  MISC_FEATURE (30)..(30): X is A, T, or M
  MISC_FEATURE (36)..(36): X is R, H, Q, or K
  MISC_FEATURE (37)..(37): X is E, D, Q, K or R
  MISC_FEATURE (40)..(40): X is L or I
  MISC_FEATURE (43)..(43): X is S, D, P, A, or C
  MISC_FEATURE (46)..(46): X is Q, H, or L
  MISC_FEATURE (47)..(47): X is I or V
  MISC_FEATURE (56)..(56): X is H, R, K, or Y
  MISC_FEATURE (58)..(58): X is G, H, E, K, Q, D, or V
  MISC_FEATURE (59)..(59): X is Q, R, or K
  MISC_FEATURE (60)..(60): X is G, R, K, S, or T
XRRKRTXIXXSQTXXLXXAFEXBRXPGXXXREELAXXTGXPEXRIXXWFQNRRARXPXXX

SEQ ID NO: 32 - protein, 60 aa, Homo sapiens; identical to SEQ ID NO: 5

SEQ ID NO: 33 - protein, 60 aa, Mus musculus; identical to SEQ ID NO: 4

SEQ ID NO: 34 - protein, 60 aa, Sus scrofa
AGRKRSFISPSQTDILRQAFERERYPGIAAREELARQTGIPEPQILVWFQNRRARHPEQK

SEQ ID NO: 35 - protein, 60 aa, Delphinus delphis
ARRKRTFISPSQTRVLMQAFERDRFPSIAAREELAHQTGIPEPRIQIWFQNRRARHPKQS

SEQ ID NO: 36 - protein, 60 aa, Bos taurus
ARRKRTVISPSQTRILVQAFTRDRFPGIAAREELARQTGIPEPRIQIWFQNRRARHPQRS

SEQ ID NO: 37 - protein, 60 aa, Equus caballus
GGRKRTHITPWQTGILLESFQKDRFPGIATREELARQTGIPEARIQVWFQNRRARHPDQS

SEQ ID NO: 38 - protein, 60 aa, Canis familiaris; identical to SEQ ID NO: 6

SEQ ID NO: 39 - protein, 60 aa, Unknown
  Megachiroptera
ARRKRTPISPSQRCVLIQAFEKNRFPSIVTREDLARRTGIPECRIQVWFQNRRARHPGQS

SEQ ID NO: 40 - protein, 60 aa, Loxodonta africana
ARRKRTRITTSQTSLLVEAFEKNRYPGNEAKEELAQRTGLPRSRIHVWFQNRRARKPVQS

SEQ ID NO: 41 - protein, 60 aa, Unknown
  Folivora
ARRKRTSITRSQTIILVEAFEQNRFPGVTTREELAKQTGLPEDRIQIWFQNRRNRYPGKT

SEQ ID NO: 42 - protein, 61 aa, Artificial Sequence
  Synthetic Polypeptide
  MISC_FEATURE (1)..(1): X is S, L, E, or N
  MISC_FEATURE (4)..(4): X is E, Q, or D
  MISC_FEATURE (8)..(8): X is A, S, or T
  MISC_FEATURE (9)..(9): X is A, D, V, S, T, or E
  MISC_FEATURE (10)..(10): X is T, D, V, P, A, or K
  MISC_FEATURE (11)..(11): X is A, E, D, Q, or G
  MISC_FEATURE (12)..(12): X is I, M, L, or F
  MISC_FEATURE (13)..(13): X is Q, E, or L
  MISC_FEATURE (14)..(14): X is D, E, G, Q, or P
  MISC_FEATURE (15)..(15): X is T, D, K, Q, or E
  MISC_FEATURE (16)..(16): X is P, V, A, S, or G
  MISC_FEATURE (17)..(17): X is W, H, G, Q, P, or K
  MISC_FEATURE (18)..(18): X is S, P, T, or A
  MISC_FEATURE (19)..(19): X is S, L, F, or P
  MISC_FEATURE (20)..(20): X is P, W, L, or V
  MISC_FEATURE (21)..(21): X is G, V, N, E, S, or D
  MISC_FEATURE (22)..(22): X is S, G, V, A, T, or P
  MISC_FEATURE (23)..(23): X is P, T, D, H, E, or Y
  MISC_FEATURE (24)..(24): X is A, L, T, P, E or S
  MISC_FEATURE (25)..(25): X is G, Q, E, P, or T
  MISC_FEATURE (26)..(26): X is E, A, G, W, or D
  MISC_FEATURE (32)..(32): X is G, E, Q, or L
  MISC_FEATURE (33)..(33): X is V, P, L, D, E, M, or N
  MISC_FEATURE (34)..(34): X is E, P, A, D, L, or S
  MISC_FEATURE (35)..(35): X is A, G, P, T, or L
  MISC_FEATURE (36)..(36): X is T, P, Q, S, F, or D
  MISC_FEATURE (39)..(39): X is T, A, L, G, S, or P
  MISC_FEATURE (42)..(42): X is S, G, or T
  MISC_FEATURE (44)..(44): X is D, E, or I
  MISC_FEATURE (45)..(45): X is E or D
  MISC_FEATURE (46)..(46): X is Y, S, or F
  MISC_FEATURE (47)..(47): X is Q, H, K, or R
  MISC_FEATURE (52)..(52): X is M, I, A, or E
  MISC_FEATURE (54)..(54): X is P, Q, S, or absent
  MISC_FEATURE (55)..(55): X is G, D, S or absent
  MISC_FEATURE (58)..(58): X is G, W, V or absent
  MISC_FEATURE (60)..(60): X is G, Q, C or absent
  MISC_FEATURE (61)..(61): X is A, V, I, P or absent
XLLXELLXXXXXXXXXXXXXXXXXXXELEASXXXXXLEXPLXEXXXXALLDXLXXSPXPXX

SEQ ID NO: 43 - protein, 57 aa, Bos taurus
SLLEEILAATAIQDTPWSSPGSPAGEEGVEATLETPLSEDEYQALLDMLPGSPGPGA

SEQ ID NO: 44 - protein, 57 aa, Canis familiaris
SFLQELFSADEMEEDVHPLWVGTLQEDEPPGPLEAPLSEDDSHALLEMLQDSLWPQA

SEQ ID NO: 45 - protein, 57 aa, Delphinus delphis
SLLDELLSDVDILDKAGPFLNVDTEEEELAATLEAPLSEEEFKALLDILSGSPVPQV

SEQ ID NO: 46 - protein, 50 aa, Loxodonta africana
SLLDELLSAPELQGKSQTFLNADPGEEDPPQLELSLGDIDFQALLDALQD

SEQ ID NO: 47 - protein, 57 aa, Equus caballus
SLLDELLAVTELQEKAQPFLNGHPPAEEPPGTLEGPLSEEEFQALLDMLQSSPGPQI

SEQ ID NO: 48 - protein, 53 aa, Homo sapiens
LLLDELLASPEFLQQAQPLLETEAPGELEASEEAASLEAPLSEEEYRALLEEL

SEQ ID NO: 49 - protein, 49 aa, Unknown
  Megachiroptera
ELLDELLSTPELQDQGKALLSGYPQEEEPEGFLESPLTEEEYQALLDML

SEQ ID NO: 50 - protein, 49 aa, Mus musculus
LFLDQLLTEVQLEEQGPAPVNVEETWEQMDTTPDLPLTSEEYQTLLDML

SEQ ID NO: 51 - protein, 57 aa, Sus scrofa
NLLEEIMAAAGILPEAGPLPDVEEQEELPLGDLEAPLSEEDFQALLDMLPSSPGPCP

SEQ ID NO: 52 - protein, 48 aa, Unknown
  Folivora
SLLDQLLSSKEFLEKAQPLLNPDSQDQNSLPVEPSLSEEEFQALLDML

SEQ ID NO: 53 - DNA, 11 bp, Artificial Sequence
  Synthetic Nucleotide
agattgaatct

SEQ ID NO: 54 - DNA, 11 bp, Artificial Sequence
  Synthetic Nucleotide
atgttgtgact

SEQ ID NO: 55 - DNA, 11 bp, Artificial Sequence
  Synthetic Nucleotide
tgattaaatta

SEQ ID NO: 56 - DNA, 11 bp, Artificial Sequence
  Synthetic Nucleotide
aaattcaatca

SEQ ID NO: 57 - DNA, 11 bp, Artificial Sequence
  Synthetic Nucleotide
agattgaatct

SEQ ID NO: 58 - DNA, 11 bp, Artificial Sequence
  Synthetic Nucleotide
tgtcaaaacaa

SEQ ID NO: 59 - DNA, 11 bp, Artificial Sequence
  Synthetic Nucleotide
aaattcaatca

SEQ ID NO: 60 - DNA, 11 bp, Artificial Sequence
  Synthetic Nucleotide3
agattgaatct

SEQ ID NO: 61 - DNA, 11 bp, Artificial Sequence
  Synthetic Nucleotide
tgattaaatta

SEQ ID NO: 62 - DNA, 11 bp, Artificial Sequence
  Synthetic Nucleotide
atgttctgaca

SEQ ID NO: 63 - DNA, 11 bp, Artificial Sequence
  Synthetic Nucleotide
atgttctgact

SEQ ID NO: 64 - DNA, 11 bp, Artificial Sequence
  Synthetic Nucleotide
tgtcaaaacaa

SEQ ID NO: 65 - DNA, 11 bp, Artificial Sequence
  Synthetic Nucleotide
atgttctgaca

SEQ ID NO: 66 - DNA, 11 bp, Artificial Sequence
  Synthetic Nucleotide
atgttctgact

SEQ ID NO: 67 - DNA, 11 bp, Artificial Sequence
  Synthetic Nucleotide
agagtcaatca

SEQ ID NO: 68 - DNA, 11 bp, Artificial Sequence
  Synthetic Nucleotide
tgattcaatca

SEQ ID NO: 69 - DNA, 11 bp, Artificial Sequence
  Synthetic Nucleotide
tgattcaattt

SEQ ID NO: 70 - DNA, 11 bp, Artificial Sequence
  Synthetic Nucleotide
tgatagaatca

SEQ ID NO: 71 - DNA, 11 bp, Artificial Sequence
  Synthetic Nucleotide
atggtctgaca

SEQ ID NO: 72 - DNA, 11 bp, Artificial Sequence
  Synthetic Nucleotide
ttgttctgaca

SEQ ID NO: 73 - DNA, 11 bp, Artificial Sequence
  Synthetic Nucleotide
ttgttctgatt

SEQ ID NO: 74 - DNA, 11 bp, Artificial Sequence
  Synthetic Nucleotide
ttgtagtgaca

SEQ ID NO: 75 - DNA, 491 bp, Artificial Sequence
  Synthetic Nucleotide
tgtagtggttattcctggttgtcaacttgacaatatttggaatgaactacaatccggaattggaaggctcagcagtgacccttatctggaggcttggagatccttatctggatcttggtttgaagatcttaagccatagtggctatggattccagaagattgaatctccgagtttaaggaacacacctttaatctgggctgcgcctttcatctgggattaaaggtgtggtgggacacacctttaatctgggctacaccttctgctggagacaatataaggacattggaagaagggagtctagctctgctcttgttccttcacctgctgctgcgtgagactgagtaattgctagatctttggacttccattcacagctgcgactgaatcattgttgggaattgggctgccaactgtaagtcatcaataaattcctttactatctagagactattcataagttctgtgactctagagaaccctgactaataca

SEQ ID NO: 76 - DNA, 12 bp, Artificial Sequence
  Synthetic Nucleotide
  misc_feature (1)..(1): N is C or G
  misc_feature (2)..(2): N is T, G, or A
  misc_feature (3)..(3): N is G or A
  misc_feature (6)..(6): N is T or C
  misc_feature (7)..(7): N is C or T
  misc_feature (11)..(11): N is C or T
  misc_feature (12)..(12): N is A or T
nnnatnnaatnn

SEQ ID NO: 77 - DNA, 12 bp, Artificial Sequence
  Synthetic Nucleotide
  misc_feature (1)..(1): N is C or G
  misc_feature (2)..(2): N is T or A
  misc_feature (6)..(6): N is T or C
  misc_feature (7)..(7): N is C, T, or G
  misc_feature (11)..(11): N is C or T
  misc_feature (12)..(12): N is A or T
nngatnnaatnn

SEQ ID NO: 78 - protein, 424 aa, Homo sapiens; identical to SEQ ID NO: 18

SEQ ID NO: 79 - protein, 673 aa, Mus musculus; identical to SEQ ID NO: 19

SEQ ID NO: 80 - DNA, 11 bp, Artificial Sequence
  Synthetic Nucleotide
  misc_feature (1)..(1): N is A, T, or G
  misc_feature (2)..(2): N is G or A
  misc_feature (4)..(4): N is T, G, or A
  misc_feature (5)..(5): N is T, C, or G
  misc_feature (6)..(6): N is C, T, or G
  misc_feature (8)..(8): N is A or C
  misc_feature (10)..(10): N is C or T
  misc_feature (11)..(11): N is A, T, or C
nnannnantnn

SEQ ID NO: 81 - DNA, 1275 bp, Homo sapiens
atggccctcccgacaccctcggacagcaccctccccgcggaagcccggggacgaggacggcgacggagactcgtttggaccccgagccaaagcgaggccctgcgagcctgctttgagcggaacccgtacccgggcatcgccaccagagaacggctggcccaggccatcggcattccggagcccagggtccagatttggtttcagaatgagaggtcacgccagctgaggcagcaccggcgggaatctcggccctggcccgggagacgcggcccgccagaaggccggcgaaagcggaccgccgtcaccggatcccagaccgccctgctcctccgagcctttgagaaggatcgctttccaggcatcgccgcccgggaggagctggccagagagacgggcctcccggagtccaggattcagatctggtttcagaatcgaagggccaggcacccgggacagggtggcagggcgcccgcgcaggcaggcggcctgtgcagcgcggcccccggcgggggtcaccctgctccctcgtgggtcgccttcgcccacaccggcgcgtggggaacggggcttcccgcaccccacgtgccctgcgcgcctggggctctcccacagggggctttcgtgagccaggcagcgagggccgcccccgcgctgcagcccagccaggccgcgccggcagaggggatctcccaacctgccccggcgcgcggggatttcgcctacgccgccccggctcctccggacggggcgctctcccaccctcaggctcctcgctggcctccgcacccgggcaaaagccgggaggaccgggacccgcagcgcgacggcctgccgggcccctgcgcggtggcacagcctgggcccgctcaagcggggccgcagggccaaggggtgcttgcgccacccacgtcccaggggagtccgtggtggggctggggccggggtccccaggtcgccggggcggcgtgggaaccccaagccggggcagctccacctccccagcccgcgcccccggacgcctccgcctccgcgcggcaggggcagatgcaaggcatcccggcgccctcccaggcgctccaggagccggcgccctggtctgcactcccctgcggcctgctgctggatgagctcctggcgagcccggagtttctgcagcaggcgcaacctctcctagaaacggaggccccgggggagctggaggcctcggaagaggccgcctcgctggaagcacccctcagcgaggaagaataccgggctctgctggaggagctttag

SEQ ID NO: 82 - DNA, 1275 bp, Homo sapiens
atggcattgcctacaccttcagactctacgctgcctgcagaggctaggggaagaggtagacggcggcgattggtgtggactccatcacaatccgaagctcttcgcgcatgcttcgagcgcaatccctatccggggattgccacaagggagaggcttgcacaggctatcggaatcccggaaccgagagtgcagatctggttccaaaatgaacgctctcggcagctcagacagcatcgcagggagtcccgcccgtggccaggaagaaggggaccacctgaaggaagaagaaaacgcacagcggtgactggcagccaaacggctctgctgctccgcgctttcgagaaagatcggttccccggaattgccgcacgcgaagaactcgccagagaaactgggctcccagaatcacgaatacagatttggttccagaaccgcagagcaagacacccaggccaggggggacgggcacctgctcaggccggtggactctgctctgctgcccctgggggcggccatccagcaccttcctgggtggctttcgctcatactggcgcttggggtaccgggctgcctgctccgcatgttccctgtgctccaggggccctcccgcagggagcgtttgtttcccaggcagctagggctgcacctgccctgcaaccatcacaggcagcgccagctgaaggcatcagccaacccgccccagcccgcggagattttgcttatgcagcgccagcacctccagacggtgccctgagccacccccaagcccccagatggccccctcaccctggtaagtcccgggaagaccgcgatccccaacgagatggactgcccggtccttgcgctgtggcccagccaggacctgctcaagccggccctcaggggcaaggagtgctggccccacctacaagccagggatctccctggtggggttggggacgcggacctcaggttgctggagccgcttgggagcctcaggccggagctgcaccgccgccacaaccggcccctcccgacgcgtcagcgtccgcccgacaaggccagatgcagggaatcccagcacctagccaagctcttcaagagcctgccccttggagcgcactgccgtgtgggctgctcctggatgaactcctggctagcccagaatttctccagcaggcacagccactcctggaaacagaagctccgggagagctcgaagcctccgaagaagcagcaagcctggaggcacctctttccgaggaggagtatagagcccttctggaagaactttga

SEQ ID NO: 83 - protein, 424 aa, Homo sapiens; identical to SEQ ID NO: 18

SEQ ID NO: 84 - protein, 60 aa, Homo sapiens; identical to SEQ ID NO: 21

SEQ ID NO: 85 - protein, 60 aa, Homo sapiens; identical to SEQ ID NO: 5

SEQ ID NO: 86 - protein, 53 aa, Homo sapiens; identical to SEQ ID NO: 48

SEQ ID NO: 87 - DNA, 2025 bp, Mus musculus
atggcagaagctggcagccctgttggtggcagtggtgtggcacgggaatcccggcggcgcaggaagacggtttggcaggcctggcaagagcaggccctgctatcaactttcaagaagaagagatacctgagcttcaaggagaggaaggagctggccaagcgaatgggggtctcagattgccgcatccgcgtgtggtttcagaaccgcaggaatcgcagtggagaggaggggcatgcctcaaagaggtccatcagaggctccaggcggctagcctcgccacagctccaggaagagcttggatccaggccacagggtagaggcatgcgctcatctggcagaaggcctcgcactcgactcacctcgctacagctcaggatcctagggcaagcctttgagaggaacccacgaccaggctttgctaccagggaggagctggcgcgtgacacagggttgcccgaggacacgatccacatatggtttcaaaaccgaagagctcggcggcgccacaggaggggcaggcccacagctcaagatcaagacttgctggcgtcacaagggtcggatggggcccctgcaggtccggaaggcagagagcgtgaaggtgcccaggagaacttgttgccacaggaagaagcaggaagtacgggcatggatacctcgagccctagcgacttgccctccttctgcggagagtcccagcctttccaagtggcacagccccgtggagcaggccaacaagaggcccccactcgagcaggcaacgcaggctctctggaacccctccttgatcagctgctggatgaagtccaagtagaagagcctgctccagcccctctgaatttggatggagaccctggtggcagggtgcatgaaggttcccaggagagcttttggccacaggaagaagcaggaagtacaggcatggatacttctagccccagcgactcaaactccttctgcagagagtcccagccttcccaagtggcacagccctgtggagcgggccaagaagatgcccgcactcaagcagacagcacaggccctctggaactcctcctccttgatcaactgctggacgaagtccaaaaggaagagcatgtgccagtcccactggattggggtagaaatcctggcagcagggagcatgaaggttcccaggacagcttactgcccctggaggaagcagtaaattcgggcatggatacctcgatccctagcatctggccaaccttctgcagagaatcccagcctccccaagtggcacagccctctggaccaggccaagcacaggcccccactcaaggtgggaacacggaccccctggagctcttcctctatcaactgttggatgaagtccaagtagaagagcatgctccagcccctctgaattgggatgtagatcctggtggcagggtgcatgaaggttcgtgggagagcttttggccacaggaagaagcaggaagtacaggcctggatacttcaagccccagcgactcaaactccttcttcagagagtccaagccttcccaagtggcacagcgccgtggagcgggccaagaagatgcccgcactcaagcagacagcacaggccctctggaactcctcctctttgatcaactgctggacgaagtccaaaaggaagagcatgtgccagccccactggattggggtagaaatcctggcagcatggagcatgaaggttcccaggacagcttactgcccctggaggaagcagcaaattcgggcagggatacctcgatccctagcatctggccagccttctgcagaaaatcccagcctccccaagtggcacagccctctggaccaggccaagcacaggcccccattcaaggtgggaacacggaccccctggagctcttccttgatcaactgctgaccgaagtccaacttgaggagcaggggcctgcccctgtgaatgtggaggaaacatgggagcaaatggacacaacacctgatctgcctctcacttcagaagaatatcagactcttctagatatgctctga

SEQ ID NO: 88 - DNA, 2025 bp, Mus musculus
atggctgaggctggctctccagtgggaggatctggagtggccagagaatcaaggagaaggaggaaaactgtctggcaagcttggcaggaacaggcactcctgagcacatttaagaaaaaaaggtatctgtcctttaaagaaagaaaggaactggcaaaaaggatgggagtttctgattgcaggatcagagtctggttccagaataggagaaataggtctggggaggaaggacatgcaagcaagagaagcataagaggttccaggaggctggcatcccctcaacttcaggaggaactgggaagtaggccccaaggcaggggcatgaggtcctcagggaggagacccagaaccaggctgacaagtctgcagctgagaatccttggtcaggcttttgaaaggaatccaaggccaggatttgccaccagagaggaactggccagggatacaggccttcctgaggatactatccatatctggttccagaacaggagggccaggagaaggcacagaaggggaagacctacagcccaggaccaggacctcctggcttcccagggttctgatggagcacctgctgggcctgaaggtagagagagagaaggagcacaggaaaatttgctgccccaggaggaggcaggatcaacagggatggacacctcaagcccttctgacctcccttcattctgtggtgaatcacagccctttcaggtggcccagcccaggggagctggacagcaggaggctcccacaagggcagggaatgctggatcattggagccactgttggaccagctcttggatgaggtccaggtggaggaacctgccccagctccactcaacctggatggtgatcctggggggagggttcatgagggtagtcaggagtccttctggccccaggaggaggctggttctactggaatggacacttcttcaccctctgacagcaatagcttttgcagggagagtcaaccctctcaggtagctcagccttgtggggctggccaggaggatgctaggacccaggctgactcaacagggcccttggagctgttgctgctggaccagctcctggatgaggtacagaaggaggaacatgtaccagtgcccctggactgggggaggaaccctggaagcagagaacatgagggtagtcaggattctctccttcctctggaagaggctgtgaattctggaatggacactagtataccaagtatttggcctacattttgcagggagtcacaacccccacaggtggctcagccttcaggacctgggcaggcccaggctcctacccaagggggtaatacagacccactggaactctttctgtatcagctgctggatgaggtccaggtggaggaacatgccccagctccactcaactgggatgtggatccagggggcagagtccatgagggttcctgggagtcattctggccccaggaggaggcaggctctacaggactggacacaagctcccctagtgacagcaactcattctttagggagagtaagccctctcaggttgctcaaaggaggggagctgggcaagaggatgccaggactcaggctgacagtacaggacccctggagctgctgttgtttgaccagctcctggatgaagtgcagaaggaggaacatgttccagctcccctggactggggaaggaaccctggttctatggaacatgagggctctcaggactctctcttgcctctggaagaagctgctaatagtggcagagatacaagtatcccaagcatttggcctgccttttgcaggaaaagccagccaccccaggtagcccagcctagtggacctggacaggctcaggcacctatacaaggaggcaacactgacccattggagttgtttctggaccagctgctcactgaggtgcaactggaggaacaagggccagcacctgtcaatgttgaagagacctgggaacagatggataccactccagacttgccactgacttctgaagagtaccagacccttcttgacatgctgtaa

SEQ ID NO: 89 - protein, 674 aa, Mus musculus
MAEAGSPVGGSGVARESRRRRKTVWQAWQEQALLSTFKKKRYLSFKERKELAKRMGVSDCRIRVWFQNRRNRSGEEGHASKRSIRGSRRLASPQLQEELGSRPQGRGMRSSGRRPRTRLTSLQLRILGQAFERNPRPGFATREELARDTGLPEDTIHIWFQNRRARRRHRRGRPTAQDQDLLASQGSDGAPAGPEGREREGAQENLLPQEEAGSTGMDTSSPSDLPSFCGESQPFQVAQPRGAGQQEAPTRAGNAGSLEPLLDQLLDEVQVEEPAPAPLNLDGDPGGRVHEGSQESFWPQEEAGSTGMDTSSPSDSNSFCRESQPSQVAQPCGAGQEDARTQADSTGPLELLLLDQLLDEVQKEEHVPVPLDWGRNPGSREHEGSQDSLLPLEEAVNSGMDTSIPSIWPTFCRESQPPQVAQPSGPGQAQAPTQGGNTDPLELFLYQLLDEVQVEEHAPAPLNWDVDPGGRVHEGSWESFWPQEEAGSTGLDTSSPSDSNSFFRESKPSQVAQRRGAGQEDARTQADSTGPLELLLFDQLLDEVQKEEHVPAPLDWGRNPGSMEHEGSQDSLLPLEEAANSGRDTSIPSIWPAFCRKSQPPQVAQPSGPGQAQAPIQGGNTDPLELFLDQLLTEVQLEEQGPAPVNVEETWEQMDTTPDLPLTSEEYQTLLDML

SEQ ID NO: 90 - protein, 60 aa, Mus musculus; identical to SEQ ID NO: 1

SEQ ID NO: 91 - protein, 60 aa, Mus musculus; identical to SEQ ID NO: 4

SEQ ID NO: 92 - protein, 49 aa, Mus musculus; identical to SEQ ID NO: 50

SEQ ID NO: 93 - DNA, 1329 bp, Canis familiaris
atggcctccagcagcacccccggcggcccactccctcgagcaccccgacgaaggaggctcgtgttgacggcaagccagaagggggccctgcaggcattcttccagaagaacccttaccccagcatcactgccagagaacacctggcccgagagctggccatctccgagtctagaatccaggtctggttccaaaaccagagaacgagacagctaaggcagagccgccgactggactccagaattccccaaggagaagggccaccgaatggaaaggcacagcctccaggtcgagtcccgaaggaaggcaggagaaaacggacatccatttctgcatcccaaaccagtatcctccttcaagcctttgaggaggagcggtttcctggcattggtatgagggaaagcctggccagaaaaacaggccttccagaagccagaattcaggtttggtttcagaacagaagagctcggcacccagggcagagcccaagtgggcccgagaatgctttggcggcaaaccacaaacccagtcctcgcgggacggtcccattggaccaaagccacctgtcaagggtccccaggagctctccaaatctggctcccttcgatcccttgggaagcatgcagacgcaggctgcagggacacctcctgtctcctccgtggttgttgtccctccagtttcttgtgggggctttgggcgcctgattccgggggcctgcctggtcacaccaaccttaggtgggcaaggaggaatcgctgctgctcccagagtcctggggagccgatgctgcccagaactgactccaggagggggcctctcaccaggtcatgctgaccttggcctcccctcccctgggagatgccagcagccgaaagagcaccccagcaaggcgcccctgccctcgcaagttggcccgcggcctccgcctgttgatcctcctcaacactggggtcatgcaggtcccccgggcaccggtcaggccacgccgaggaggggccaaagttcccaggcagtcatgggcacagcagggtcccaggatgggacagggcagcagcccgcccccggggagagccccgcttggtggcaacagcctccccctcctgcagggccatgtgtcccgctgcccccacaacaccagctgtgtgcggacacctccagtttcctacaagagcttttctcagccgatgagatggaagaagatgtccaccccttgtgggtggggactctgcaggaggacgaacctccaggacccctggaagcacccctcagcgaggacgattctcacgctctgctggaaatgctacaggactccttgtggcctcaggcctag

SEQ ID NO: 94 - protein, 442 aa, Canis familiaris
MASSSTPGGPLPRAPRRRRLVLTASQKGALQAFFQKNPYPSITAREHLARELAISESRIQVWFQNQRTRQLRQSRRLDSRIPQGEGPPNGKAQPPGRVPKEGRRKRTSISASQTSILLQAFEEERFPGIGMRESLARKTGLPEARIQVWFQNRRARHPGQSPSGPENALAANHKPSPRGTVPLDQSHLSRVPRSSPNLAPFDPLGSMQTQAAGTPPVSSVVVVPPVSCGGFGRLIPGACLVTPTLGGQGGIAAAPRVLGSRCCPELTPGGGLSPGHADLGLPSPGRCQQPKEHPSKAPLPSQVGPRPPPVDPPQHWGHAGPPGTGQATPRRGQSSQAVMGTAGSQDGTGQQPAPGESPAWWQQPPPPAGPCVPLPPQHQLCADTSSFLQELFSADEMEEDVHPLWVGTLQEDEPPGPLEAPLSEDDSHALLEMLQDSLWPQA

SEQ ID NO: 95 - protein, 60 aa, Canis familiaris; identical to SEQ ID NO: 3

SEQ ID NO: 96 - protein, 60 aa, Canis familiaris; identical to SEQ ID NO: 6

SEQ ID NO: 97 - protein, 57 aa, Canis familiaris; identical to SEQ ID NO: 44

SEQ ID NO: 98 - DNA, 1329 bp, Artificial Sequence
  Synthetic Nucleotide
atggctgaggctggctctccagtgggaggatctggagtggccagagaatcaaggagaaggaggaaaactgtctggcaagcttggcaggaacaggcactcctgagcacatttaagaaaaaaaggtatctgtcctttaaagaaagaaaggaactggcaaaaaggatgggagtttctgattgcaggatcagagtctggttccagaataggagaaataggtctggggaggaaggacatgcaagcaagagaagcataagaggttccaggaggctggcatcccctcaacttcaggaggaactgggaagtaggccccaaggcaggggcatgaggtcctcagggaggagacccagaaccaggctgacaagtctgcagctgagaatccttggtcaggcttttgaaaggaatccaaggccaggatttgccaccagagaggaactggccagggatacaggccttcctgaggatactatccatatctggttccagaacaggagggccaggagaaggcacagaaggggaagacctcctgctcaggccggtggactctgctctgctgcccctgggggcggccatccagcaccttcctgggtggctttcgctcatactggcgcttggggtaccgggctgcctgctccgcatgttccctgtgctccaggggccctcccgcagggagcgtttgtttcccaggcagctagggctgcacctgccctgcaaccatcacaggcagcgccagctgaaggcatcagccaacccgccccagcccgcggagattttgcttatgcagcgccagcacctccagacggtgccctgagccacccccaagcccccagatggccccctcaccctggtaagtcccgggaagaccgcgatccccaacgagatggactgcccggtccttgcgctgtggcccagccaggacctgctcaagccggccctcaggggcaaggagtgctggccccacctacaagccagggatctccctggtggggttggggacgcggacctcaggttgctggagccgcttgggagcctcaggccggagctgcaccgccgccacaaccggcccctcccgacgcgtcagcgtccgcccgacaaggccagatgcagggaatcccagcacctagccaagctcttcaagagcctgccccttggagcgcactgccgtgtgggctgctcctggatgaactcctggctagcccagaatttctccagcaggcacagccactcctggaaacagaagctccgggagagctcgaagcctccgaagaagcagcaagcctggaggcacctctttccgaggaggagtatagagcccttctggaagaactttga

SEQ ID NO: 99 - protein, 442 aa, Artificial Sequence
  Synthetic Polypeptide
MAEAGSPVGGSGVARESRRRRKTVWQAWQEQALLSTFKKKRYLSFKERKELAKRMGVSDCRIRVWFQNRRNRSGEEGHASKRSIRGSRRLASPQLQEELGSRPQGRGMRSSGRRPRTRLTSLQLRILGQAFERNPRPGFATREELARDTGLPEDTIHIWFQNRRARRRHRRGRPPAQAGGLCSAAPGGGHPAPSWVAFAHTGAWGTGLPAPHVPCAPGALPQGAFVSQAARAAPALQPSQAAPAEGISQPAPARGDFAYAAPAPPDGALSHPQAPRWPPHPGKSREDRDPQRDGLPGPCAVAQPGPAQAGPQGQGVLAPPTSQGSPWWGWGRGPQVAGAAWEPQAGAAPPPQPAPPDASASARQGQMQGIPAPSQALQEPAPWSALPCGLLLDELLASPEFLQQAQPLLETEAPGELEASEEAASLEAPLSEEEYRALLEEL

SEQ ID NO: 100 - DNA, 2025 bp, Artificial Sequence
  Synthetic Nucleotide
atggctgaggctggctctccagtgggaggatctggagtggccagagaatcaaggagaaggaggaaaactgtctggcaagcttggcaggaacaggcactcctgagcacatttaagaaaaaaaggtatctgtcctttaaagaaagaaaggaactggcaaaaaggatgggagtttctgattgcaggatcagagtctggttccagaataggagaaataggtctggggaggaaggacatgcaagcaagagaagcataagaggttccaggaggctggcatcccctcaacttcaggaggaactgggaagtaggccccaaggcaggggcatgaggtcctcaggaagaagaaaacgcacagcggtgactggcagccaaacggctctgctgctccgcgctttcgagaaagatcggttccccggaattgccgcacgcgaagaactcgccagagaaactgggctcccagaatcacgaatacagatttggttccagaaccgcagagcaagacacccaggccaggggggaagacctacagcccaggaccaggacctcctggcttcccagggttctgatggagcacctgctgggcctgaaggtagagagagagaaggagcacaggaaaatttgctgccccaggaggaggcaggatcaacagggatggacacctcaagcccttctgacctcccttcattctgtggtgaatcacagccctttcaggtggcccagcccaggggagctggacagcaggaggctcccacaagggcagggaatgctggatcattggagccactgttggaccagctcttggatgaggtccaggtggaggaacctgccccagctccactcaacctggatggtgatcctggggggagggttcatgagggtagtcaggagtccttctggccccaggaggaggctggttctactggaatggacacttcttcaccctctgacagcaatagcttttgcagggagagtcaaccctctcaggtagctcagccttgtggggctggccaggaggatgctaggacccaggctgactcaacagggcccttggagctgttgctgctggaccagctcctggatgaggtacagaaggaggaacatgtaccagtgcccctggactgggggaggaaccctggaagcagagaacatgagggtagtcaggattctctccttcctctggaagaggctgtgaattctggaatggacactagtataccaagtatttggcctacattttgcagggagtcacaacccccacaggtggctcagccttcaggacctgggcaggcccaggctcctacccaagggggtaatacagacccactggaactctttctgtatcagctgctggatgaggtccaggtggaggaacatgccccagctccactcaactgggatgtggatccagggggcagagtccatgagggttcctgggagtcattctggccccaggaggaggcaggctctacaggactggacacaagctcccctagtgacagcaactcattctttagggagagtaagccctctcaggttgctcaaaggaggggagctgggcaagaggatgccaggactcaggctgacagtacaggacccctggagctgctgttgtttgaccagctcctggatgaagtgcagaaggaggaacatgttccagctcccctggactggggaaggaaccctggttctatggaacatgagggctctcaggactctctcttgcctctggaagaagctgctaatagtggcagagatacaagtatcccaagcatttggcctgccttttgcaggaaaagccagccaccccaggtagcccagcctagtggacctggacaggctcaggcacctatacaaggaggcaacactgacccattggagttgtttctggaccagctgctcactgaggtgcaactggaggaacaagggccagcacctgtcaatgttgaagagacctgggaacagatggataccactccagacttgccactgacttctgaagagtaccagacccttcttgacatgctgtaa

SEQ ID NO: 101 - protein, 674 aa, Artificial Sequence
  Synthetic Polypeptide
MAEAGSPVGGSGVARESRRRRKTVWQAWQEQALLSTFKKKRYLSFKERKELAKRMGVSDCRIRVWFQNRRNRSGEEGHASKRSIRGSRRLASPQLQEELGSRPQGRGMRSSGRRKRTAVTGSQTALLLRAFEKDRFPGIAAREELARETGLPESRIQIWFQNRRARHPGQGGRPTAQDQDLLASQGSDGAPAGPEGREREGAQENLLPQEEAGSTGMDTSSPSDLPSFCGESQPFQVAQPRGAGQQEAPTRAGNAGSLEPLLDQLLDEVQVEEPAPAPLNLDGDPGGRVHEGSQESFWPQEEAGSTGMDTSSPSDSNSFCRESQPSQVAQPCGAGQEDARTQADSTGPLELLLLDQLLDEVQKEEHVPVPLDWGRNPGSREHEGSQDSLLPLEEAVNSGMDTSIPSIWPTFCRESQPPQVAQPSGPGQAQAPTQGGNTDPLELFLYQLLDEVQVEEHAPAPLNWDVDPGGRVHEGSWESFWPQEEAGSTGLDTSSPSDSNSFFRESKPSQVAQRRGAGQEDARTQADSTGPLELLLFDQLLDEVQKEEHVPAPLDWGRNPGSMEHEGSQDSLLPLEEAANSGRDTSIPSIWPAFCRKSQPPQVAQPSGPGQAQAPIQGGNTDPLELFLDQLLTEVQLEEQGPAPVNVEETWEQMDTTPDLPLTSEEYQTLLDML

SEQ ID NO: 102 - DNA, 2025 bp, Artificial Sequence
  Synthetic Nucleotide
atggctgaggctggctctccagtgggaggatctggagtggccagagaatcaggtagacggcggcgattggtgtggactccatcacaatccgaagctcttcgcgcatgcttcgagcgcaatccctatccggggattgccacaagggagaggcttgcacaggctatcggaatcccggaaccgagagtgcagatctggttccaaaatgaacgctctcggcagctcagacagcatcatgcaagcaagagaagcataagaggttccaggaggctggcatcccctcaacttcaggaggaactgggaagtaggccccaaggcaggggcatgaggtcctcagggaggagacccagaaccaggctgacaagtctgcagctgagaatccttggtcaggcttttgaaaggaatccaaggccaggatttgccaccagagaggaactggccagggatacaggccttcctgaggatactatccatatctggttccagaacaggagggccaggagaaggcacagaaggggaagacctacagcccaggaccaggacctcctggcttcccagggttctgatggagcacctgctgggcctgaaggtagagagagagaaggagcacaggaaaatttgctgccccaggaggaggcaggatcaacagggatggacacctcaagcccttctgacctcccttcattctgtggtgaatcacagccctttcaggtggcccagcccaggggagctggacagcaggaggctcccacaagggcagggaatgctggatcattggagccactgttggaccagctcttggatgaggtccaggtggaggaacctgccccagctccactcaacctggatggtgatcctggggggagggttcatgagggtagtcaggagtccttctggccccaggaggaggctggttctactggaatggacacttcttcaccctctgacagcaatagcttttgcagggagagtcaaccctctcaggtagctcagccttgtggggctggccaggaggatgctaggacccaggctgactcaacagggcccttggagctgttgctgctggaccagctcctggatgaggtacagaaggaggaacatgtaccagtgcccctggactgggggaggaaccctggaagcagagaacatgagggtagtcaggattctctccttcctctggaagaggctgtgaattctggaatggacactagtataccaagtatttggcctacattttgcagggagtcacaacccccacaggtggctcagccttcaggacctgggcaggcccaggctcctacccaagggggtaatacagacccactggaactctttctgtatcagctgctggatgaggtccaggtggaggaacatgccccagctccactcaactgggatgtggatccagggggcagagtccatgagggttcctgggagtcattctggccccaggaggaggcaggctctacaggactggacacaagctcccctagtgacagcaactcattctttagggagagtaagccctctcaggttgctcaaaggaggggagctgggcaagaggatgccaggactcaggctgacagtacaggacccctggagctgctgttgtttgaccagctcctggatgaagtgcagaaggaggaacatgttccagctcccctggactggggaaggaaccctggttctatggaacatgagggctctcaggactctctcttgcctctggaagaagctgctaatagtggcagagatacaagtatcccaagcatttggcctgccttttgcaggaaaagccagccaccccaggtagcccagcctagtggacctggacaggctcaggcacctatacaaggaggcaacactgacccattggagttgtttctggaccagctgctcactgaggtgcaactggaggaacaagggccagcacctgtcaatgttgaagagacctgggaacagatggataccactccagacttgccactgacttctgaagagtaccagacccttcttgacatgctgtaa

SEQ ID NO: 103 - protein, 674 aa, Artificial Sequence
  Synthetic Polypeptide
MAEAGSPVGGSGVARESGRRRRLVWTPSQSEALRACFERNPYPGIATRERLAQAIGIPEPRVQIWFQNERSRQLRQHHASKRSIRGSRRLASPQLQEELGSRPQGRGMRSSGRRPRTRLTSLQLRILGQAFERNPRPGFATREELARDTGLPEDTIHIWFQNRRARRRHRRGRPTAQDQDLLASQGSDGAPAGPEGREREGAQENLLPQEEAGSTGMDTSSPSDLPSFCGESQPFQVAQPRGAGQQEAPTRAGNAGSLEPLLDQLLDEVQVEEPAPAPLNLDGDPGGRVHEGSQESFWPQEEAGSTGMDTSSPSDSNSFCRESQPSQVAQPCGAGQEDARTQADSTGPLELLLLDQLLDEVQKEEHVPVPLDWGRNPGSREHEGSQDSLLPLEEAVNSGMDTSIPSIWPTFCRESQPPQVAQPSGPGQAQAPTQGGNTDPLELFLYQLLDEVQVEEHAPAPLNWDVDPGGRVHEGSWESFWPQEEAGSTGLDTSSPSDSNSFFRESKPSQVAQRRGAGQEDARTQADSTGPLELLLFDQLLDEVQKEEHVPAPLDWGRNPGSMEHEGSQDSLLPLEEAANSGRDTSIPSIWPAFCRKSQPPQVAQPSGPGQAQAPIQGGNTDPLELFLDQLLTEVQLEEQGPAPVNVEETWEQMDTTPDLPLTSEEYQTLLDML

SEQ ID NO: 104 - DNA, 1275 bp, Artificial Sequence
  Synthetic Nucleotide
atggcattgcctacaccttcagactctacgctgcctgcagaggctaggggaagaggtagacggcggcgattggtgtggactccatcacaatccgaagctcttcgcgcatgcttcgagcgcaatccctatccggggattgccacaagggagaggcttgcacaggctatcggaatcccggaaccgagagtgcagatctggttccaaaatgaacgctctcggcagctcagacagcatcgcagggagtcccgcccgtggccaggaagaaggggaccacctgaagggaggagacccagaaccaggctgacaagtctgcagctgagaatccttggtcaggcttttgaaaggaatccaaggccaggatttgccaccagagaggaactggccagggatacaggccttcctgaggatactatccatatctggttccagaacaggagggccaggagaaggcacagaaggggacgggcacctgctcaggccggtggactctgctctgctgcccctgggggcggccatccagcaccttcctgggtggctttcgctcatactggcgcttggggtaccgggctgcctgctccgcatgttccctgtgctccaggggccctcccgcagggagcgtttgtttcccaggcagctagggctgcacctgccctgcaaccatcacaggcagcgccagctgaaggcatcagccaacccgccccagcccgcggagattttgcttatgcagcgccagcacctccagacggtgccctgagccacccccaagcccccagatggccccctcaccctggtaagtcccgggaagaccgcgatccccaacgagatggactgcccggtccttgcgctgtggcccagccaggacctgctcaagccggccctcaggggcaaggagtgctggccccacctacaagccagggatctccctggtggggttggggacgcggacctcaggttgctggagccgcttgggagcctcaggccggagctgcaccgccgccacaaccggcccctcccgacgcgtcagcgtccgcccgacaaggccagatgcagggaatcccagcacctagccaagctcttcaagagcctgccccttggagcgcactgccgtgtgggctgctcctggatgaactcctggctagcccagaatttctccagcaggcacagccactcctggaaacagaagctccgggagagctcgaagcctccgaagaagcagcaagcctggaggcacctctttccgaggaggagtatagagcccttctggaagaactttga

SEQ ID NO: 105 - protein, 424 aa, Artificial Sequence
  Synthetic Polypeptide
MALPTPSDSTLPAEARGRGRRRRLVWTPSQSEALRACFERNPYPGIATRERLAQAIGIPEPRVQIWFQNERSRQLRQHRRESRPWPGRRGPPEGRRPRTRLTSLQLRILGQAFERNPRPGFATREELARDTGLPEDTIHIWFQNRRARRRHRRGRAPAQAGGLCSAAPGGGHPAPSWVAFAHTGAWGTGLPAPHVPCAPGALPQGAFVSQAARAAPALQPSQAAPAEGISQPAPARGDFAYAAPAPPDGALSHPQAPRWPPHPGKSREDRDPQRDGLPGPCAVAQPGPAQAGPQGQGVLAPPTSQGSPWWGWGRGPQVAGAAWEPQAGAAPPPQPAPPDASASARQGQMQGIPAPSQALQEPAPWSALPCGLLLDELLASPEFLQQAQPLLETEAPGELEASEEAASLEAPLSEEEYRALLEEL

SEQ ID NO: 106 - DNA, 1275 bp, Artificial Sequence
  Synthetic Nucleotide
atggcattgcctacaccttcagactctacgctgcctgcagaggctaggggaagaaggagaaggaggaaaactgtctggcaagcttggcaggaacaggcactcctgagcacatttaagaaaaaaaggtatctgtcctttaaagaaagaaaggaactggcaaaaaggatgggagtttctgattgcaggatcagagtctggttccagaataggagaaataggtctggggaggaaggacgcagggagtcccgcccgtggccaggaagaaggggaccacctgaaggaagaagaaaacgcacagcggtgactggcagccaaacggctctgctgctccgcgctttcgagaaagatcggttccccggaattgccgcacgcgaagaactcgccagagaaactgggctcccagaatcacgaatacagatttggttccagaaccgcagagcaagacacccaggccaggggggacgggcacctgctcaggccggtggactctgctctgctgcccctgggggcggccatccagcaccttcctgggtggctttcgctcatactggcgcttggggtaccgggctgcctgctccgcatgttccctgtgctccaggggccctcccgcagggagcgtttgtttcccaggcagctagggctgcacctgccctgcaaccatcacaggcagcgccagctgaaggcatcagccaacccgccccagcccgcggagattttgcttatgcagcgccagcacctccagacggtgccctgagccacccccaagcccccagatggccccctcaccctggtaagtcccgggaagaccgcgatccccaacgagatggactgcccggtccttgcgctgtggcccagccaggacctgctcaagccggccctcaggggcaaggagtgctggccccacctacaagccagggatctccctggtggggttggggacgcggacctcaggttgctggagccgcttgggagcctcaggccggagctgcaccgccgccacaaccggcccctcccgacgcgtcagcgtccgcccgacaaggccagatgcagggaatcccagcacctagccaagctcttcaagagcctgccccttggagcgcactgccgtgtgggctgctcctggatgaactcctggctagcccagaatttctccagcaggcacagccactcctggaaacagaagctccgggagagctcgaagcctccgaagaagcagcaagcctggaggcacctctttccgaggaggagtatagagcccttctggaagaactttga

SEQ ID NO: 107 - protein, 424 aa, Artificial Sequence
  Synthetic Polypeptide
MALPTPSDSTLPAEARGRRRRRKTVWQAWQEQALLSTFKKKRYLSFKERKELAKRMGVSDCRIRVWFQNRRNRSGEEGRRESRPWPGRRGPPEGRRKRTAVTGSQTALLLRAFEKDRFPGIAAREELARETGLPESRIQIWFQNRRARHPGQGGRAPAQAGGLCSAAPGGGHPAPSWVAFAHTGAWGTGLPAPHVPCAPGALPQGAFVSQAARAAPALQPSQAAPAEGISQPAPARGDFAYAAPAPPDGALSHPQAPRWPPHPGKSREDRDPQRDGLPGPCAVAQPGPAQAGPQGQGVLAPPTSQGSPWWGWGRGPQVAGAAWEPQAGAAPPPQPAPPDASASARQGQMQGIPAPSQALQEPAPWSALPCGLLLDELLASPEFLQQAQPLLETEAPGELEASEEAASLEAPLSEEEYRALLEEL

SEQ ID NO: 108 - DNA, 2240 bp, Bos taurus
accatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaccggaagcggtgcaagcagcggatcatccagtaccagccggggtcctattgcaacgggctcaaggcggcggcgcttggttctgaaacctagccaaaaagatgctcttcaagccttgtttcaacagaatccatacccaggcatagcgacccgcgagagattggctcgagagttgggtatcgacgagagtagggtgcaagtctggtttcaaaatcagcgacggaggagaagtaagcagagtaggccgccttctgaacatgtgcgacaggagggagagggggggcctacttctacacctcgccccccaagcccgcctccgaggccacagagtagcagtcaaggcaaactcgcgagcgtcctctcaaaagggaaggaggcacggaggaaacgaaccgtgatctcaccgagccaaacacgcatactcgtgcaagctttcacgagggacagattcccggggatagccgctcgcgaggaattggcacgacaaaccggcattccggaaccgcgcatacagatttggtttcaaaatagacgagcccgccacccgcaacgctccccgtctggtcctgggaatggcagagcacaggggcccggtggtgctcccgcaacgaccactacaccagcccccgaagatcgccgagctccacccgctgttcaatcaacaagtcctccgcttaggccatcccaaccacaggaaagcatgccccctcttgcagcagcggccccttttggagcacctaccttctgggtgctaggagctgcaagcggagtctgtgtgggtcaacccttgatgatctttgtggtgcagcccagcccagccgcgttgcaaccgtctgggaggcctcctcctcctcctcagggtgccgcaccatgggcggcttgcagccccgcggtgacagcgcccggtctgccaggtcagggcgcgattctgccaccgggacaaccggagactcacatcccccgatggccggaatccccctccggtgaagggaccgcaccccctctcgagccccaaccacaagccccgagtctccccagctccacttctcttctcgatgagctcctggccgctactggggttcccgacacccaagcgcccagtcctggggcagctgcggatgaaggtgttgggcccgctctcccgggagctccgagcttcttggatgagctgctggctgcaacgggaacgccggatactcccgggccgtcccttgggccaagcgcagacgagcgagcccacctggcgctccccggcgaattgctcgcggccgctggacttcctggttcacccggcccaagtcctggctcatctcctgtcgtcgctggccctcaccctgcgctgcctggtcccccatctcttctggaagagatacttgctgcaaccgctatacaggacacaccatggagcagcccgggaagtcccgccggggaagaaggtgttgaagcgaccttggaaactccattgagtgaagatgaataccaagctctgctcgacatgctgcccggctctccagggcccggtgcg

SEQ ID NO: 109 - protein, 507 aa, Bos taurus
MSGASSGSSSTSRGPIATGSRRRRLVLKPSQKDALQALFQQNPYPGIATRERLARELGIDESRVQVWFQNQRRRRSKQSRPPSEHVRQEGEGGPTSTPRPPSPPPRPQSSSQGKLASVLSKGKEARRKRTVISPSQTRILVQAFTRDRFPGIAAREELARQTGIPEPRIQIWFQNRRARHPQRSPSGPGNGRAQGPGGAPATTTTPAPEDRRAPPAVQSTSPPLRPSQPQESMPPLAAAAPFGAPTFWVLGAASGVCVGQPLMIFVVQPSPAALQPSGRPPPPPQGAAPWAACSPAVTAPGLPGQGAILPPGQPETHIPRWPESPSGEGTAPPLEPQPQAPSLPSSTSLLDELLAATGVPDTQAPSPGAAADEGVGPALPGAPSFLDELLAATGTPDTPGPSLGPSADERAHLALPGELLAAAGLPGSPGPSPGSSPVVAGPHPALPGPPSLLEEILAATAIQDTPWSSPGSPAGEEGVEATLETPLSEDEYQALLDMLPGSPGPGA

SEQ ID NO: 110 - protein, 60 aa, Bos taurus; identical to SEQ ID NO: 25

SEQ ID NO: 111 - protein, 60 aa, Bos taurus; identical to SEQ ID NO: 36

SEQ ID NO: 112 - protein, 57 aa, Bos taurus; identical to SEQ ID NO: 43

SEQ ID NO: 113 - DNA, 1434 bp, Equus caballus
atggcctgtgcggagacggtcctgggcgctgtcaagaggccctggctgtcgtgcccgcagacggcggctgccgctcagggaaaccacctgcagacgaggcgtcctggtggcagcggtggaggcgtggcagctggcccgcatcagagaggatcccgacgcaggaggattgttttgaaggcgagtcagagggacgctctgcgagcagcgtttcaacagaacccttaccctgggatcgccaccagagaacgcctggcccaagagattgacattccggaatgcagagtccaggtttggtttcaaaaccaacgcagaagacatctaaggcagagccggtcgggctcggcgagctccgtgggagaagggcaatcgcctggagaggagcagccccaagctcgggccgcagaaggcggaagaaagcggacacacatcactccgtggcaaaccgggatcctccttgagagcttccagaaggaccgatttcctggcatcgctaccagggaagaactggccagacaaacgggcatcccagaggcgagaattcaggtgtggtttcagaaccgaagagctcggcacccagaccagagtggaagcggcccggtgaatgccttggcggaaggccccagtcccagggctcccctgactgccctccaggaccaagccaacctgtcctctgtccccagcagctctccgcatctgcctccctggaaccctcctgggctcttgccatcgcccgcgacagccgctcctccactctgcccggtgttcttcgttccttgggttccctctggggcctgtgtgggccggccaccggagcccctggtggtcatgacagcccagcctgtgctgggaaaggagaacgttcaccctccttggacacttctgtgtccctgctcaaccgggccgcctctggcaggcggtctctcagcgatgcagcctcctctccggcccacgcccggaggaaaatgccaggagcacgacgggcacgctggcgggagggggctgcccttcccacactcccctcagcctcaccctgaccgtcctcagcaacagtggcagcacctgggtgggccaggagccttccccgctatgcagccttggggcgagtggcctcaggtcctcccggccccagaggagcctcagggaagggcggttcagcagtctgcgcaccctgacacacacgtgtggccatgggaggagccatcagccggagagccctctgctcagccgggcccacagcagcagcactctgcgcaaacccccagcctcctagatgagctgctcgcagtcacagagctgcaggaaaaggcacagccgttcctgaacgggcatccgccggcagaggagcctccgggaacactggaaggtcccctcagcgaggaggaatttcaggctctgctcgacatgctgcaaagctcaccagggcctcagatttag

SEQ ID NO: 114 - protein, 477 aa, Equus caballus
MACAETVLGAVKRPWLSCPQTAAAAQGNHLQTRRPGGSGGGVAAGPHQRGSRRRRIVLKASQRDALRAAFQQNPYPGIATRERLAQEIDIPECRVQVWFQNQRRRHLRQSRSGSASSVGEGQSPGEEQPQARAAEGGRKRTHITPWQTGILLESFQKDRFPGIATREELARQTGIPEARIQVWFQNRRARHPDQSGSGPVNALAEGPSPRAPLTALQDQANLSSVPSSSPHLPPWNPPGLLPSPATAAPPLCPVFFVPWVPSGACVGRPPEPLVVMTAQPVLGKENVHPPWTLLCPCSTGPPLAGGLSAMQPPLRPTPGGKCQEHDGHAGGRGLPFPHSPQPHPDRPQQQWQHLGGPGAFPAMQPWGEWPQVLPAPEEPQGRAVQQSAHPDTHVWPWEEPSAGEPSAQPGPQQQHSAQTPSLLDELLAVTELQEKAQPFLNGHPPAEEPPGTLEGPLSEEEFQALLDMLQSSPGPQI

SEQ ID NO: 115 - protein, 60 aa, Equus caballus; identical to SEQ ID NO: 26

SEQ ID NO: 116 - protein, 60 aa, Equus caballus; identical to SEQ ID NO: 37

SEQ ID NO: 117 - protein, 57 aa, Equus caballus; identical to SEQ ID NO: 47

SEQ ID NO: 118 - DNA, 1437 bp, Sus scrofa
atgcccctcaagttggcagtgttggctctttgcttggcctcatgccagcaatcatttttcctaatgggctcactttctagaggatcacggagaaggaggcttgttctgaaacagagtcagcgggatgctctgcaagcagtctttcaagagaagccctaccctggtataacgaccagagaacgactggccagagaacttagcatcccagaaagccgaattcagatgtggttccaaaaccaaagaaaacgacgtctcaagcagcagagcagagggccacctgagactatcccccaaccagggccaccacagcgggagcaacagcttcagacttctcccactcctgcaatcccaaaagaggctgggagaaagcggtcattcatctctccctcacaaacagacatccttcggcaagcctttgagcgggaacgatacccaggcattgccgccagggaagaactggcacgtcaaacagggattccagaacctcagattctggtgtggtttcagaaccgacgagctcggcacccagagcagaagggaagtgggtctgccaatgtgcccggagtagaccccaattctgcaaaaggcctaccacttccatcggaccagggcatgccaaccactgcccacagcagccctactcacagtgctcctcctcctccctctaacccaccaagggagaacatgctgtccatcacccccatggtggccactgctgcgatcgcccccaaattcatagttcctggggctcccacagcaggctgtgagggccagagcctgcccatgatcttcatcatggcccagccaagtccagttctgcaggcaatagtgaaccctcccatgctttggacgcttcctctgactcagtcctcaccagggccaatgcccattcctgcagggggtctcacacctattcacacagggctctggccaacatcccaagaaggaccatggcaggagaacaatctgcacactatgccagcagaaaaatgcctcccacacatccctcagccaccccttgccagtcgtgcagagcccctgccactgctggacccagtgaagacctgcacttatgccaggccagaatgggcccaggcatcctcagctcaagtcaccagtgggaagcctgtgcatggggccatgctgcagcctgcacaggctgacacacttatctgcccctctcatctggccccctcaaatgaagagctgtgccctcccattgacctgcagcagaacaagccctcagccttccagggctcatcaaacctccttgaggaaattatggcagctgcaggcattctgcctgaggcagggcctcttccagacgtggaggaacaggaagagcttcccctaggagacctggaagcacccctcagtgaggaagatttccaggccctcctcgacatgctgccaagctccccaggtccttgtccttag

SEQ ID NO: 119 - protein, 478 aa, Sus scrofa
MPLKLAVLALCLASCQQSFFLMGSLSRGSRRRRLVLKQSQRDALQAVFQEKPYPGITTRERLARELSIPESRIQMWFQNQRKRRLKQQSRGPPETIPQPGPPQREQQLQTSPTPAIPKEAGRKRSFISPSQTDILRQAFERERYPGIAAREELARQTGIPEPQILVWFQNRRARHPEQKGSGSANVPGVDPNSAKGLPLPSDQGMPTTAHSSPTHSAPPPPSNPPRENMLSITPMVATAAIAPKFIVPGAPTAGCEGQSLPMIFIMAQPSPVLQAIVNPPMLWTLPLTQSSPGPMPIPAGGLTPIHTGLWPTSQEGPWQENNLHTMPAEKCLPHIPQPPLASRAEPLPLLDPVKTCTYARPEWAQASSAQVTSGKPVHGAMLQPAQADTLICPSHLAPSNEELCPPIDLQQNKPSAFQGSSNLLEEIMAAAGILPEAGPLPDVEEQEELPLGDLEAPLSEEDFQALLDMLPSSPGPCP

SEQ ID NO: 120 - protein, 60 aa, Sus scrofa; identical to SEQ ID NO: 23

SEQ ID NO: 121 - protein, 60 aa, Sus scrofa; identical to SEQ ID NO: 34

SEQ ID NO: 122 - protein, 57 aa, Sus scrofa; identical to SEQ ID NO: 51

SEQ ID NO: 123 - DNA, 1299 bp, Loxodonta africana
atggatccgaccggcgcttcgagtcgctctcaaaatccacgaggccgacgagagaggttggttttgaagcccagtcaaagagagaccctgcaagcagcgtttgaacagaacccctaccctggtataactaccagagaagaactcgccagagaaaccggcatcgcggaggatcgcattcagacttggtttggaaaccgcagagcaggtcacctaaggaagagccgctcggcctctggacaggcctccgaagaagagccgtcccagggacagggagagcctcagccttggtctccggaaaatttccccaaagcggccagacgaaaacgcacacgcatcaccacatcgcaaacgagtctcctagtcgaggccttcgagaagaaccggtaccctggtaacgaggccaaggaagaactggctcaacgaactggccttccgcgatcccgaattcacgtatggtttcagaaccgaagagctcggaagccggtgcagagcgcgagtgcaccgccgaagtccttggcagacagcccgactcctgcggccacgcttccactcgaccaaagcgacctgtcctctgtacagagcacctaccctctcggcccaccctcccatccttctagcagcaaccaagccatcctacctgttctcactgagtcccgtacaccatttcttccttcggaacccacccagggctgtgccggccaagcaccgggtgccgtgttggaccagcccgccctgattgtgaagaagacagcagagacctctcacgcgccggggacacacctgaaccaatcgccaacaggacccactgtgggagacaggctgtcagaccctcaggctcctttctggccccaatacccaggaaattaccaggatcgcgaccaacatgctgtctcggcagggtggctcgcccaagacccttctcggcctgacaattcaaagacgcaagggcaggttccggctcagcaagtcacagctcccttcacgcaatggggctgtgaggtggcccagggtgtgaccgcccgatgggaacccagccaagagacactccagcagcccggacactccgaggcacacctgtggccagagccggcacaatcggctcaagagtcatctcatccaccagaccaagactgccaggaaaccgagagccttttagatgaactcctctccgccccagagttgcagggaaagtcccaaacctttctgaacgcggatccacaggaggaggaccctccacaactcgaactctccctcggcgacattgactttcaggctctgcttgacgcgctgcaagattga

SEQ ID NO: 124 - protein, 432 aa, Loxodonta africana
MDPTGASSRSQNPRGRRERLVLKPSQRETLQAAFEQNPYPGITTREELARETGIAEDRIQTWFGNRRAGHLRKSRSASGQASEEEPSQGQGEPQPWSPENFPKAARRKRTRITTSQTSLLVEAFEKNRYPGNEAKEELAQRTGLPRSRIHVWFQNRRARKPVQSASAPPKSLADSPTPAATLPLDQSDLSSVQSTYPLGPPSHPSSSNQAILPVLTESRTPFLPSEPTQGCAGQAPGAVLDQPALIVKKTAETSHAPGTHLNQSPTGPTVGDRLSDPQAPFWPQYPGNYQDRDQHAVSAGWLAQDPSRPDNSKTQGQVPAQQVTAPFTQWGCEVAQGVTARWEPSQETLQQPGHSEAHLWPEPAQSAQESSHPPDQDCQETESLLDELLSAPELQGKSQTFLNADPQEEDPPQLELSLGDIDFQALLDALQD

SEQ ID NO: 125 - protein, 60 aa, Loxodonta africana; identical to SEQ ID NO: 29

SEQ ID NO: 126 - protein, 60 aa, Loxodonta africana; identical to SEQ ID NO: 40

SEQ ID NO: 127 - protein, 50 aa, Loxodonta africana
SLLDELLSAPELQGKSQTFLNADPQEEDPPQLELSLGDIDFQALLDALQD

SEQ ID NO: 128 - DNA, 1296 bp, Unknown
  Folivora
atgcggatgacccgaatcgccatctccctggtgtccgctgatgacagccttccaagtaccctgaaaggagtggcccgaagaaagaggatctttttgaacccaactcaaattgatgtcctgcaagcatcgtttcaaaagaacccctaccctggtatagcttccagggaacaactggctaatgaaattggtgttccagagtctcgaattcaggtttggtttcagaaccggagagtaagacgccaaaagcagcatcaaccgcagtctggatcctgctcagaagattgtttacccaaagaagcccgtcgtaagcgcacatccatcaccagatcccaaaccatcattctggttgaggcctttgagcagaaccgattccctggtgttacaaccagagaagaacttgctaaacaaacaggccttccagaagatagaattcagatatggtttcagaatcggagaaatcggtacccagggaagacaccaagcggacacagaaattccgcggcaggtgccccaaatcggaggcctcatctgaccattgggcaggagaaaactcacctgatcactgtcccaagaaggccccatcatcttgcttcctgcaatattttccacgagacatgcataattccctccactattcttttgtgcctcacaacctctgctcttaaggattcaaatgtgaactgcatgagtcaggcaccccatttcctggaggcccagcccacactgactgcacaggcaggggcaaacgcttaccccacacagactattatcagtcactgcccagcagagcaacctctgggaatggggttctcagataagccaaataatttcaagctccctttccagggaaaatgccaggatcaagatgaatccactggaaggggagtggtgcagttgaaagacaatcccctgacacaaactgacaatgaaaaacaacaattacatgatgttggtcgggcagacacatctcacaacatgcagtggtgcagcgaggagttgcaaagtgtgaatgcagaaggagaaactcctgaagggaaacttcatcagcctagacactctgagatgcagccagggcagcagcaggcagaatcagctgaagagccatcacttccccctgcccaggagcaccagcaagatctggagtcctggagccttctggaccaactgctgtcgagcaaagaatttctggaaaaggcccaacctcttctcaatccagattcccaggaccagaattctctaccagttgaaccatccctcagtgaggaagagtttcaggctctgcttgacatgctgtga

SEQ ID NO: 129 - protein, 431 aa, Unknown
  Folivora
MRMTRIAISLVSADDSLPSTLKGVARRKRIFLNPTQIDVLQASFQKNPYPGIASREQLANEIGVPESRIQVWFQNRRVRRQKQHQPQSGSCSEDCLPKEARRKRTSITRSQTIILVEAFEQNRFPGVTTREELAKQTGLPEDRIQIWFQNRRNRYPGKTPSGHRNSAAGAPNRRPHLTIGQEKTHLITVPRRPHHLASCNIFHETCIIPSTILLCLTTSALKDSNVNCMSQAPHFLEAQPTLTAQAGANAYPTQTIISHCPAEQPLGMGFSDKPNNFKLPFQGKCQDQDESTGRGVVQLKDNPLTQTDNEKQQLHDVGRADTSHNMQWCSEELQSVNAEGETPEGKLHQPRHSEMQPGQQQAESAEEPSLPPAQEHQQDLESWSLLDQLLSSKEFLEKAQPLLNPDSQDQNSLPVEPSLSEEEFQALLDML

SEQ ID NO: 130 - protein, 60 aa, Unknown; identical to SEQ ID NO: 30
  Folivora

SEQ ID NO: 131 - protein, 60 aa, Unknown; identical to SEQ ID NO: 41
  Folivora

SEQ ID NO: 132 - protein, 48 aa, Unknown; identical to SEQ ID NO: 52
  Folivora

SEQ ID NO: 133 - DNA, 23 bp, Artificial Sequence
  Synthetic Nucleotide
aactcctcctccttgatcaactg

SEQ ID NO: 134 - DNA, 21 bp, Artificial Sequence
  Synthetic Nucleotide
cttctctctgtggccaaaagc

SEQ ID NO: 135 - DNA, 21 bp, Artificial Sequence
  Synthetic Nucleotide
cttctgcagagagtcccagac

SEQ ID NO: 136 - DNA, 20 bp, Artificial Sequence
  Synthetic Nucleotide
ggcagatcaggtgttgtgtc

SEQ ID NO: 137 - protein, 11 aa, Artificial Sequence
  Synthetic Polypeptide
WGATTYAATCW